(19) United States
(12) Patent Application Publication
(10) Pub. No.: US 2024/0156920 A1
(43) Pub. Date: May 16, 2024

(54) COMPOSITIONS COMPRISING HYALURONIDASE AND/OR COLLAGENASE AND/OR 4-METHYLUMBELLIFERONE (4-MU) AND METHODS OF TREATMENT USING SAME

(71) Applicant: STANDARD OF CARE CORPORATION, Chicago, IL (US)

(72) Inventors: Iliana E. SWEIS, Chicago, IL (US); Bryan C. CRESSEY, Chicago, IL (US)

(21) Appl. No.: 17/769,268

(22) PCT Filed: Oct. 15, 2020
(86) PCT No.: PCT/US20/55830

§ 371 Date: Apr. 14, 2022

Related U.S. Application Data
(60) Provisional application No. 62/915,437, filed on Oct. 15, 2019.
(60) Provisional application No. 62/940,746, filed on Nov. 26, 2019.
(60) Provisional application No. 62/966,770, filed on Jan. 28, 2020.
(60) Provisional application No. 62/966,773, filed on Jan. 28, 2020.
(60) Provisional application No. 62/966,804, filed on Jan. 28, 2020.
(60) Provisional application No. 62/967,781, filed on Jan. 30, 2020.
(60) Provisional application No. 63/032,119, filed on May 29, 2020.
(60) Provisional application No. 63/032,125, filed on May 29, 2020.
(60) Provisional application No. 63/055,838, filed on Jul. 23, 2020.

(51) Int. Cl. A61K 38/47; A61P 7/10

(52) U.S. Cl. CPC A61K 38/47; A61P 7/10; C12Y 302/01035

(57) ABSTRACT

The present disclosure provides methods of treating various conditions, diseases, and/or disorders in a subject in need thereof by administering a composition that comprises a protein having hyaluronidase, collagenase, and/or 4-methylumbelliferone (4-MU) activity, with or without one or more additional active pharmaceutical ingredients (APIs). The various conditions, diseases, and/or disorders comprise edema, inflammation, eyelid puffiness/fullness, fibrosis, and remyelinating axons. Also provided are methods of assessing peri-orbital puffiness in a subject.

### CROSS REFERENCE TO RELATED APPLICATIONS

The present application is the U.S. national stage of International Patent Application No. PCT/US2020/055830 filed Oct. 12, 2020, which claims priority to U.S. Provisional Patent Application Ser. No. 62/915,437, filed Oct. 15, 2019; U.S. Provisional Patent Application Ser. No. 62/940,746, filed Nov. 26, 2019; U.S. Provisional Patent Application Ser. No. 62/966,770, filed Jan. 28, 2020; U.S. Provisional Patent Application Ser. No. 62/966,773, filed Jan. 28, 2020; U.S. Provisional Patent Application Ser. No. 62/966,804, filed Jan. 28, 2020; U.S. Provisional Patent Application Ser. No. 62/967,781, filed Jan. 30, 2020; U.S. Provisional Patent Application Ser. No. 63/032,119, filed May 29, 2020; U.S. Provisional Patent Application Ser. No. 63/032,125, filed May 29, 2020; and, U.S. Provisional Patent Application Ser. No. 63/055,838, filed Jul. 23, 2020; all of which are incorporated herein by reference in their entirety.

### FIELD

The present disclosure generally relates to compositions comprising a protein having hyaluronidase, collagenase, and/or 4-methylumbelliferone (4-MU) activity and methods of use thereof for the treatment of numerous conditions and/or diseases and/or disorders.

### BACKGROUND

Hyaluronidase is an enzyme that modifies the permeability of connective tissue through the hydrolysis of hyaluronic acid, a polysaccharide found in the intercellular ground substance of connective tissue. It hydrolyzes hyaluronic acid by splitting the glucosaminidic bond between C1 of an N-acetylglucosamine moiety and C4 of a glucuronic acid moiety.

Hyaluronic acid (HA) is an extracellular matrix glycosaminoglycan. It has many roles in normal tissue function and development, including providing support and anchorage for cells, facilitating cell-cell signaling, and facilitating cell movement and migration. HA in circulation is rapidly degraded while HA bound to proteins and incorporated into tissues such as joints, basement membranes, and the vitreous of the eye is longer lived.

HA levels are greatly elevated in injured tissues, with production increasing by as much as 80-fold. Because HA is highly hygroscopic, this increased HA production is likely to drive inflammation at sites of injury. Consistent with this, HA has been implicated in vascular permeability changes, leukocyte adhesion and egress, and migration. Additionally, many chronic disease processes associated with unremitting inflammation are associated with prolonged increases in HA, including type 2 diabetes, liver cirrhosis, asthma, and cancer.

In light of these contributions of HA to inflammation, autoimmunity, and to tumor growth/metastasis, there has been a great interest in identifying pharmacologic drugs to inhibit HA synthesis. One such agent is 4-methylumbelliferone (4-MU). However, such drugs are often associated with a number of side effects. As such, there exists a need for alternatives to address the treatment of inflammation, autoimmunity, and tumor growth/metastasis due to elevated levels of HA.

HYLENEX is a recombinant preparation of human hyaluronidase. It is produced by genetically engineered Chinese Hamster Ovary (CHO) cells containing a DNA plasm id encoding for a soluble fragment of human hyaluronidase (PH20). The purified hyaluronidase glycoprotein contains 447 amino acids with an approximate molecular weight of 61,000 Daltons. Presently, hyaluronidase is only indicated as an adjuvant in subcutaneous fluid administration for achieving hydration, as an adjuvant to increase the dispersion and absorption of other injected drugs, and as an adjunct in subcutaneous urography for improving resorption of radiopaque agents.

### SUMMARY

The present disclosure provides a method of treating or preventing a condition such as a cosmetic condition in a subject in need thereof by administering a composition that comprises a protein having hyaluronidase activity (e.g., a hyaluronidase such as Hylenex, Amphadase, or Vitrase) to the peri-orbital region and/or mid-face of the subject. The cosmetic condition may be due to edema (e.g., peri-orbital puffiness due to peri-orbital edema).

The present disclosure provides a method of treating peri-orbital puffiness due to peri-orbital edema in a subject in need thereof by administering a composition that comprises a protein having hyaluronidase activity to the peri-orbital region of the subject.

In some embodiments of each or any of the above or below mentioned embodiments, the step of administering is performed by one or more injections.

In some embodiments of each or any of the above or below mentioned embodiments, the step of administering is performed by one or more injections to the peri-orbital region.

In some embodiments of each or any of the above or below mentioned embodiments, the step of administering is performed by one or more injections into a peri-orbital soft tissue and/or one or more injections into one or more peri-orbital fat pads.

The present disclosure provides a method of treating cellulite in a subject in need thereof, the method comprising administering a composition that comprises a protein having hyaluronidase activity to a region of skin having cellulite on the subject.

The present disclosure provides a method of reducing the appearance of cellulite in a subject in need thereof, the method comprising administering a composition that comprises a protein having hyaluronidase activity to a region of skin having cellulite on the subject, wherein the administration results in a reduction in a grade of severity of the cellulite.

In some embodiments of each or any of the above or below mentioned embodiments, the region of skin is located on the back of a leg, a buttock, an arm, a thigh, and/or an abdomen.

In some embodiments of each or any of the above or below mentioned embodiments, the region of skin is located on the back of a leg or a buttock.

In some embodiments of each or any of the above or below mentioned embodiments, the method further comprises tightening the region of skin having cellulite after administration of the composition.

In some embodiments of each or any of the above or below mentioned embodiments, the skin is tightened by treatment with a radiofrequency.

In some embodiments of each or any of the above or below mentioned embodiments, the skin is tightened by treatment with ultrasound.

In some embodiments of each or any of the above or below mentioned embodiments, the skin is tightened by high intensity focused electromagnetic technology.

In some embodiments of each or any of the above or below mentioned embodiments, the step of administering the composition is performed by one or more injections to the region of skin having cellulite.

In some embodiments of each or any of the above or below mentioned embodiments, the step of administering the composition comprises applying a patch or a cream to the region of skin having cellulite.

In some embodiments of each or any of the above or below mentioned embodiments, the method further comprises administering retinol cream to the region of skin having cellulite.

In some embodiments of each or any of the above or below mentioned embodiments, the method further comprises treating the region of skin having cellulite with acoustic wave therapy, laser treatment, ultrasonic liposculpting, laser-assisted liposuction, and/or radiotherapy.

In some embodiments of each or any of the above or below mentioned embodiments, the administration of the composition reduces the appearance of the cellulite.

In some embodiments of each or any of the above or below mentioned embodiments, the administration reduces a number of depressions, depth of depressions, clinical appearance of evident raised lesions, presence of flaccidity, and/or grade of the cellulite.

In some embodiments of each or any of the above or below mentioned embodiments, the cellulite prior to treatment with the composition is classified as mild, moderate, or severe cellulite.

In some embodiments of each or any of the above or below mentioned embodiments, the cellulite after treatment with the composition is classified as mild or moderate cellulite.

In some embodiments of each or any of the above or below mentioned embodiments, the severity grade is based on the Cellulite Severity Scale (CSS).

In some embodiments of each or any of the above or below mentioned embodiments, the method further comprises administering a retinol cream.

In some embodiments of each or any of the above or below mentioned embodiments, treating the area of skin having cellulite with an acoustic wave therapy, laser treatment, ultrasonic liposculpting, laser-assisted liposuction, and/or radiotherapy.

In some embodiments of each or any of the above or below mentioned embodiments, the region of skin in the subject in need thereof is located on the back of a leg, a buttock, an arm, a thigh, and/or an abdomen.

The present disclosure provides a method of assessing peri-orbital fullness in a subject, the method comprising: a. determining if the subject exhibits pseudoherniation of one or more upper eyelid fat pads and/or one or more lower eyelid fat pads; b. determining if the subject exhibits edema in one or more of the upper eyelid fat pads and/or the one or more of the lower eyelid fat pads; c. determining if the subject exhibits edema in the peri-orbital tissue; and d. scoring the peri-orbital fullness from the determinations of steps a. to c.

In some embodiments of each or any of the above or below mentioned embodiments, the peri-orbital fullness is due to peri-orbital edema.

In some embodiments of each or any of the above or below mentioned embodiments, the peri-orbital fullness is unrelated to the use of a hyaluronic acid filler in the peri-orbital region.

In some embodiments of each or any of the above or below mentioned embodiments, the lower eyelid fat pad is a lower lateral fat pad, a lower middle fat pad, or a lower medial fat pad.

In some embodiments of each or any of the above or below mentioned embodiments, the lower eyelid fat pad is selected from one or more of a lower lateral fat pad, a lower middle fat pad, or a lower medial fat pad.

In some embodiments of each or any of the above or below mentioned embodiments, the upper eyelid fat pad is an upper middle fat pad or an upper medial fat pad.

In some embodiments of each or any of the above or below mentioned embodiments, the subject is scored as Grade 0 where the subject does not exhibit pseudoherniation of any of the upper or lower eyelid fat pads.

In some embodiments of each or any of the above or below mentioned embodiments, the subject exhibits edema of the one or more upper and/or lower eyelid fat pads, edema along the soft tissue of the upper and/or lower eyelids, presence of malar mounds due to edema, presence of malar edema, presence of mild festoons, or any combination thereof.

In some embodiments of each or any of the above or below mentioned embodiments, the subject is scored as Grade 1 where the subject exhibits pseudoherniation of one or more of the upper and/or lower eyelid fat pads without any peri-orbital malar or eyelid edema.

In some embodiments of each or any of the above or below mentioned embodiments, the subject is scored as Grade 1E where the subject exhibits pseudoherniation of one or more of the upper and/or lower eyelid fat pads, and the subject exhibits edema of the one or more upper and/or lower fat pads, presence of edema along the soft tissue of the upper or lower eyelids, presence of malar mounds due to edema, presence of malar edema, presence of mild festoons, or any combination thereof.

In some embodiments of each or any of the above or below mentioned embodiments, the subject is scored as Grade 2 where the subject exhibits pseudoherniation of two of the upper and/or lower eyelid fat pads without peri-orbital, malar or eyelid edema.

In some embodiments of each or any of the above or below mentioned embodiments, the subject is scored as Grade 2E where the subject exhibits pseudoherniation of two of the upper and/or lower eyelid fat pads, and the subject exhibits edema of the one or more upper and/or lower fat pads, presence of edema along the soft tissue of the upper or lower eyelids, presence of malar mounds due to edema, presence of malar edema, presence of mild festoons, or any combination thereof.

In some embodiments of each or any of the above or below mentioned embodiments, the subject is scored as Grade 3 where the subject exhibits pseudoherniation of three lower eyelid fat pads without any associated peri-orbital, malar or eyelid edema.

In some embodiments of each or any of the above or below mentioned embodiments, the subject is scored as Grade 3E where the subject exhibits pseudoherniation of three lower eyelid fat pads, and the subject exhibits edema of the fat pads, presence of edema along the soft tissue of the upper and/or lower eyelids, presence of malar mounds due to edema, presence of malar edema, presence of mild festoons, or any combination thereof.

In some embodiments of each or any of the above or below mentioned embodiments, the subject is scored as Grade 3+ where the subject exhibits pseudoherniation of the three lower eyelid fat pads with moderate to severe festoons and/or malar mounds secondary to fat deposition or hypertrophy of the orbicularis oculi muscle, without any peri-orbital, malar or eyelid edema.

In some embodiments of each or any of the above or below mentioned embodiments, the subject is scored as 3E+ where the subject exhibits pseudoherniation of the three lower eyelid fat pads with moderate to severe festoons and/or malar mounds secondary to fat deposition or descent/hypertrophy of the orbicularis oculi muscle, and the subject exhibits edema of the fat pad, presence of edema along the soft tissue of the upper or lower eyelids, presence of malar mounds due to edema, presence of malar edema, presence of mild festoons, or any combination thereof.

In some embodiments of each or any of the above or below mentioned embodiments, the subject scored as Grade 0 is administered a composition that comprises a protein having hyaluronidase activity to the peri-orbital region of the subject.

In some embodiments of each or any of the above or below mentioned embodiments, the subject scored as Grade 1, Grade 2 or Grade 3 is treated by surgical removal of a portion of one or more of the upper and/or lower fat pads.

In some embodiments of each or any of the above or below mentioned embodiments, the subject scored as Grade 1E, 2E or 3E is administered a composition that comprises a protein having hyaluronidase activity to the peri-orbital region of the subject, and optionally treated by surgical removal of a portion of one or more of the upper and/or lower fat pads.

The present disclosure provides a method of determining if peri-orbital fullness is due to edema or a structural change in a subject, the method comprising: a. administering a composition that comprises a protein having hyaluronidase activity to the peri-orbital region of the subject; b. assessing the peri-orbital region at a predetermined amount of time after the administration of the composition; and c. determining whether there is an improvement in the peri-orbital fullness.

In some embodiments of each or any of the above or below mentioned embodiments, the structural change is a herniation.

In some embodiments of each or any of the above or below mentioned embodiments, the predetermined time is 5 minutes, 15 minutes, 30 minutes, 1 hour, 24 hours, or 1 week after administration of the composition.

In some embodiments of each or any of the above or below mentioned embodiments, no improvement in peri-orbital puffiness indicates that the peri-orbital puffiness is due to a structural change.

In some embodiments of each or any of the above or below mentioned embodiments, a partial improvement in peri-orbital puffiness indicates that the peri-orbital puffiness is secondary to both edema and a structural change.

In some embodiments of each or any of the above or below mentioned embodiments, improvement in peri-orbital puffiness indicates that the peri-orbital puffiness is secondary to edema.

In some embodiments of each or any of the above or below mentioned embodiments, an improvement includes a reduction in the peri-orbital puffiness.

The present disclosure provides a method for treating a subject with peri-orbital fullness, the method comprising: a. determining if peri-orbital fullness is due to edema or a structural change in a subject by: i. administering a composition that comprises a protein having hyaluronidase activity to the peri-orbital region of the subject; ii. assessing the peri-orbital region at a predetermined amount of time after the administration of the composition; and iii. determining whether there is an improvement in the peri-orbital fullness; and b. surgically resecting a portion of one or more of an upper and/or lower eyelid fat pads where there is no improvement in peri-orbital fullness after administration of the composition.

The present disclosure provides a method for minimizing peri-orbital hollowness from surgical resection of one or more peri-orbital fat pads in a subject, the method comprising: a. administering a composition that comprises a protein having hyaluronidase activity to the peri-orbital region of the subject; b. assessing the peri-orbital region at a predetermined amount of time after administration of the composition; and c. determining an amount of fat to surgically resect from the one or more eyelid fat pads.

In some embodiments of each or any of the above or below mentioned embodiments, the method further comprises surgically resecting an amount of the one or more peri-orbital fat fads.

In some embodiments of each or any of the above or below mentioned embodiments, the one or more peri-orbital fat pads include the upper eyelid fat pads and the lower eyelid fat pads.

In some embodiments of each or any of the above or below mentioned embodiments, the amount of fat to surgically resect from the one or more fat pads is determined by a visual examination of the one or more fat pads after administration of the composition.

In some embodiments of each or any of the above or below mentioned embodiments, the step of administering the composition is performed by one or more injections to the region of the subject having edema.

The present disclosure provides a method for determining an amount of fat to be resected surgically from one or more per-orbital fat pads, the method comprising: administering a composition that comprises a protein having hyaluronidase activity to the peri-orbital region of the subject having peri-orbital puffiness; and determining an amount of fat to surgically resect from the one or more fat pads at a predetermined amount of time after administration of the composition.

The present disclosure provides a method for surgically resecting one or more eyelid fat pads, the method comprising: a. administering a composition that comprises a protein having hyaluronidase activity to the peri-orbital region of the subject; b. assessing the peri-orbital region at a predetermined amount of time after administration of the composition; c. determining an amount of fat to surgically resect from the one or more eyelid fat pads; and d. resecting a portion of the one or more eyelid fat pads.

In some embodiments of each or any of the above or below mentioned embodiments, the one or more eyelid fat pads include the upper eyelid fat pads and the lower eyelid fat pads.

In some embodiments of each or any of the above or below mentioned embodiments, the lower eyelid fat pad is selected from one or more of a lower lateral fat pad, a lower middle fat pad, a lower medial fat pad.

The present disclosure provides a method for determining if a subject with peri-orbital fullness is a candidate for treatment with a glycosaminoglycan based dermal filler along a depressed tear trough, the method comprising: a. administering a composition that comprises a protein having hyaluronidase activity to the peri-orbital region of the subject; b. assessing the peri-orbital region of the subject at a predetermined period of time after administration of the composition; and c. determining if there is an improvement in the peri-orbital puffiness, wherein the subject is a candidate for treatment with the glycosaminoglycan based dermal filler along the depressed tear trough where the subject does not exhibit an improvement in peri-orbital fullness after administration of the composition.

In some embodiments of each or any of the above or below mentioned embodiments, the peri-orbital region includes the eyelid fat pads.

In some embodiments of each or any of the above or below mentioned embodiments, the one or more eyelid fat pads include the upper eyelid fat pads and/or the lower eyelid fat pads.

In some embodiments of each or any of the above or below mentioned embodiments, the step of administering the composition is performed by one or more injections to the peri-orbital region of the subject.

The present disclosure provides a method for treating a subject having peri-orbital puffiness and a depressed tear trough, the method comprising: a. administering a composition that comprises a protein having hyaluronidase activity to the peri-orbital region of the subject; b. assessing the peri-orbital region of the subject at a predetermined period of time after administration of the composition; c. determining if there is an improvement in the peri-orbital puffiness; and d. injecting a glycosaminoglycan based filler to and area of skin having the depressed tear trough if the subject does not exhibit an improvement in peri-orbital puffiness after administration of the composition.

The present disclosure provides a method for diagnosing an etiology of upper and/or lower eyelid puffiness, the method comprising: a. examining a subject with squinted eyes; and b. determining if upper and/or lower eyelid puffiness does not improve, improves, partially improves, or worsens, wherein the etiology of the upper and/or lower eyelid puffiness is diagnosed to be anterior to the orbicularis oculi muscle if the puffiness does not improve, wherein the etiology of the upper and/or lower eyelid puffiness is diagnosed to be posterior to the orbicularis oculi muscle if the puffiness improves, wherein the etiology of the upper and/or lower eyelid puffiness is diagnosed to be anterior and posterior to the orbicularis oculi muscle if the puffiness partially improves, or wherein the puffiness is diagnosed to be secondary to hypertrophy of the orbicularis muscle or if the puffiness worsens.

In some embodiments of each or any of the above or below mentioned embodiments, the subject is in an upright position with head in a Frankfort horizontal plane.

In some embodiments of each or any of the above or below mentioned embodiments, the method further comprises the step of instructing the subject to squint or tighten the orbicularis oculi muscle.

In some embodiments of each or any of the above or below mentioned embodiments, the etiology of the upper and/or lower eyelid puffiness is determined to be anterior to the orbicularis oculi muscle, and wherein the method further comprises administering a protein having hyaluronidase activity into the soft tissue anterior to the orbicularis oculi muscle.

In some embodiments of each or any of the above or below mentioned embodiments, the etiology of the upper and/or lower eyelid puffiness is determined to be posterior to the orbicularis oculi muscle, and wherein the method further comprises the step of determining if the upper and/or lower eyelid puffiness is secondary to pseudoherniation of upper and/or lower eyelid fat pads, edema of upper and/or lower eyelid fat pads, or upper and/or lower eyelid fat pad pseudoherniation and edema.

In some embodiments of each or any of the above or below mentioned embodiments, the puffiness of the lower eyelid fat pads are assessed by asking the subject to look straight up, look up and to the right, and look up and to the left.

In some embodiments of each or any of the above or below mentioned embodiments, the puffiness of the lower eyelid fat pads is due to pseudoherniation of the lower eyelid fat pads and surgery is indicated if the lower eyelid fat pads protrude and are individually isolated.

In some embodiments of each or any of the above or below mentioned embodiments, the puffiness is due to pseudoherniation and edema of the lower eyelid fat pads if the lower eyelid fat pads protrude and are not individually isolated.

In some embodiments of each or any of the above or below mentioned embodiments, a protein having hyaluronidase activity is injected into the lower eyelid fat pads to determine the extent of edema of the lower eyelid fat pads.

In some embodiments of each or any of the above or below mentioned embodiments, the method further comprises resecting a portion of the lower eyelid fat pads.

In some embodiments of each or any of the above or below mentioned embodiments, the puffiness of the upper eyelid fat pads are assessed by asking the subject to look straight down, look down and to the right, and look down and to the left.

In some embodiments of each or any of the above or below mentioned embodiments, the puffiness of the upper eyelid fat pads is due to pseudoherniation of upper eyelid fat pads and surgery is indicated if the upper eyelid fat pads protrude and are individually isolated.

In some embodiments of each or any of the above or below mentioned embodiments, the puffiness is due to pseudoherniation and edema of upper eyelid fat pads if the upper eyelid fat pads protrude and are not individually isolated.

In some embodiments of each or any of the above or below mentioned embodiments, a protein having hyaluronidase activity is injected into the upper eyelid fat pads to determine the extent of edema of the eyelid fat pads.

In some embodiments of each or any of the above or below mentioned embodiments, the method further comprises resecting a portion of the upper eyelid fat pads.

In some embodiments of each or any of the above or below mentioned embodiments, the etiology of the upper and/or lower eyelid puffiness is determined to be anterior and posterior to the orbicularis oculi muscle, and wherein the method further comprises injecting a protein having hyaluronidase activity into the upper and/or lower eyelid fat pads.

In some embodiments of each or any of the above or below mentioned embodiments, the etiology of the upper and/or lower eyelid puffiness is determined to be anterior and posterior to the orbicularis oculi muscle, and wherein the method further comprises assessing whether the puffiness is partially due to pseudoherniation of eyelid fat pads or edema of the fat pads, or eyelid fat pad pseudoherniation and edema.

In some embodiments of each or any of the above or below mentioned embodiments, the puffiness is due to pseudoherniation of the lower eyelid fat pads and surgery is indicated if the lower eyelid fat pads protrude and are individually isolated.

In some embodiments of each or any of the above or below mentioned embodiments, a protein having hyaluronidase activity is injected into the lower eyelid fat pads to determine the extent of edema of the eyelid fat pads.

In some embodiments of each or any of the above or below mentioned embodiments, the puffiness of the upper eyelid fat pads is due to pseudoherniation of the upper eyelid fat pads and surgery is indicated if the upper eyelid fat pads protrude and are individually isolated.

In some embodiments of each or any of the above or below mentioned embodiments, the puffiness of the upper eyelid fat pads is due to pseudoherniation and edema of the upper eyelid fat pads if the upper eyelid fat pads protrude and are not individually isolated.

In some embodiments of each or any of the above or below mentioned embodiments, a protein having hyaluronidase activity is injected into the upper eyelid fat pads to determine the extent of edema of the upper eyelid fat pads.

In some embodiments of each or any of the above or below mentioned embodiments, a neuromodulator is indicated if the puffiness is determined to be secondary to hypertrophy of the orbicularis muscle or if the puffiness worsens.

The present disclosure provides a method for determining an etiology of peri-orbital puffiness, the method comprising: performing an eyelid squint test; and observing an impact of a movement of an orbicularis oculi muscle on protrusion of eyelid fat pads, wherein the etiology of the upper and/or lower eyelid puffiness is diagnosed to be anterior to the orbicularis oculi muscle if the puffiness does not improve, wherein the etiology of the upper and/or lower eyelid puffiness is diagnosed to be posterior to the orbicularis oculi muscle if the puffiness improves, wherein the etiology of the upper and/or lower eyelid puffiness is diagnosed to be anterior and posterior to the orbicularis oculi muscle if the puffiness partially improves, or wherein the puffiness is diagnosed to be secondary to hypertrophy of the orbicularis muscle or if the puffiness worsens.

The present disclosure provides a method of treating lower extremity edema in a subject in need thereof, the method comprising administering a composition that comprises a protein having hyaluronidase activity to a region of skin having the lower extremity edema on the subject, wherein the lower extremity edema is from venous stasis disease.

In some embodiments of each or any of the above or below mentioned embodiments, the edema is pitting edema.

In some embodiments of each or any of the above or below mentioned embodiments, the edema is non-pitting edema.

The present disclosure provides a method of reducing lower extremity edema in a subject in need thereof, the method comprising administering a composition that comprises a protein having hyaluronidase activity to the region of the subject having edema, wherein the administration of the composition reduces severity of the lower extremity edema, wherein the lower extremity edema is from venous stasis disease.

The present disclosure provides a method of treating edema in a subject in need thereof, the method comprising administering a composition that comprises a protein having hyaluronidase activity to a region of the subject having edema.

In some embodiments of each or any of the above or below mentioned embodiments, the peri-orbital puffiness is unrelated to the use of a hyaluronic acid filler in the peri-orbital region.

In some embodiments of each or any of the above or below mentioned embodiments, the peri-orbital puffiness is unrelated to the prior use of a hyaluronic acid filler in the peri-orbital region.

In some embodiments of each or any of the above or below mentioned embodiments, the protein having hyaluronidase activity is hyaluronidase.

In some embodiments of each or any of the above or below mentioned embodiments, the protein having hyaluronidase activity is Hylenex, Amphadase, or Vitrase.

In some embodiments of each or any of the above or below mentioned embodiments, the protein having hyaluronidase activity is Hylenex.

In some embodiments of each or any of the above or below mentioned embodiments, the protein having hyaluronidase activity is Amphadase.

In some embodiments of each or any of the above or below mentioned embodiments, the protein having hyaluronidase activity is Vitrase.

In some embodiments of each or any of the above or below mentioned embodiments, the hyaluronidase is a recombinant hyaluronidase.

In some embodiments of each or any of the above or below mentioned embodiments, the hyaluronidase is a bovine or a human hyaluronidase.

In some embodiments of each or any of the above or below mentioned embodiments, the hyaluronidase is a human hyaluronidase.

In some embodiments of each or any of the above or below mentioned embodiments, the hyaluronidase is a bovine hyaluronidase.

In some embodiments of each or any of the above or below mentioned embodiments, the one or more peri-orbital fat pads include at least one lower peri-orbital fat pad.

In some embodiments of each or any of the above or below mentioned embodiments, the lower peri-orbital fat pad is a lower lateral fat pad, a lower middle fat pad, or a lower medial fat pad.

In some embodiments of each or any of the above or below mentioned embodiments, the lower peri-orbital fat pad is a lower lateral fat pad, a lower middle fat pad, and a lower medial fat pad.

In some embodiments of each or any of the above or below mentioned embodiments, the one or more peri-orbital fat pads include at least one upper peri-orbital fat pad.

In some embodiments of each or any of the above or below mentioned embodiments, the upper peri-orbital fat pad is an upper middle fat pad or an upper medial fat pad.

In some embodiments of each or any of the above or below mentioned embodiments, the upper peri-orbital fat pad is an upper middle fat pad and an upper medial fat pad.

In some embodiments of each or any of the above or below mentioned embodiments, the step of administering comprises applying a patch, an ointment or a cream to a surface of the skin in the peri-orbital region.

In some embodiments of each or any of the above or below mentioned embodiments, the subject has not previously been treated with a dermal filler in the orbital region.

In some embodiments of each or any of the above or below mentioned embodiments, the dermal filler is a hyaluronic acid filler.

In some embodiments of each or any of the above or below mentioned embodiments, the subject has not been treated with a dermal filler 1 month, 2 months, 3 months, 4 months, 5 months, 6 months, 7 months, 8 months, 9 months, 10 months, 11 months, 12 months, 2 years, 3 years, 4 years, or 5 years before the administration of the composition that comprises the protein having hyaluronidase activity.

In some embodiments of each or any of the above or below mentioned embodiments, the protein having hyaluronidase activity is administered in a therapeutically effective amount.

In some embodiments of each or any of the above or below mentioned embodiments, the composition is administered in an amount effective to treat the peri-orbital puffiness due to peri-orbital edema.

In some embodiments of each or any of the above or below mentioned embodiments, the treatment reduces the peri-orbital puffiness due to peri-orbital edema.

In some embodiments of each or any of the above or below mentioned embodiments, each injection into the peri-orbital soft tissue and/or the one or more peri-orbital fat pads includes about 1 to about 50 Units of the protein having hyaluronidase activity.

In some embodiments of each or any of the above or below mentioned embodiments, each injection into the peri-orbital soft tissue and/or the one or more peri-orbital fat pads includes about 5 to about 15 Units of the protein having hyaluronidase activity.

In some embodiments of each or any of the above or below mentioned embodiments, each injection is performed using a 0.5 mL syringe.

In some embodiments of each or any of the above or below mentioned embodiments, the 0.5 mL syringe comprises a 32-gauge needle.

In some embodiments of each or any of the above or below mentioned embodiments, the treatment lasts from about 4 to about 12 weeks.

The present disclosure also provides a method of assessing peri-orbital puffiness due to peri-orbital edema in a subject by determining if the subject exhibits pseudoherniation of one or more upper peri-orbital fat pads and one or more lower peri-orbital fat pads; determining if the subject exhibits edema in one or more of the upper peri-orbital fat pads and the one or more of the lower peri-orbital fat pads; determining if the subject exhibits edema in the peri-orbital tissue; and scoring the peri-orbital puffiness due to peri-orbital edema from each of the prior three determinations.

In some embodiments of each or any of the above or below mentioned embodiments, the lower peri-orbital fat pad is selected from one or more of a lower lateral fat pad, a lower middle fat pad, a lower medial fat pad.

In some embodiments of each or any of the above or below mentioned embodiments, the subject is scored as Grade 0 where the subject does not exhibit pseudoherniation of any of the upper or lower peri-orbital fat pads.

In some embodiments of each or any of the above or below mentioned embodiments, the subject is scored as Grade 0 where the subject exhibits edema of one or more of the upper or lower peri-orbital fat pads.

In some embodiments of each or any of the above or below mentioned embodiments, the subject is scored as Grade 0 where the subject exhibits edema of the peri-orbital tissue.

In some embodiments of each or any of the above or below mentioned embodiments, the subject is scored as Grade 0 where the subject exhibits festoons due to edema.

In some embodiments of each or any of the above or below mentioned embodiments, the subject is scored as Grade 0 where the subject exhibits localized malar puffiness due to edema.

In some embodiments of each or any of the above or below mentioned embodiments, the subject is scored as Grade 1 where the subject exhibits pseudoherniation of the upper medial fat pad or the lower medial fat pad.

In some embodiments of each or any of the above or below mentioned embodiments, the subject is scored as Grade 1E where the subject exhibits pseudoherniation of the upper medial fat pad or the lower medial fat pad, and exhibits edema of any of the per-orbital fat pads and/or peri-orbital tissue.

In some embodiments of each or any of the above or below mentioned embodiments, the subject is scored as Grade 2 where the subject exhibits pseudoherniation of the upper eyelid and/or lower eyelid medial and central fat pads.

In some embodiments of each or any of the above or below mentioned embodiments, the subject is scored as Grade 2E where the subject exhibits pseudoherniation of the upper eyelid and/or lower eyelid medial and central fat pads, and exhibits edema of the fat pads and/or peri-orbital tissue.

In some embodiments of each or any of the above or below mentioned embodiments, the subject is scored as Grade 3 where the subject exhibits pseudoherniation of the medial, central and lateral lower eyelid fat pads.

In some embodiments of each or any of the above or below mentioned embodiments, the subject is scored as Grade 3E where the subject exhibits pseudoherniation of the medial, central and lateral lower eyelid fat pads, and exhibits edema of the fat pads and/or peri-orbital tissue.

In some embodiments of each or any of the above or below mentioned embodiments, the subject scored as Grade 1, Grade 2 or Grade 3 is treated by surgical removal of a portion of one or more of the fat pads.

In some embodiments of each or any of the above or below mentioned embodiments, the subject scored as Grade 1E, 2E or 3E is administered a composition that comprises a protein having hyaluronidase activity to the peri-orbital region of the subject, and optionally treated by surgical removal of a portion of one or more of the fat pads to increase the degree of improvement in the peri-orbital puffiness.

The present disclosure also provides a method to assess whether surgical intervention is needed to treat peri-orbital puffiness not due to peri-orbital edema in a subject in need thereof, the method comprising administering (e.g., injecting) a composition comprising a hyaluronidase to the peri-orbital region of the subject and determining that surgical intervention is required to treat the peri-orbital puffiness not due to peri-orbital edema where the administration of the composition comprising hyaluronidase does not treat (e.g., reduce) the peri-orbital puffiness not due to peri-orbital edema. Such methods may additionally comprise the step of surgically removing a portion of one or more fat pads in the upper and/or lower eyelids of the subject.

The present disclosure also provides a method of treating a cosmetic condition in a subject in need thereof due to (e.g., caused by) edema by administering a composition that comprises a protein having hyaluronidase activity to a region on the subject's face having the condition (e.g., a peri-orbital region and/or a mid-face region of the subject).

In some embodiment of each or any of the above or below mentioned embodiments, the cosmetic condition is a festoon, malar puffiness, or peri-orbital puffiness.

In some embodiment of each or any of the above or below mentioned embodiments, the subject has not previously been treated with a dermal filler in the peri-orbital region and/or the mid-face.

In some embodiment of each or any of the above or below mentioned embodiments, after the administration of the composition the edema presents as a 2 mm depression with an immediate rebound time.

In some embodiment of each or any of the above or below mentioned embodiments, after the administration of the composition the edema presents as a 3 to 4 mm depression with a rebound time of 15 seconds or less.

In some embodiment of each or any of the above or below mentioned embodiments, after administration of the composition the edema presents as a 5 to 6 mm depression with a rebound time of 10 to 30 seconds.

In some embodiment of each or any of the above or below mentioned embodiments, before administration of the composition the edema presents as a 8 mm depression with a rebound time of more than 20 seconds.

In some embodiment of each or any of the above or below mentioned embodiments, the one or more peri-orbital fat pads include the upper eyelid fat pads and the lower eyelid fat pads.

In some embodiments of each or any of the above or below mentioned embodiments, the edema is peripheral edema.

In some embodiments of each or any of the above or below mentioned embodiments, the peripheral edema is present in a leg, foot, ankle, and/or arm.

In some embodiments of each or any of the above or below mentioned embodiments, the peripheral edema is present in a foot.

In some embodiments of each or any of the above or below mentioned embodiments, the edema is pedal edema.

In some embodiments of each or any of the above or below mentioned embodiments, the edema is present is a lower leg and/or a foot.

In some embodiments of each or any of the above or below mentioned embodiments, the protein having hyaluronidase activity is a hyaluronidase.

In some embodiments of each or any of the above or below mentioned embodiments, the step of administering is performed by one or more injections to the region of the subject having edema.

In some embodiments of each or any of the above or below mentioned embodiments, the step of administering comprises applying a patch or a cream to a region of the subject having edema.

In some embodiments of each or any of the above or below mentioned embodiments, each injection includes about 1 to about 1,000 Units of the protein having hyaluronidase activity.

In some embodiments of each or any of the above or below mentioned embodiments, each injection includes about 1 to about 50 Units of the protein having hyaluronidase activity.

In some embodiments of each or any of the above or below mentioned embodiments, each injection includes about 5 to about 15 Units of the protein having hyaluronidase activity.

The present disclosure also provides a method of reducing edema in a subject in need thereof, the method comprising administering a composition that comprises a protein having hyaluronidase activity to the region of the subject having edema, wherein the administration of the composition reduces severity of the edema.

In some embodiments of each or any of the above or below mentioned embodiments, the severity of the edema is reduced by at least one grade.

In some embodiments of each or any of the above or below mentioned embodiments, the severity of the edema is reduced by at least two grades.

In some embodiments of each or any of the above or below mentioned embodiments, after the administration of the composition the edema is scored as grade 3.

In some embodiments of each or any of the above or below mentioned embodiments, after the administration of the composition the edema is scored as grade 2.

In some embodiments of each or any of the above or below mentioned embodiments, after the administration of the composition the edema is scored as grade 1.

In some embodiments of each or any of the above or below mentioned embodiments, after the administration of the composition the edema is characterized as having a 2 mm depression with an immediate rebound time.

In some embodiments of each or any of the above or below mentioned embodiments, after the administration of the composition the edema is characterized as having a 3 to 4 mm depression with a rebound time of 15 seconds or less.

In some embodiments of each or any of the above or below mentioned embodiments, after administration of the composition the edema is characterized as having a 5 to 6 mm depression with a rebound time of 10 to 30 seconds.

In some embodiments of each or any of the above or below mentioned embodiments, before administration of the composition the edema is characterized as having a 8 mm depression with a rebound time of more than 20 seconds.

In some embodiments of each or any of the above or below mentioned embodiments, the methods further comprise administering a diuretic to the subject.

The present disclosure provides compositions that comprise a protein having hyaluronidase activity and an additional active pharmaceutical ingredient (API) and methods of use thereof including, for example, to treat and/or prevent a disease or disorder.

In some embodiments of each or any of the above or below mentioned embodiments, the active pharmaceutical ingredient is a small molecule or a biologic.

In some embodiments of each or any of the above or below mentioned embodiments, the additional active pharmaceutical ingredient is used to treat acne, ADHD, AIDS/HIV, allergies, Alzheimer's, angina, anxiety, arthritis, asthma, bipolar disorder, bronchitis, cancer, cholesterol, colds & flu, constipation, COPD, depression, diabetes (Type 1), diabetes (Type 2), diarrhea, eczema, erectile dysfunction, fibromyalgia, gastrointestinal, GERE (Heartburn), gout, hair loss, hayfever, heart disease, hepatitis A, hepatitis B, herpes, hypertension, hypothyroidism, incontinence, IBD (Bowel), insomnia, menopause, mental health, migraine, osteoarthritis, osteoporosis, pain, psoriasis, rheumatoid arthritis, schizophrenia, seizures, sexual health, stroke, swine flu, UTI, or weight loss.

In some embodiments of each or any of the above or below mentioned embodiments, the additional active pharmaceutical ingredient (API) is selected from the group consisting of: 0.5-alpha-reductase inhibitors, 5-am inosalicylates, 5HT3 receptor antagonists, ACE inhibitors with calcium channel blocking agents, ACE inhibitors with thiazides, adamantane antivirals, adrenal cortical steroids, adrenal corticosteroid inhibitors, adrenergic bronchodilators, agents for hypertensive emergencies, agents for pulmonary hypertension, aldosterone receptor antagonists, alkylating agents, allergenics, alpha-glucosidase inhibitors, alternative medicines, amebicides, aminoglycosides, aminopenicillins, am inosalicylates, AMPA receptor antagonists, amylin analogs, analgesic combinations, analgesics, androgens and anabolic steroids, Angiotensin Converting Enzyme Inhibitors, angiotensin II inhibitors with calcium channel blockers, angiotensin II inhibitors with thiazides, angiotensin receptor blockers, angiotensin receptor blockers and neprilysin inhibitors, anorectal preparations, anorexiants, antacids, anthelmintics, anti-angiogenic ophthalmic agents, anti-CTLA-4 monoclonal antibodies, anti-infectives, anti-PD-1 monoclonal antibodies, antiadrenergic agents (central) with thiazides, antiadrenergic agents (peripheral) with thiazides, antiadrenergic agents, centrally acting, antiadrenergic agents, peripherally acting, antiandrogens, antianginal agents, antiarrhythmic agents, antiasthmatic combinations, antibiotics/antineoplastics, anticholinergic antiemetics, anticholinergic antiparkinson agents, anticholinergic bronchodilators, anticholinergic chronotropic agents, anticholinergics/antispasmodics, anticoagulant reversal agents, anticoagulants, anticonvulsants, antidepressants, antidiabetic agents, antidiabetic combinations, antidiarrheals, antidiuretic hormones, antidotes, antiemetic/antivertigo agents, antifungals, antigonadotropic agents, antigout agents, antihistamines, antihyperlipidemic agents, antihyperlipidemic combinations, antihypertensive combinations, antihyperuricemic agents, antimalarial agents, antimalarial combinations, antimalarial quinolones, antimanic agents, antimetabolites, antimigraine agents, antineoplastic combinations, antineoplastic detoxifying agents, antineoplastic interferons, antineoplastics, antiparkinson agents, antiplatelet agents, antipseudomonal penicillins, antipsoriatics, antipsychotics, antirheumatics, antiseptic and germicides, antithyroid agents, antitoxins and antivenins, antituberculosis agents, antituberculosis combinations, antitussives, antiviral agents, antiviral boosters, antiviral combinations, antiviral interferons, anxiolytics, sedatives, and hypnotics, aromatase inhibitors, atypical antipsychotics, azole antifungals, Abilify, Ablysinol, Absorica, Acanya, Accolate, Accrufer, AccuNeb, Accupril, Accuretic, Acetadote, Achromycin V, Aciphex, Aciphex Sprinkle, Acthrel, Acticlate, Actigall, Actiq, Activella, Actonel, ActoPlus Met, Actos, Acular, Acular LS, Acuvail, Aczone, Adalat CC, Adasuve, Adcirca, Adderall, Adderall XR, Addyi, Adempas, Adhansia XR, Adlyxin, Admelog, Adrenaclick, AdreView, Advair Diskus, Advair HFA, Adzenys ER, Adzenys XR-ODT, Aemcolo, Afinitor, Afinitor Disperz, Aggrastat, Aggrenox, Agrylin, AirDuo Digihaler, AirDuo Respiclick, AK-Fluor, Aklief, Akovaz, Akten, Akynzeo, Albenza, Aldactazide, Aldactone, Aldara, Alecensa, Alfenta, Alimta, Alinia, Aliqopa, Alkeran, Alocril, Alomide, Aloprim, Alora, Aloxi, Alphagan, Alrex, Altabax, Altace, Altafluor Benox, Altoprev, Altreno, Alunbrig, Alvesco, Amaryl, Ambien, Ambien CR, Ameluz, Amerge, Amicar, Amidate, Amitiz, Ammonul, Amphadase, Ampyra, Amrix, Amyvid, Amzeeq, Anadrol-50, Anafranil, Anaprox-DS, Ancobon, Androderm, AndroGel, Anectine, Angeliq, bacterial vaccines, barbiturate anticonvulsants, barbiturates, BCR-ABL tyrosine kinase inhibitors, benzodiazepine anticonvulsants, benzodiazepines, beta blockers with calcium channel blockers, beta blockers with thiazides, beta-adrenergic blocking agents, beta-lactamase inhibitors, bile acid sequestrants, biologicals, bisphosphonates, bone morphogenetic proteins, bone resorption inhibitors, bronchodilator combinations, bronchodilators, Bactrim, Bactrim DS, Balcoltra, Balversa, Banzel, Baqsimi, Baraclude, Basaglar, Baxdela, Belbuca, Beleodaq, Belrapzo, Belsomra, Belviq, Belviq XR, Bendeka, Benicar, Benicar HCT, Bentyl, Benzaclin, Benzamycin, Bepreve, Besivance, Beta-Val, Betagan, Betapace, Betapace AF, Bethkis, Betimol, Betoptic, Betoptic S, Bevespi Aerosphere, Bevyxxa, Beyaz, Bicillin L-A, BiCNU, BiDil, Bijuva, Biktarvy, Biltricide, Binosto, Biorphen, Blephamide, Bloxiverz, Boniva, Bonjesta, Bonsity, Bosulif, Braftovi, Breo Ellipta, Brethine, Brevibloc, Brevital Sodium, Bridion, Brilinta, Brisdelle, Briviact, BromSite, Brovana, Bryhali, Bumex, Bunavail, Buphenyl, Buprenex, Busulfex, Butisol Sodium, Butrans, Bydureon, Bydureon BCise, Byetta, Bystolic, Calcimimetics, calcineurin inhibitors, calcitonin, calcium channel blocking agents, carbamate anticonvulsants, carbapenems, carbapenems/beta-lactamase inhibitors, carbonic anhydrase inhibitor anticonvulsants, carbonic anhydrase inhibitors, cardiac stressing agents, cardioselective beta blockers, cardiovascular agents, catecholamines, cation exchange resins, CD20 monoclonal antibodies, CD30 monoclonal antibodies, CD33 monoclonal antibodies, CD38 monoclonal antibodies, CD52 monoclonal antibodies, CDK 4/6 inhibitors, central nervous system agents, cephalosporins, cephalosporins/beta-lactamase inhibitors, cerumenolytics, CFTR combinations, CFTR potentiators, CGRP inhibitors, chelating agents, chemokine receptor antagonist, chloride channel activators, cholesterol absorption inhibitors, cholinergic agonists, cholinergic muscle stimulants, cholinesterase inhibitors, CNS stimulants, coagulation modifiers, colony stimulating factors, contraceptives, corticotropin, coumarins and indandiones, cox-2 inhibitors, Cabometyx, Caduet, Cafcit, Calan, Calan SR, Calcium Disodium Versenate, Caldolor, Calquence, Cambia, Campral, Camptosar, Canasa, Cancidas, Capastat Sulfate, Capex, Caprelsa, Carac, Carafate, Carbaglu, Carbatrol, Carbocaine, Cardiogen-82, Cardizem, Cardizem CD, Cardizem LA, Cardura, Cardura XL, Carnitor, Carnitor SF, CaroSpir, Casodex, Casporyn HC, Catapres, Catapres-TTS, Caverject, Caverject Impulse, Cayston, Cefotan, Ceftin, Celebrex, Celestone Soluspan, Celexa, CellCept, Celontin, Centany, Cequa, Cerdelga, Cerebyx, Cerezyme, Cesamet, Cetraxal, Cetrotide, Chantix, Chemet, ChiRhoStim, Cholbam, Cialis, Ciloxan, Cimduo, Cinvanti, Cipro, Cipro HC, Ciprodex, Clarinex, Clarinex-D 12 Hour, Clenpiq, Cleocin Phosphate, Cleocin T, Cleocin Vaginal, Cleviprex, Climara, Climara Pro, lindagel, Clindesse, Clobex, Cloderm, Clolar, Clomid, Clorotekal, Clozaril, Coartem, Cogentin, Colazal, Colcrys, Colestid, Coly Mycin M, Coly-Mycin S, Combigan, CombiPatch, Combivent Respimat, Combivir, Cometriq, Complera, Comtan, Concerta, Condylox, Conray, Consensi, Contrave, ConZipD, Copaxone, Copegus, Copiktra, Cordarone, Cordran, Cordran SP, Coreg, Coreg CR, Corgard, Corlanor, Corlopam, Corphedra, Cortef, Cortenema, Cortifoam, Cortisporin Cream, Cortisporin Ointment, Cortrosyn, Corvert, Corzide, Cosmegen, Cosopt, Cosopt PF, Cotellic, Cotempla XR-ODT, Coumadin, Cozaar, Cresemba, Crestor, Crixivan, Cubicin, Cubicin RF, Cuprimine, Curosurf, Cutivate, Cuvposa, Cyanokit, Cyclessa, Cycloset, Cyklokapron, Cymbalta, Cystadane, Cystagon, Cystaran, Cysto-Conray II, Cystografin, Cytomel, Cytotec, Cytovene, Decongestants, dermatological agents, diagnostic radiopharmaceuticals, diarylquinolines, dibenzazepine anticonvulsants, digestive enzymes, dipeptidyl peptidase 4 inhibitors, diuretics, dopaminergic, antiparkinsonism agents, drugs used in alcohol dependence, D.H.E. 45, Dacogen, Daliresp, Dalvance, Dantrium, Daraprim, DaTscan, Daurismo, Daypro, Daytrana, DDAVP, Definity, Defitelio, Delestrogen, Delstrigo, Delzicol, Demadex, Demerol, Demser, Denavir, Depacon, Depakote, Depakote ER, Depen, Depo-Medrol, Depo-Provera, Depo-subQ provera 104, Derma-Smoothe/FS, Dermatop, DermOtic Oil, Descovy, Desferal, Desonate, DesOwen, Desoxyn, Detrol, Detrol LA, Dexedrine, Dexilant, Dextenza, DiaBeta, Diabinese, Diacomit, Diastat, Diastat AcuDial, Dibenzyline, Diclegis, Differin, Dificid, Diflucan, Dilantin, Dilatrate-SR, Dilaudid, Diovan, Diovan HCT, Dipentum, Diprivan, Diprolene, Diprolene AF, Ditropan XL, Diuril, Divigel, Docefrez, Dolophine, Dopram, Doptelet, Doral, Doryx, Doryx MPC, Dotarem, Dovato, Dovonex, Drisdol, Drizalma Sprinkle, Droxia, Dsuvia, Duac, Duaklir Pressair, Duavee, Duetact, Duexis, Dulera, Duobrii, DuoDote, Duopa, Duraclon, Duragesic, Duramorph PF, Durezol, Durlaza, Dutoprol, Dyanavel XR, Dyazide, Dyrenium, Echinocandins, EGFR inhibitors, estrogen receptor antagonists, estrogens, expectorants, E-Z-HD, E-Z-Paque, EC-Naprosyn, Ecoza, Edarbi, Edarbyclor, Edecrin, Edex, Edluar, Edurant, Effexor, Effexor XR, Effient, Efudex, Egaten, Egrifta, Elcys, Elelyso, Elepsia XR, Elestat, Elestrin, Elidel, Eligard, Elimite, Eliquis, Ella, Ellence, Elmiron, Elocon, Eloxatin, Embeda, Emcyt, Emend, Emflaza, Emla, Emsam, Emtriva, Enablex, Endari, Endometrin, Enstilar, Entereg, Entocort EC, Entresto, Envarsus XR, Eovist, Epaned, Epclusa, Epidiolex, Epiduo, Epiduo Forte, EpiPen, EpiPen Jr, Epivir, Epivir-HBV, Epzicom, Equetro, Eraxis, Erivedge, Erleada, Ertaczo, Eryc, Erygel, EryPed, Erythrocin, Esbriet, Estrogel, Estrostep Fe, Ethamolin, Ethyol, Eucrisa, Eurax, Euthyrox, Evamist, Evekeo ODT, Evista, Evoclin, Evomela, Evotaz, Evoxac, Evzio, Exelderm, Exelon, Exforge, Exforge HCT, Exjade, Exondys 51, Extina, Ezallor, factor Xa inhibitors, fatty acid derivative anticonvulsants, fibric acid derivatives, first generation cephalosporins, fourth generation cephalosporins, functional bowel disorder agents, Fabior, Factive, Famvir, Fanapt, Fareston, Farxiga, Farydak, Faslodex, Felbatol, Feldene, Femara, Femhrt, Femring, Fenoglide, Fentora, Feraheme, Ferriprox, Ferrlecit, Fetzima, Fiasp, Fibricor, Finacea, Fiorinal, Firazyr, Firdapse, Firmagon, Flagyl, Flarex, Flector Patch, Flolan, FloLipid, Flomax, Flovent HFA, Flumadine, Fluorescite, Fluoroplex, FML, FML Forte Liquifilm, Focalin, Focalin XR, Follistim AQ, Folotyn, Forane, Forfivo XL, Fortamet, Fortaz, Forteo, Fortesta, Fosamax, Fosamax Plus D, Foscavir, Fosrenol, Fragmin, Frova, Furadantin, Fusilev, Fuzeon, Fycompa, gallstone solubilizing agents, gamma-aminobutyric acid analogs, gamma-aminobutyric acid reuptake inhibitors, gastrointestinal agents, general anesthetics, genitourinary tract agents, GI stimulants, glucocorticoids, glucose elevating agents, glycopeptide antibiotics, glycoprotein platelet inhibitors, glycylcyclines, gonadotropin releasing hormones, gonadotropin-releasing hormone antagonists, gonadotropins, group I antiarrhythmics, group II antiarrhythmics, group III antiarrhythmics, group IV antiarrhythmics, group V antiarrhythmics, growth hormone receptor blockers, growth hormones, guanylate cyclase-C agonists, Gabitril, Gablofen, Gadavist, Galafold, Galzin, Gastrocrom, Gastrografin, Gelnique, Gemzar, Genotropin, Genvoya, Geodon, Giapreza, Gilenya, Gilotrif, Gleevec, Gleolan, Gleostine, Gliadel, Gloperba, GlucaGen, Glucophage, Glucophage XR, Glucotrol, Glucotrol XL, Glumetza, Glynase, Glyrx-PF, Glyset, Glyxambi, Gocovri, Gonal-f, Gonal-f RFF, Gonal-f RFF Redi-ject, GoNitro, Gralise, Gris-PEG, Gvoke, H. pylori eradication agents, H2 antagonists, hedgehog pathway inhibitors, hematopoietic stem cell mobilizer, heparin antagonists, heparins, HER2 inhibitors, herbal products, histone deacetylase inhibitors, hormones, hormones/antineoplastics, hydantoin anticonvulsants, hydrazide derivatives, H.P. Acthar Gel, Halaven, Halcion, Haldol, Halog, Harvoni, Hectorol, Hemabate, Hemady, Hemangeol, Hepsera, Hetlioz, Hicon, Hiprex, Horizant, Humalog, Humalog KwikPen, Humalog Mix 50/50, Humalog Mix 50/50 KwikPen, Humalog Mix 75/25, Humalog Mix 75/25 KwikPen, Humatrope, Hycamtin, Hycodan, Hydrea, Hysingla ER, Hyzaar, illicit (street) drugs, immune globulins, immunologic agents, immunostimulants, immunosuppressive agents, impotence agents, in vivo diagnostic biologicals, incretin mimetics, inhaled anti-infectives, inhaled corticosteroids, inotropic agents, insulin, insulin-like growth factors, integrase strand transfer inhibitor, interferons, interleukin inhibitors, interleukins, intravenous nutritional products, iodinated contrast media, ionic iodinated contrast media, iron products, Ibrance, Ibsrela, IC-Green, Iclusig, Idamycin PFS, Idhifa, Ifex, Ilevro, Iluvien, Imbruvica, Imdur, Imitrex, Imitrex Statdose, Impavido, Impoyz, Imuran, Inapsine, Inbrija, Increlex, Incruse Ellipta, Inderal LA, Indocin, Infed, Infugem, Infumorph, Ingrezza, Injectafer, Inlyta, InnoPran XL, INOmax, Inrebic, Inspra, Integrilin, Intelence, Intuniv, Invanz Invega, Invega Sustenna, Invega Trinza, Inveltys, Invirase, Invokamet, Invokamet XR, Invokana, Iopidine, Iressa, Isentress, Isentress HD, Isopto Atropine, Isopto Carpine, Isordil, Isovue-200, Isovue-250, Isovue-300, Isovue-370, Istalol, Istodax, Isuprel, Jadenu, Jadenu Sprinkle, Jakafi, Jalyn, Janumet, Janumet XR, Januvia, Jardiance, Jatenzo, Jentadueto, Jentadueto XR, Jornay PM, Jublia, Juluca, Juxtapid, Jynarque, Ketolides, K-Tab, Kadian, Kaletra, Kalydeco, Kapspargo Sprinkle, Kapvay, Karbinal ER, Katerzia, Kazano, Keflex, Kenalog, Kenalog-10, Kenalog-40, Kengreal, Keppra, Keppra XR, Kerydin, Ketalar, Keveyis, Khapzory, Kinevac, Kisqali, Kitabis Pak, Klaron, Klonopin, Klor-Con, Kombiglyze XR, Korlym, Krintafel, Kuvan, Kybella, Kyleena, Kyprolis, Laxatives, leprostatics, leukotriene modifiers, lincomycin derivatives, local injectable anesthetics, local injectable anesthetics with corticosteroids, loop diuretics, lung surfactants, lymphatic staining agents, lysosomal enzymes, Lacrisert, Lamictal, Lamictal CD, Lamictal ODT, Lamictal XR, Lamisil, Lanoxin, Lantus, Lantus SoloStar, Lasix, Lastacaft, Latisse, Latuda, Lazanda, Lenvima, Lescol XL, Letairis, Leukeran, Levemir, Levitra, Levlite, Levophed, Levulan Kerastick, Lexapro, Lexette, Lexiscan, Lexiva, Lialda, Librax, Lidex, Lidex-E, Lidoderm, Liletta, Lincocin, Linzess, Lioresal, Lipiodol, Lipitor, Lipofen, Lithobid, Lithostat Livalo, Lo Loestrin Fe, Locoid, Locoid Lipocream, Lodosyn, Loestrin 21 1.5/30, Loestrin 21.1/20, Loestrin 24 Fe, Loestrin Fe 1.5/30, Loestrin Fe 1/20, Lokelma, Lomotil, Lonsurf, Lopid, Lopressor, Lopressor HCT, Loprox, Lorbrena, LoSeasonique, Lotemax, Lotemax SM, Lotensin, Lotensin HCT, Lotrel, Lotrisone, Lotronex, Lovaza, Lovenox, Lucemyra, Lumason, Lumigan, Lunesta, Lupaneta Pack, Lupron Depot, Lupron Depot-PED, Lutathera, Luvox, Luxiq, Luzu, Lybrel, Lynparza, Lyrica, Lyrica CR, Lysodren, Lysteda, macrolide derivatives, macrolides, magnetic resonance imaging contrast media, malignancy photosensitizers, mast cell stabilizers, medical gas, meglitinides, metabolic agents, methylxanthines, mineralocorticoids, minerals and electrolytes, miscellaneous agents, miscellaneous analgesics, miscellaneous antibiotics, miscellaneous anticonvulsants, miscellaneous antidepressants, miscellaneous antidiabetic agents, miscellaneous antiemetics, miscellaneous antifungals, miscellaneous, ntihyperlipidemic agents, miscellaneous antihypertensive combinations, miscellaneous antimalarials, miscellaneous antineoplastics, miscellaneous antiparkinson agents, miscellaneous antipsychotic agents, miscellaneous antituberculosis agents, miscellaneous antivirals, miscellaneous anxiolytics, sedatives and hypnotics, miscellaneous bone resorption inhibitors, miscellaneous cardiovascular agents, miscellaneous central nervous system agents, miscellaneous coagulation modifiers, miscellaneous diagnostic dyes, miscellaneous diuretics, miscellaneous genitourinary tract agents, miscellaneous GI agents, miscellaneous hormones, miscellaneous metabolic agents, miscellaneous ophthalmic agents, miscellaneous otic agents, miscellaneous respiratory agents, miscellaneous sex hormones, miscellaneous topical agents, miscellaneous uncategorized agents, miscellaneous vaginal agents, mitotic inhibitors, monoamine oxidase inhibitors, mouth and throat products, mTOR inhibitors, mucolytics, multikinase inhibitors, muscle relaxants, mydriatics, Macrilen, Macrobid, Macrodantin, Makena, Malarone, Malarone Pediatric, Marcaine, Marinol, Marplan, Matulane, Mavenclad, Mavyret, Maxalt, Maxalt-MLT, Maxidex, Maxipime, Maxitrol, Maxzide, Maxzide-25, Mayzent, Medrol, Megace ES, Mekinist, Mektovi, MembraneBlue, Menostar, Mentax, Mephyton, Mepron, Merrem, Mesnex, Mestinon, Metadate CD, Metastron, Methadose, Methergine, Methylin, Metopirone, MetroCream, MetroGel, MetroGel-Vaginal, MetroLotion, Miacalcin, Micardis, Micardis HCT, Micro-K, Micro-K 10, Microzide, Midamor, Mifeprex, Migranal, Minastrin 24 Fe, Minipress, Minirin, Minivelle, Minocin, Minolira, Miochol-E, Miostat, Mirapex, Mirapex ER, Mircette, Mirena, Mirvaso, Mitigare, Mobic, Monistat 3, Monoket, Monurol, MorphaBond ER, Motegrity, Motofen, Movantik, Moxeza, Mozobil, MS Contin, Mulpleta, Multaq, Multihance, Muse, Myambutol, Mycamine, Mycobutin, Mydayis, Myfortic, Myleran, Myrbetriq, Mysoline, Mytesi, narcotic analgesic combinations, narcotic analgesics, nasal anti-infectives, nasal antihistamines and decongestants, nasal lubricants and irrigations, nasal preparations, nasal steroids, natural penicillins, neprilysin inhibitors, neuraminidase inhibitors, neuromuscular blocking agents, neuronal potassium channel openers, next generation cephalosporins, NHE3 inhibitors, nicotinic acid derivatives, NK1 receptor antagonists, NNRTIs, non-cardioselective beta blockers, non-iodinated contrast media, non-ionic iodinated contrast media, non-sulfonylureas, Nonsteroidal anti-inflammatory drugs, NS5A inhibitors, nucleoside reverse transcriptase inhibitors (NRTIs), nutraceutical products, nutritional products, Naftin, Nalfon, Namenda, Namenda XR, Namzaric, Naprelan, Naprosyn, Narcan Nasal Spray, Nardil, Naropin, Nascobal, Natacyn, Natazia, Natesto, Natroba, Nayzilam, Nebupent, NeoProfen, Neoral, Nerlynx, Nesacaine, Nesacaine-MPF, Nesina, Netspot, Neupro, Neuraceq, Neurontin, Nevanac, Nexavar, Nexium, Nexium IV, Nexplanon, Nexterone, Niaspan, Nicotrol Inhaler, Nilandron, Nimbex, Ninlaro, Nipent, Niravam, Nithiodote, Nitro-Dur, Nitrolingual Pumpspray, NitroMist, Nitrostat, Nityr, Nizoral Topical, Nocdurna, Nor-QD, Nordette-28, Norditropin FlexPro, Noritate, Norpace, Norpace CR, Norpramin, Northera, Norvasc, Norvir, Nourianz, Novolog, NovoLog FlexPen, NovoLog Mix 70/30, NovoLog Mix 70/30 FlexPen, NovoLog PenFill, Noxafil, Nubeqa, Nucynta, Nucynta ER, Nuedexta, Nuplazid, Nutropin AQ, NuvaRing, Nuvessa, Nuvigil, Nuzyra, Nymalize, ophthalmic anesthetics, ophthalmic anti-infectives, ophthalmic anti-inflammatory agents, ophthalmic antihistamines and decongestants, ophthalmic diagnostic agents, ophthalmic glaucoma agents, ophthalmic lubricants and irrigations, ophthalmic preparations, ophthalmic steroids, ophthalmic steroids with anti-infectives, ophthalmic surgical agents, oral nutritional supplements, other immunostimulants, other immunosuppressants, otic anesthetics, otic anti-infectives, otic preparations, otic steroids, otic steroids with anti-infectives, oxazolidinedione anticonvulsants, oxazolidinone antibiotics, Ocaliva, Octreoscan, Ocufen, Ocuflox, Odefsey, Odomzo, Ofev, Ofirmev, Olumiant, Olux, Omnicef, Omnipaque 12, Omnipaque 140, Omnipaque 180, Omnipaque 240, Omnipaque 300, Omnipaque 350, Omnipaque 9, Omnipred, Omniscan, Omnitrope, Onexton, Onfi, Onglyza, Onmel, Onpattro, Onzetra Xsail, Opana, Opsumit, Optiray 240, Optiray 300, Optiray 320, Optiray 350, Orabloc, Oracea, Oraltag, Orapred ODT, Oraqix, OraVerse, Oravig, Orbactiv, Orenitram, Orfadin, Orilissa, Orkambi, Ortho Tri-Cyclen, Ortho Tri-Cyclen Lo, Ortho-Novum 7/7/7, Oseni, Osmolex ER, OsmoPrep, Osphena, Otezla, Otiprio, Otovel, Otrexup, Ovidrel, Oxaydo, Oxistat, Oxsoralen-Ultra, Oxtellar XR, OxyContin, Oxytrol, Ozempic, Ozobax, Ozurdex, parathyroid hormone and analogs, PARP inhibitors, PCSK9 inhibitors, penicillinase resistant penicillins, penicillins, peripheral opioid receptor antagonists, peripheral opioid receptor mixed gonists/antagonists, peripheral vasodilators, peripherally acting antiobesity agents, phenothiazine antiemetics, phenothiazine antipsychotics, phenylpiperazine antidepressants, phosphate binders, PI3K inhibitors, plasma expanders, platelet aggregation inhibitors, platelet-stimulating agents, polyenes, potassium sparing diuretics with thiazides, potassium-sparing diuretics, probiotics, progesterone receptor modulators, progestins, prolactin inhibitors, prostaglandin D2 antagonists, protease inhibitors, protease-activated receptor-1 antagonists, proteasome inhibitors, proton pump inhibitors, psoralens, psychotherapeutic agents, psychotherapeutic combinations, purine nucleosides, pyrrolidine anticonvulsants, Pamelor, Pandel, Panretin, Paremyd, Parlodel, Parnate, Parsabiv, Pataday, Patanol, Paxil, Paxil CR, Pazeo, PediaPred, Peganone, Penlac, Pennsaid, Pentam, Pentasa, Pepcid, Percodan, Perforomist, Peridex, PerioChip, Persantine, Pexeva, Phoslyra, Phospholine Iodide, Photofrin, Picato, Pifeltro, Piqray, Pitocin, Plaquenil, Plavix, Pliaglis, Polytrim, Pomalyst, Ponstel, Pradaxa, Pravachol, Pre-Pen, Precedex, Pred Forte, Pred Mild, Pred-G, Pregnyl, Premarin, Premphase 14/14, Prempro, Prepidil, Prestalia, Prevacid, Prevymis, Prezcobix, Prezista, Prialt, Priftin, Prilosec, Primsol, Prinivil, Pristiq, ProAir Digihaler, ProAir HFA, ProAir RespiClick, Probuphine, Procardia, Procardia XL, Procysbi, Proglycem, Prograf, Prohance, Prolensa, Promacta, Prometrium, Propecia, Proscar, Prostin E2, Prostin VR Pediatric, Protonix, Protonix IV, Protopam Chloride, Protopic, Provayblue, Provera, Provigil, Provocholine, Prozac, Prozac Weekly, Pulmicort Flexhaler, Pulmicort Respules, Purinethol, Purixan, Pylerac, Quinolones, Qbrelis, Qbrexza, Qmiiz ODT, QNASL, Qoliana, Qsymia, Qtern, Qternmet XR, Quadramet, Qualaquin, Quartette, Qudexy XR, Quelicin, QuilliChew ER, Quillivant XR, Qutenza, Quzyttir, Qvar RediHaler, radiocontrast agents, radiologic adjuncts, radiologic agents, radiologic conjugating agents, radiopharmaceuticals, recombinant human erythropoietins, renin inhibitors, respiratory agents, respiratory inhalant products, rifamycin derivatives, R-Gene 10, Radicava, Ranexa, Rapaflo, Rapamune, Rapivab, Rasuvo, Ravicti, Rayaldee, Rayos, Razadyne, Razadyne ER, Readi-Cat 2, Rebetol, Recarbrio, Reclast, Rectiv, Reglan, Regonol, Relenza, Relistor, Relpax, Remeron, Remeron SolTab, Remodulin, Renagel, Renova, Renvela, Requip, Requip XL, Rescriptor, Restasis, Restoril, Retin-A, Retisert, Retrovir, Revatio, Revlimid, Rexulti, Reyataz, Rhofade, Rhopressa, Ridaura, Rifadin, Rifater, Rilutek, Rimactane, Rimso-50, Rinvoq, Riomet, Riomet ER, Risperdal, Risperdal Consta, Ritalin, Ritalin LA, Robaxin, Robaxin-750, Rocaltrol, Rocklatan, Rowasa, Roxicodone, Rozerem, Rozlytrek, Rubraca, Ruby-Fill, Ruzurgi, Ryanodex, Rybelsus, Rydapt, Rytary, Rythmol, Rythmol SR, salicylates, sclerosing agents, second generation cephalosporins, selective estrogen receptor modulators, selective immunosuppressants, selective phosphodiesterase-4 inhibitors, selective serotonin reuptake inhibitors, serotonin-norepinephrine reuptake inhibitors, serotoninergic neuroenteric modulators, sex hormone combinations, sex hormones, SGLT-2 inhibitors, skeletal muscle relaxant combinations, skeletal muscle relaxants, smoking cessation agents, somatostatin and somatostatin analogs, spermicides, statins, sterile irrigating solutions, streptogramins, streptomyces derivatives, succinimide anticonvulsants, sulfonamides, sulfonylureas, synthetic ovulation stimulants, Sabril, Safyral, Saizen, Salagen, Samsca, Sancuso, Sandimmune, Sandostatin, Saphris, Sarafem, Savaysa, Savella, Saxenda, Scenesse, Sclerosol, Seasonale, Seasonique, Secuado, Segluromet, Seizalam, Selzentry, Semprex-D, Sensipar, Sensorcaine, Septocaine, Septra, Septra DS, Serevent Diskus, Sernivo, Seroquel, Seroquel XR, Serostim, Seysara, Signifor, Siklos, Silenor, SilQ Vanilla, Silvadene, Simbrinza, Sinemet, Sinemet CR, Singulair, Sirturo, Sitavig, Sivextro, Skelaxin, Sklice, Skyla, Slynd, Smoothie Readi-Cat 2, Solaraze, Soliqua, Solodyn, Solosec, Soltamox, Solu-Cortef, Solu-Medrol, Soma, Somatuline Depot, Somavert, Sonata, Soolantra, Soriatane, Sorilux, Sotylize, Sovaldi, Spectazole, Spinraza, Spiriva, Spiriva Respimat, Sporanox, Spritam, Sprycel, SSD, Stalevo 100, Stalevo 125, Stalevo 150, Stalevo 200, Stalevo 50, Stalevo 75, Staxyn, Steglatro, Steglujan, Stendra, Steritalc, Stiolto Respimat, Stivarga, Strattera, Stribild, Striverdi Respimat, Stromectol, Sublocade, Suboxone, Subsys, Sucraid, Sular, Sulfamylon, Sulfatrim Pediatric, Sunosi, Supprelin LA, Suprane, Suprane, Suprax, Suprep Bowel Prep Kit, Survanta, Sustiva, Sustol, Sutent, Symbicort, Symbyax, Symfi, Symfi Lo, Symjepi, Symlin, Symmetrel, Sympazan, Symproic, Symtuza, Synalar, Synarel, Syndros, Synera, Synercid, Synjardy, Synjardy XR, Synribo, SyprineSuprax, Suprep Bowel Prep Kit, Survanta, Sustiva, Sustol, Sutent, Symbicort, Symbyax, Symfi, Symfi Lo, Symjepi, Symlin, Symmetrel, Sympazan, Symproic, Symtuza, Synalar, Synarel, Syndros, Synera, Synercid, Synjardy, Synjardy XR, Synribo, Syprine, tetracyclic antidepressants, tetracyclines, therapeutic radiopharmaceuticals, therapeutic vaccines, thiazide diuretics, thiazolidinediones, thioxanthenes, third generation cephalosporins, thrombin inhibitors, thrombolytics, thyroid drugs, TNF alfa inhibitors, tocolytic agents, topical acne agents, topical agents, topical allergy diagnostic agents, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical depigmenting agents, topical emollients, topical keratolytics, topical non-steroidal anti-inflammatories, topical photochemotherapeutics, topical rubefacient, topical steroids, topical steroids with anti-infectives, transthyretin stabilizers, triazine anticonvulsants, tricyclic antidepressants, trifunctional monoclonal antibodies, Taclonex, Tafinlar, Tagitol V, Tagrisso, Talzenna, Tambocor, Tamiflu, Tarceva, Targretin, Targretin Gel, Tarka, Tasigna, Tasmar, Tavalisse, Taxotere, Taytulla, Tazorac, Tecfidera, Teflaro, Tegretol, Tegretol XR, Tegsedi, Tekturna, Tekturna HCT, Temixys, Temodar, Tenoretic 100, Tenoretic 50, Tenormin, Tepadina, Tessalon, Testim, Thalomid, Thermazene, Thiola, Thiola EC, Thyrogen, Tiazac, Tibsovo, Tigan, Tikosyn, Timoptic, Timoptic-XE, Tindamax, Tirosint, Tirosint-Sol, Tivicay, Tivorbex, Tobi, TOBI Podhaler, TobraDex, Tobradex ST, Tobrex, Tolak, Tolsura, Topamax, Topicort, Toprol-XL, Torisel, Tosymra, Totect, Toujeo Max SoloStar, Toujeo SoloStar, Toviaz, TPDXX, Tracleer, Tradjenta, Trandate, Transderm-Scop, Tranxene, Travatan Z, Treanda, Trecator, Trelegy Ellipta, Trelstar, Tresiba, Treximet, Tri-Luma, Tribenzor, TriCor, Triferic, Triglide, Trileptal, Trilipix, Trintellix, Triostat, Triphasil-28, Trisenox, Triumeq, Trizivir, Trokendi XR, Trulance, Trusopt, Truvada, Tudorza Pressair, Turalio, Tuxarin ER, Tuzistra XR, Twynsta, Tybost, ultrasound contrast media, upper respiratory combinations, urea anticonvulsants, urea cycle disorder agents, urinary anti-infectives, urinary antispasmodics, urinary pH modifiers, uterotonic agents, Uceris, Uloric, Ultane, Ultiva, Ultracet, Ultram, Ultravate, Ultravist, Unasyn, Uptravi, Urex, Urocit-K, Uroxatral, Urso, Urso Forte, Uvadex, vaccine combinations, vaginal anti-infectives, vaginal preparations, vasodilators, vasopressin antagonists, vasopressors, VEGF/VEGFR inhibitors, viral vaccines, viscosupplementation agents, vitamin and mineral combinations, vitamins, VMAT2 inhibitors, Vabomere, Vagifem, Valchlor, Valcyte, Valium, Valstar, Valtrex, Vandazole, Vaniqa, Vanos, Vantas, Varibar Honey, Varibar Nectar, Varibar Pudding, Varibar Thin Honey, Varubi, Vascepa, Vaseretic, Vasostrict, Vasotec, Vazculep, Vectical, Velcade, Veletri, Velphoro, Veltassa, Veltin, Vemlidy, Venclexta, Venofer, Ventavis, Ventolin HFA, Verdeso, Veregen, Verelan, Verelan PM, Versacloz, Verzenio, VESIcare, Vfend, Viagra, Vibativ, Viberzi, Vibramycin, Victoza, Vidaza, Videx, Videx EC, Vigamox, Viibryd, Vimovo, Vimpat, Viracept, Viramune, Viramune XR, Virazole, Viread, Viroptic, VisionBlue, Vistaril, Vistogard, Visudyne, Vitrakvi, Vitrase, Vivelle-Dot, Vivitrol, Vivlodex, Vizamyl, Vizimpro, Vogelxo, Voltaren Ophthalmic, Vosevi, Vosol, Vosol HC, Votrient, VPRIV, Vraylar, Vumerity, Vyndamax, Vyndaqel, Vytorin, Vyvanse, Vyzulta, Wakix, Welchol, Wellbutrin, Wellbutrin SR, Wellbutrin XL, Xadago, Xalatan, Xalkori, Xanax, Xanax XR, Xarelto, Xatmep, Xeljanz, Xeljanz XR, Xeloda, Xelpros, Xenazine, Xenical, Xenleta, Xepi, Xerava, Xerese, Xermelo, Xifaxan, Xigduo XR, Xiidra, Ximino, Xofigo, Xofluza, Xolegel, Xopenex, Xopenex HFA, Xospata, Xpovio, Xtampza ER, Xtandi, Xultophy, Xuriden, Xyrem, Xyzal, Yasm in, Yaz, Yondelis, Yonsa, Yosprala, Yupelri, Yutiq, Zanaflex, Zanosar, Zantac, Zantac 300, Zarontin, Zaroxolyn, Zavesca, Zegerid, Zejula, Zelapar, Zelboraf, Zelnorm, Zembrace SymTouch, Zemdri, Zemplar, Zepatier, Zerbaxa, Zerit, Zerviate, Zestoretic, Zestril, Zetia, Zetonna, Ziac, Ziagen, Ziana, Zilretta, Zinacef, Zinecard, Zioptan, Zipsor, Zirgan, Zithromax, Zocor Zofran, Zofran ODT, Zohydro ER, Zoladex, Zolinza, Zoloft, Zolpimist, Zomacton, Zometa, Zomig, Zomig-ZMT, Zonalon, Zonegran, Zontivity, Zorbtive, Zortress, Zorvolex, Zosyn, Zovirax, Zovirax Ointment, ZTlido, Zubsolv, Zulresso, Zuplenz, Zyclara, Zydelig, Zyflo, Zyflo CR, Zykadia, Zylet, Zyloprim, Zymar, Zymaxid, Zypitamag, Zyprexa, Zyprexa Relprevv, Zyprexa Zydis, Zytiga, or Zyvox.

In some embodiments of each or any of the above or below mentioned embodiments, the active ingredient is prostaglandin or a prostaglandin analog.

In some embodiments of each or any of the above or below mentioned embodiments, the prostaglandin analog is selected from the group consisting of: bimatoprost, latanoprost, tafluprost, and travoprost.

In some embodiments of each or any of the above or below mentioned embodiments, the protein having hyaluronidase activity is a human recombinant hyaluronidase such as HYLENEX and the active ingredient is a prostaglandin analog selected from the group consisting of: bimatoprost, latanoprost, tafluprost, and travoprost.

The present disclosure provides a composition comprising an amount of a protein having hyaluronidase activity and an amount of an additional active pharmaceutical ingredient (API).

In some embodiments of each or any of the above or below mentioned embodiments, the additional API is used to treat acne, ADHD, AIDS/HIV, allergies, Alzheimer's, angina, anxiety, arthritis, asthma, bipolar disorder, bronchitis, cancer, cholesterol, colds & flu, constipation, COPD, depression, diabetes (Type 1), diabetes (Type 2), diarrhea, eczema, erectile dysfunction, fibromyalgia, gastrointestinal, GERE (Heartburn), gout, hair loss, hayfever, heart disease, hepatitis A, hepatitis B, herpes, hypertension, hypothyroidism, incontinence, IBD (Bowel), insomnia, menopause, mental health, migraine, osteoarthritis, osteoporosis, pain, psoriasis, rheumatoid arthritis, schizophrenia, seizures, sexual health, stroke, swine flu, UTI, or weight loss.

The present disclosure provides a method of treating and/or preventing a disease or disorder, the method comprising administering to a subject in need thereof any of the compositions disclosed herein.

The present disclosure provides a method of treating or preventing a condition such as a cosmetic condition in a subject in need thereof by administering a composition that comprises 4-methylumbelliferone (4-MU) to the peri-orbital region and/or mid-face of the subject. The cosmetic condition may be due to edema (e.g., peri-orbital puffiness due to peri-orbital edema).

The present disclosure provides a method of treating peri-orbital puffiness due to peri-orbital edema in a subject in need thereof by administering a composition that comprises 4-methylumbelliferone (4-MU) to the peri-orbital region of the subject.

In some embodiments of each or any of the above or below mentioned embodiments, the subject has not been treated with a dermal filler 1 month, 2 months, 3 months, 4 months, 5 months, 6 months, 7 months, 8 months, 9 months, 10 months, 11 months, 12 months, 2 years, 3 years, 4 years, or 5 years before the administration of the composition that comprises 4-methylumbelliferone (4-MU).

In some embodiments of each or any of the above or below mentioned embodiments, the 4-methylumbelliferone (4-MU) is administered in a therapeutically effective amount.

The present disclosure provides a method of treating peri-orbital puffiness due to peri-orbital edema in a subject in need thereof by administering a composition that comprises 4-methylumbelliferone (4-MU) to an eye of the subject.

In some embodiments of each or any of the above or below mentioned embodiments, an eye drop that comprises 4-methylumbelliferone (4-MU) is administered to the eye of the subject, including for example directly to the surface of the eye.

In some embodiments of each or any of the above or below mentioned embodiments, the subject scored as Grade 0 is administered a composition that comprises 4-methylumbelliferone (4-MU) to the peri-orbital region of the subject.

In some embodiments of each or any of the above or below mentioned embodiments, the subject scored as Grade 1E, 2E or 3E is administered a composition that comprises 4-methylumbelliferone (4-MU) to the peri-orbital region of the subject, and optionally treated by surgical removal of a portion of one or more of the fat pads to increase the degree of improvement in the peri-orbital puffiness.

The present disclosure also provides a method to assess whether surgical intervention is needed to treat peri-orbital puffiness not due to peri-orbital edema in a subject in need thereof, the method comprising administering (e.g., injecting) a composition comprising 4-methylumbelliferone (4-MU) to the peri-orbital region of the subject and determining that surgical intervention is required to treat the peri-orbital puffiness not due to peri-orbital edema where the administration of the composition comprising 4-methylumbelliferone (4-MU) does not treat (e.g., reduce) the peri-orbital puffiness not due to peri-orbital edema. Such methods may additionally comprise the step of surgically removing a portion of one or more fat pads in the upper and/or lower eyelids of the subject.

The present disclosure also provides a method of treating a cosmetic condition in a subject in need thereof due to (e.g., caused by) edema by administering a composition that comprises 4-methylumbelliferone (4-MU) to a region on the subject's face having the condition (e.g., a peri-orbital region and/or a mid-face region of the subject).

In some embodiments of each or any of the above or below mentioned embodiments, the peri-orbital puffiness due to peri-orbital edema is unrelated to the use of a hyaluronic acid filler in the peri-orbital region.

In some embodiments of each or any of the above or below mentioned embodiments, the one or more peri-orbital fat pads include at least one lower peri-orbital fat pad.

In some embodiments of each or any of the above or below mentioned embodiments, the step of administering comprises applying a patch or a cream to a surface of the skin in the peri-orbital region.

In some embodiments of each or any of the above or below mentioned embodiments, the composition is administered in an amount effective to treat the peri-orbital puffiness due to peri-orbital edema.

The present disclosure provides a method of assessing peri-orbital puffiness due to peri-orbital edema in a subject, the method comprising: a. determining if the subject exhibits pseudoherniation of one or more upper eyelid fat pads and one or more lower eyelid fat pads; b. determining if the subject exhibits edema in one or more of the upper eyelid fat pads and the one or more of the lower eyelid fat pads; c. determining if the subject exhibits edema in the peri-orbital tissue; and d. scoring the peri-orbital puffiness due to peri-orbital edema from the determinations made in steps a. to c.

In some embodiments of each or any of the above or below mentioned embodiments, the peri-orbital puffiness due to peri-orbital edema is unrelated to the use of a hyaluronic acid filler in the peri-orbital region.

In some embodiments of each or any of the above or below mentioned embodiments, the upper eyelid fat pad is an upper middle fat pad and an upper medial fat pad.

In some embodiments of each or any of the above or below mentioned embodiments, the subject is scored as Grade 0 where the subject does not exhibit pseudoherniation of any of the upper or lower eyelid fat pads.

In some embodiments of each or any of the above or below mentioned embodiments, the subject is scored as Grade 0 where the subject exhibits edema of one or more of the upper or lower eyelid fat pads.

In some embodiments of each or any of the above or below mentioned embodiments, the subject is scored as Grade 1 where the subject exhibits pseudoherniation of one of the upper eyelid fat pads and/or one of the lower eyelid fat pads.

In some embodiments of each or any of the above or below mentioned embodiments, the subject is scored as Grade 1E where the subject exhibits pseudoherniation of one of the upper eyelid fat pads and/or one of the lower eyelid fat pads and exhibits edema of the fat pads and/or peri-orbital tissue.

In some embodiments of each or any of the above or below mentioned embodiments, the subject is scored as Grade 2 where the subject exhibits pseudoherniation of two of the upper eyelid and/or two of the lower eyelid fat pads.

In some embodiments of each or any of the above or below mentioned embodiments, the subject is scored as Grade 2E where the subject exhibits pseudoherniation of two of the upper eyelid fat pads and/or two of the lower eyelid fat pads and exhibits edema of the fat pads and/or peri-orbital tissue.

In some embodiments of each or any of the above or below mentioned embodiments, the subject is scored as Grade 3 where the subject exhibits pseudoherniation of three of the lower eyelid fat pads.

In some embodiments of each or any of the above or below mentioned embodiments, the subject is scored as Grade 3E where the subject exhibits pseudoherniation of the three lower eyelid fat pads and exhibits edema of the fat pads and/or peri-orbital tissue.

The present disclosure provides a method of treating a cosmetic condition due to edema in a subject in need thereof, the method comprising administering a composition that comprises 4-methylumbelliferone (4-MU) to the peri-orbital region and/or mid-face of the subject.

In some embodiments of each or any of the above or below mentioned embodiments, the subject has not previously been treated with a dermal filler in the peri-orbital region and/or the mid-face.

The present disclosure provides a method of treating peri-orbital puffiness due to peri-orbital edema in a subject in need thereof, the method comprising administering a composition that comprises 4-methylumbelliferone (4-MU) to an eye of the subject.

The present disclosure provides a method of treating and/or preventing inflammation in a subject in need thereof by administering a composition that comprises a protein having hyaluronidase activity (e.g., a hyaluronidase such as Hylenex, Amphadase, or Vitrase) to a region of the subject having inflammation. Such methods advantageously may be used to treat inflammation and/or inflammation associated with stasis dermatitis.

In some embodiments of each or any of the above or below mentioned embodiments, the inflammation is associated with stasis dermatitis.

In some embodiments of each or any of the above or below mentioned embodiments, the stasis dermatitis is present in a leg, foot, and/or ankle.

In some embodiments of each or any of the above or below mentioned embodiments, the step of administering is performed by one or more injections to the region of the subject having inflammation.

In some embodiments of each or any of the above or below mentioned embodiments, the step of administering comprises applying a patch or a cream to a region of the subject having inflammation.

The present disclosure also provides methods of reducing inflammation in a subject in need thereof by administering a composition that comprises a protein having hyaluronidase activity to the region of the subject having inflammation, wherein the administration of the composition reduces severity of the inflammation.

In some embodiments of each or any of the above or below mentioned embodiments, the severity of the inflammation is reduced by at least one grade.

In some embodiments of each or any of the above or below mentioned embodiments, the severity of the inflammation is reduced by at least two grades.

In some embodiments of each or any of the above or below mentioned embodiments, after the administration of the composition the inflammation is scored as moderate.

In some embodiments of each or any of the above or below mentioned embodiments, after the administration of the composition the inflammation is scored as mild.

In some embodiments of each or any of the above or below mentioned embodiments, after the administration of the composition the inflammation is scored as quiescent.

The present disclosure provides a method of treating fibrosis in a subject in need thereof, the method comprising administering a composition that comprises a protein having hyaluronidase activity to the subject.

The present disclosure provides a composition comprising a protein having hyaluronidase activity and a collagenase and its use in methods for the treatment of upper and/or lower eyelid puffiness/fullness.

In some embodiments of each or any of the above or below mentioned embodiments, the collagenase is a bovine or human recombinant collagenase.

In some embodiments of each or any of the above or below mentioned embodiments, the protein having hyaluronidase activity is present in an amount of about 1 to about 1,000 pg/mL.

In some embodiments of each or any of the above or below mentioned embodiments, the protein having hyaluronidase activity is present in an amount of about 1 to about 1,000 U/mL.

In some embodiments of each or any of the above or below mentioned embodiments, the collagenase is present in an amount of about 1 to about 1,000 pg/mL.

In some embodiments of each or any of the above or below mentioned embodiments, the collagenase is present in an amount of about 1 to about 1,000 U/mL.

The present disclosure also provides a method of treating upper and/or lower eyelid puffiness/fullness in a subject, the method comprising administering a therapeutically effective amount of a composition having a protein with hyaluronidase activity and a collagenase to one or more of the upper and/or lower eyelid fat pads.

In some embodiments, the methods may reduce (or eliminate) the appearance of upper and/or lower eyelid puffiness/fullness.

In some embodiments of each or any of the above or below mentioned embodiments, the composition includes about 1 to about 1,000 Units of the protein having hyaluronidase activity.

In some embodiments of each or any of the above or below mentioned embodiments, the composition includes about 5 to about 15 Units of the protein having hyaluronidase activity.

The present disclosure provides a method for treating and/or preventing fibrosis (e.g., dermal fibrosis) in a subject in need thereof by administering a composition that comprises a protein having hyaluronidase activity (e.g., a hyaluronidase such as Hylenex, Amphadase, or Vitrase) to the subject.

In some embodiments of each or any of the above or below mentioned embodiments, the fibrosis is a fibrotic skin disease such as scleroderma, nephrogenic fibrosing dermopathy, mixed connective tissue disease, scleromyxedema, scleredema, or eosinophilic fasciitis.

In some embodiments of each or any of the above or below mentioned embodiments, the fibrosis is selected from the group consisting of: pulmonary fibrosis, idiopathic pulmonary fibrosis, cirrhosis, endomyocardial fibrosis, mediastinal fibrosis, myelofibrosis, retroperitoneal fibrosis, progressive massive fibrosis, nephrogenic systemic fibrosis, Crohn's Disease intestine, keloid, myocardial infarction, scleroderma/systemic sclerosis, arthrofibrosis, and adhesive capsulitis.

In some embodiments of each or any of the above or below mentioned embodiments, the protein having hyaluronidase activity region is administered to a region of the subject having the fibrosis.

In some embodiments of each or any of the above or below mentioned embodiments, the protein having hyaluronidase activity region is injected into the fibrosis.

In some embodiments of each or any of the above or below mentioned embodiments, the protein having hyaluronidase activity region is applied to the fibrosis.

The present disclosure also provides a method of reducing fibrosis in a subject in need thereof by administering a composition that comprises a protein having hyaluronidase activity to a region of fibrosis on the subject, wherein the administration results in a reduction in a grade of severity of the fibrosis.

In some embodiments of each or any of the above or below mentioned embodiments, the fibrosis prior to treatment with the composition is classified as mild fibrosis, moderate fibrosis, or severe fibrosis.

In some embodiments of each or any of the above or below mentioned embodiments, the fibrosis after treatment with the composition is classified as no fibrosis, mild fibrosis, or moderate fibrosis.

In some embodiments of each or any of the above or below mentioned embodiments, the methods and compositions disclosed herein are used to cover trap door scars in a subject in need thereof. In some embodiments, trap door scars trap fluid.

In some embodiments of each or any of the above or below mentioned embodiments, the methods and compositions disclosed herein are used at surgical sites in a subject in need thereof.

In some embodiments of each or any of the above or below mentioned embodiments, the methods and compositions disclosed herein are used for treating survical wounds in a subject in need thereof.

In some embodiments of each or any of the above or below mentioned embodiments, the methods and compositions disclosed herein are used to decrease the degree of burn classification in a subject in need thereof.

In some embodiments of each or any of the above or below mentioned embodiments, the decrease in the degree of burn classification is from fourth to third.

In some embodiments of each or any of the above or below mentioned embodiments, the decrease in the degree of burn classification is from third to second.

In some embodiments of each or any of the above or below mentioned embodiments, the decrease in the degree of burn classification is from second to first.

In some embodiments of each or any of the above or below mentioned embodiments, the methods and compositions disclosed herein are used to treat and/or reduce scarring from burns in a subject in need thereof.

In some embodiments of each or any of the above or below mentioned embodiments, the methods and compositions disclosed herein are used during interventional cardiology and radiology in a subject in need thereof.

In some embodiments of each or any of the above or below mentioned embodiments, the methods and compositions disclosed herein are used to dissolve fibrosis by limiting the execution of the procedure to access and remove a medical device in a subject in need thereof.

In some embodiments of each or any of the above or below mentioned embodiments, the methods and compositions disclosed herein are used to permit improved access to the target organ, including removal of a foreign object from the body of the subject in need thereof.

In some embodiments of each or any of the above or below mentioned embodiments, the methods and compositions disclosed herein are used during or instead of a fasciotomy to reduce swelling and/or to save one or more limbs.

In some embodiments of each or any of the above or below mentioned embodiments, the methods and compositions disclosed herein do not comprise hyaluronidase.

In some embodiments of each or any of the above or below mentioned embodiments, the methods and compositions disclosed herein comprise elastinase and do not comprise hyaluronidase.

In some embodiments of each or any of the above or below mentioned embodiments, the methods and compositions disclosed herein are used in a subject who is HIV positive and/or has AIDS.

In some embodiments of each or any of the above or below mentioned embodiments, the methods and compositions disclosed herein comprise protease inhibitors that cause fat atrophy in the body of a subject thereof.

In some embodiments of each or any of the above or below mentioned embodiments, the methods and compositions disclosed herein comprising protease inhibitors that cause fat atrophy in the body of a subject thereof are used to decrease fullness around the eyes.

The present disclosure provides a method for of restoring myelination to axons (or neurons) in a subject in need thereof, the method comprising administering a composition that comprises a protein having hyaluronidase activity (e.g., a hyaluronidase such as Hylenex, Amphadase, or Vitrase) to a region of the subject having demyelinated axons (or neurons).

In some embodiments of each or any of the above or below mentioned embodiments, the protein having hyaluronidase activity region is injected into the region of the subject having demyelinated axons.

In some embodiments of each or any of the above or below mentioned embodiments, the protein having hyaluronidase activity region is applied to the region of the subject having demyelinated axons.

The present disclosure also provides methods of treating a disease or disorder characterized by demyelinated neurons in a subject in need thereof by administering a composition that comprises a protein having hyaluronidase activity to a region of the subject having demyelinated neurons.

In some embodiments of each or any of the above or below mentioned embodiments, the disease is a neurodegenerative disease.

In some embodiments of each or any of the above or below mentioned embodiments, the neurodegenerative disease is multiple sclerosis, acute disseminated encephalomyelitis, neuromyelitis optica, transverse myelitis, chronic inflammatory demyelinating polyneuropathy, Guillain-Barré syndrome, or central pontine myelinosis.

The present disclosure provides a method of treating and/or preventing anterior facial fullness, a jowl, and/or a labiomandibular fold in a subject in need thereof by administering a composition that comprises a protein having hyaluronidase activity (e.g., a hyaluronidase such as Hylenex, Amphadase, or Vitrase) to a region of the subject's face having anterior facial fullness, jowl, or labiomandibular fold, respectively. Advantageously, such methods may be used to restore symmetry to a face where one side exhibits more fullness than the other side by administering the protein having hyaluronidase activity to only one side of the face of the subject.

In some embodiments of each or any of the above or below mentioned embodiments, the protein having hyaluronidase activity is administered to both sides of the face of the subject.

In some embodiments of each or any of the above or below mentioned embodiments, the anterior facial fullness is severe fullness.

In some embodiments of each or any of the above or below mentioned embodiments, the anterior facial fullness is moderate fullness.

In some embodiments of each or any of the above or below mentioned embodiments, the anterior facial fullness is mild fullness.

In some embodiments of each or any of the above or below mentioned embodiments, the jowl is a severe jowl.

In some embodiments of each or any of the above or below mentioned embodiments, the jowl is a moderate jowl.

In some embodiments of each or any of the above or below mentioned embodiments, the jowl is a mild jowl.

In some embodiments of each or any of the above or below mentioned embodiments, the labiomandibular fold is a severe labiomandibular fold.

In some embodiments of each or any of the above or below mentioned embodiments, the labiomandibular fold is a moderate labiomandibular fold.

In some embodiments of each or any of the above or below mentioned embodiments, the labiomandibular fold is a mild labiomandibular fold.

In some embodiments of each or any of the above or below mentioned embodiments, the step of administering is performed by one or more injections to the area of skin on the subject having the anterior facial fullness, the jowl, and/or the labiomandibular fold.

In some embodiments of each or any of the above or below mentioned embodiments, the step of administering comprises applying a patch or a cream to the area of skin of on subject having the anterior facial fullness, the jowl, and/or the labiomandibular fold.

In some embodiments of each or any of the above or below mentioned embodiments, the protein having hyaluronidase activity is administered in a therapeutically effective amount to the area of skin having the anterior facial fullness, the jowl, and/or the labiomandibular fold.

The present disclosure provides methods of reducing or improving the appearance of anterior facial fullness in a subject in need thereof, the method comprising administering a composition that comprises a protein having hyaluronidase activity to an area of skin on the subject, wherein the area of skin is along an anterior cheek bordered medially by a nasolabial fold, laterally by a midface groove, superiorly by a nasojugal groove and inferiorly by a jowl, wherein the administration of the composition reduces severity (e.g., improves the appearance) of the anterior facial fullness.

The present disclosure provides methods of reducing or improving the appearance of a jowl in a subject in need thereof, the method comprising administering a composition that comprises a protein having hyaluronidase activity to an area of skin on the subject having the jowl, wherein the administration of the composition reduces severity (e.g., improves the appearance) of the jowl.

The present disclosure provides methods of reducing or improving the appearance of a labiomandibular fold in a subject in need thereof, the method comprising administering a composition that comprises a protein having hyaluronidase activity to an area of skin on the subject having the labiomandibular fold, wherein the administration of the composition reduces severity (e.g., improves) of the labiomandibular fold.

In some embodiments of each or any of the above or below mentioned embodiments, the severity of the anterior facial fullness, the jowl, and/or the labiomandibular fold is reduced by at least one grade on a scale that measures the severity of the anterior facial fullness, the jowl, and/or the labiomandibular fold, respectively.

In some embodiments of each or any of the above or below mentioned embodiments, the severity of the anterior facial fullness, the jowl, and/or the labiomandibular fold is reduced from a severe grade to a moderate grade.

In some embodiments of each or any of the above or below mentioned embodiments, the severity of the anterior facial fullness, the jowl, and/or the labiomandibular fold is reduced from a severe grade to a mild grade.

In some embodiments of each or any of the above or below mentioned embodiments, the severity of the anterior facial fullness, the jowl, and/or the labiomandibular fold is reduced from a moderate grade to a mild grade.

The present disclosure provides a method of treating anterior facial fullness in a subject in need thereof, the method comprising administering a composition that comprises a protein having hyaluronidase activity to an area of skin on the subject, wherein the area of skin is along an anterior cheek bordered medially by a nasolabial fold, laterally by a midface groove, superiorly by a nasojugal groove and inferiorly by a jowl.

In some embodiments of each or any of the above or below mentioned embodiments, the step of administering is performed by one or more injections to the area of skin on the subject.

In some embodiments of each or any of the above or below mentioned embodiments, the step of administering comprises applying a patch or a cream to the area of skin of on subject.

The present disclosure provides a method of treating a jowl in a subject in need thereof, the method comprising administering a composition that comprises a protein having hyaluronidase activity to an area of skin on the subject having the jowl.

In some embodiments of each or any of the above or below mentioned embodiments, the step of administering comprises applying a patch or a cream to the area of skin of on subject having the jowl.

The present disclosure provides a method of treating a labiomandibular fold in a subject in need thereof, the method comprising administering a composition that comprises a protein having hyaluronidase activity to an area of skin on the subject having the labiomandibular fold.

In some embodiments of each or any of the above or below mentioned embodiments, the step of administering is performed by one or more injections to the area of skin on the subject having the labiomandibular fold.

In some embodiments of each or any of the above or below mentioned embodiments, the severity of the anterior facial fullness is reduced by at least one grade.

In some embodiments of each or any of the above or below mentioned embodiments, the severity of the anterior facial fullness is reduced from severe to moderate fullness.

In some embodiments of each or any of the above or below mentioned embodiments, the severity of the anterior facial fullness is reduced from severe to mild fullness.

In some embodiments of each or any of the above or below mentioned embodiments, the severity of the anterior facial fullness is reduced from moderate to mild fullness.

The present disclosure provides formulations for cosmetic and/or non-cosmetic applications that comprise a protein having hyaluronidase activity (e.g., a hyaluronidase such as HYLENEX, Amphadase, or Vitrase) and/or a prostaglandin analog and methods of using same to treat and/or prevent a cosmetic conditions such as peri-orbital puffiness (peri-orbital fullness). For example, the cosmetic condition may be due to edema (e.g., peri-orbital puffiness due to peri-orbital edema).

The present disclosure provides a formulation comprising a protein having hyaluronidase activity, wherein the formulation is free of or substantially free of albumin.

In some embodiments of each or any of the above or below mentioned embodiments, the albumin is human albumin.

In some embodiments of each or any of the above or below mentioned embodiments, the proteins having hyaluronidase activity are hyaluronidases.

In some embodiments of each or any of the above or below mentioned embodiments, the hyaluronidases are recombinant hyaluronidases.

In some embodiments of each or any of the above or below mentioned embodiments, the protein have hyaluronidase activity is selected from one or more of Hyaluronidase 1 (Hyal1), Hyaluronidase 2 (Hyal2), Hyaluronidase 3 (Hyal3), Hyaluronidase 4 (Hyal4), Hyaluronidase 5 (Hyal5), and Hyaluronidase 6 (Hyal1).

In some embodiments of each or any of the above or below mentioned embodiments, the protein having hyaluronidase activity is crosslinked (e.g., between about 10% to about 90% of the protein having hyaluronidase activity comprise inter-protein cross-links).

In some embodiments of each or any of the above or below mentioned embodiments, the protein having hyaluronidase activity is crosslinked with 1,4-butanediol diglycidyl ether (BDDE) or lysine.

In some embodiments of each or any of the above or below mentioned embodiments, the inter-protein cross-links comprise disulfide bonds.

In some embodiments of each or any of the above or below mentioned embodiments, the inter-protein cross-links are formed by covalently linking two or more reactive groups on the proteins using one or more crosslinkers.

In some embodiments of each or any of the above or below mentioned embodiments, the two or more reactive groups on the proteins are selected from lysine residues, aspartic acid residues, glutamic acid residues, and cysteine residues.

In some embodiments of each or any of the above or below mentioned embodiments, the one or more crosslinkers are selected from glutaraldehyde, genipin, methylgloxal, proanthrocyanidin, tannic acid, and 1-ethyl-3-(3-dimethylaminopropyl)-carbodiimide.

In some embodiments of each or any of the above or below mentioned embodiments, the formulation further comprises one or more of an anesthetic agent, a prostaglandin, a prostaglandin analog, and a vasoconstrictor.

In some embodiments of each or any of the above or below mentioned embodiments, the anesthetic agent is lidocaine.

In some embodiments of each or any of the above or below mentioned embodiments, the prostaglandin analog is bimatoprost.

In some embodiments of each or any of the above or below mentioned embodiments, the vasoconstrictor is epinephrine.

In some embodiments of each or any of the above or below mentioned embodiments, the formulation further comprises a surfactant.

In some embodiments of each or any of the above or below mentioned embodiments, the surfactant is polysorbate 80.

In some embodiments of each or any of the above or below mentioned embodiments, the formulation further comprises a buffer.

In some embodiments of each or any of the above or below mentioned embodiments, the buffer is a histidine buffer, a citrate buffer, a gluconate buffer, a succinate buffer, or a phosphate buffer.

In some embodiments of each or any of the above or below mentioned embodiments, the formulation further comprises a stabilizer.

In some embodiments of each or any of the above or below mentioned embodiments, the stabilizer is an amino acid or a saccharide.

The present disclosure also provides a formulation that comprises a prostaglandin analog such as bimatoprost and the use thereof for the treatment of peri-orbital fullness.

The present disclosure also provides methods of treating peri-orbital puffiness in a subject in need thereof, the method comprising: administering a formulation disclosed herein to a peri-orbital region of the subject.

In some embodiments of each or any of the above or below mentioned embodiments, the peri-orbital puffiness is due to peri-orbital edema.

In some embodiments of each or any of the above or below mentioned embodiments, the peri-orbital puffiness is unrelated to a use of a hyaluronic acid filler in the peri-orbital region.

In some embodiments of each or any of the above or below mentioned embodiments, the step of administering is performed by one or more injections into a peri-orbital soft tissue and/or one or more injections into one or more peri-orbital fat pads.

In some embodiments of each or any of the above or below mentioned embodiments, the one or more peri-orbital fat pads include at least one lower peri-orbital fat pad.

In some embodiments of each or any of the above or below mentioned embodiments, the lower peri-orbital fat pad is a lower lateral fat pad, a lower middle fat pad, or a lower medial fat pad.

In some embodiments of each or any of the above or below mentioned embodiments, the one or more peri-orbital fat pads include at least one upper peri-orbital fat pad.

In some embodiments of each or any of the above or below mentioned embodiments, the upper peri-orbital fat pad is an upper middle fat pad or an upper medial fat pad.

In some embodiments of each or any of the above or below mentioned embodiments, each injection into the peri-orbital soft tissue and/or the one or more peri-orbital fat pads includes about 1 to about 1,000 Units of the protein having hyaluronidase activity.

In some embodiments of each or any of the above or below mentioned embodiments, the peri-orbital puffiness is reduced for up to 6 months.

In some embodiments of each or any of the above or below mentioned embodiments, the hyaluronidase is selected from one or more of Hyaluronidase 1 (Hyal1), Hyaluronidase 2 (Hyal2), Hyaluronidase 3 (Hyal3), Hyaluronidase 4 (Hyal4), Hyaluronidase 5 (Hyal5), and Hyaluronidase 6 (Hyal1).

In some embodiments of each or any of the above or below mentioned embodiments, about 10% to about 90% of the protein having hyaluronidase activity comprises inter-protein cross-links.

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIG. 1 shows a patient with eye puffiness before (A) and 24 hours after subcutaneous injections of a total of 40 U of Hylenex to four injection sites including: the medial, central and lateral aspects of each of the lower eyelids and the lateral aspect of each of the upper eyelids (10 U per site) (B).

FIGS. 2A and 2B show a patient seated in the Frankfort horizontal plane with eye puffiness before (Panel A) and 3 months after (Panel B) subcutaneous injections of a total of 40 U of HYLENEX to four injection sites including: the medial, central and lateral aspects of each of the lower eyelids and the lateral aspect of each of the upper eyelids (10 U per site).

FIGS. 3A and 3B show a patient seated in the Frankfort horizontal plane with eye puffiness before (Panel A) and 3 months after (Panel B) subcutaneous injections of a total of 40 U of HYLENEX to four injection sites including: the medial, central and lateral aspects of each of the lower eyelids and the lateral aspect of each of the upper eyelids (10 U per site).

FIGS. 4A and 4B show a patient seated in the Frankfort horizontal plane with eye puffiness before (Panel A) and approximately 1 month after (Panel B) subcutaneous injections of a total of 40 U of HYLENEX to four injection sites including: the medial, central and lateral aspects of each of the lower eyelids and the lateral aspect of each of the upper eyelids (10 U per site).

FIGS. 5A and 5B show a patient seated in the Frankfort horizontal plane with eye puffiness before (Panel A) and 2 months after (Panel B) subcutaneous injections of a total of 40 U of HYLENEX to four injection sites including: the medial, central and lateral aspects of each of the lower eyelids and the lateral aspect of each of the upper eyelids (10 U per site).

FIGS. 6A-6H show a patient seated in the Frankfort horizontal plane with eye puffiness before (Panel A; 08-15-2019) and approximately 1 month after (Panel B; 09-04-2019) subcutaneous injections of a total of 40 U of HYLENEX to four injection sites including: the medial, central and lateral aspects of each of the lower eyelids and the lateral aspect of each of the upper eyelids (10 U per site). Panels C1-1 provide magnified images of the patients' eyes before (Panels C, E, and G) and after (Panels D, F, and H) treatment with HYLENEX. Panels C and D depict a magnified view of the patient's right eye before and after treatment with HYLENEX, respectively, while asked to look straight ahead level to the ground. Panels E and F depict a magnified view of the patient's right eye before and after treatment with HYLENEX, respectively, while asked to look upwards while maintaining her head in the Frankfort horizontal plane. Panels G and H depict a magnified view of the patient's left eye before and after treatment with HYLENEX, respectively, while asked to look upwards while maintaining her head in the Frankfort horizontal plane.

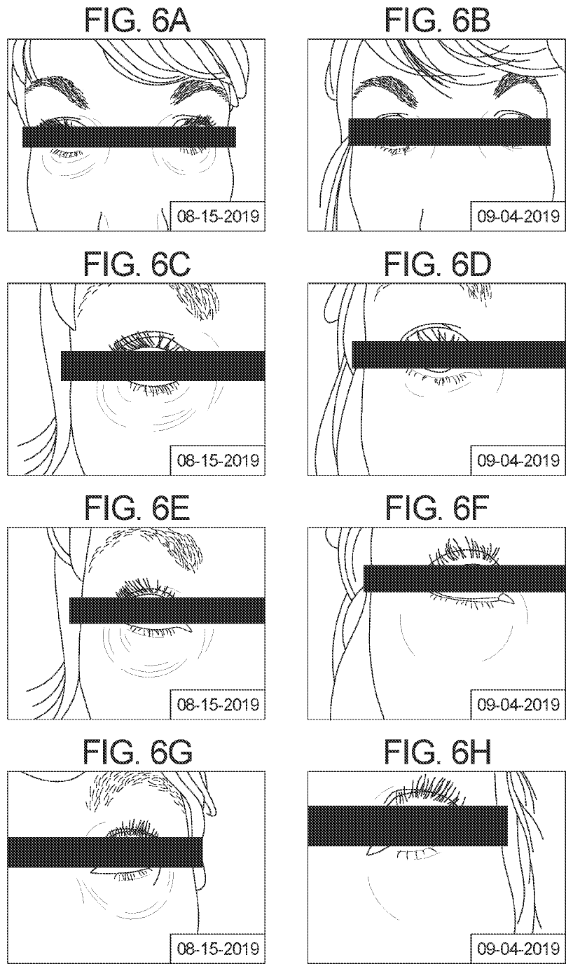

### DETAILED DESCRIPTION

It is estimated that at least 32 million Americans over twenty-five years of age suffer from peri-orbital puffiness or bags around their eyes. This condition is generally attributed to pseudoherniation of the fat pads located around their eyes. Consequently, these patients often undergo a surgical blepharoplasty procedure to lessen their extent of fullness or bags by removing and/or repositioning the fat pads located around the eyes. However, many patients with peri-orbital puffiness or bags do not have pseudoherniation of the fat pads located around their eyes. Instead, the puffiness or bags are due to swelling (edema) of the tissues in that region. Similarly, many suffer from malar puffiness and festoons which are often due to edema of the tissues along the eyelid, tear trough, lid-cheek junction and malar region. These conditions have been very challenging to treat as usually surgery has been used to improve the condition.

The inventors have discovered that a protein having hyaluronidase activity such as a recombinant human hyaluronidase can be used to treat or prevent a condition due to (caused by) edema including, for example, peri-orbital puffiness due to peri-orbital edema particularly where the peri-orbital puffiness is not due to a hyaluronic acid filler (e.g., not due to a prior injection of a hyaluronic acid filler to the peri-orbital region). Advantageously, a protein having hyaluronidase activity may be used to treat peri-orbital puffiness due to peri-orbital edema arising from any number of underlying medical conditions, including allergies, genetic predisposition, sinus problems, hypothyroidism, and other systemic diseases causing swelling around the eyes.

The inventors have discovered that a protein having hyaluronidase activity such as a recombinant human hyaluronidase can be used to treat or prevent a condition due to (caused by) edema including, for example, peri-orbital puffiness, particularly where the peri-orbital puffiness is not due to prior treatment of the peri-orbital region, with a hyaluronic acid filler. Advantageously, a protein having hyaluronidase activity may be used to treat peri-orbital puffiness arising from any number of underlying medical conditions, including allergies, genetic predisposition, sinus problems, hypothyroidism, and other systemic diseases causing swelling around the eyes. Without wishing to be bound by a theory of the invention, it is believed that hyaluronidase acts to break up hyaluronic acid that traps drainage from the eye and/or fluid. In this manner, drainage from the eye and/or fluid may be released and eliminated from the peri-orbital region.

The present disclosure generally relates to the treatment of a condition (e.g., a cosmetic condition) associated with edema such as peri-orbital puffiness or mid-face puffiness (e.g., puffiness of the eyes or “bags” under the eyes, festoons, malar puffiness) using a composition comprising a protein having hyaluronidase activity including, for example, a hyaluronidase such as Hylenex, Amphadase, or Vitrase. Such methods may comprise administering the protein having hyaluronidase activity to the peri-orbital soft tissues and/or edematous orbital fat pads. The methods disclosed herein improve the appearance of the eyes in those patients whose puffiness or “bags” under the eyes and/or puffiness along the upper eyelids are due to edema of the fat pads or soft tissues and not due to protruding (pseudoherniation) of the fat pads normally found around the eye. The methods disclosed herein may also be used for the cosmetic improvement of primary lower eyelid and/or upper eyelid fullness and puffiness, malar puffiness and festoons due to edema, where the primary lower eyelid and/or upper eyelid fullness and puffiness, malar puffiness and festoons are not due to a hyaluronic acid filler (e.g., not due to a prior injection of a hyaluronic acid filler).

The present disclosure also provides a means to classify the etiology of upper and/or lower eyelid puffiness as caused by localized anatomical changes or systemic etiology leading to inflammatory changes that lead to peri-orbital puffiness due to peri-orbital edema. The classification may be used to direct the treatment. If the puffiness is secondary to edema, simply removing some of the affected fat pad will have some initial improvement but will most likely lead to hollowness when the edema in the remaining portion of the fat pad diminishes. When the puffiness is due to the protruding fat pad that has herniated forward in the orbit, then removing part of it is the appropriate course of action. If the etiology of the puffiness is edema of the fat pads or edema of the soft tissue in the eyelids, then treatment should be aimed to decrease the edema in these structures and not to remove part of the fat pads.

The Peri-orbital and Eyelid Fullness Assessment Scale (PEFAS or Sweis-Scale) defines the etiology of the upper and/or lower eyelid fullness to determine the best and safest course of treatment. It determines whether the fullness is secondary to pseudoherniation of the upper and/or lower eyelid fat pads, secondary to edema of the eyelid soft tissues, secondary to edema of the fat pads, or secondary to a combination of pseudoherniation of the fat pads with edema of the fat pads, and/or edema of the peri-orbital soft tissues. The PEFAS score is represented as the findings in the right upper eyelid/the findings in the lower eyelid and the findings in the left upper eyelid/the findings in the lower eyelid.

TABLE 1
Peri-orbital and Eyelid Fullness Assessment Scale (PEFAS)
Peri-orbital and Eyelid Fullness Assessment Scale (PEFAS)

| | |
|---|---|
| Grade 0 | No pseudoherniation of the upper and/or lower eyelid fat |
| | pads and no associated peri-orbital, malar or eyelid |
| | edema |
| Grade 0E | No pseudoherniation of the eyelid fat pads, |
| | and one or more of the following: |
| | i. presence of edema of the eyelid fat pads; |
| | ii. presence of edema along the soft tissue of the |
| | upper or lower eyelids; |
| | iii. presence of malar mounds due to edema; |
| | iv. presence of malar edema; |
| | v. presence of festoons aggravated by edema |
| Grade 1 | Pseudoherniation of only one of the eyelid fat pads without |
| | any associated peri-orbital, malar and/or eyelid edema |
| Grade 1E | Pseudoherniation of only one of the eyelid fat, and one or |
| | more of the following: |
| | i. presence of edema of the fat pad; |
| | ii. presence of edema along the soft tissue of the |
| | upper or lower eyelids; |
| | iii. presence of malar mounds due to edema; |
| | iv. presence of malar edema; |
| | v. presence of festoons aggravated by edema |
| Grade 2 | Pseudoherniation of only two of the eyelid fat pads without |
| | any associated peri-orbital, malar and/or eyelid edema |
| Grade 2E | Pseudoherniation of only two of the eyelid fat pads, and |
| | one or more of the following: |
| | i. presence of edema of one or more of the fat pads; |
| | ii. presence of edema along the soft tissue of the |
| | upper or lower eyelids; |
| | iii. presence of malar mounds due to edema; |
| | iv. presence of malar edema; |
| | v. presence of festoons aggravated by edema |
| Grade 3 | Pseudoherniation of the three lower eyelid fat pads without |
| | any associated peri-orbital, malar and/or eyelid edema |
| Grade 3E | Pseudoherniation of the three lower eyelid fat pads, and |
| | one or more of the following: |
| | i. presence of edema of one or more of the lower fat |
| | pads; |
| | ii. presence of edema along the soft tissue of the |
| | upper or lower eyelids; |
| | iii. presence of malar mounds aggravated by |
| | edema; |
| | iv. presence of malar edema; |
| | v. presence of festoons aggravated by edema |
| Grade 3+ | Pseudoherniation of the three lower eyelid fat pads with |
| | moderate to severe festoons and/or malar mounds |
| | secondary to fat deposition or descent/hypertrophy of the |
| | orbicularis oculi muscle, and without any associated |
| | peri-orbital, malar or eyelid edema |
| Grade 3 + E | Pseudoherniation of the three lower eyelid fat pads with |
| | moderate to severe festoons and/or malar mounds |
| | secondary to fat deposition or descent/hypertrophy of the |
| | orbicularis oculi muscle with one or more of the following: |
| | i. presence of edema of one or more of the fat pads; |
| | ii. presence of edema along the soft tissue of the |
| | upper or lower eyelids; |
| | iii. presence of malar mounds aggravated by |
| | edema; |
| | iv. presence of localized malar edema; |
| | v. presence of festoons aggravated by edema |

Attorney Docket No. 129914-5029-US PATENT

In an embodiment, a subject is scored as Grade 0 where the subject does not exhibit pseudoherniation of any of the upper or lower peri-orbital fat pads. In another embodiment, a subject is scored as Grade 0 where the subject exhibits edema of one or more of the upper or lower peri-orbital fat pads. In yet another embodiment, a subject is scored as Grade 0 where the subject exhibits edema of the peri-orbital tissue. In another embodiment, a subject is scored as Grade 0 where the subject exhibits festoons due to edema. In yet another embodiment, a subject is scored as Grade 0 where the subject exhibits localized malar fullness due to edema.

In another embodiment, a subject is scored as Grade 1 where the subject exhibits pseudoherniation of one fat pad; the upper eyelid medial or central fat pad and/or the lower eyelid medial, central or lateral fat pad. In yet another embodiment, a subject is scored as Grade 1 E where the subject exhibits pseudoherniation of one fat pad; the upper eyelid medial or central fat pad and/or the lower eyelid medial, central or lateral fat pad, and exhibits edema of the fat pads and/or peri-orbital tissue. In another embodiment, a subject is scored as Grade 2 where the subject exhibits pseudoherniation of two fat pads; the upper eyelid medial and central fat pads and/or lower eyelid medial and central, or medial and lateral, or central and lateral fat pads. In yet another embodiment, a subject is scored as Grade 2E where the subject exhibits pseudoherniation of two fat pads; the upper eyelid medial and central fat pads and/or lower eyelid medial and central, or medial and lateral, or central and lateral fat pads and exhibits edema of the fat pads and/or peri-orbital tissue.

In yet another embodiment, a subject is scored as Grade 3 where the subject exhibits pseudoherniation of the three lower eyelid medial, central and lateral fat pads. In another embodiment, a subject is scored as Grade 3E where the subject exhibits pseudoherniation of the lower eyelid medial, central and lateral fat pads, and exhibits edema of the fat pads and/or peri-orbital tissue.

A subject scored as Grade 0 may be administered a composition that comprises a protein having hyaluronidase activity to the peri-orbital region of the subject. A subject scored as Grade 1, Grade 2 or Grade 3 may be treated by surgical removal of a portion of one or more of the fat pads. The subject scored as Grade 1E, 2E or 3E may be administered a composition that comprises a protein having hyaluronidase activity to the peri-orbital region of the subject, and optionally treated by surgical removal of a portion of one or more of the fat pads.

As used herein, “hyaluronidase” refers to an enzyme that degrades hyaluronic acid. Hyaluronidases include bacterial hyaluronidases (EC 4.2.99.1), hyaluronidases from leeches, spiders, snakes, parasites, and crustaceans (EC 3.2.1.36), and mammalian-type hyaluronidases (EC 3.2.1.35). Hyaluronidases also include any of non-human origin including, but not limited to, murine, canine, feline, leporine, avian, bovine, ovine, porcine, equine, piscine, ranine, bacterial, and any from leeches, other parasites, and crustaceans. Hyaluronidases also include those of human origin. Also included amongst hyaluronidases are soluble hyaluronidases. Hyaluronidases include Hyaluronidase 1 (Hyal1), Hyaluronidase 2 (Hyal2), Hyaluronidase 3 (Hyal3), Hyaluronidase 4 (Hyal4), Hyaluronidase 5 (Hyal5), and Hyaluronidase 6 (Hyal1).

Reference to hyaluronidases includes precursor hyaluronidase polypeptides and mature hyaluronidase polypeptides (such as those in which a signal sequence has been removed), truncated forms thereof that have activity, and includes allelic variants and species variants, variants encoded by splice variants, and other variants. Hyaluronidases also include those that contain chemical or posttranslational modifications and those that do not contain chemical or posttranslational modifications. Such modifications include, but are not limited to, pegylation, albumination, glycosylation, farnesylation, carboxylation, hydroxylation, phosphorylation, and other polypeptide modifications known in the art.

As used herein, a soluble hyaluronidase refers to a polypeptide characterized by its solubility under physiologic conditions. Soluble hyaluronidases can be distinguished, for example, by its partitioning into the aqueous phase of a Triton X-114 solution warmed to 37° C. (Bordier et al., (1981) J. Biol. Chem., 256:1604-7). Membrane-anchored, such as lipid anchored hyaluronidases, will partition into the detergent rich phase, but will partition into the detergent-poor or aqueous phase following treatment with Phospholipase-C. Included among soluble hyaluronidases are membrane anchored hyaluronidases in which one or more regions associated with anchoring of the hyaluronidase to the membrane has been removed or modified, where the soluble form retains hyaluronidase activity. Soluble hyaluronidases include recombinant soluble hyaluronidases and those contained in or purified from natural sources, such as, for example, testes extracts from sheep or cows.

As used herein, “hyaluronidase activity” refers to the ability of a protein to cleave hyaluronic acid. In vitro assays to determine the hyaluronidase activity of hyaluronidases are known in the art and described herein. Exemplary assays include the microturbidity assay that measures cleavage of hyaluronic acid by hyaluronidase indirectly by detecting the insoluble precipitate formed when the uncleaved hyaluronic acid binds with serum albumin.

The terms, “treating” or “treatment” of a disease, disorder, or condition includes at least partially: (1) preventing the disease, disorder, or condition, i.e. causing the clinical symptoms of the disease, disorder, or condition not to develop in a mammal that is exposed to or predisposed to the disease, disorder, or condition but does not yet experience or display symptoms of the disease, disorder, or condition; (2) inhibiting the disease, disorder, or condition, i.e., arresting or reducing the development of the disease, disorder, or condition or its clinical symptoms; or (3) relieving the disease, disorder, or condition, i.e., causing regression of the disease, disorder, or condition or its clinical symptoms. The term “treating,” includes to reducing any detectable amount or eliminating in an individual puffiness. In some embodiments, puffiness may be reduced at least about 20%, at least about 30%, at least about 40%, at least about 50%, at least about 60%, at least about 70%, at least about 80%, at least about 90% or at least about 100%.

The terms “prevention”, “prevent”, “preventing”, “suppression”, “suppress”, “suppressing”, “inhibit” and “inhibition” as used herein refer to a course of action initiated in a manner so as to prevent, suppress or reduce, either temporarily or permanently, the onset of a clinical manifestation of the disease state or condition. Such preventing, suppressing or reducing need not be absolute to be useful.

As used herein, the term “peri-orbital puffiness” also known as swelling or fullness around the eyes is the appearance of swelling in the tissues around the eyes, called the orbits. It may be caused by fluid buildup around the eyes, or peri-orbital edema including edema in the peri-orbital fat pads and soft tissues. Peri-orbital puffiness may also be due to swelling or fullness of the malar region.

As used herein, the term “puffiness” or “fullness” also known as swelling or fullness around the eyes, is the appearance of swelling in the tissues around the eyes, called the orbits. It is almost exclusively caused by fluid buildup around the eyes, or peri-orbital edema.

The terms “improvement” or “improving” as used herein in reference to peri-orbital puffiness refers to a reduction in peri-orbital puffiness.

The term “reducing” as used herein refers to a lowering in the amount, mass, or volume of puffiness. Such reduction can be measured and determined by measuring the amount of puffiness according to one or more of the methods described herein at an initial time point prior to the administering of the compounds described herein and then measuring the amount of puffiness at various time points (e.g. during the period of administering the compounds described herein as well after the administering has ceased). For example, a subject's puffiness can be measured prior to beginning a treatment regimen with the compounds described herein and then measured during and after the treatment regimen. A decrease in puffiness is indicative of a reduction in puffiness. Additionally, the reduction of puffiness can be determined qualitatively such as by photographing the face or eyes, at various time points before, during, and after a treatment regimen where the reduction in puffiness can be determined by visual inspection of the images. A reduction in puffiness includes, for example, a 30%, 35%, 40%, 45%, 50%, 55%, 60%, 65%, 70%, 75%, 80%, 85%, 90%, 95%, 100%, or greater lowering or decrease in the amount, mass, and/or volume of peri-orbital puffiness. Alternatively, a subject's puffiness is given a score of 10 (on a scale of 1 to 10 with 1 being no peri-orbital puffiness) prior to treatment with the composition disclosed herein. The subject and/or medical practitioner that administered the composition then scores the puffiness after treatment with a subjective grade based on the original score of 10 (e.g., the score is given based on a visual assessment of the subject from a before photograph of the peri-orbital puffiness). A decrease in puffiness measured as a score of 6 or less indicates a reduction in peri-orbital puffiness. Alternatively, a reduction in puffiness may be determined by a reduction in in a PEFAS grade (e.g., a score of L 0/3E [left eye; upper eyelid over lower eyelid] and a R 0/3E [right eye; upper eyelid over lower eyelid] to a score of L 0/1E and L 0/1E). A reduction in PEFAS grade corresponds to a reduction in the Grade number (e.g., a 3 to a 2).

The terms “partially improve” or partial improvement” as used herein in reference to peri-orbital puffiness refers to a small (or minor) reduction in peri-orbital puffiness such as a 5%, 10%, 15%, 20%, or 25% lowering or decrease in the amount, mass, and/or volume of puffiness. Alternatively, a subject's puffiness is given a score of 10 (on a scale of 1 to 10 with 1 being no peri-orbital puffiness) prior to treatment with the composition disclosed herein. The subject and/or medical practitioner that administered the composition then scores the puffiness after treatment with a subjective grade based on the original score of 10 (e.g., the score is given based on a visual assessment of the subject from a before photograph of the peri-orbital puffiness). A decrease in puffiness measured as a score of 7 to 9 indicates a partial improvement in peri-orbital puffiness.

The terms “worsens” or “worse” as used herein in reference to peri-orbital puffiness refer to an increase in peri-orbital puffiness.

The terms “increased” or “increase” as used herein in reference to peri-orbital puffiness refers to an increase in the amount, mass, and/or volume of peri-orbital puffiness. Such increase can be measured and determined by measuring the amount of puffiness according to one or more of the methods described herein including, for example, at an initial time point prior to the administering of the compounds described herein and then measuring the amount of puffiness at various time points (e.g. during the period of administering the compounds described herein as well after the administering has ceased). For example, a subject's puffiness can be measured prior to beginning a treatment regimen with the compounds described herein and then measured during and after the treatment regimen. Additionally, the increase in puffiness can be determined qualitatively such as by photographing the face or eyes including, for example, at various time points before, during, and after a treatment regimen where the reduction in puffiness can be determined by visual inspection of the images. An increase in puffiness incudes, for example, a 5%, 10%, 15%, 20%, 25%, 30%, 35%, 40%, 45%, 50%, 55%, 60%, 65%, 70%, 75%, 80%, 85% 90%, 95%, 100, or increase in the amount, mass, and/or volume of peri-orbital puffiness. Alternatively, an increase in puffiness may be determined by an increase in a PEFAS grade (e.g., a score of L 0/1E [left eye; upper eyelid over lower eyelid] and a R 0/1E [right eye; upper eyelid over lower eyelid] to a score of L 0/3E and L 0/3E). An increase in PEFAS grade corresponds to an increase in the Grade number (e.g., a 1 to a 3).

As used herein, the term “subject” refers to an animal, including a mammal, such as a human being.

As used herein, a “patient” refers to a human subject.

As used herein, amelioration of the symptoms by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, prevention or prophylaxis refers to methods in which the risk of developing disease or condition is reduced.

As used herein, a “therapeutically effective amount” or a “therapeutically effective dose” refers to the quantity of an agent, compound, material, or composition containing a compound that is at least sufficient to produce a therapeutic effect. Hence, it is the quantity necessary for preventing, curing, ameliorating, arresting or partially arresting a symptom of a disease, disorder, or condition.

As used herein, the singular forms “a,” “an” and “the” include plural referents unless the context clearly dictates otherwise. Thus, for example, reference to a compound, comprising “an extracellular domain” includes compounds with one or a plurality of extracellular domains.

As used herein, ranges and amounts can be expressed as “about” a particular value or range. About also includes the exact amount. Hence “about 5 bases” means “about 5 bases” and also “5 bases.”

As used herein, the term “pharmaceutical formulation” refers to a preparation which is in such form as to permit the biological activity of the active ingredient to be effective, and which contains no additional components which are unacceptably toxic to a subject to which the formulation would be administered. Such formulations are sterile.

As used herein, the term “sterile” refers to a formulation that is aseptic or free from all living microorganisms and their spores.

As used herein, the term “stable” refers to a formulation that is one in which all the proteins therein essentially retain their physical stability and/or chemical stability and/or biological activity upon storage at the intended storage temperature, e.g., 2-8° C. Preferably, the formulation essentially retains its physical and chemical stability, as well as its biological activity upon storage. The storage period is generally selected based on the intended shelf-life of the formulation. Furthermore, the formulation is preferably stable following freezing (to, e.g., −20° C.) and thawing of the formulation, for example following 1 or more cycles of freezing and thawing. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography or capillary zone electrophoresis; SDS-PAGE analysis to compare reduced and intact antibody; evaluating biological activity or antigen binding function of the antibody; etc. Instability may involve any one or more of: aggregation, deamidation (e.g. Asn deamidation), oxidation (e.g. Met oxidation), isomerization (e.g. Asp isomeriation), clipping/hydrolysis/fragmentation (e.g. hinge region fragmentation), succinimide formation, unpaired cysteine(s), etc.

Therapeutic formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed.

As used herein, the term “surfactant” refers to a pharmaceutically acceptable surface-active agent. In the formulation of the invention, the amount of surfactant is described a percentage expressed in weight/volume. The most commonly used weight/volume unit is mg/mL. Suitable examples of pharmaceutically acceptable surfactants include polyoxyethylen-sorbitan fatty acid esters (Tween), polyethylene-polypropylene glycols, polyoxyethylene-stearates, polyoxyethylene alkyl ethers, e.g. polyoxyethylene monolauryl ether, alkylphenylpolyoxy-ethylene ethers (Triton-X), polyoxyethylene-polyoxypropylene copolymer (Poloxamer, Pluronic), and sodium dodecyl sulphate (SDS). Most suitable polyoxyethylenesorbitan-fatty acid esters are polysorbate 20, (sold under the trademark Tween 20™) and polysorbate 80 (sold under the trademark Tween 80™). Most suitable polyethylene-polypropylene copolymers are those sold under the names Pluronic® F68 or Poloxamer 188™. Preferred polyoxyethylene-stearates are those sold under the trademark Myrj™. Most suitable polyoxy-ethylene alkyl ethers are those sold under the trademark Brij™ Most suitable alkylphenolpoly-oxyethylene ethers are sold under the trade name Triton-X.

As used herein, the term “buffer” refers to a pharmaceutically acceptable buffer. As used herein the term “buffering agent providing a pH of 5.5±2.0” refers to an agent which provides that the solution comprising it resists changes in pH by the action of its acid/base conjugate components. Suitable pharmaceutically acceptable buffers according to the invention comprise but are not limited to histidine-buffers, citrate-buffers, gluconate-buffers, succinate-buffers, acetate-buffers glycylglycine and other organic acid buffers, and phosphate-buffers. Preferred buffers comprise L-histidine or mixtures of L-histidine with L-histidine hydrochloride with isotonicity agents and potentially pH adjustment with an acid or a base known in the art. Most preferred is L-histidine.

As used herein, the term “isotonic” refers to a formulation that has essentially the same osmotic pressure as human blood. Isotonic formulations will generally have an osmolality of ^{˜}300 mOsm/kg. Isotonicity can be measured using a vapor pressure or freezing-point depression type osmometer.

As used herein, the term “isotonicity agents” refers to pharmaceutically acceptable isotonicity agents. Isotonicity agents are used to provide an isotonic formulation. An isotonic formulation is liquid or liquid reconstituted from a solid form, e.g. a lyophilized form and denotes a solution having the same tonicity as some other solution with which it is compared, such as physiologic salt solution and the blood serum. Suitable isotonicity agents comprise but are not limited to salts, including but not limited to sodium chloride (NaCl) or potassium chloride, sugars and sugar alcohols including but not limited to glucose, sucrose, trehalose or glycerol and any component from the group of amino acids, sugars, salts and combinations thereof. Isotonicity agents are generally used in a total amount of about 5 mM to about 350 mM.

As used herein, the term “liquid” refers to a formulation which is liquid at a temperature of at least about 2 to about 8° C.

As used herein, the term “lyophilized” refers to a formulation which is dried by freezing the formulation and subsequently subliming the ice from the frozen content by any freeze-drying methods known in the art, for example commercially available freeze-drying devices.

As used herein, the term “salts” refers to a salt in an amount of about 1 mM to about 500 mM. Non-limiting examples of salts include salts of any combinations of the cations sodium potassium, calcium or magnesium with anions chloride, phosphate, citrate, succinate, sulphate or mixtures thereof.

As used herein, the term “amino acid” refers to an amino acid in an amount of about 1 to about 100 mg/mL comprising but not limited to arginine, glycine, ornithine, glutamine, asparagine, lysine, histidine, glutamic acid, asparagic acid, isoleucine, leucine, alanine, phenylalanine, tyrosine, tryptophane, methionine, serine, proline.

As used herein, the term “saccharide” refers to the general composition (CH2O)n and derivatives thereof, including monosaccharides, disaccharides, trisaccharides, polysaccharides, sugar alcohols, reducing sugars, nonreducing sugars, etc. Examples of saccharides herein include glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol, iso-maltulose, etc. Also included in the definition according to the invention are glucosamine, N-Methylglucosamine (so-called “Meglumine”), galactosamine and neuraminic acid and combinations of the saccharides according to the invention. The preferred saccharide herein is a non-reducing disaccharide, such as trehalose or sucrose. The most preferred saccharide in accordance with the present invention is trehalose.

As used herein, the term “stabilizer” refers to pharmaceutically acceptable stabilizers, like for example but not limited to amino acids and sugars as described in the above sections as well as commercially available dextrans of any kind and molecular weight as known in the art.

As used herein, the term “antioxidant” refers to a pharmaceutically acceptable antioxidant. This may include excipients such as methionine, benzylalcohol or any other excipient used to minimize oxidation.

### Hyaluronidase

Hyaluronidases are a family of enzymes that degrade hyaluronic acid. There are three general classes of hyaluronidases; mammalian hyaluronidase, bacterial hyaluronidase and hyaluronidase from leeches, other parasites and crustaceans. Mammalian-type hyaluronidases (EC 3.2.1.35) are endo-β-N-acetyl-hexosaminidases that hydrolyze the β1→4 glycosidic bond of hyaluronan into various oligosaccharide lengths such as tetrasaccharides and hexasaccharides. They have both hydrolytic and transglycosidase activities, and can degrade hyaluronan and chondroitin sulfates (CS), generally C4-S and C6-S. Hyaluronidases of this type include, but are not limited to, hyaluronidases from cows (bovine), mouse, pig, rat, rabbit, sheep (ovine), orangutan, cynomolgus monkey, guinea pig, and human hyaluronidases.

Mammalian hyaluronidases can be further subdivided into those that are neutral active, predominantly found in testes extracts, and acid active, predominantly found in organs such as the liver. Exemplary neutral active hyaluronidases include PH20. Human PH20 (also known as SPAM1 or sperm surface protein PH20), is generally locked to the plasma membrane via a glycosylphosphatidyl inositol (GPI) anchor. It is naturally involved in sperm-egg adhesion and aids penetration by sperm of the layer of cumulus cells by digesting hyaluronic acid. Alignment of bovine PH20 with the human PH20 shows only weak homology, with multiple gaps existing from amino acid 470 through to the respective carboxy termini due to the absence of a GPI anchor in the bovine polypeptide (see e.g., Frost GI (2007) Expert Opin. Drug. Deliv. 4: 427-440). In fact, no clear GPI anchor is predicted in any other PH20 species besides humans. Thus, PH20 polypeptides produced from ovine and bovine exist as soluble forms. Though bovine PH20 exists very loosely attached to the plasma membrane, it is not anchored via a phospholipase sensitive anchor (Lalancette et al, Biol Reprod. 2001 August; 65(2):628-36.). This unique feature of bovine hyaluronidase has permitted the use of the soluble bovine testes hyaluronidase enzyme as an extract for clinical use (Wydase™, Hyalase™)

Besides human PH20 (also termed SPAM1), five hyaluronidase-like genes have been identified in the human genome, HYAL1, HYAL2, HYAL3, HYAL4 and HYALP1. HYALP1 is a pseudogene, and HYAL3 has not been shown to possess enzyme activity toward any known substrates. The hyaluronidase-like enzymes can also be characterized by those which are generally locked to the plasma membrane via a glycosylphosphatidyl inositol anchor such as human HYAL2 and human PH20 (Danilkovitch-Miagkova, et al. (2003) Proc Natl Acad Sci USA. 100(8):4580-5), and those which are generally soluble such as human HYAL1 (Frost et al, (1997) Biochem Biophys Res Commun. 236(1):10-5).

In a preferred embodiment, the hyaluronidase is HYLENEX (having the amino acid sequence as set forth in SEQ ID NO: 1).

Glycosylation, including N- and O-linked glycosylation, of some hyaluronidases can be very important for their catalytic activity and stability. While altering the type of glycan modifying a glycoprotein can have dramatic affects on a protein's antigenicity, structural folding, solubility, and stability, most enzymes are not thought to require glycosylation for optimal enzyme activity. Such hyaluronidases are unique in this regard, in that removal of N-linked glycosylation can result in near complete inactivation of the hyaluronidase activity. For such hyaluronidases, the presence of N-linked glycans is critical for generating an active enzyme.

N-linked oligosaccharides fall into several major types (oligomannose, complex, hybrid, sulfated), all of which have (Man) 3-GlcNAc-GlcNAc-cores attached via the amide nitrogen of Asn residues that fall within-Asn-Xaa-Thr/Ser-sequences (where Xaa is not Pro). Glycosylation at an-Asn-Xaa-Cys-site has been reported for coagulation protein C. In some instances, the hyaluronidase can contain both N-glycosidic and 0-glycosidic linkages.

Soluble hyaluronidases include any that exist in soluble form, including, but not limited to, Hyal1, bovine PH20 and ovine PH20, allelic variants thereof and other variants. Also included among soluble hyaluronidase are any hyaluronidase that has been modified to be soluble. For example, human PH20, which is normally membrane anchored via a GPI anchor, can be made soluble by truncation of and removal of all or a portion of the GPI anchor at the C-terminus. Soluble hyaluronidases also include neutral active and acid active hyaluronidases, however, neutral active hyaluronidases are contemplated for use herein for purposes of subcutaneous administration.

Polypeptides of a soluble hyaluronidase set forth herein, can be obtained by methods well known in the art for protein purification and recombinant protein expression. Any method known to those of skill in the art for identification of nucleic acids that encode desired genes can be used. Any method available in the art can be used to obtain a full length (i.e., encompassing the entire coding region) cDNA or genomic DNA clone encoding a hyaluronidase, such as from a cell or tissue source. Modified or variant soluble hyaluronidases, can be engineered from a wildtype polypeptide, such as by site-directed mutagenesis.

Polypeptides can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

Methods for amplification of nucleic acids can be used to isolate nucleic acid molecules encoding a desired polypeptide, including for example, polymerase chain reaction (PCR) methods. A nucleic acid containing material can be used as a starting material from which a desired polypeptide-encoding nucleic acid molecule can be isolated. For example, DNA and mRNA preparations, cell extracts, tissue extracts, fluid samples (e.g. blood, serum, saliva), samples from healthy and/or diseased subjects can be used in amplification methods. Nucleic acid libraries also can be used as a source of starting material. Primers can be designed to amplify a desired polypeptide. For example, primers can be designed based on expressed sequences from which a desired polypeptide is generated. Primers can be designed based on back-translation of a polypeptide amino acid sequence. Nucleic acid molecules generated by amplification can be sequenced and confirmed to encode a desired polypeptide.

Additional nucleotide sequences can be joined to a polypeptide-encoding nucleic acid molecule, including linker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to a polypeptide-encoding nucleic acid molecule. Examples of such sequences include, but are not limited to, promoter sequences designed to facilitate intracellular protein expression, and secretion sequences, for example heterologous signal sequences, designed to facilitate protein secretion. Such sequences are known to those of skill in the art. Additional nucleotide residues sequences such as sequences of bases specifying protein binding regions also can be linked to enzyme-encoding nucleic acid molecules. Such regions include, but are not limited to, sequences of residues that facilitate or encode proteins that facilitate uptake of an enzyme into specific target cells, or otherwise alter pharmacokinetics of a product of a synthetic gene. For example, enzymes can be linked to PEG moieties.

In addition, tags or other moieties can be added, for example, to aid in detection or affinity purification of the polypeptide. For example, additional nucleotide residues sequences such as sequences of bases specifying an epitope tag or other detectable marker also can be linked to enzyme-encoding nucleic acid molecules. Exemplary of such sequences include nucleic acid sequences encoding a His tag (e.g., 6×His) or Flag Tag.

The identified and isolated nucleic acids can then be inserted into an appropriate cloning vector. A large number of vector-host systems known in the art can be used. Possible vectors include, but are not limited to, plasmids or modified viruses, but the vector system must be compatible with the host cell used. Such vectors include, but are not limited to, bacteriophages such as lambda derivatives, or plasm ids such as pCMV4, pBR322 or pUC plasmid derivatives or the Bluescript vector (Stratagene, La Jolla, Calif.).

Other expression vectors include the HZ24 expression vector exemplified herein. The insertion into a cloning vector can, for example, be accomplished by ligating the DNA fragment into a cloning vector which has complementary cohesive termini. Insertion can be effected using TOPO cloning vectors (INVITROGEN, Carlsbad, Calif.). If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized oligonucleotides encoding restriction endonuclease recognition sequences. In an alternative method, the cleaved vector and protein gene can be modified by homopolymeric tailing. Recombinant molecules can be introduced into host cells via, for example, transformation, transfection, infection, electroporation and sonoporation, so that many copies of the gene sequence are generated.

In specific embodiments, transformation of host cells with recombinant DNA molecules that incorporate the isolated protein gene, cDNA, or synthesized DNA sequence enables generation of multiple copies of the gene. Thus, the gene can be obtained in large quantities by growing transformants, isolating the recombinant DNA molecules from the transformants and, when necessary, retrieving the inserted gene from the isolated recombinant DNA.

For recombinant expression of one or more of the desired proteins, such as any described herein, the nucleic acid containing all or a portion of the nucleotide sequence encoding the protein can be inserted into an appropriate expression vector, i.e., a vector that contains the necessary elements for the transcription and translation of the inserted protein coding sequence. The necessary transcriptional and translational signals also can be supplied by the native promoter for enzyme genes, and/or their flanking regions.

Also provided are vectors that contain a nucleic acid encoding the enzyme. Cells containing the vectors also are provided. The cells include eukaryotic and prokaryotic cells, and the vectors are any suitable for use therein.

Prokaryotic and eukaryotic cells, including endothelial cells, containing the vectors are provided. Such cells include bacterial cells, yeast cells, fungal cells, Archea, plant cells, insect cells and animal cells. The cells are used to produce a protein thereof by growing the above-described cells under conditions whereby the encoded protein is expressed by the cell, and recovering the expressed protein. For purposes herein, for example, the enzyme can be secreted into the medium.

Also provided are vectors that contain a sequence of nucleotides that encodes the soluble hyaluronidase polypeptide coupled to the native or heterologous signal sequence, as well as multiple copies thereof. The vectors can be selected for expression of the enzyme protein in the cell or such that the enzyme protein is expressed as a secreted protein.

A variety of host-vector systems can be used to express the protein coding sequence. These include but are not limited to mammalian cell systems infected with virus (e.g. vaccinia virus, adenovirus and other viruses); insect cell systems infected with virus (e.g. baculovirus); microorganisms such as yeast containing yeast vectors; or bacteria transformed with bacteriophage, DNA, plasm id DNA, or cosmid DNA. The expression elements of vectors vary in their strengths and specificities. Depending on the host-vector system used, any one of a number of suitable transcription and translation elements can be used.

Any methods known to those of skill in the art for the insertion of DNA fragments into a vector can be used to construct expression vectors containing a chimeric gene containing appropriate transcriptional/translational control signals and protein coding sequences. These methods can include in vitro recombinant DNA and synthetic techniques and in vivo recombinants (genetic recombination). Expression of nucleic acid sequences encoding protein, or domains, derivatives, fragments or homologs thereof, can be regulated by a second nucleic acid sequence so that the genes or fragments thereof are expressed in a host transformed with the recombinant DNA molecule(s). For example, expression of the proteins can be controlled by any promoter/enhancer known in the art. In a specific embodiment, the promoter is not native to the genes for a desired protein. Promoters which can be used include but are not limited to the SV40 early promoter (Bernoist and Chambon, Nature 290:304-310 (1981)), the promoter contained in the 3′ long terminal repeat of Rous sarcoma virus (Yamamoto et al. Cell 22:787-797 (1980)), the herpes thymidine kinase promoter (Wagner et al., Proc. Natl. Acad. Sci. USA 78:1441-1445 (1981)), the regulatory sequences of the metallothionein gene (Brinster et al., Nature 296:39-42 (1982)); prokaryotic expression vectors such as the β-lactamase promoter (Jay et al., (1981) Proc. Natl. Acad. Sci. USA 78:5543) or the tac promoter (DeBoer et al., Proc. Natl. Acad. Sci. USA 80:21-25 (1983)); see also “Useful Proteins from Recombinant Bacteria”: in Scientific American 242:79-94 (1980)); plant expression vectors containing the nopaline synthetase promoter (Herrara-Estrella et al., Nature 303:209-213 (1984)) or the cauliflower mosaic virus 35S RNA promoter (Garder et al., Nucleic Acids Res. 9:2871 (1981)), and the promoter of the photosynthetic enzyme ribulose bisphosphate carboxylase (Herrera-Estrella et al., Nature 310:115-120 (1984)); promoter elements from yeast and other fungi such as the Ga14 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 38:639-646 (1984); Ornitz et al., Cold Spring Harbor Symp. Quant. Biol. 50:399-409 (1986); MacDonald, Hepatology 7:425-515 (1987)); insulin gene control region which is active in pancreatic beta cells (Hanahan et al., Nature 315:115-122 (1985)), immunoglobulin gene control region which is active in lymphoid cells (Grosschedl et al., Cell 38:647-658 (1984); Adams et al., Nature 318:533-538 (1985); Alexander et al., Mol. Cell Biol. 7:1436-1444 (1987)), mouse mammary tumor virus control region which is active in testicular, breast, lymphoid and mast cells (Leder et al., Cell 45:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-1648 (1985); Hammer et al., Science 235:53-58 1987)), alpha-1 antitrypsin gene control region which is active in liver (Kelsey et al., Genes and Devel. 1:161-171 (1987)), beta globin gene control region which is active in myeloid cells (Magram et al., Nature 315:338-340 (1985); Kollias et al., Cell 46:89-94 (1986)), myelin basic protein gene control region which is active in oligodendrocyte cells of the brain (Readhead et al., Cell 48:703-712 (1987)), myosin light chain-2 gene control region which is active in skeletal muscle (Shani, Nature 314:283-286 (1985)), and gonadotrophic releasing hormone gene control region which is active in gonadotrophs of the hypothalamus (Mason et al., Science 234:1372-1378 (1986)).

In a specific embodiment, a vector is used that contains a promoter operably linked to nucleic acids encoding a desired protein, or a domain, fragment, derivative or homolog, thereof, one or more origins of replication, and optionally, one or more selectable markers (e.g., an antibiotic resistance gene). Exemplary plasmid vectors for transformation of E. coli cells, include, for example, the pQE expression vectors (available from Qiagen, Valencia, Calif.; see also literature published by Qiagen describing the system). pQE vectors have a phage T5 promoter (recognized by E. coli RNA polymerase) and a double lac operator repression module to provide tightly regulated, high-level expression of recombinant proteins in E. coli, a synthetic ribosomal binding site (RBS II) for efficient translation, a 6×His tag coding sequence, t0 and T1 transcriptional terminators, ColE1 origin of replication, and a beta-lactamase gene for conferring ampicillin resistance. The pQE vectors enable placement of a 6×His tag at either the N- or C-terminus of the recombinant protein. Such plasm ids include pQE 32, pQE 30, and pQE 31 which provide multiple cloning sites for all three reading frames and provide for the expression of N-terminally 6×His-tagged proteins. Other exemplary plasmid vectors for transformation of E. coli cells, include, for example, the pET expression vectors (see, U.S. Pat. No. 4,952,496; available from NOVAGEN, Madison, Wis.; see, also literature published by Novagen describing the system). Such plasmids include pET 11 a, which contains the T7lac promoter, T7 terminator, the inducible E. coli lac operator, and the lac repressor gene; pET 12a-c, which contains the T7 promoter, T7 terminator, and the E. coli ompT secretion signal; and pET 15b and pET19b (NOVAGEN, Madison, Wis.), which contain a His-Tag™ leader sequence for use in purification with a His column and a thrombin cleavage site that permits cleavage following purification over the column, the T7-lac promoter region and the T7 terminator.

Soluble hyaluronidase polypeptides can be produced by any method known to those of skill in the art including in vivo and in vitro methods. Desired proteins can be expressed in any organism suitable to produce the required amounts and forms of the proteins, such as for example, needed for administration and treatment. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

Many expression vectors are available and known to those of skill in the art and can be used for expression of proteins. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

Soluble hyaluronidase polypeptides also can be utilized or expressed as protein fusions. For example, an enzyme fusion can be generated to add additional functionality to an enzyme. Examples of enzyme fusion proteins include, but are not limited to, fusions of a signal sequence, a tag such as for localization, e.g. a his6 tag or a myc tag, or a tag for purification, for example, a GST fusion, and a sequence for directing protein secretion and/or membrane association.

Prokaryotes, especially E. coli, provide a system for producing large amounts of proteins. Transformation of E. coli is simple and rapid technique well known to those of skill in the art. Expression vectors for E. coli can contain inducible promoters, such promoters are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated APL promoter.

Proteins, such as any provided herein, can be expressed in the cytoplasmic environment of E. coli. The cytoplasm is a reducing environment and for some molecules, this can result in the formation of insoluble inclusion bodies. Reducing agents such as dithiothreitol and p-mercaptoethanol and denaturants, such as guanidine-HCl and urea can be used to resolubilize the proteins. An alternative approach is the expression of proteins in the periplasmic space of bacteria which provides an oxidizing environment and chaperonin-like and disulfide isomerases and can lead to the production of soluble protein. Typically, a leader sequence is fused to the protein to be expressed which directs the protein to the periplasm. The leader is then removed by signal peptidases inside the periplasm. Examples of periplasmic-targeting leader sequences include the pelB leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Typically, bacteria produce aglycosylated proteins. Thus, if proteins require glycosylation for function, glycosylation can be added in vitro after purification from host cells.

Yeasts such as Saccharomyces cerevisae, Schizosaccharomyces pombe, Yarrowia lipolytica, Kluyveromyces lactis and Pichia pastoris are well known yeast expression hosts that can be used for production of proteins, such as any described herein. Yeast can be transformed with episomal replicating vectors or by stable chromosomal integration by homologous recombination. Typically, inducible promoters are used to regulate gene expression. Examples of such promoters include GAL1, GALT and GALS and metallothionein promoters, such as CUP1, AOX1 or other Pichia or other yeast promoter. Expression vectors often include a selectable marker such as LEU2, TRP1, HIS3 and URA3 for selection and maintenance of the transformed DNA. Proteins expressed in yeast are often soluble. Co-expression with chaperonins such as Bip and protein disulfide isomerase can improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site such as for the Kex-2 protease, can be engineered to remove the fused sequences from the expressed polypeptides as they exit the secretion pathway. Yeast also is capable of glycosylation at Asn-X-Ser/Thr motifs.

Insect cells, particularly using baculovirus expression, are useful for expressing polypeptides such as hyaluronidase polypeptides. Insect cells express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter of baculovirus. Commonly used baculovirus systems include the baculoviruses such as Autographa californica nuclear polyhedrosis virus (AcNPV), and the Bombyx mori nuclear polyhedrosis virus (BmNPV) and an insect cell line such as Sf9 derived from Spodoptera frugiperda, Pseudaletia unipuncta (A7S) and Danaus plexippus (DpN1). For high-level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schneider 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

Mammalian expression systems can be used to express proteins including soluble hyaluronidase polypeptides. Expression constructs can be transferred to mammalian cells by viral infection such as adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. IRES elements also can be added to permit bicistronic expression with another gene, such as a selectable marker. Such vectors often include transcriptional promoter-enhancers for high-level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression.

Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase (DHFR) and thymidine kinase. For example, expression can be performed in the presence of methotrexate to select for only those cells expressing the DHFR gene. Fusion with cell surface signaling molecules such as TCR-ζ and FcϵRI-γ can direct expression of the proteins in an active state on the cell surface.

Many cell lines are available for mammalian expression including mouse, rat human, monkey, chicken and hamster cells. Exemplary cell lines include but are not limited to CHO, Balb/3T3, HeLa, MT2, mouse NSO (nonsecreting) and other myeloma cell lines, hybridoma and heterohybridoma cell lines, lymphocytes, fibroblasts, Sp2/0, COS, NIH3T3, HEK293, 293S, 2B8, and HKB cells. Cell lines also are available adapted to serum-free media which facilitates purification of secreted proteins from the cell culture media. Examples include CHO-S cells (Invitrogen, Carlsbad, Calif., cat #11619-012) and the serum free EBNA-1 cell line (Pham et al., (2003) Biotechnol. Bioeng. 84:332-42.). Cell lines also are available that are adapted to grow in special mediums optimized for maximal expression. For example, DG44 CHO cells are adapted to grow in suspension culture in a chemically defined, animal product-free medium.

Method for purification of polypeptides, including soluble hyaluronidase polypeptides or other proteins, from host cells will depend on the chosen host cells and expression systems. For secreted molecules, proteins are generally purified from the culture media after removing the cells. For intracellular expression, cells can be lysed and the proteins purified from the extract. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary, the proteins can be extracted and further purified using standard methods in the art.

Proteins, such as soluble hyaluronidase polypeptides, can be purified using standard protein purification techniques known in the art including but not limited to, SDS-PAGE, size fraction and size exclusion chromatography, ammonium sulfate precipitation and ionic exchange chromatography, such as anion exchange. Affinity purification techniques also can be utilized to improve the efficiency and purity of the preparations. For example, antibodies, receptors and other molecules that bind hyaluronidase enzymes can be used in affinity purification. Expression constructs also can be engineered to add an affinity tag to a protein such as a myc epitope, GST fusion or His6 and affinity purified with myc antibody, glutathione resin and Ni-resin, respectively. Purity can be assessed by any method known in the art including gel electrophoresis and staining and spectrophotometric techniques.

Hyaluronidase activity can be assessed using methods well known in the art. In one example, activity is measured using a microturbidity assay. This is based on the formation of an insoluble precipitate when hyaluronic acid binds with serum albumin. The activity is measured by incubating hyaluronidase with sodium hyaluronate (hyaluronic acid) for a set period of time (e.g. 10 minutes) and then precipitating the undigested sodium hyaluronate with the addition of acidified serum albumin. The turbidity of the resulting sample is measured at 640 nm after an additional development period. The decrease in turbidity resulting from hyaluronidase activity on the sodium hyaluronate substrate is a measure of hyaluronidase enzymatic activity. In another example, hyaluronidase activity is measured using a microtiter assay in which residual biotinylated hyaluronic acid is measured following incubation with hyaluronidase (see e.g. Frost and Stern (1997) Anal. Biochem. 251:263-269, U.S. Patent Publication No. 20050260186). The free carboxyl groups on the glucuronic acid residues of hyaluronic acid are biotinylated, and the biotinylated hyaluronic acid substrate is covalently couple to a microtiter plate. Following incubation with hyaluronidase, the residual biotinylated hyaluronic acid substrate is detected using an avidin-peroxidase reaction, and compared to that obtained following reaction with hyaluronidase standards of known activity. Other assays to measure hyaluronidase activity also are known in the art and can be used in the methods herein (see e.g. Delpech et al., (1995) Anal. Biochem. 229:35-41; Takahashi et al., (2003) Anal. Biochem. 322:257-263).

Crosslinking is known in the art to have effects on the mechanical properties of drug release. Thus, when certain types of crosslinkers are used, the bioavailability of drugs, such as proteins, can be modulated upon administration of a formulation of a drug to a patient. Thus, in embodiments herein, between about 1% to about 100% (e.g., 5%, 10%, 15%, 20%, 25%, 30%, 35%, 40%, 45%, 50%, 55%, 60%, 65%, 70%, 75%, 80%, 85%, 90%, 95%, or 100%) of the proteins having hyaluronidase activity in the formulations disclosed herein are crosslinked (e.g., have inter-protein cross-links). In some embodiments, between about 10% to about 20% of the proteins comprise inter-protein cross-links. In some embodiments, between about 20% to about 30% of the proteins comprise inter-protein cross-links. In some embodiments, between about 30% to about 40% of the proteins comprise inter-protein cross-links. In some embodiments, between about 40% to about 50% of the proteins comprise inter-protein cross-links. In some embodiments, between about 50% to about 60% of the proteins comprise inter-protein cross-links. In some embodiments, between about 60% to about 70% of the proteins comprise inter-protein cross-links. In some embodiments, between about 70% to about 80% of the proteins comprise inter-protein cross-links. In some embodiments, between about 80% to about 90% of the proteins comprise inter-protein cross-links.

In embodiments, the proteins having hyaluronidase activity are crosslinked with BDDE or lysine.

In embodiments, the inter-protein cross-links comprise disulfide bonds. In some embodiments, the inter-protein cross-links are formed by covalently linking two or more reactive groups on the proteins using one or more crosslinkers. In embodiments, the two or more reactive groups on the proteins are selected from lysine residues, aspartic acid residues, glutamic acid residues, and cysteine residues. In some embodiments, the two or more reactive groups are lysine residues. In some embodiments, the two or more reactive groups are aspartic acid residues. In some embodiments, the two or more reactive groups are glutamic acid residues. In some embodiments, the two or more reactive groups are cysteine residues.

In embodiments, the one or more crosslinkers are selected from glutaraldehyde, genipin, methylgloxal, proanthrocyanidin, tannic acid, and 1-ethyl-3-(3-dimethylaminopropyl)-carbodiimide. In some embodiments, the one or more crosslinkers are glutaraldehyde. In some embodiments, the one or more crosslinkers are genipin. In some embodiments, the one or more crosslinkers are methylgloxal. In some embodiments, the one or more crosslinkers are proanthrocyanidin. In some embodiments, the one or more crosslinkers are tannic acid. In some embodiments, the one or more crosslinkers are 1-ethyl-3-(3-dimethylaminopropyl)-carbodiimide. In some embodiments, the crosslinker are chosen in order to modulate the release time of un-crosslinked hyaluronidases upon administration of the formulation to the patient.

In some embodiments, the ratio of un-crosslinked hyaluronidases to crosslinked hyaluronidase is optimized for effectiveness and/or time release of the formulation. For example, a formulation that includes crosslinked hyaluronidase may release the hyaluronidase over 1 week, 2 weeks, 3 weeks, 4 weeks, 2 months, 3 months, 4 months, 5 months, 6 months, 7 months, 8 months, 9 months, 10 months, 11 months, 12 months or longer. In some embodiments, the ratio of un-crosslinked hyaluronidases to crosslinked hyaluronidase is optimized for slow release of bioavailable hyaluronidase to the patient. For example, the formulation may comprise 5% uncrosslinked hyaluronidase and 95% crosslinked hyaluronidase; 10% uncrosslinked hyaluronidase and 90% crosslinked hyaluronidase; 15% uncrosslinked hyaluronidase and 85% crosslinked hyaluronidase; 20% uncrosslinked hyaluronidase and 80% crosslinked hyaluronidase; 25% uncrosslinked hyaluronidase and 75% crosslinked hyaluronidase; 30% uncrosslinked hyaluronidase and 70% crosslinked hyaluronidase; 35% uncrosslinked hyaluronidase and 65% crosslinked hyaluronidase; 40% uncrosslinked hyaluronidase and 60% crosslinked hyaluronidase; 45% uncrosslinked hyaluronidase and 55% crosslinked hyaluronidase; 50% uncrosslinked hyaluronidase and 50% crosslinked hyaluronidase; 55% uncrosslinked hyaluronidase and 45% crosslinked hyaluronidase; 60% uncrosslinked hyaluronidase and 40% crosslinked hyaluronidase; 65% uncrosslinked hyaluronidase and 35% crosslinked hyaluronidase; 70% uncrosslinked hyaluronidase and 30% crosslinked hyaluronidase; 75% uncrosslinked hyaluronidase and 25% crosslinked hyaluronidase; 80% uncrosslinked hyaluronidase and 20% crosslinked hyaluronidase; 85% uncrosslinked hyaluronidase and 15% crosslinked hyaluronidase; 90% uncrosslinked hyaluronidase and 10% crosslinked hyaluronidase; or 95% uncrosslinked hyaluronidase and 5% crosslinked hyaluronidase.

### Collagenase

One area of particular concern to those wishing to retain a youthful appearance is the occurrence of upper/lower eyelid fullness or puffiness. It is estimated that at least 32 million Americans over twenty-five years of age suffer from peri-orbital puffiness or bags around their eyes. Consequently, these patients often undergo a surgical blepharoplasty procedure to lessen their extent of fullness or bags by removing and/or repositioning the fat pads located around the eyes. However, many patients with peri-orbital puffiness or bags do not have pseudoherniation of the fat pads located around their eyes. Instead, the puffiness or bags are due to swelling (edema) of the tissues in that region. The inventors have surprisingly discovered that a composition that comprises both a protein having hyaluronidase activity and a collagenase is able to reduce or eliminate the appearance of upper/lower eyelid fullness or puffiness.

The present disclosure provides a method for treating upper/lower eyelid puffiness or fullness in a subject by administering a therapeutically effective amount of a composition having a protein with hyaluronidase activity (e.g., human recombinant hyaluronidase) and a collagenase to one or more upper and/or lower eyelid fat pads. Also provided herein are compositions that comprise a protein with hyaluronidase activity (e.g., human recombinant hyaluronidase) and a collagenase.

Collagenase for use according to the disclosure may be obtained from any convenient source, including mammalian (e.g., human, porcine), crustacean (e.g., crab, shrimp), fungal, and bacterial (e.g., from the fermentation of Clostridium, Streptomyces, Pseudomonas, Vibrio or Achromobacter iophagus). Collagenase can be isolated from a natural source or can be genetically engineered/recombinant. One common source of crude collagenase is from a bacterial fermentation process, specifically the fermentation of Clostridium histolyticum. The crude collagenase obtained from C. histolyticum can be purified using any of a number of techniques known in the art of protein purification, including chromatographic techniques. Collagenase compositions useful for the invention also can be prepared using any commercially available or isolated collagenase activity, or by mixing such activities. For example, purified collagenase can be provided by Biospecifics Technologies, Lynbrook, N.Y.

Preferred collagenases for use in the invention are from C. histolyticum, i.e., collagenase class I and class II. A practical advantage of using C. histolyticum for the production of collagenases is that it can be cultured in large quantities in simple liquid media, and it regularly produces amounts of proteolytic enzymes which are secreted into the culture medium. Bovine products have been used in culture media in the fermentation of C. histolyticum, but these run the risk of contamination by agents which cause transmissible spongiform encephalopathies (TSEs; e.g., prions associated with bovine spongiform encephalopathy or “mad cow disease”). Therefore, it is preferred to avoid such bovine products. An animal-product-free system is preferred. The H4 strain of Clostridium histolyticum, originally developed in 1956 can serve as a source for cells for culture. This strain, and a strain derived from the H4 strain, named the ABC Clostridium histolyticum master cell bank (deposited as ATCC 21000) were developed using animal products, but are suitable to use in the invention.

U.S. Pat. No. 7,811,560, which is incorporated herein by reference in its entirety, discloses methods of producing collagenases. Using soybean derived fermentation medium, the methods described therein generated separately highly purified collagenase I and II. This patent also discloses methods of producing highly purified collagenases using culture media containing porcine-derived products. Any of these methods are suitable for use with the invention. U.S. Patent Publication 2010/0086971, which is also incorporated herein by reference in its entirety, discloses numerous fermentation recipes which are based on vegetable peptone, including soybean-derived peptone, or vegetable-derived peptone plus fish gelatin. The methods described in this publication are suitable to produce growth of Clostridium and collagenase activities. These methods also are suitable and contemplated for use with the invention, however any method known in the art of producing collagenase enzyme activity may be used.

The collagenase contemplated for use with the invention can be any collagenase which is active under the necessary conditions. However, preferred compositions contain a mass ratio of collagenase I and collagenase II which is modified or optimized to produce a desired or even a maximal synergistic effect. Preferably, collagenase I and collagenase II are purified separately from the crude collagenase mixture produced in culture, and the collagenase I and collagenase II are recombined in an optimized fixed mass ratio. Preferred embodiments contain a collagenase I to collagenase II mass ratio of about 0.5 to 1.5, more preferably 0.6 to 1.3, even more preferably 0.8 to 1.2, and most preferably, 1 to 1, however any combination or any single collagenase activity may be used.

Both collagenase I and collagenase II are metalloproteases and require tightly bound zinc and loosely bound calcium for their. Both collagenases have broad specificity toward all types of collagen. Collagenase I and Collagenase II digest collagen by hydrolyzing the triple-helical region of collagen under physiological conditions. Each collagenase shows different specificity (e.g. each have a different preferred target amino sequence for cleavage), and together they have synergistic activity toward collagen. Collagenase II has a higher activity towards all kinds of synthetic peptide substrates than collagenase I as reported for class II and class I collagenase in the literatures.

The preferred collagenase consists of two microbial collagenases, referred to as Collagenase ABC I and Collagenase ABC II. The terms “Collagenase I”, “ABC I”, and “collagenase ABC I” mean the same and can be used interchangeably. Similarly, the terms “Collagenase II”, “ABC II”, and “collagenase ABC II” refer to the same enzyme and can also be used interchangeably. These collagenases are secreted by bacterial cells. Preferably, they are isolated and purified from Clostridium histolyticum culture supernatant by chromatographic methods. Both collagenases are special proteases and share the same EC number (E.C 3.4.24.3). However, a collagenase or a combination of collagenases from other sources are contemplated for use with the invention. Collagenase ABC I has a single polypeptide chain consisting of approximately 1000 amino acids with a molecular weight of 115 kDa. Collagenase ABC II has also a single polypeptide chain consisting of about 1000 amino acids with a molecular weight of 110 kDa.

Collagenase acts by hydrolyzing the peptide bond between Gly-Pro-X, wherein X is often proline or hydroxyproline. Collagenase I acts at loci at ends of triple-helical domains, whereas Collagenase II cleaves internally. Hydrolysis continues over time until all bonds are cleaved.

Preferably, the collagenase product is at least 95% pure collagenase(s) and is substantially free of any contaminating proteases. More preferably, the collagenase product is 97% pure and most preferably 98% pure or more as determined by one or more of the following: sodium dodecyl sulfate polyacrylamide gel electrophoresis (SDS-PAGE); high performance liquid chromatography (HPLC); reverse-phase HPLC; or by enzymatic assays. The preferred collagenase product is essentially clostripain-free, and the purification preferably is performed in the absence of leupeptin.

One form of purified collagenase used for injection includes two microbial collagenases, referred to as “Collagenase ABC I” and “Collagenase ABC II”. Both collagenases are isolated and purified from the fermentation of the bacterium Clostridium histolyticum and belong to the same metalloprotease.

Collagenase ABC I is a single polypeptide chain consisting of approximately 1000 amino acids of known sequence. It has an observed molecular weight of 115 kiloDalton (kD), an isoelectric point (pi) between 5.63-5.68, and an extinction coefficient of 1.480. From its activity behavior toward synthetic substrate, it has been determined that Collagenase ABC I is class I Clostridium histolyticum collagenase. Collagenase ABC II is also a single polypeptide chain consisting of about 1000 amino acids of deduced sequence. It has an observed molecular weight of 110 kD, an isoelectric point between 5.46-5.57 and an extinction coefficient of 1.576. Collagenase ABC II functionally belongs to the class II Clostridium histolyticum collagenase.

A preferred collagenase composition includes a mixture of collagenase I and collagenase II in a mass ratio of about one to one and having specific activity from about 500 SRC units/mg to about 15,000 SRC units/mg, preferably of at least about 700 SRC units/mg, more preferably of at least about 1000 SRC units/mg, more preferably at least about 1500 SRC units/mg. One SRC unit will solubilize rat tail collagen into ninhydrin reaction material equivalent to 1 nanomole of leucine per minute, at 25 degrees C., pH 7.4. Collagenase has been described in ABC units as well, with 10,000 ABC of approximately 0.58 mg. The potency assay of collagenase is based on the digestion of undenatured collagen (from bovine tendon) at pH 7.2 and 37 degrees C. for 20-24 hours. The number of peptide bonds cleaved are measured by reaction with ninhydrin. Amino groups released by a solubilized digestion control are subtracted. One net ABC unit of collagenase will solubilize ninhydrin reactive material equivalent to 1.09 nanomoles of leucine per minute. One SRC unit equals approximately 6.3 ABC units.

Commercially available sources of collagenase are useful in the methods of this invention. For example, purified collagenase contains minimal secondary proteolytic activities, but with high collagenase activity. Purified collagenase can be collagenase H (Cat #1 087 789) from Boerhinger Mannheim (Indianapolis, Ind.). A stock solution of 0.5 mg/ml collagenase is prepared in DPBS containing 0.1% BSA, and stored-20 C. Other commercially available sources are Dispase (Boehringer Mannheim), Liberase (Boehringer Mannheim) or collagenase (Serva). The range of collagenase used can be from 100-1000 pg/ml (18-180 mU/ml), preferably between 300-700 pg/ml, (54-126 mU/ml) most preferably about 500 pg/ml (90 mU/ml).

The collagenase compositions according this invention are designed to administer to a patient in need thereof a therapeutically effective amount of a collagenase composition as described, or a therapeutically effective amount of a pharmaceutical collagenase formulation as described. A “therapeutically effective amount” of a compound, composition or formulation is an amount of the compound which confers a therapeutic effect on the treated subject, at a reasonable benefit/risk ratio applicable to any medical treatment. A therapeutic effect includes but is not limited to a shrinkage or reduction in the size of one or more uterine fibroids (including elimination of the fibroid), liquification, partial liquification, or reduction in stiffness (increase in softness) or pressure in or around a uterine fibroid, a change in viscoelastic properties, or reduction in symptoms such as pain, hemorrhage and the like.

Polypeptides of a soluble hyaluronidase or collagenase set forth herein, can be obtained by methods well known in the art for protein purification and recombinant protein expression. Any method known to those of skill in the art for identification of nucleic acids that encode desired genes can be used. Any method available in the art can be used to obtain a full length (i.e., encompassing the entire coding region) cDNA or genomic DNA clone encoding a hyaluronidase, such as from a cell or tissue source. Modified or variant soluble hyaluronidases, can be engineered from a wildtype polypeptide, such as by site-directed mutagenesis.

4-methylumbelliferone (4-MU)

The inventors have discovered that 4-methylumbelliferone (4-MU) can be used to treat or prevent a condition due to (caused by) edema including, for example, peri-orbital puffiness due to peri-orbital edema particularly where the peri-orbital puffiness is not due to a hyaluronic acid filler (e.g., not due to a prior injection of a hyaluronic acid filler to the peri-orbital region). Advantageously, 4-methylumbelliferone (4-MU) may be used to treat peri-orbital puffiness due to peri-orbital edema arising from any number of underlying medical conditions, including allergies, genetic predisposition, sinus problems, hypothyroidism, and other systemic diseases causing swelling around the eyes.

The present disclosure generally relates to the treatment of a condition (e.g., a cosmetic condition) associated with edema such as peri-orbital puffiness or mid-face puffiness (e.g., puffiness of the eyes or “bags” under the eyes, festoons, malar puffiness) using a composition comprising 4-methylumbelliferone (4-MU). Such methods may comprise administering 4-methylumbelliferone (4-MU) to the peri-orbital soft tissues and/or edematous orbital fat pads. The methods disclosed herein improve the appearance of the eyes in those patients whose puffiness or “bags” under the eyes and/or puffiness along the upper eyelids are due to edema of the fat pads or soft tissues and not due to protruding (pseudoherniation) of the fat pads normally found around the eye. The methods disclosed herein may also be used for the cosmetic improvement of primary lower eyelid and/or upper eyelid fullness and puffiness, malar puffiness and festoons due to edema, where the primary lower eyelid and/or upper eyelid fullness and puffiness, malar puffiness and festoons are not due to a hyaluronic acid filler (e.g., not due to a prior injection of a hyaluronic acid filler).

A subject scored as Grade 0 may be administered a composition that comprises 4-methylumbelliferone (4-MU) to the peri-orbital region of the subject. A subject scored as Grade 1, Grade 2 or Grade 3 may be treated by surgical removal of a portion of one or more of the fat pads. The subject scored as Grade 1E, 2E or 3E may be administered a composition that comprises 4-methylumbelliferone (4-MU) to the peri-orbital region of the subject, and optionally treated by surgical removal of a portion of one or more of the fat pads.

As used herein, “4-methylumbelliferone (4-MU)” refers to a chemical having a ChEBI ID of 17224, PubChem CID: 5280567, and/or the structure provided below:

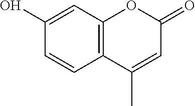

The present disclosure provides methods of administering 4-methylumbelliferone (4-MU) to a subject in need thereof to treat or prevent a condition (e.g., a cosmetic condition) due to (caused by) peri-orbital or mid-face edema. Such conditions may include peri-ocular puffiness, festoons, and malar puffiness. The compositions can be formulated in lyophilized or liquid form. Where the compositions are provided in lyophilized form they can be reconstituted just prior to use by an appropriate buffer, for example, a sterile saline solution. The compositions can be provided together or separately. The compositions can be packaged as a kit.

Also provided are methods of treating peri-orbital puffiness due to peri-orbital edema in a subject in need thereof by administering a composition that comprises 4-methylumbelliferone (4-MU) to an eye of the subject. In an embodiment, an eye drop that comprises 4-methylumbelliferone (4-MU) is administered to the eye of the subject, including for example directly to the surface of the eye.

Provided herein are method of treating or preventing a condition such as a cosmetic condition in a subject in need thereof by administering a composition that comprises 4-methylumbelliferone (4-MU) to a region of a subject's face having said cosmetic condition (e.g., to the peri-orbital region and/or mid-face of the subject). The cosmetic condition may be due to edema (e.g., peri-orbital puffiness due to peri-orbital edema).

Also provided are methods of treating or preventing peri-ocular puffiness in a subject in need thereof by administering a composition that comprises 4-methylumbelliferone (4-MU) to a region of a subject's face having said peri-ocular puffiness (e.g., to the peri-orbital region and/or mid-face of the subject). The peri-ocular puffiness may be due to edema (e.g., peri-orbital puffiness due to peri-orbital edema).

The disclosure also provides methods of treating or preventing festoons in a subject in need thereof by administering a composition that comprises 4-methylumbelliferone (4-MU) to a region of a subject's face having said festoons. The festoons may be due to edema.

Additionally, provided herein are methods of treating or preventing malar puffiness in a subject in need thereof by administering a composition that comprises 4-methylumbelliferone (4-MU) to a region of a subject's face having malar puffiness. The malar puffiness may be due to edema.

Classifying and/or Diagnosing the Etiology of Peri-Orbital Puffiness

The present disclosure also provides methods to classify the etiology of upper and/or lower eyelid puffiness as caused by localized anatomical changes or systemic etiology leading to inflammatory changes that lead to peri-orbital puffiness due to peri-orbital edema. The classification may be used to direct treatment of peri-orbital puffiness. If the puffiness is secondary to edema, simply removing some of the affected peri-orbital fat pad(s) will have some initial improvement but will most likely lead to hollowness when the edema in the remaining portion of the fat pad(s) diminishes. When the puffiness is due to the protruding fat pad(s) that has herniated forward in the orbit, then removing part of it is the appropriate course of action. If the etiology of the puffiness is edema of the fat pad(s) or edema of the soft tissue in the eyelids, then treatment should be aimed to decrease the edema in these structures and not to remove part of the fat pad(s).

The Peri-orbital and Eyelid Fullness Assessment Scale (PEFAS) defines the etiology of the upper eyelid fullness, lower eyelid fullness, festoons and malar edema to determine the best and safest course of treatment. It determines whether the fullness is secondary to pseudoherniation of the upper and/or lower eyelid fat pads, secondary to edema of the eyelid soft tissues, secondary to edema of the fat pads, or secondary to a combination of pseudoherniation of the fat pads with edema of the fat pads, and/or edema of the peri-orbital soft tissues. The PEFAS score is represented as the findings in the right upper eyelid/the findings in the lower eyelid and the findings in the left upper eyelid/the findings in the lower eyelid. For example, a patient with pseudoherniation of the medial upper eyelid fat pads bilaterally without any edema of the upper eyelids, edema of the right lower eyelid region without pseudoherniation of the fat pads, and edema with pseudoherniation of the 3 left lower eyelid fat pads would have an PEFAS score of R: 1/0E and L: 1/3E. Thus, the PEFAS score provides a listing the number of herniated fat pads first (1, 2, or 3), anatomical reasons for festoons and malar fullness, if present, (+), and presence of edema last (E).

In an embodiment, a subject is scored as Grade 0 where the subject does not exhibit pseudoherniation of the upper or lower eyelid fat pads and does not exhibit any associated peri-orbital, malar or eyelid edema. In a further embodiment, a subject is scored as Grade 0E where the subject does not exhibit pseudoherniation of any of the upper or lower peri-orbital fat pads but does exhibit one or more of the following: presence of edema of the upper/lower fat pads, presence of edema along the soft tissue of the upper and/or lower eyelids, presence of malar mounds due to edema, presence of malar edema, and presence of mild festoons aggravated by edema.

In another embodiment, a subject is scored as Grade 1 where the subject exhibits pseudoherniation of only one of the eyelid fat pads (e.g., the upper eyelid medial or central fat pad and/or the lower eyelid medial, central or lateral fat pad) without any associated peri-orbital, malar and/or eyelid edema.

In yet another embodiment, a subject is scored as Grade 1 E where the subject exhibits pseudoherniation of one of the eyelid fat pads (e.g., the upper eyelid medial or central fat pad and/or the lower eyelid medial, central or lateral fat pad), and one or more of the following: presence of edema of the fat pad, presence of edema along the soft tissue of the upper and/or lower eyelids, presence of malar mounds due to edema, presence of malar edema, and presence of mild festoons aggravated by edema.

In another embodiment, a subject is scored as Grade 2 where the subject exhibits pseudoherniation of only two eyelid fat pads (e.g., the upper eyelid medial or central fat pad and/or the lower eyelid medial, central or lateral fat pad) without any associated peri-orbital, malar and/or eyelid edema.

In yet another embodiment, a subject is scored as Grade 2E where the subject exhibits pseudoherniation of only two eyelid fat pads (e.g., the upper eyelid medial or central fat pad and/or the lower eyelid medial, central or lateral fat pad), and one or more of the following: presence of edema of one or more of the fat pads, presence of edema along the soft tissue of the upper and/or lower eyelids, presence of malar mounds due to edema, presence of malar edema, and presence of mild festoons aggravated by edema.

In yet another embodiment, a subject is scored as Grade 3 where the subject exhibits pseudoherniation of the three lower eyelid fat pads (e.g., lower eyelid medial, central or lateral fat pads) without any associated peri-orbital, malar and/or eyelid edema.

In another embodiment, a subject is scored as Grade 3E where the subject exhibits pseudoherniation of the three lower eyelid fat pads (e.g., lower eyelid medial, central or lateral fat pads), and one or more of the following: presence of edema of one or more of the fat pads, presence of edema along the soft tissue of the upper and/or lower eyelids, presence of malar mounds due to edema, presence of malar edema, and presence of mild festoons aggravated by edema.

In another embodiment, a subject is scored as Grade 3+ where the subject exhibits pseudoherniation of the three lower eyelid fat pads with moderate to severe festoons and/or malar mounds secondary to fat deposition or descent/hypertrophy of the orbicularis oculi muscle, and without any associated peri-orbital, malar or eyelid edema.

In another embodiment, a subject is scored as Grade 3+E where the subject exhibits pseudoherniation of the three lower eyelid fat pads with moderate to severe festoons and/or malar mounds secondary to fat deposition or descent/hypertrophy of the orbicularis oculi muscle with one or more of the following: presence of edema of one or more of the fat pads, presence of edema along the soft tissue of the upper and/or lower eyelids, presence of malar mounds aggravated by edema, presence of localized malar edema, and presence of festoons aggravated by edema.

Malar mounds or festoons are aggravated by edema in a subject where they are more pronounced (larger in surface area and/or volume) due to thyroid disease, allergies, medications, nephrotic syndrome, inflammatory disorders, dietary sensitives (e.g., alcohol, salt, gluten, dairy, or processed foods). Additionally or alternatively, malar mounds or festoons may be aggravated by edema depending on the time of day such as morning versus evening. In some embodiments, the festoons are mild festoons.

A subject scored as Grade 0 may be administered a composition that comprises a protein having hyaluronidase activity to the peri-orbital region of the subject. A subject scored as Grade 1, Grade 2 or Grade 3 may be treated by surgical removal of a portion of one or more of the fat pads. The subject scored as Grade 0E, 1E, 2E, 3E, or 3+E may be administered a composition that comprises a protein having hyaluronidase activity to the peri-orbital region of the subject, and optionally treated by surgical removal of a portion of one or more of the fat pads.

In some embodiments, a subject's left eye is given a PEFAS score and the subject's right eye is given a PEFAS score. In other embodiments, only a subject's left or right eye is given a PEFAS score.

As used herein the terms mild, moderate, and severe refer to the degree of severity of a condition exhibited by the bottom, middle, and top third of patients, respectively, that have the condition. The lower third of patients have the least severe degree of the condition and the top third of patients have the highest degree of severity of the condition. For example, a patient with a mild festoons has a severity of festoons that falls within the bottom third of patients examined by a medical practitioner. Additionally, for example, a patient with moderate festoons has a severity of festoons that falls within the middle third of patients examined by a medical practitioner. Further, for example, a patient with severe festoons has a severity of festoons that falls within the top third of patients examined by a medical practitioner.

Alternatively, the present disclosure provides methods for diagnosing an etiology of upper and/or lower eyelid puffiness based on an eyelid squint test. Such methods comprise examining a subject with squinted eye; and determining if upper and/or lower eyelid puffiness does not improve, improves, partially improves, or worsens. The etiology of the upper and/or lower eyelid puffiness is diagnosed to be anterior to the orbicularis oculi muscle if the puffiness does not improve, the etiology of the upper and/or lower eyelid puffiness is diagnosed to be posterior to the orbicularis oculi muscle if the puffiness improves, the etiology of the upper and/or lower eyelid puffiness is diagnosed to be anterior and posterior to the orbicularis oculi muscle if the puffiness partially improves, or the puffiness is diagnosed to be secondary to hypertrophy of the orbicularis muscle or if the puffiness worsens.

In an embodiment, the subject is in an upright position with head in a Frankfort horizontal plane. In a further embodiment, the methods further comprise the step of instructing the subject to squint or tighten the orbicularis oculi muscle.

In an embodiment where the etiology of the upper and/or lower eyelid puffiness is determined to be anterior to the orbicularis oculi muscle the method further comprises administering a protein having hyaluronidase activity (e.g., as described herein) into the soft tissue anterior to the orbicularis oculi muscle.

In an embodiment where the etiology of the upper and/or lower eyelid puffiness is determined to be posterior to the orbicularis oculi muscle, the method further comprises the step of determining if the upper and/or lower eyelid puffiness is secondary to pseudoherniation of upper and/or lower eyelid fat pads, edema of upper and/or lower eyelid fat pads, or upper and/or lower eyelid fat pad pseudoherniation and edema. Such method includes the following steps. First, puffiness of the lower eyelid fat pads are assessed by asking the subject to look straight up, look up and to the right, and look up and to the left. The puffiness of the lower eyelid fat pads is due to pseudoherniation of the lower eyelid fat pads and surgery is indicated if the lower eyelid fat pads protrude and are individually isolated. In contrast, the puffiness is due to pseudoherniation and edema of the lower eyelid fat pads if the lower eyelid fat pads protrude and are not individually isolated. Second, a protein having hyaluronidase activity is then injected into the lower eyelid fat pads to determine the extent of edema of the lower eyelid fat pads. Third, the method may further comprise resecting a portion of the lower eyelid fat pads after assessment of the extent of edema to avoid over resection of the fat pad(s). Next, the puffiness of the upper eyelid fat pads are assessed by asking the subject to look straight down, look down and to the right, and look down and to the left. The puffiness of the upper eyelid fat pads is due to pseudoherniation of upper eyelid fat pads and surgery is indicated if the upper eyelid fat pads protrude and are individually isolated. In contrast, the puffiness is due to pseudoherniation and edema of upper eyelid fat pads if the upper eyelid fat pads protrude and are not individually isolated. Second, a protein having hyaluronidase activity is then injected into the upper eyelid fat pads to determine the extent of edema of the eyelid fat pads. Third, the method may further comprise resecting a portion of the lower eyelid fat pads after assessment of the extent of edema to avoid over resection of the fat pad(s).

In an embodiment where the etiology of the upper and/or lower eyelid puffiness is determined to be anterior and posterior to the orbicularis oculi muscle, the method further comprises injecting a protein having hyaluronidase activity into the upper and/or lower eyelid fat pads.

In an embodiment where the etiology of the upper and/or lower eyelid puffiness is determined to be anterior and posterior to the orbicularis oculi muscle, and wherein the method further comprises assessing whether the puffiness is partially due to pseudoherniation of eyelid fat pads or edema of the fat pads, or eyelid fat pad pseudoherniation and edema. Such method includes the following steps. First, puffiness of the lower eyelid fat pads are assessed by asking the subject to look straight up, look up and to the right, and look up and to the left. The puffiness of the lower eyelid fat pads is due to pseudoherniation of the lower eyelid fat pads and surgery is indicated if the lower eyelid fat pads protrude and are individually isolated. In contrast, the puffiness is due to pseudoherniation and edema of the lower eyelid fat pads if the lower eyelid fat pads protrude and are not individually isolated. Second, a protein having hyaluronidase activity is then injected into the lower eyelid fat pads to determine the extent of edema of the lower eyelid fat pads. Third, the method may further comprise resecting a portion of the lower eyelid fat pads after assessment of the extent of edema to avoid over resection of the fat pad(s). Next, the puffiness of the upper eyelid fat pads are assessed by asking the subject to look straight down, look down and to the right, and look down and to the left. The puffiness of the upper eyelid fat pads is due to pseudoherniation of upper eyelid fat pads and surgery is indicated if the upper eyelid fat pads protrude and are individually isolated. In contrast, the puffiness is due to pseudoherniation and edema of upper eyelid fat pads if the upper eyelid fat pads protrude and are not individually isolated. Second, a protein having hyaluronidase activity is then injected into the upper eyelid fat pads to determine the extent of edema of the eyelid fat pads. Third, the method may further comprise resecting a portion of the lower eyelid fat pads after assessment of the extent of edema to avoid over resection of the fat pad(s).

The present disclosure also provides methods for determining an etiology of peri-orbital puffiness by performing an eyelid squint test (e.g., asking or having a patient squint their eyes); and observing an impact of a movement of an orbicularis oculi muscle on protrusion of eyelid fat pads, wherein the etiology of the upper and/or lower eyelid puffiness is diagnosed to be anterior to the orbicularis oculi muscle if the puffiness does not improve, wherein the etiology of the upper and/or lower eyelid puffiness is diagnosed to be posterior to the orbicularis oculi muscle if the puffiness improves, wherein the etiology of the upper and/or lower eyelid puffiness is diagnosed to be anterior and posterior to the orbicularis oculi muscle if the puffiness partially improves, or wherein the puffiness is diagnosed to be secondary to hypertrophy of the orbicularis muscle or if the puffiness worsens.

### Edema and its Characterization

Pharmacological agents have been proposed for use in treating localized edema. Such agents are typically selected from those drugs normally used in the treatment of generalized inflammation, e.g., NSAIDs such as aspirin, ibuprofen, and the like, corticosteroids, and antihistamines. These agents can provide some degree of improvement, but relief is often minimal and short-lived. The inventors have discovered that a protein having hyaluronidase activity such as a recombinant human hyaluronidase can be used to treat and/or prevent edema without many of the drawbacks of existing treatments.

The methods disclosed herein may be used to treat edema including, for example, reduce the severity of edema. According to the conventional “pitting” method of measuring edema, a health-care provider presses on the skin of a patient with his or her finger and provides a score for the patient's edema according to a relative scale, typically from 1+(slight) to 4+(severe). The score is assigned based on one or more criteria including the depth of the pitting, the general appearance of the extremities (e.g., the feet, ankles, hands, etc.), the amount of time required for the pit to disappear, and the amount of force required to form the pit. Table 2 below shows the criteria typically used to assess the grade of pitting edema.

TABLE 2
Grades of Edema

| Grades | | |
|---|---|---|
| of | | |
| edema | Description | Score |
| 0 | No edema | 0 |
| 1+ | 2 mm or less: slight pitting, no | 1 |
| | visible distortion, disappears rapidly | |
| 2+ | 2-4 mm indent: somewhat deeper pit, no | 2 |
| | readably detectable distortion. | |
| | disappears in 10-25 s | |
| 3+ | 4-6 mm: pit is noticeably deep. May last more | 3 |
| | than a minute. Dependent | |
| | extremity looks swollen and fuller | |
| 4+ | 6-8 mm: pit is very deep. Lasts for 2-5 min. | 4 |
| | Dependent extremity is | |
| | grossly distorted | |

### Methods of Treating: Edema

The present disclosure provides methods of administering a protein having hyaluronidase, collagenase, and/or 4-methylumbelliferone (4-MU) activity, with or without one or more additional active pharmaceutical ingredients (APIs), to a subject in need thereof to treat and/or prevent edema, and/or a condition (e.g., a cosmetic condition) due to (caused by) peri-orbital or mid-face edema. Such conditions may include peri-ocular puffiness, festoons, and malar puffiness. The compositions can be formulated in lyophilized or liquid form. Where the compositions are provided in lyophilized form they can be reconstituted just prior to use by an appropriate buffer, for example, a sterile saline solution. The compositions can be provided together or separately. The compositions can be packaged as a kit.

Also provided herein are methods of treating or preventing a condition such as a cosmetic condition in a subject in need thereof by administering a composition that comprises a protein having hyaluronidase activity (e.g., a hyaluronidase such as Hylenex, Amphadase, or Vitrase), collagenase activity, and/or 4-MU activity, with or without one or more additional APIs, to a region of a subject's face having said cosmetic condition (e.g., to the peri-orbital region and/or mid-face of the subject). The cosmetic condition may be due to edema (e.g., peri-orbital puffiness due to peri-orbital edema).

The methods disclosed herein may be used to treat edema including, for example, reduce the severity of edema. In some embodiments, the methods may reduce the depth of the pitting, the general appearance of the extremities (e.g., the feet, ankles, hands, etc.), the amount of time required for the pit to disappear, and/or the amount of force required to form the pit. The severity of the edema may be reduced from 4+(severe) to 3+, 2+, 1+ or 0. 1+ (slight) to 4+ (severe). In another embodiment, the severity of the edema may be reduced from 3+ to 2+, 1+, or 0. In an alternative embodiment, the severity of the edema may be reduced from 3+ to 2+, 1+, or 0. In yet another embodiment, the severity of the edema may be reduced from 2+ to 1+, or 0. In another embodiment, the severity of the edema may be reduced from 1+ to 0.

The present disclosure also provides methods of treating peri-orbital puffiness in a subject in need thereof by administering a composition that comprises a protein having hyaluronidase, collagenase, and/or 4-MU activity, with or without one or more additional APIs, activity to a peri-orbital region of the subject. The methods may comprise administering, including by injection, a protein having hyaluronidase, collagenase, and/or 4-MU activity, with or without one or more additional APIs, activity to one or more peri-orbital fat pads.

Also provided are methods of treating peri-orbital puffiness in a subject in need thereof by administering a composition that comprises a protein having hyaluronidase, collagenase, and/or 4-MU activity, with or without one or more additional APIs, to an eye of the subject. In an embodiment, an eye drop that comprises the protein having hyaluronidase, collagenase, and/or 4-MU activity, with or without one or more additional APIs, is administered to the eye of the subject, including, for example, directly to the surface of the eye.

Also provided are methods of treating or preventing peri-ocular puffiness in a subject in need thereof by administering a composition that comprises a protein having hyaluronidase activity (e.g., a hyaluronidase such as Hylenex, Amphadase, or Vitrase), collagenase activity, and/or 4-MU activity, with or without one or more additional APIs, to a region of a subject's face having said peri-ocular puffiness (e.g., to the peri-orbital region and/or mid-face of the subject). The peri-ocular puffiness may be due to edema (e.g., peri-orbital puffiness due to peri-orbital edema).

The disclosure also provides methods of treating or preventing festoons in a subject in need thereof by administering a composition that comprises a protein having hyaluronidase activity (e.g., a hyaluronidase such as Hylenex, Amphadase, or Vitrase), collagenase activity, and/or 4-MU activity, with or without one or more additional APIs, to a region of a subject's face having said festoons. The festoons may be due to edema.

Additionally, provided herein are methods of treating or preventing malar puffiness in a subject in need thereof by administering a composition that comprises a protein having hyaluronidase activity (e.g., a hyaluronidase such as Hylenex, Amphadase, or Vitrase), collagenase activity, and/or 4-MU activity, with or without one or more additional APIs, to a region of a subject's face having malar puffiness. The malar puffiness may be due to edema.

In an embodiment, the methods may comprise a step of determining if the peri-orbital puffiness is due to edema or a structural change (e.g., a herniation of one or more of the eyelid fats pads located in the peri-orbital region) in the subject's peri-orbital region by administering a composition that comprises a protein having hyaluronidase, collagenase, and/or 4-MU activity, with or without one or more additional APIs, to the peri-orbital region of the subject, assessing the peri-orbital region at a predetermined amount of time after the administration of the composition, and determining whether there is an improvement in the peri-orbital puffiness (e.g., a decrease in the puffiness including, for example, a visible decrease in puffiness). The method of determining if the puffiness is due to edema or a structural change may be conducted prior to treatment of the subject with a composition that comprises a protein having hyaluronidase, collagenase, and/or 4-MU activity, with or without one or more additional APIs. For example, a subject may first be administered a small dose of the composition comprising the protein having hyaluronidase, collagenase, and/or 4-MU activity, with or without one or more additional APIs, and then be treated with a larger dose of the same composition where the peri-orbital puffiness exhibited an improvement (e.g., a reduction in peri-orbital puffiness). If the puffiness does not improve, it is due to a structural change and the subject is not treated with the composition comprising the protein having hyaluronidase, collagenase, and/or 4-MU activity, with or without one or more additional APIs.

The present disclosure also provides compositions that comprise a combination of a hyaluronidase, collagenause, and/or 4-MU, and one or more APIs. The inventors have surprisingly discovered that a protein having hyaluronidase, collagenause, and/or 4-MU activity can synergistically increase the potency and/or efficacy of an API when administered with the API. As such, these combinations may be useful for treating and/or preventing a disease or disorder including, for example, a disease or disorder for which the API is indicated or is not presently indicated.

The present disclosure further provides methods for treating and/or preventing a disease or disorder in a subject in need thereof. Such methods may comprise a step of administering a composition comprising a protein having hyaluronidase activity such as Hylenex and an additional API to the subject. Advantageously such compostions havesynergistically improved potency and/or efficacy as compared to otherwise identical compostions that lack the protein having hyaluronidase activity.

In an embodiment, the additional active pharmaceutical ingredient is used to treat acne, ADHD, AIDS/HIV, allergies, Alzheimer's, angina, anxiety, arthritis, asthma, bipolar disorder, bronchitis, cancer, cholesterol, colds & flu, constipation, COPD, depression, diabetes (Type 1), diabetes (Type 2), diarrhea, eczema, erectile dysfunction, fibromyalgia, gastrointestinal, GERE (Heartburn), gout, hair loss, hayfever, heart disease, hepatitis A, hepatitis B, herpes, hypertension, hypothyroidism, incontinence, IBD (Bowel), insomnia, menopause, mental health, migraine, osteoarthritis, osteoporosis, pain, psoriasis, rheumatoid arthritis, schizophrenia, seizures, sexual health, stroke, swine flu, UTI, or weight loss.

In an embodiment, the additional active pharmaceutical ingredient (API) may be selected from the group consisting of: 0.5-alpha-reductase inhibitors, 5-aminosalicylates, 5HT3 receptor antagonists, ACE inhibitors with calcium channel blocking agents, ACE inhibitors with thiazides, adamantane antivirals, adrenal cortical steroids, adrenal corticosteroid inhibitors, adrenergic bronchodilators, agents for hypertensive emergencies, agents for pulmonary hypertension, aldosterone receptor antagonists, alkylating agents, allergenics, alpha-glucosidase inhibitors, alternative medicines, amebicides, am inoglycosides, am inopenicillins, am inosalicylates, AMPA receptor antagonists, amylin analogs, analgesic combinations, analgesics, androgens and anabolic steroids, Angiotensin Converting Enzyme Inhibitors, angiotensin II inhibitors with calcium channel blockers, angiotensin II inhibitors with thiazides, angiotensin receptor blockers, angiotensin receptor blockers and neprilysin inhibitors, anorectal preparations, anorexiants, antacids, anthelmintics, anti-angiogenic ophthalmic agents, anti-CTLA-4 monoclonal antibodies, anti-infectives, anti-PD-1 monoclonal antibodies, antiadrenergic agents (central) with thiazides, antiadrenergic agents (peripheral) with thiazides, antiadrenergic agents, centrally acting, antiadrenergic agents, peripherally acting, antiandrogens, antianginal agents, antiarrhythmic agents, antiasthmatic combinations, antibiotics/antineoplastics, anticholinergic antiemetics, anticholinergic antiparkinson agents, anticholinergic bronchodilators, anticholinergic chronotropic agents, anticholinergics/antispasmodics, anticoagulant reversal agents, anticoagulants, anticonvulsants, antidepressants, antidiabetic agents, antidiabetic combinations, antidiarrheals, antidiuretic hormones, antidotes, antiemetic/antivertigo agents, antifungals, antigonadotropic agents, antigout agents, antihistamines, antihyperlipidemic agents, antihyperlipidemic combinations, antihypertensive combinations, antihyperuricemic agents, antimalarial agents, antimalarial combinations, antimalarial quinolones, antimanic agents, antimetabolites, antimigraine agents, antineoplastic combinations, antineoplastic detoxifying agents, antineoplastic interferons, antineoplastics, antiparkinson agents, antiplatelet agents, antipseudomonal penicillins, antipsoriatics, antipsychotics, antirheumatics, antiseptic and germicides, antithyroid agents, antitoxins and antivenins, antituberculosis agents, antituberculosis combinations, antitussives, antiviral agents, antiviral boosters, antiviral combinations, antiviral interferons, anxiolytics, sedatives, and hypnotics, aromatase inhibitors, atypical antipsychotics, azole antifungals, Abilify, Ablysinol, Absorica, Acanya, Accolate, Accrufer, AccuNeb, Accupril, Accuretic, Acetadote, Achromycin V, Aciphex, Aciphex Sprinkle, Acthrel, Acticlate, Actigall, Actiq, Activella, Actonel, ActoPlus Met, Actos, Acular, Acular LS, Acuvail, Aczone, Adalat CC, Adasuve, Adcirca, Adderall, Adderall XR, Addyi, Adempas, Adhansia XR, Adlyxin, Admelog, Adrenaclick, AdreView, Advair Diskus, Advair HFA, Adzenys ER, Adzenys XR-ODT, Aemcolo, Afinitor, Afinitor Disperz, Aggrastat, Aggrenox, Agrylin, AirDuo Digihaler, AirDuo Respiclick, AK-Fluor, Aklief, Akovaz, Akten, Akynzeo, Albenza, Aldactazide, Aldactone, Aldara, Alecensa, Alfenta, Alimta, Alinia, Aliqopa, Alkeran, Alocril, Alomide, Aloprim, Alora, Aloxi, Alphagan, Alrex, Altabax, Altace, Altafluor Benox, Altoprev, Altreno, Alunbrig, Alvesco, Amaryl, Ambien, Ambien CR, Ameluz, Amerge, Amicar, Amidate, Amitiz, Ammonul, Amphadase, Ampyra, Amrix, Amyvid, Amzeeq, Anadrol-50, Anafranil, Anaprox-DS, Ancobon, Androderm, AndroGel, Anectine, Angeliq, bacterial vaccines, barbiturate anticonvulsants, barbiturates, BCR-ABL tyrosine kinase inhibitors, benzodiazepine anticonvulsants, benzodiazepines, beta blockers with calcium channel blockers, beta blockers with thiazides, beta-adrenergic blocking agents, beta-lactamase inhibitors, bile acid sequestrants, biologicals, bisphosphonates, bone morphogenetic proteins, bone resorption inhibitors, bronchodilator combinations, bronchodilators, Bactrim, Bactrim DS, Balcoltra, Balversa, Banzel, Baqsimi, Baraclude, Basaglar, Baxdela, Belbuca, Beleodaq, Belrapzo, Belsomra, Belviq, Belviq XR, Bendeka, Benicar, Benicar HCT, Bentyl, Benzaclin, Benzamycin, Bepreve, Besivance, Beta-Val, Betagan, Betapace, Betapace AF, Bethkis, Betimol, Betoptic, Betoptic S, Bevespi Aerosphere, Bevyxxa, Beyaz, Bicillin L-A, BiCNU, BiDil, Bijuva, Biktarvy, Biltricide, Binosto, Biorphen, Blephamide, Bloxiverz, Boniva, Bonjesta, Bonsity, Bosulif, Braftovi, Breo Ellipta, Brethine, Brevibloc, Brevital Sodium, Bridion, Brilinta, Brisdelle, Briviact, BromSite, Brovana, Bryhali, Bumex, Bunavail, Buphenyl, Buprenex, Busulfex, Butisol Sodium, Butrans, Bydureon, Bydureon BCise, Byetta, Bystolic, Calcimimetics, calcineurin inhibitors, calcitonin, calcium channel blocking agents, carbamate anticonvulsants, carbapenems, carbapenems/beta-lactamase inhibitors, carbonic anhydrase inhibitor anticonvulsants, carbonic anhydrase inhibitors, cardiac stressing agents, cardioselective beta blockers, cardiovascular agents, catecholamines, cation exchange resins, CD20 monoclonal antibodies, CD30 monoclonal antibodies, CD33 monoclonal antibodies, CD38 monoclonal antibodies, CD52 monoclonal antibodies, CDK 4/6 inhibitors, central nervous system agents, cephalosporins, cephalosporins/beta-lactamase inhibitors, cerumenolytics, CFTR combinations, CFTR potentiators, CGRP inhibitors, chelating agents, chemokine receptor antagonist, chloride channel activators, cholesterol absorption inhibitors, cholinergic agonists, cholinergic muscle stimulants, cholinesterase inhibitors, CNS stimulants, coagulation modifiers, colony stimulating factors, contraceptives, corticotropin, coumarins and indandiones, cox-2 inhibitors, Cabometyx, Caduet, Cafcit, Calan, Calan SR, Calcium Disodium Versenate, Caldolor, Calquence, Cambia, Campral, Camptosar, Canasa, Cancidas, Capastat Sulfate, Capex, Caprelsa, Carac, Carafate, Carbaglu, Carbatrol, Carbocaine, Cardiogen-82, Cardizem, Cardizem CD, Cardizem LA, Cardura, Cardura XL, Carnitor, Carnitor SF, CaroSpir, Casodex, Casporyn HC, Catapres, Catapres-TTS, Caverject, Caverject Impulse, Cayston, Cefotan, Ceftin, Celebrex, Celestone Soluspan, Celexa, CellCept, Celontin, Centany, Cequa, Cerdelga, Cerebyx, Cerezyme, Cesamet, Cetraxal, Cetrotide, Chantix, Chemet, ChiRhoStim, Cholbam, Cialis, Ciloxan, Cimduo, Cinvanti, Cipro, Cipro HC, Ciprodex, Clarinex, Clarinex-D 12 Hour, Clenpiq, Cleocin Phosphate, Cleocin T, Cleocin Vaginal, Cleviprex, Climara, Climara Pro, lindagel, Clindesse, Clobex, Cloderm, Clolar, Clom id, Clorotekal, Clozaril, Coartem, Cogentin, Colazal, Colcrys, Colestid, Coly Mycin M, Coly-Mycin S, Combigan, CombiPatch, Combivent Respimat, Combivir, Cometriq, Complera, Comtan, Concerta, Condylox, Conray, Consensi, Contrave, ConZipD, Copaxone, Copegus, Copiktra, Cordarone, Cordran, Cordran SP, Coreg, Coreg CR, Corgard, Corlanor, Corlopam, Corphedra, Cortef, Cortenema, Cortifoam, Cortisporin Cream, Cortisporin Ointment, Cortrosyn, Corvert, Corzide, Cosmegen, Cosopt, Cosopt PF, Cotellic, Cotempla XR-ODT, Coumadin, Cozaar, Cresemba, Crestor, Crixivan, Cubicin, Cubicin RF, Cuprimine, Curosurf, Cutivate, Cuvposa, Cyanokit, Cyclessa, Cycloset, Cyklokapron, Cymbalta, Cystadane, Cystagon, Cystaran, Cysto-Conray II, Cystografin, Cytomel, Cytotec, Cytovene, Decongestants, dermatological agents, diagnostic radiopharmaceuticals, diarylquinolines, dibenzazepine anticonvulsants, digestive enzymes, dipeptidyl peptidase 4 inhibitors, diuretics, dopaminergic, antiparkinsonism agents, drugs used in alcohol dependence, D.H.E. 45, Dacogen, Daliresp, Dalvance, Dantrium, Daraprim, DaTscan, Daurismo, Daypro, Daytrana, DDAVP, Definity, Defitelio, Delestrogen, Delstrigo, Delzicol, Demadex, Demerol, Demser, Denavir, Depacon, Depakote, Depakote ER, Depen, Depo-Medrol, Depo-Provera, Depo-subQ provera 104, Derma-Smoothe/FS, Dermatop, DermOtic Oil, Descovy, Desferal, Desonate, DesOwen, Desoxyn, Detrol, Detrol LA, Dexedrine, Dexilant, Dextenza, DiaBeta, Diabinese, Diacomit, Diastat, Diastat AcuDial, Dibenzyline, Diclegis, Differin, Dificid, Diflucan, Dilantin, Dilatrate-SR, Dilaudid, Diovan, Diovan HCT, Dipentum, Diprivan, Diprolene, Diprolene AF, Ditropan XL, Diuril, Divigel, Docefrez, Dolophine, Dopram, Doptelet, Doral, Doryx, Doryx MPC, Dotarem, Dovato, Dovonex, Drisdol, Drizalma Sprinkle, Droxia, Dsuvia, Duac, Duaklir Pressair, Duavee, Duetact, Duexis, Dulera, Duobrii, DuoDote, Duopa, Duraclon, Duragesic, Duramorph PF, Durezol, Durlaza, Dutoprol, Dyanavel XR, Dyazide, Dyrenium, Echinocandins, EGFR inhibitors, estrogen receptor antagonists, estrogens, expectorants, E-Z-HD, E-Z-Paque, EC-Naprosyn, Ecoza, Edarbi, Edarbyclor, Edecrin, Edex, Edluar, Edurant, Effexor, Effexor XR, Effient, Efudex, Egaten, Egrifta, Elcys, Elelyso, Elepsia XR, Elestat, Elestrin, Elidel, Eligard, Elimite, Eliquis, Ella, Ellence, Elmiron, Elocon, Eloxatin, Embeda, Emcyt, Emend, Emflaza, Emla, Emsam, Emtriva, Enablex, Endari, Endometrin, Enstilar, Entereg, Entocort EC, Entresto, Envarsus XR, Eovist, Epaned, Epclusa, Epidiolex, Epiduo, Epiduo Forte, EpiPen, EpiPen Jr, Epivir, Epivir-HBV, Epzicom, Equetro, Eraxis, Erivedge, Erleada, Ertaczo, Eryc, Erygel, EryPed, Erythrocin, Esbriet, Estrogel, Estrostep Fe, Ethamolin, Ethyol, Eucrisa, Eurax, Euthyrox, Evamist, Evekeo ODT, Evista, Evoclin, Evomela, Evotaz, Evoxac, Evzio, Exelderm, Exelon, Exforge, Exforge HCT, Exjade, Exondys 51, Extina, Ezallor, factor Xa inhibitors, fatty acid derivative anticonvulsants, fibric acid derivatives, first generation cephalosporins, fourth generation cephalosporins, functional bowel disorder agents, Fabior, Factive, Famvir, Fanapt, Fareston, Farxiga, Farydak, Faslodex, Felbatol, Feldene, Femara, Femhrt, Femring, Fenoglide, Fentora, Feraheme, Ferriprox, Ferrlecit, Fetzima, Fiasp, Fibricor, Finacea, Fiorinal, Firazyr, Firdapse, Firmagon, Flagyl, Flarex, Flector Patch, Flolan, FloLipid, Flomax, Flovent HFA, Flumadine, Fluorescite, Fluoroplex, FML, FML Forte Liquifilm, Focalin, Focalin XR, Follistim AQ, Folotyn, Forane, Forfivo XL, Fortamet, Fortaz, Forteo, Fortesta, Fosamax, Fosamax Plus D, Foscavir, Fosrenol, Fragmin, Frova, Furadantin, Fusilev, Fuzeon, Fycompa, gallstone solubilizing agents, gamma-aminobutyric acid analogs, gamma-aminobutyric acid reuptake inhibitors, gastrointestinal agents, general anesthetics, genitourinary tract agents, GI stimulants, glucocorticoids, glucose elevating agents, glycopeptide antibiotics, glycoprotein platelet inhibitors, glycylcyclines, gonadotropin releasing hormones, gonadotropin-releasing hormone antagonists, gonadotropins, group I antiarrhythmics, group II antiarrhythmics, group III antiarrhythmics, group IV antiarrhythmics, group V antiarrhythmics, growth hormone receptor blockers, growth hormones, guanylate cyclase-C agonists, Gabitril, Gablofen, Gadavist, Galafold, Galzin, Gastrocrom, Gastrografin, Gelnique, Gemzar, Genotropin, Genvoya, Geodon, Giapreza, Gilenya, Gilotrif, Gleevec, Gleolan, Gleostine, Gliadel, Gloperba, GlucaGen, Glucophage, Glucophage XR, Glucotrol, Glucotrol XL, Glumetza, Glynase, Glyrx-PF, Glyset, Glyxambi, Gocovri, Gonal-f, Gonal-f RFF, Gonal-f RFF Redi-ject, GoNitro, Gralise, Gris-PEG, Gvoke, H. pylori eradication agents, H2 antagonists, hedgehog pathway inhibitors, hematopoietic stem cell mobilizer, heparin antagonists, heparins, HER2 inhibitors, herbal products, histone deacetylase inhibitors, hormones, hormones/antineoplastics, hydantoin anticonvulsants, hydrazide derivatives, H.P. Acthar Gel, Halaven, Halcion, Haldol, Halog, Harvoni, Hectorol, Hemabate, Hemady, Hemangeol, Hepsera, Hetlioz, Hicon, Hiprex, Horizant, Humalog, Humalog KwikPen, Humalog Mix 50/50, Humalog Mix 50/50 KwikPen, Humalog Mix 75/25, Humalog Mix 75/25 KwikPen, Humatrope, Hycamtin, Hycodan, Hydrea, Hysingla ER, Hyzaar, illicit (street) drugs, immune globulins, immunologic agents, immunostimulants, immunosuppressive agents, impotence agents, in vivo diagnostic biologicals, incretin mimetics, inhaled anti-infectives, inhaled corticosteroids, inotropic agents, insulin, insulin-like growth factors, integrase strand transfer inhibitor, interferons, interleukin inhibitors, interleukins, intravenous nutritional products, iodinated contrast media, ionic iodinated contrast media, iron products, Ibrance, Ibsrela, IC-Green, Iclusig, Idamycin PFS, Idhifa, Ifex, Ilevro, Iluvien, Imbruvica, Imdur, Imitrex, Imitrex Statdose, Impavido, Impoyz, Imuran, Inapsine, Inbrija, Increlex, Incruse Ellipta, Inderal LA, Indocin, Infed, Infugem, Infumorph, Ingrezza, Injectafer, Inlyta, InnoPran XL, INOmax, Inrebic, Inspra, Integrilin, Intelence, Intuniv, Invanz Invega, Invega Sustenna, Invega Trinza, Inveltys, Invirase, Invokamet, Invokamet XR, Invokana, Iopidine, Iressa, Isentress, Isentress HD, Isopto Atropine, Isopto Carpine, Isordil, Isovue-200, Isovue-250, Isovue-300, Isovue-370, Istalol, Istodax, Isuprel, Jadenu, Jadenu Sprinkle, Jakafi, Jalyn, Janumet, Janumet XR, Januvia, Jardiance, Jatenzo, Jentadueto, Jentadueto XR, Jornay PM, Jublia, Juluca, Juxtapid, Jynarque, Ketolides, K-Tab, Kadian, Kaletra, Kalydeco, Kapspargo Sprinkle, Kapvay, Karbinal ER, Katerzia, Kazano, Keflex, Kenalog, Kenalog-10, Kenalog-40, Kengreal, Keppra, Keppra XR, Kerydin, Ketalar, Keveyis, Khapzory, Kinevac, Kisqali, Kitabis Pak, Klaron, Klonopin, Klor-Con, Kombiglyze XR, Korlym, Krintafel, Kuvan, Kybella, Kyleena, Kyprolis, Laxatives, leprostatics, leukotriene modifiers, lincomycin derivatives, local injectable anesthetics, local injectable anesthetics with corticosteroids, loop diuretics, lung surfactants, lymphatic staining agents, lysosomal enzymes, Lacrisert, Lamictal, Lamictal CD, Lamictal ODT, Lamictal XR, Lamisil, Lanoxin, Lantus, Lantus SoloStar, Lasix, Lastacaft, Latisse, Latuda, Lazanda, Lenvima, Lescol XL, Letairis, Leukeran, Levemir, Levitra, Levlite, Levophed, Levulan Kerastick, Lexapro, Lexette, Lexiscan, Lexiva, Lialda, Librax, Lidex, Lidex-E, Lidoderm, Liletta, Lincocin, Linzess, Lioresal, Lipiodol, Lipitor, Lipofen, Lithobid, Lithostat Livalo, Lo Loestrin Fe, Locoid, Locoid Lipocream, Lodosyn, Loestrin 21 1.5/30, Loestrin 211/20, Loestrin 24 Fe, Loestrin Fe 1.5/30, Loestrin Fe 1/20, Lokelma, Lomotil, Lonsurf, Lopid, Lopressor, Lopressor HCT, Loprox, Lorbrena, LoSeasonique, Lotemax, Lotemax SM, Lotensin, Lotensin HCT, Lotrel, Lotrisone, Lotronex, Lovaza, Lovenox, Lucemyra, Lumason, Lumigan, Lunesta, Lupaneta Pack, Lupron Depot, Lupron Depot-PED, Lutathera, Luvox, Luxiq, Luzu, Lybrel, Lynparza, Lyrica, Lyrica CR, Lysodren, Lysteda, macrolide derivatives, macrolides, magnetic resonance imaging contrast media, malignancy photosensitizers, mast cell stabilizers, medical gas, meglitinides, metabolic agents, methylxanthines, mineralocorticoids, minerals and electrolytes, miscellaneous agents, miscellaneous analgesics, miscellaneous antibiotics, miscellaneous anticonvulsants, miscellaneous antidepressants, miscellaneous antidiabetic agents, miscellaneous antiemetics, miscellaneous antifungals, miscellaneous, ntihyperlipidemic agents, miscellaneous antihypertensive combinations, miscellaneous antimalarials, miscellaneous antineoplastics, miscellaneous antiparkinson agents, miscellaneous antipsychotic agents, miscellaneous antituberculosis agents, miscellaneous antivirals, miscellaneous anxiolytics, sedatives and hypnotics, miscellaneous bone resorption inhibitors, miscellaneous cardiovascular agents, miscellaneous central nervous system agents, miscellaneous coagulation modifiers, miscellaneous diagnostic dyes, miscellaneous diuretics, miscellaneous genitourinary tract agents, miscellaneous GI agents, miscellaneous hormones, miscellaneous metabolic agents, miscellaneous ophthalmic agents, miscellaneous otic agents, miscellaneous respiratory agents, miscellaneous sex hormones, miscellaneous topical agents, miscellaneous uncategorized agents, miscellaneous vaginal agents, mitotic inhibitors, monoamine oxidase inhibitors, mouth and throat products, mTOR inhibitors, mucolytics, multikinase inhibitors, muscle relaxants, mydriatics, Macrilen, Macrobid, Macrodantin, Makena, Malarone, Malarone Pediatric, Marcaine, Marinol, Marplan, Matulane, Mavenclad, Mavyret, Maxalt, Maxalt-MLT, Maxidex, Maxipime, Maxitrol, Maxzide, Maxzide-25, Mayzent, Medrol, Megace ES, Mekinist, Mektovi, MembraneBlue, Menostar, Mentax, Mephyton, Mepron, Merrem, Mesnex, Mestinon, Metadate CD, Metastron, Methadose, Methergine, Methylin, Metopirone, MetroCream, MetroGel, MetroGel-Vaginal, MetroLotion, Miacalcin, Micardis, Micardis HCT, Micro-K, Micro-K 10, Microzide, Midamor, Mifeprex, Migranal, Minastrin 24 Fe, Minipress, Minirin, Minivelle, Minocin, Minolira, Miochol-E, Miostat, Mirapex, Mirapex ER, Mircette, Mirena, Mirvaso, Mitigare, Mobic, Monistat 3, Monoket, Monurol, MorphaBond ER, Motegrity, Motofen, Movantik, Moxeza, Mozobil, MS Contin, Mulpleta, Multaq, Multihance, Muse, Myambutol, Mycamine, Mycobutin, Mydayis, Myfortic, Myleran, Myrbetriq, Mysoline, Mytesi, narcotic analgesic combinations, narcotic analgesics, nasal anti-infectives, nasal antihistamines and decongestants, nasal lubricants and irrigations, nasal preparations, nasal steroids, natural penicillins, neprilysin inhibitors, neuraminidase inhibitors, neuromuscular blocking agents, neuronal potassium channel openers, next generation cephalosporins, NHE3 inhibitors, nicotinic acid derivatives, NK1 receptor antagonists, NNRTIs, non-cardioselective beta blockers, non-iodinated contrast media, non-ionic iodinated contrast media, non-sulfonylureas, Nonsteroidal anti-inflammatory drugs, NS5A inhibitors, nucleoside reverse transcriptase inhibitors (NRTIs), nutraceutical products, nutritional products, Naftin, Nalfon, Namenda, Namenda XR, Namzaric, Naprelan, Naprosyn, Narcan Nasal Spray, Nardil, Naropin, Nascobal, Natacyn, Natazia, Natesto, Natroba, Nayzilam, Nebupent, NeoProfen, Neoral, Nerlynx, Nesacaine, Nesacaine-MPF, Nesina, Netspot, Neupro, Neuraceq, Neurontin, Nevanac, Nexavar, Nexium, Nexium IV, Nexplanon, Nexterone, Niaspan, Nicotrol Inhaler, Nilandron, Nimbex, Ninlaro, Nipent, Niravam, Nithiodote, Nitro-Dur, Nitrolingual Pumpspray, NitroMist, Nitrostat, Nityr, Nizoral Topical, Nocdurna, Nor-QD, Nordette-28, Norditropin FlexPro, Noritate, Norpace, Norpace CR, Norpramin, Northera, Norvasc, Norvir, Nourianz, Novolog, NovoLog FlexPen, NovoLog Mix 70/30, NovoLog Mix 70/30 FlexPen, NovoLog PenFill, Noxafil, Nubeqa, Nucynta, Nucynta ER, Nuedexta, Nuplazid, Nutropin AQ, NuvaRing, Nuvessa, Nuvigil, Nuzyra, Nymalize, ophthalmic anesthetics, ophthalmic anti-infectives, ophthalmic anti-inflammatory agents, ophthalmic antihistamines and decongestants, ophthalmic diagnostic agents, ophthalmic glaucoma agents, ophthalmic lubricants and irrigations, ophthalmic preparations, ophthalmic steroids, ophthalmic steroids with anti-infectives, ophthalmic surgical agents, oral nutritional supplements, other immunostimulants, other immunosuppressants, otic anesthetics, otic anti-infectives, otic preparations, otic steroids, otic steroids with anti-infectives, oxazolidinedione anticonvulsants, oxazolidinone antibiotics, Ocaliva, Octreoscan, Ocufen, Ocuflox, Odefsey, Odomzo, Ofev, Ofirmev, Olumiant, Olux, Omnicef, Omnipaque 12, Omnipaque 140, Omnipaque 180, Omnipaque 240, Omnipaque 300, Omnipaque 350, Omnipaque 9, Omnipred, Omniscan, Omnitrope, Onexton, Onfi, Onglyza, Onmel, Onpattro, Onzetra Xsail, Opana, Opsumit, Optiray 240, Optiray 300, Optiray 320, Optiray 350, Orabloc, Oracea, Oraltag, Orapred ODT, Oraqix, OraVerse, Oravig, Orbactiv, Orenitram, Orfadin, Orilissa, Orkambi, Ortho Tri-Cyclen, Ortho Tri-Cyclen Lo, Ortho-Novum 7/7/7, Oseni, Osmolex ER, OsmoPrep, Osphena, Otezla, Otiprio, Otovel, Otrexup, Ovidrel, Oxaydo, Oxistat, Oxsoralen-Ultra, Oxtellar XR, OxyContin, Oxytrol, Ozempic, Ozobax, Ozurdex, parathyroid hormone and analogs, PARP inhibitors, PCSK9 inhibitors, penicillinase resistant penicillins, penicillins, peripheral opioid receptor antagonists, peripheral opioid receptor mixed gonists/antagonists, peripheral vasodilators, peripherally acting antiobesity agents, phenothiazine antiemetics, phenothiazine antipsychotics, phenylpiperazine antidepressants, phosphate binders, PI3K inhibitors, plasma expanders, platelet aggregation inhibitors, platelet-stimulating agents, polyenes, potassium sparing diuretics with thiazides, potassium-sparing diuretics, probiotics, progesterone receptor modulators, progestins, prolactin inhibitors, prostaglandin D2 antagonists, protease inhibitors, protease-activated receptor-1 antagonists, proteasome inhibitors, proton pump inhibitors, psoralens, psychotherapeutic agents, psychotherapeutic combinations, purine nucleosides, pyrrolidine anticonvulsants, Pamelor, Pandel, Panretin, Paremyd, Parlodel, Parnate, Parsabiv, Pataday, Patanol, Paxil, Paxil CR, Pazeo, PediaPred, Peganone, Penlac, Pennsaid, Pentam, Pentasa, Pepcid, Percodan, Perforomist, Peridex, PerioChip, Persantine, Pexeva, Phoslyra, Phospholine Iodide, Photofrin, Picato, Pifeltro, Piqray, Pitocin, Plaquenil, Plavix, Pliaglis, Polytrim, Pomalyst, Ponstel, Pradaxa, Pravachol, Pre-Pen, Precedex, Pred Forte, Pred Mild, Pred-G, Pregnyl, Premarin, Premphase 14/14, Prempro, Prepidil, Prestalia, Prevacid, Prevymis, Prezcobix, Prezista, Prialt, Priftin, Prilosec, Primsol, Prinivil, Pristiq, ProAir Digihaler, ProAir HFA, ProAir RespiClick, Probuphine, Procardia, Procardia XL, Procysbi, Proglycem, Prograf, Prohance, Prolensa, Promacta, Prometrium, Propecia, Proscar, Prostin E2, Prostin VR Pediatric, Protonix, Protonix IV, Protopam Chloride, Protopic, Provayblue, Provera, Provigil, Provocholine, Prozac, Prozac Weekly, Pulmicort Flexhaler, Pulmicort Respules, Purinethol, Purixan, Pylerac, Quinolones, Qbrelis, Qbrexza, Qmiiz ODT, QNASL, Qoliana, Qsymia, Qtern, Qternmet XR, Quadramet, Qualaquin, Quartette, Qudexy XR, Quelicin, QuilliChew ER, Quillivant XR, Qutenza, Quzyttir, Qvar RediHaler, radiocontrast agents, radiologic adjuncts, radiologic agents, radiologic conjugating agents, radiopharmaceuticals, recombinant human erythropoietins, renin inhibitors, respiratory agents, respiratory inhalant products, rifamycin derivatives, R-Gene 10, Radicava, Ranexa, Rapaflo, Rapamune, Rapivab, Rasuvo, Ravicti, Rayaldee, Rayos, Razadyne, Razadyne ER, Readi-Cat 2, Rebetol, Recarbrio, Reclast, Rectiv, Reglan, Regonol, Relenza, Relistor, Relpax, Remeron, Remeron SolTab, Remodulin, Renagel, Renova, Renvela, Requip, Requip XL, Rescriptor, Restasis, Restoril, Retin-A, Retisert, Retrovir, Revatio, Revlimid, Rexulti, Reyataz, Rhofade, Rhopressa, Ridaura, Rifadin, Rifater, Rilutek, Rimactane, Rimso-50, Rinvoq, Riomet, Riomet ER, Risperdal, Risperdal Consta, Ritalin, Ritalin LA, Robaxin, Robaxin-750, Rocaltrol, Rocklatan, Rowasa, Roxicodone, Rozerem, Rozlytrek, Rubraca, Ruby-Fill, Ruzurgi, Ryanodex, Rybelsus, Rydapt, Rytary, Rythmol, Rythmol SR, salicylates, sclerosing agents, second generation cephalosporins, selective estrogen receptor modulators, selective immunosuppressants, selective phosphodiesterase-4 inhibitors, selective serotonin reuptake inhibitors, serotonin-norepinephrine reuptake inhibitors, serotoninergic neuroenteric modulators, sex hormone combinations, sex hormones, SGLT-2 inhibitors, skeletal muscle relaxant combinations, skeletal muscle relaxants, smoking cessation agents, somatostatin and somatostatin analogs, spermicides, statins, sterile irrigating solutions, streptogramins, Streptomyces derivatives, succinimide anticonvulsants, sulfonamides, sulfonylureas, synthetic ovulation stimulants, Sabril, Safyral, Saizen, Salagen, Samsca, Sancuso, Sandimmune, Sandostatin, Saphris, Sarafem, Savaysa, Savella, Saxenda, Scenesse, Sclerosol, Seasonale, Seasonique, Secuado, Segluromet, Seizalam, Selzentry, Semprex-D, Sensipar, Sensorcaine, Septocaine, Septra, Septra DS, Serevent Diskus, Sernivo, Seroquel, Seroquel XR, Serostim, Seysara, Signifor, Siklos, Silenor, SilQ Vanilla, Silvadene, Simbrinza, Sinemet, Sinemet CR, Singulair, Sirturo, Sitavig, Sivextro, Skelaxin, Sklice, Skyla, Slynd, Smoothie Readi-Cat 2, Solaraze, Soliqua, Solodyn, Solosec, Soltamox, Solu-Cortef, Solu-Medrol, Soma, Somatuline Depot, Somavert, Sonata, Soolantra, Soriatane, Sorilux, Sotylize, Sovaldi, Spectazole, Spinraza, Spiriva, Spiriva Respimat, Sporanox, Spritam, Sprycel, SSD, Stalevo 100, Stalevo 125, Stalevo 150, Stalevo 200, Stalevo 50, Stalevo 75, Staxyn, Steglatro, Steglujan, Stendra, Steritalc, Stiolto Respimat, Stivarga, Strattera, Stribild, Striverdi Respimat, Stromectol, Sublocade, Suboxone, Subsys, Sucraid, Sular, Sulfamylon, Sulfatrim Pediatric, Sunosi, Supprelin LA, Suprane, Suprane, Suprax, Suprep Bowel Prep Kit, Survanta, Sustiva, Sustol, Sutent, Symbicort, Symbyax, Symfi, Symfi Lo, Symjepi, Symlin, Symmetrel, Sympazan, Symproic, Symtuza, Synalar, Synarel, Syndros, Synera, Synercid, Synjardy, Synjardy XR, Synribo, SyprineSuprax, Suprep Bowel Prep Kit, Survanta, Sustiva, Sustol, Sutent, Symbicort, Symbyax, Symfi, Symfi Lo, Symjepi, Symlin, Symmetrel, Sympazan, Symproic, Symtuza, Synalar, Synarel, Syndros, Synera, Synercid, Synjardy, Synjardy XR, Synribo, Syprine, tetracyclic antidepressants, tetracyclines, therapeutic radiopharmaceuticals, therapeutic vaccines, thiazide diuretics, thiazolidinediones, thioxanthenes, third generation cephalosporins, thrombin inhibitors, thrombolytics, thyroid drugs, TNF alfa inhibitors, tocolytic agents, topical acne agents, topical agents, topical allergy diagnostic agents, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical depigmenting agents, topical emollients, topical keratolytics, topical non-steroidal anti-inflammatories, topical photochemotherapeutics, topical rubefacient, topical steroids, topical steroids with anti-infectives, transthyretin stabilizers, triazine anticonvulsants, tricyclic antidepressants, trifunctional monoclonal antibodies, Taclonex, Tafinlar, Tagitol V, Tagrisso, Talzenna, Tambocor, Tamiflu, Tarceva, Targretin, Targretin Gel, Tarka, Tasigna, Tasmar, Tavalisse, Taxotere, Taytulla, Tazorac, Tecfidera, Teflaro, Tegretol, Tegretol XR, Tegsedi, Tekturna, Tekturna HCT, Temixys, Temodar, Tenoretic 100, Tenoretic 50, Tenormin, Tepadina, Tessalon, Testim, Thalomid, Thermazene, Thiola, Thiola EC, Thyrogen, Tiazac, Tibsovo, Tigan, Tikosyn, Timoptic, Timoptic-XE, Tindamax, Tirosint, Tirosint-Sol, Tivicay, Tivorbex, Tobi, TOBI Podhaler, TobraDex, Tobradex ST, Tobrex, Tolak, Tolsura, Topamax, Topicort, Toprol-XL, Torisel, Tosymra, Totect, Toujeo Max SoloStar, Toujeo SoloStar, Toviaz, TPDXX, Tracleer, Tradjenta, Trandate, Transderm-Scop, Tranxene, Travatan Z, Treanda, Trecator, Trelegy Ellipta, Trelstar, Tresiba, Treximet, Tri-Luma, Tribenzor, TriCor, Triferic, Triglide, Trileptal, Trilipix, Trintellix, Triostat, Triphasil-28, Trisenox, Triumeq, Trizivir, Trokendi XR, Trulance, Trusopt, Truvada, Tudorza Pressair, Turalio, Tuxarin ER, Tuzistra XR, Twynsta, Tybost, ultrasound contrast media, upper respiratory combinations, urea anticonvulsants, urea cycle disorder agents, urinary anti-infectives, urinary antispasmodics, urinary pH modifiers, uterotonic agents, Uceris, Uloric, Ultane, Ultiva, Ultracet, Ultram, Ultravate, Ultravist, Unasyn, Uptravi, Urex, Urocit-K, Uroxatral, Urso, Urso Forte, Uvadex, vaccine combinations, vaginal anti-infectives, vaginal preparations, vasodilators, vasopressin antagonists, vasopressors, VEGF/VEGFR inhibitors, viral vaccines, viscosupplementation agents, vitamin and mineral combinations, vitamins, VMAT2 inhibitors, Vabomere, Vagifem, Valchlor, Valcyte, Valium, Valstar, Valtrex, Vandazole, Vaniqa, Vanos, Vantas, Varibar Honey, Varibar Nectar, Varibar Pudding, Varibar Thin Honey, Varubi, Vascepa, Vaseretic, Vasostrict, Vasotec, Vazculep, Vectical, Velcade, Veletri, Velphoro, Veltassa, Veltin, Vemlidy, Venclexta, Venofer, Ventavis, Ventolin HFA, Verdeso, Veregen, Verelan, Verelan PM, Versacloz, Verzenio, VESIcare, Vfend, Viagra, Vibativ, Viberzi, Vibramycin, Victoza, Vidaza, Videx, Videx EC, Vigamox, Viibryd, Vimovo, Vimpat, Viracept, Viramune, Viramune XR, Virazole, Viread, Viroptic, VisionBlue, Vistaril, Vistogard, Visudyne, Vitrakvi, Vitrase, Vivelle-Dot, Vivitrol, Vivlodex, Vizamyl, Vizimpro, Vogelxo, Voltaren Ophthalmic, Vosevi, Vosol, Vosol HC, Votrient, VPRIV, Vraylar, Vumerity, Vyndamax, Vyndagel, Vytorin, Vyvanse, Vyzulta, Wakix, Welchol, Wellbutrin, Wellbutrin SR, Wellbutrin XL, Xadago, Xalatan, Xalkori, Xanax, Xanax XR, Xarelto, Xatmep, Xeljanz, Xeljanz XR, Xeloda, Xelpros, Xenazine, Xenical, Xenleta, Xepi, Xerava, Xerese, Xermelo, Xifaxan, Xigduo XR, Xiidra, Ximino, Xofigo, Xofluza, Xolegel, Xopenex, Xopenex HFA, Xospata, Xpovio, Xtampza ER, Xtandi, Xultophy, Xuriden, Xyrem, Xyzal, Yasm in, Yaz, Yondelis, Yonsa, Yosprala, Yupelri, Yutiq, Zanaflex, Zanosar, Zantac, Zantac 300, Zarontin, Zaroxolyn, Zavesca, Zegerid, Zejula, Zelapar, Zelboraf, Zelnorm, Zembrace SymTouch, Zemdri, Zemplar, Zepatier, Zerbaxa, Zerit, Zerviate, Zestoretic, Zestril, Zetia, Zetonna, Ziac, Ziagen, Ziana, Zilretta, Zinacef, Zinecard, Zioptan, Zipsor, Zirgan, Zithromax, Zocor Zofran, Zofran ODT, Zohydro ER, Zoladex, Zolinza, Zoloft, Zolpimist, Zomacton, Zometa, Zomig, Zomig-ZMT, Zonalon, Zonegran, Zontivity, Zorbtive, Zortress, Zorvolex, Zosyn, Zovirax, Zovirax Ointment, ZTlido, Zubsolv, Zulresso, Zuplenz, Zyclara, Zydelig, Zyflo, Zyflo CR, Zykadia, Zylet, Zyloprim, Zymar, Zymaxid, Zypitamag, Zyprexa, Zyprexa Relprevv, Zyprexa Zydis, Zytiga, or Zyvox.

In an embodiment, the active ingredient is prostaglandin or a prostaglandin analog. In a further embodiment, the prostaglandin analog is selected from the group consisting of: bimatoprost, latanoprost, tafluprost, and travoprost. In another embodiment, the protein having hyaluronidase activity is a human recombinant hyaluronidase such as HYLENEX and the active ingredient is a prostaglandin analog selected from the group consisting of: bimatoprost, latanoprost, tafluprost, and travoprost.

### Methods of Treating: Cancer and Other Disorders

The present disclosure provides methods of administering a protein having hyaluronidase, collagenase, and/or 4-methylumbelliferone (4-MU) activity, with or without one or more additional active pharmaceutical ingredients (APIs), to a subject in need thereof to treat and/or prevent a disease or disorder.

In one embodiment, the disease or disorder may be cancer. In one embodiment the cancer may be selected from the group consisting of: oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, glioma; parathyroid cancer, penile cancer, vaginal cancer, thyroid cancer, pancreatic cancer, esophageal cancer, Hodgkin's lymphoma, leukemia-related disorders, mycosis fungoides, and myelodysplasia syndrome.

In another embodiment, the cancer may be non-small cell lung cancer, pancreatic cancer, breast cancer, ovarian cancer, colorectal cancer, or head and neck cancer. In yet another embodiment the cancer may be a carcinoma, a tumor, a neoplasm, a lymphoma, a melanoma, a glioma, a sarcoma, or a blastoma.

In one embodiment, the carcinoma may be selected from the group consisting of: carcinoma, adenocarcinoma, adenoid cystic carcinoma, adenosquamous carcinoma, adrenocortical carcinoma, well differentiated carcinoma, squamous cell carcinoma, serous carcinoma, small cell carcinoma, invasive squamous cell carcinoma, large cell carcinoma, islet cell carcinoma, oat cell carcinoma, squamous carcinoma, undifferentiatied carcinoma, verrucous carcinoma, renal cell carcinoma, papillary serous adenocarcinoma, merkel cell carcinoma, hepatocellular carcinoma, soft tissue carcinomas, bronchial gland carcinomas, capillary carcinoma, bartholin gland carcinoma, basal cell carcinoma, carcinosarcoma, papilloma/carcinoma, clear cell carcinoma, endometrioid adenocarcinoma, mesothelial, metastatic carcinoma, mucoepidermoid carcinoma, cholangiocarcinoma, actinic keratoses, cystadenoma, and hepatic adenomatosis.

In another embodiment, the tumor may be selected from the group consisting of: astrocytic tumors, malignant mesothelial tumors, ovarian germ cell tumors, supratentorial primitive neuroectodermal tumors, Wilms tumors, pituitary tumors, extragonadal germ cell tumors, gastrinoma, germ cell tumors, gestational trophoblastic tumors, brain tumors, pineal and supratentorial primitive neuroectodermal tumors, pituitary tumors, somatostatin-secreting tumors, endodermal sinus tumors, carcinoids, central cerebral astrocytoma, glucagonoma, hepatic adenoma, insulinoma, medulloepithelioma, plasmacytoma, vipoma, and pheochromocytoma.

In yet another embodiment, the neoplasm may be selected from the group consisting of: intraepithelial neoplasia, multiple myeloma/plasma cell neoplasm, plasma cell neoplasm, interepithelial squamous cell neoplasia, endometrial hyperplasia, focal nodular hyperplasia, hemangioendothelioma, and malignant thymoma. In a further embodiment the lymphoma may be selected from the group consisting of: nervous system lymphoma, AIDS-related lymphoma, cutaneous T-cell lymphoma, non-Hodgkin's lymphoma, lymphoma, and Waldenstrom's macroglobulinemia. In another embodiment the melanoma may be selected from the group consisting of: acral lentiginous melanoma, superficial spreading melanoma, uveal melanoma, lentigo maligna melanomas, melanoma, intraocular melanoma, adenocarcinoma nodular melanoma, and hemangioma. In yet another embodiment the sarcoma may be selected from the group consisting of: adenomas, adenosarcoma, chondosarcoma, endometrial stromal sarcoma, Ewing's sarcoma, Kaposi's sarcoma, leiomyosarcoma, rhabdomyosarcoma, sarcoma, uterine sarcoma, osteosarcoma, and pseudosarcoma. In one embodiment the glioma may be selected from the group consisting of: glioma, brain stem glioma, and hypothalamic and visual pathway glioma. In another embodiment, the blastoma may be selected from the group consisting of: pulmonary blastoma, pleuropulmonary blastoma, retinoblastoma, neuroblastoma, medulloblastoma, glioblastoma, and hemangiblastomas.

In some embodiments, the protein having hyaluronidase, collagenase, and/or 4-methylumbelliferone (4-MU) activity, with or without one or more additional active pharmaceutical ingredients (APIs), can be administrated locally or topically, such as, a transdermal patch or topical cream or topical ointment to the area of cellulite or can be administered via an implant, such as, microcapsules or microspheres which release the protein having hyaluronidase, collagenase, and/or 4-methylumbelliferone (4-MU) activity, with or without one or more additional active pharmaceutical ingredients (APIs), over time.

### Methods of Treating: Inflammation

Pharmacological agents have been proposed for use in treating localized inflammation. Such agents are typically selected from NSAIDs such as aspirin, ibuprofen, and the like, corticosteroids, and antihistamines. These agents can provide some degree of improvement, but relief is often minimal and short-lived. The inventors have discovered that a protein having hyaluronidase activity such as a recombinant human hyaluronidase can be used to treat and/or prevent inflammation including, reducing redness, heat, pain, swelling, and/or immobility, without many of the drawbacks of existing treatments.

Thus, the present disclosure provides compositions comprising a protein having hyaluronidase, collagenase, and/or 4-methylumbelliferone (4-MU) activity, with or without one or more additional active pharmaceutical ingredients (APIs), and the use of such compositions for the treatment and/or prevention of inflammation (e.g., inflammation of the skin). Such methods may comprise administering to a subject in need thereof a composition that comprises a protein having hyaluronidase activity (e.g., a hyaluronidase such as Hylenex, Amphadase, or Vitrase), collagenase activity, and/or 4-methylumbelliferone (4-MU) activity, with or without one or more additional active pharmaceutical ingredients (APIs), to a region of the subject having inflammation.

The methods disclosed herein may be used to reduce the severity of inflammation including reducing the severity by one or more grades. In an embodiment, inflammation classified as severe (4) is reduced to moderate (3), mild (2), quiescent (1), or normal (0). In another embodiment, inflammation classified as moderate is reduced to mild, quiescent, or normal. In yet another embodiment, inflammation classified as mild is reduced to quiescent or normal. In a further embodiment, inflammation classified as quiescent is reduced to normal.

In an embodiment, the methods disclosed herein may reduce or eliminate redness, heat, swelling, pain, and/or immobility associated with inflammation.

The present disclosure provides compositions comprising a protein having hyaluronidase, collagenase, and/or 4-methylumbelliferone (4-MU) activity, with or without one or more additional active pharmaceutical ingredients (APIs), and the use of such compositions for the treatment and/or prevention of inflammation (e.g., inflammation of the skin). Such methods may comprise administering to a subject in need thereof a composition that comprises a protein having hyaluronidase activity (e.g., a hyaluronidase such as Hylenex, Amphadase, or Vitrase), collagenase activity, and/or 4-methylumbelliferone (4-MU) activity, with or without one or more additional active pharmaceutical ingredients (APIs), to a region of the subject having inflammation.

The methods disclosed herein may be used to reduce the severity of inflammation including reducing the severity by one or more grades. In an embodiment, inflammation classified as severe (4) is reduced to moderate (3), mild (2), quiescent (1), or normal (0). In another embodiment, inflammation classified as moderate is reduced to mild, quiescent, or normal. In yet another embodiment, inflammation classified as mild is reduced to quiescent or normal. In a further embodiment, inflammation classified as quiescent is reduced to normal. Additionally or alternatively, the methods disclosed herein may reduce or eliminate redness, heat, swelling, pain, and/or immobility associated with inflammation.

The present disclosure provides methods of administering a protein having hyaluronidase, collagenase, and/or 4-methylumbelliferone (4-MU) activity, with or without one or more additional active pharmaceutical ingredients (APIs), to a subject in need thereof to treat and/or prevent inflammation. The compositions can be formulated in lyophilized or liquid form. Where the compositions are provided in lyophilized form they can be reconstituted just prior to use by an appropriate buffer, for example, a sterile saline solution. The compositions can be packaged as a kit.

The actual amount of the hyaluronidase, collagenase, and/or 4-methylumbelliferone (4-MU), with or without one or more additional active pharmaceutical ingredients (APIs), to be administered in any given case will be determined by a physician or other skilled person taking into account the relevant circumstances, such as the amount of inflammation in the tissues, the desired reduction in the puffiness, the potential fat reduction, the age and weight of the patient, the patient's general physical condition, the cause of the condition, and the route of administration.

Other therapeutically efficient amounts of a hyaluronidase, collagenase, and/or 4-MU, with or without one or more additional APIs, will be apparent to a skilled person upon a reading of the present disclosure. For example, a skilled person can determine the maximum safe dosage for healthy subjects based on the dosages used in animal studies by routine methods (see, e.g. Dept. of Health and Human Services “Guidance For Industry: Estimating the Maximum Safe Starting Dose in Initial Clinical Trials for Therapeutics in Adult Healthy Volunteers”), and then administer to subjects in need thereof various dosages below the maximum safe dosage by routine methods and experimentation until a dosage which results in a desirable effect (e.g. reduction in the extent of peri-orbital puffiness, festoons, or malar puffiness due to inflammation) is reached.

### Methods of Treating: Fibrosis

Fibrosis is characterized by the overgrowth, hardening, and/or scarring of various tissues and is attributed to excess deposition of extracellular matrix components including collagen. Current treatments for fibrotic diseases such as scleroderma target the inflammatory response. The inventors have surprisingly discovered that a protein having hyaluronidase activity such as a recombinant human hyaluronidase can be used to treat and/or prevent fibrosis, including, for example, reduce the severity of fibrosis (e.g., reduce moderate fibrosis to mild fibrosis).

In an embodiment, the fibrosis is dermal fibrosis that is a result of a pathologic wound healing response including, for example, scleroderma, nephrogenic fibrosing dermopathy, mixed connective tissue disease, scleromyxedema, scleredema, and eosinophilic fasciitis.

The methods disclosed herein may reduce the amount or severity of fibrosis. For example, the amount or severity of fibrosis may be reduced from severe, moderate, or mild fibrosis to no fibrosis. Alternatively, the amount or severity of the fibrosis may be reduced from severe or moderate fibrosis to mild fibrosis. In yet another example, the amount or severity of the fibrosis may be reduced from moderate fibrosis to mild fibrosis.

The present disclosure provides methods of treating, including reducing the severity fibrosis (e.g., dermal fibrosis) in a subject in need thereof. Such methods may comprise administering a composition that comprises a protein having hyaluronidase, collagenase, and/or 4-MU activity, with or without one or more additional APIs, activity to the subject. In some embodiments, the fibrosis is selected from the group consisting of: pulmonary fibrosis, idiopathic pulmonary fibrosis, cirrhosis, endomyocardial fibrosis, mediastinal fibrosis, myelofibrosis, retroperitoneal fibrosis, progressive massive fibrosis, nephrogenic systemic fibrosis, Crohn's Disease intestine, keloid, myocardial infarction, scleroderma/systemic sclerosis, arthrofibrosis, and adhesive capsulitis.

Preferably, the fibrosis is a fibrotic skin disease such as scleroderma, nephrogenic fibrosing dermopathy, mixed connective tissue disease, scleromyxedema, scleredema, or eosinophilic fasciitis.

The present disclosure also provides methods of reducing fibrosis in a subject in need thereof by administering a composition that comprises a protein having hyaluronidase, collagenase, and/or 4-MU activity, with or without one or more additional APIs, to a region of fibrosis on the subject, wherein the administration results in a reduction in a grade of severity of the fibrosis.

The fibrosis prior to treatment with the composition may be classified as mild fibrosis, moderate fibrosis, or severe fibrosis. After treatment with the composition, the fibrosis may be classified as no fibrosis, mild fibrosis, moderate fibrosis or severe fibrosis.

In some embodiments, the methods and compositions disclosed herein are used to cover trap door scars in a subject in need thereof. In some embodiments, trap door scars trap fluid.

In some embodiments, the methods and compositions disclosed herein are used at surgical sites in a subject in need thereof.

In some embodiments, the methods and compositions disclosed herein are used for treating survical wounds in a subject in need thereof.

In some embodiments, the methods and compositions disclosed herein are used to decrease the degree of burn classification in a subject in need thereof. In some embodiments, the decrease in the degree of burn classification is from fourth to third. In some embodiments, the decrease in the degree of burn classification is from third to second. In some embodiments, the decrease in the degree of burn classification is from second to first.

In some embodiments, the methods and compositions disclosed herein are used to treat and/or reduce scarring from burns in a subject in need thereof.

In some embodiments, the methods and compositions disclosed herein are used during interventional cardiology and radiology in a subject in need thereof. In some embodiments, the methods and compositions disclosed herein are used to dissolve fibrosis by limiting the execution of the procedure to access and remove a medical device in a subject in need thereof. In some embodiments, the methods and compositions disclosed herein are used to permit improved access to the target organ, including removal of a foreign object from the body of the subject in need thereof.

In some embodiments, the methods and compositions disclosed herein are used during or instead of a fasciotomy to reduce swelling and/or to save one or more limbs.

In some embodiments, the methods and compositions disclosed herein do not comprise hyaluronidase. In some embodiments, the methods and compositions disclosed herein comprise elastinase and do not comprise hyaluronidase.

In some embodiments, the methods and compositions disclosed herein are used in a subject who is HIV positive and/or has AIDS.

In some embodiments, the methods and compositions disclosed herein comprise protease inhibitors that cause fat atrophy in the body of a subject thereof. In some embodiments, the methods and compositions disclosed herein comprising protease inhibitors that cause fat atrophy in the body of a subject thereof are used to decrease fullness around the eyes.

Methods of Treating: Anterior Facial Fullness, Jowls, and/or Labiomandibular Folds

With age, the skin typically loses and fails to replenish its stores of collagen and elastin which are both important for maintaining flexibility and elasticity. As a result, skin begins to sag and droop. The inventors have discovered that a protein having hyaluronidase activity such as a recombinant human hyaluronidase can be used to treat and/or prevent anterior facial fullness, jowls, and/or labiomandibular folds without many of the drawbacks of existing treatments.

The present disclosure provides methods of treating anterior facial fullness, jowls, and/or labiomandibular folds in a subject in need thereof, the method comprising administering a composition that comprises a protein having hyaluronidase, collagenase, and/or 4-MU activity, with or without one or more additional APIs, to an area of skin on the subject exhibiting the anterior facial fullness, jowls, and/or labiomandibular folds, respectively. In an embodiment, the anterior facial fullness is treating by administering the protein having hyaluronidase, collagenase, and/or 4-MU activity, with or without one or more additional APIs, to an area of skin along an anterior cheek bordered medially by a nasolabial fold, laterally by a midface groove, superiorly by a nasojugal groove and inferiorly by a jowl.

The present disclosure provides methods of administering a protein having hyaluronidase, collagenase, and/or 4-MU activity, with or without one or more additional APIs, to a subject in need thereof to treat and/or prevent anterior facial fullness, jowls, and/or labiomandibular folds. The compositions can be formulated in lyophilized or liquid form. Where the compositions are provided in lyophilized form they can be reconstituted just prior to use by an appropriate buffer, for example, a sterile saline solution. The compositions can be packaged as a kit.

The methods disclosed herein may be used to treat jowls including, for example, reduce the severity of jowls.

In an embodiment, a jaw line is graded as follows. Grade 1: tight jaw line; Grade 2: softening of jaw line definition; Grade 3: some blurring of jaw line and loosening of tissues with mild jowl formation; Grade 4: indistinct jaw line with quite obvious jowls; Grade 5: significant sagging eliminating jaw line definition; severe jowls. The methods disclosed herein may be used to reduce the grading of a jaw line including, for example, i.) from a Grade 5 to Grade 4, Grade 3, Grade 2, or Grade 1, ii.) a Grade 4 to a Grade 3, Grade 2 or Grade 1, iii.) a Grade 3 to a Grade 2 or Grade 1, or iv.) a Grade 2 to a Grade 1.

### Methods of Treating Puffiness/Fullness Using Hyaluronidase and Collagenase

The present disclosure provides methods for treating including, reducing and/or eliminating upper and/or lower eyelid puffiness/fullness in a subject by administering a therapeutically effective amount of a composition having a protein with hyaluronidase activity (e.g., human recombinant hyaluronidase such as Hylenex) and a collagenase to one or more of the upper and/or lower eyelid fat pads.

In some embodiments, the reduction or elimination of the puffiness or fullness is maintained for 1 month, 2 months, 3 months, 4 months, 5 months, 6 months, 7 months, 8 months, 9 months, 10 months, 11 months, 1 year, 2 years, 3 years or more.

In some embodiments, the composition can be administrated locally or topically, such as, a transdermal patch or topical cream or topical ointment to the area of skin having the puffiness or can be administered via an implant, such as, microcapsules or microspheres which release the protein having hyaluronidase activity and collagenase over time.

### Methods for Remyelinating an Axon

Multiple sclerosis (MS) afflicts approximately 400,000 people in the United States and 2.5 million worldwide. MS is an inflammatory disease in which myelin sheaths around the axons of the brain and spinal cord are damaged. In MS as well as other demyelinating diseases, autoimmune inflammatory attack against myelin and oligodendrocytes causes demyelination. The thinning or loss of myelin surrounding axons impairs the ability of the axons to effectively conduct signals and results in progressive neuronal damage. Although a multitude of therapies exist, most of them do not provide a lasting effect and require multiple treatments on an ongoing basis to maintain their effect at significant expense and with mixed results. The inventors have discovered that a protein having hyaluronidase activity such as a recombinant human hyaluronidase can be used to treat and/or prevent diseases and disorders associated with demyelinated neurons and advantageously lead to remyelination of neurons.

The methods disclosed herein may increase the amount of myelination of an axon including, an axon of a neuron. Such methods may be used to restore a fully demyelinated axon and to a partially or fully myelinated neuron. Alternatively, the methods may be used to restore a partially demyelinated axon to a fully myelinated neuron.

The present disclosure provides methods for remyelination of an axon including remyelinating an axon of a neuron. Such methods may preferably be used to remyelinate neurons in a subject and thereby treat diseases or disorders associated with demyelinated neurons such as MS. Such methods may comprise administering a protein having hyaluronidase, collagenase, and/or 4-MU activity, with or without one or more additional APIs, to a region of the subject having demyelinated axons.

### Formulations, Dosage, and Administration

The present disclosure provides formulations and compositions of a protein having hyaluronidase, collagenase, and/or 4-methylumbelliferone (4-MU) activity, with or without one or more additional active pharmaceutical ingredients (APIs). Preferably, the formulations are sterile and/or stable. The compositions can be formulated in lyophilized or liquid form. Where the compositions are provided in lyophilized form they can be reconstituted just prior to use by an appropriate buffer, for example, a sterile saline solution. The compositions can be provided together or separately. The pharmaceutical composition can be formulated in lyophilized or liquid form. The compositions can be packaged as a kit.

In some embodiments, the protein having hyaluronidase, collagenase, and/or 4-MU activity, with or without one or more additional APIs, can be administrated locally or topically, such as, a transdermal patch or topical cream or topical ointment to the area of fibrosis or can be administered via an implant, such as, microcapsules or microspheres which release the protein having hyaluronidase, collagenase, and/or 4-MU activity, with or without one or more additional APIs, over time.

In some embodiments, a formulation can comprise a therapeutically effective amount of crosslinked and uncrosslinked hyaluronidase, wherein the ratio of crosslinked to uncrosslinked hyaluronidase is between about 1:9 and about 9:1. In some embodiments, the ratio is between about 1:9 and about 1:8. In some embodiments, the ratio is between about 1:8 and about 1:7. In some embodiments, the ratio is between about 1:7 and about 1:6. In some embodiments, the ratio is between about 1:6 and about 1:5. In some embodiments, the ratio is between about 1:5 and about 1:4. In some embodiments, the ratio is between about 1:4 and about 1:3. In some embodiments, the ratio is between about 1:3 and about 1:2. In some embodiments, the ratio is between about 1:2 and about 1:1. In some embodiments, the ratio is between about 1:1 and about 2:1. In some embodiments, the ratio is between about 2:1 and about 3:1. In some embodiments, the ratio is between about 3:1 and about 4:1. In some embodiments, the ratio is between about 4:1 and about 5:1. In some embodiments, the ratio is between about 5:1 and about 6:1. In some embodiments, the ratio is between about 6:1 and about 7:1. In some embodiments, the ratio is between about 7:1 and about 8:1. In some embodiments, the ratio is between about 8:1 and about 9:1.

In cases where results of a single treatment are considered inadequate, the same methods, total amount of protein having hyaluronidase, collagenase, and/or 4-MU activity, with or without one or more additional APIs, and concentration may be repeated at 4-6 weeks intervals.

The compositions can be formulated into any suitable pharmaceutical preparations for subcutaneous administration such as solutions, suspensions, powders, or sustained release formulations. Typically, the compositions are formulated into pharmaceutical compositions using techniques and procedures well known in the art (see e.g., Ansel Introduction to Pharmaceutical Dosage Forms, Fourth Edition, 1985, 126). Pharmaceutically acceptable compositions are prepared in view of approvals for a regulatory agency or other agency prepared in accordance with generally recognized pharmacopeia for use in animals and in humans. The formulation should suit the mode of administration.

Pharmaceutical compositions can include carriers such as a diluent, adjuvant, excipient, or vehicle with which a hyaluronidase, collagenase, and/or 4-MU activity, with or without one or more additional APIs, or IG is administered. Examples of suitable pharmaceutical carriers are described in “Remington's Pharmaceutical Sciences” by E. W. Martin. Such compositions will contain a therapeutically effective amount of the compound, generally in purified form or partially purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyvinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrine derivatives, sorbitan monolaurate, triethanolamine sodium acetate, triethanolamine oleate, and other such agents.

Pharmaceutically therapeutically active compounds and derivatives thereof are typically formulated and administered in unit dosage forms or multiple dosage forms. Each unit dose contains a predetermined quantity of therapeutically active compound sufficient to produce the desired therapeutic effect, in association with the required pharmaceutical carrier, vehicle or diluent. Examples of unit dose forms include ampoules and syringes and individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging. Generally, dosage forms or compositions containing active ingredient in the range of 0.005% to 100% with the balance made up from non-toxic carrier can be prepared.

Compositions provided herein typically are formulated for administration by subcutaneous route, although other routes of administration are contemplated, such as any route known to those of skill in the art. Formulations suited for such routes are known to one of skill in the art. Administration can be local, topical or systemic depending upon the locus of treatment. Local administration to an area in need of treatment can be achieved by, for example, but not limited to, local infusion during surgery, topical application, transdermal patch, or by injection. Compositions also can be administered with other biologically active agents, either sequentially, intermittently or in the same composition. Thus, in one example, local administration can be achieved by injection, such as from a syringe or other article of manufacture containing a injection device such as a needle or an injection device containing multiple needles. In another example, local administration can be achieved by infusion, which can be facilitated by the use of a pump or other similar device, or by a transdermal patch. Pharmaceutical compositions can be formulated in dosage forms appropriate for each route of administration.

Subcutaneous administration, generally characterized by injection or infusion, is contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. The pharmaceutical compositions may contain other minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. The percentage of active compound contained in such compositions is highly dependent on the specific nature thereof, as well as the activity of the compound and the needs of the subject.

Injectables are designed for local and systemic administration. For purposes herein, local administration is desired for direct administration to the affected area. The solutions may be either aqueous or nonaqueous.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers, which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose.

Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEENs 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art. The unit-dose parenteral preparations are packaged in an ampoule, a vial or a syringe with a needle. The volume of liquid solution or reconstituted powder preparation, containing the pharmaceutically active compound, is a function of the disease to be treated and the particular article of manufacture chosen for package.

In one example, a pharmaceutical preparation can be in liquid form, for example, solutions, syrups or suspensions. If provided in liquid form, the pharmaceutical preparations can be provided as a concentrated preparation to be diluted to a therapeutically effective concentration before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). In another example, pharmaceutical preparations can be presented in lyophilized form for reconstitution with water or other suitable vehicle before use.

Administration methods can be employed to decrease the exposure of the hyaluronidase, collagenase, and/or 4-MU activity, with or without one or more additional APIs, to degradative processes, such as proteolytic degradation and immunological intervention via antigenic and immunogenic responses. Examples of such methods include local administration at the site of treatment. Pegylation of therapeutics has been reported to increase resistance to proteolysis, increase plasma half-life, and decrease antigenicity and immunogenicity. Examples of pegylation methodologies are known in the art (see for example, Lu and Felix, Int. J. Peptide Protein Res., 43: 127-138, 1994; Lu and Felix, Peptide Res., 6: 142-6, 1993; Felix et al., Int. J. Peptide Res., 46: 253-64, 1995; Benhar et al., J. Biol. Chem., 269: 13398-404, 1994; Brumeanu et al., J Immunol., 154: 3088-95, 1995; see also, Caliceti et al. (2003) Adv. Drug Deliv. Rev. 55(10):1261-77 and Molineux (2003) Pharmacotherapy 23 (8 Pt 2):3S-8S). Pegylation also can be used in the delivery of nucleic acid molecules in vivo. For example, pegylation of adenovirus can increase stability and gene transfer (see, e.g., Cheng et al. (2003) Pharm. Res. 20(9): 1444-2. Dosage and Administration

Typically, a therapeutically effective dose is at or about 10 Units to 100,000 Units of a soluble hyaluronidase, collagenase, and/or 4-MU, with or without one or more additional APIs,. For example, soluble hyaluronidase, collagenase, and/or 4-MU, with or without one or more additional APIs, can be administered subcutaneously at or about 10 units, 20 Units, 50 Units, 100 Units, 200 Units, 500 Units, 1000 Units, 2000 Units, 5000 Units, 10,000 Units, 30,000 Units, 40,000 Units, 50,000 Units, 60,000 Units, 70,000 Units, 80,000 Units, 90,000 Units, 100,000 Units or more. Typically, volumes of injections or infusions of hyaluronidase, collagenase, and/or 4-MU, with or without one or more additional APIs, contemplated herein are from at or about 0.1 ml, 0.2 ml, 0.3 ml, 0.5 ml, 1 ml, 2 ml, 3 ml, 4 ml, 5 ml, 6 ml, 7 ml, 8 ml, 9 ml, 10 ml, 15 ml, 20 ml, 30 ml, 40 ml, 50 ml or more. The hyaluronidase, collagenase, and/or 4-MU, with or without one or more additional APIs, can be provided as a stock solution at or about 50 U/ml, 100 U/ml, 150 U/ml, 200 U/ml, 400 U/ml or 500 U/ml or can be provided in a more concentrated form, for example at or about 1000 U/ml, 1500 Units/ml, 2000 U/ml, 4000 U/ml or 5000 U/ml for use directly or for dilution to the effective concentration prior to use.

The actual amount of the hyaluronidase, collagenase, and/or 4-MU, with or without one or more additional APIs, to be administered in any given case will be determined by a physician or other skilled person taking into account the relevant circumstances, such as the amount of edema in the tissues, the desired reduction in the puffiness, the potential fat reduction, the age and weight of the patient, the patient's general physical condition, the cause of the condition, and the route of administration.

Other therapeutically efficient amounts of a hyaluronidase, collagenase, and/or 4-MU, with or without one or more additional APIs, will be apparent to a skilled person upon a reading of the present disclosure. For example, a skilled person can determine the maximum safe dosage for healthy subjects based on the dosages used in animal studies by routine methods (see, e.g. Dept. of Health and Human Services “Guidance For Industry: Estimating the Maximum Safe Starting Dose in Initial Clinical Trials for Therapeutics in Adult Healthy Volunteers”), and then administer to subjects in need thereof various dosages below the maximum safe dosage by routine methods and experimentation until a dosage which results in a desirable effect (e.g. reduction in the extent of peri-orbital puffiness, festoons, or malar puffiness due to edema) is reached.

The therapeutically efficient amount of a hyaluronidase, collagenase, and/or 4-MU, with or without one or more additional APIs, can be present in a formulation (e.g. for topical administration) at between about 0.01 and about 5% (w/v). In some embodiments, the therapeutically effective amount in the formulation can be from about 0.01 to about 1%, about 0.01 to about 2%, about 0.01 to about 3%, and about 0.01 to about 4%. In other embodiments, the therapeutically effective amount in the formulation can be from about 0.01 to about 1%, about 1 to about 2%, about 2 to about 3%, about 3 to about 4%, about 4 to about 5%.

In other embodiments, the therapeutically effective amount of a hyaluronidase, collagenase, and/or 4-MU, with or without one or more additional APIs, in the formulation can be from about 0.01 to about 0.06%, about 0.06 to about 0.11%, about 0.11 to about 0.16%, about 0.16 to about 0.21%, about 0.21 to about 0.26%, about 0.26 to about 0.31%, about 0.31 to about 0.36%, about 0.36 to about 0.41%, about 0.41 to about 0.46%, about 0.46 to about 0.51%, about 0.51 to about 0.56%, about 0.56 to about 0.61%, about 0.61 to about 0.66%, about 0.66 to about 0.71%, about 0.71 to about 0.76%, about 0.76 to about 0.81%, about 0.81 to about 0.86%, about 0.86 to about 0.91%, about 0.91 to about 0.96%, about 0.96 to about 1.01%, about 1.01 to about 1.06%, about 1.06 to about 1.11%, about 1.11 to about 1.16%, about 1.16 to about 1.21%, about 1.21 to about 1.26%, about 1.26 to about 1.31%, about 1.31 to about 1.36%, about 1.36 to about 1.41%, about 1.41 to about 1.46%, about 1.46 to about 1.51%, about 1.51 to about 1.56%, about 1.56 to about 1.61%, about 1.61 to about 1.66%, about 1.66 to about 1.71%, about 1.71 to about 1.76%, about 1.76 to about 1.81%, about 1.81 to about 1.86%, about 1.86 to about 1.91%, about 1.91 to about 1.96%, about 1.96 to about 2.01%, about 2.01 to about 2.06%, about 2.06 to about 2.11%, about 2.11 to about 2.16%, about 2.16 to about 2.21%, about 2.21 to about 2.26%, about 2.26 to about 2.31%, about 2.31 to about 2.36%, about 2.36 to about 2.41%, about 2.41 to about 2.46%, about 2.46 to about 2.51%, about 2.51 to about 2.56%, about 2.56 to about 2.61%, about 2.61 to about 2.66%, about 2.66 to about 2.71%, about 2.71 to about 2.76%, about 2.76 to about 2.81%, about 2.81 to about 2.86%, about 2.86 to about 2.91%, about 2.91 to about 2.96%, about 2.96 to about 3.01%, about 3.01 to about 3.06%, about 3.06 to about 3.11%, about 3.11 to about 3.16%, about 3.16 to about 3.21%, about 3.21 to about 3.26%, about 3.26 to about 3.31%, about 3.31 to about 3.36%, about 3.36 to about 3.41%, about 3.41 to about 3.46%, about 3.46 to about 3.51%, about 3.51 to about 3.56%, about 3.56 to about 3.61%, about 3.61 to about 3.66%, about 3.66 to about 3.71%, about 3.71 to about 3.76%, about 3.76 to about 3.81%, about 3.81 to about 3.86%, about 3.86 to about 3.91%, about 3.91 to about 3.96%, about 3.96 to about 4.01%, about 4.01 to about 4.06%, about 4.06 to about 4.11%, about 4.11 to about 4.16%, about 4.16 to about 4.21%, about 4.21 to about 4.26%, about 4.26 to about 4.31%, about 4.31 to about 4.36%, about 4.36 to about 4.41%, about 4.41 to about 4.46%, about 4.46 to about 4.51%, about 4.51 to about 4.56%, about 4.56 to about 4.61%, about 4.61 to about 4.66%, about 4.66 to about 4.71%, about 4.71 to about 4.76%, about 4.76 to about 4.81%, about 4.81 to about 4.86%, about 4.86 to about 4.91%, about 4.91 to about 4.96%, and about 4.96 to about 5% (w/v).

The therapeutically effective amount can be administered according to a dosing frequency that is identifiable to a skilled person during a time period that is also identifiable to a skilled person. The term “dosing frequency” as used herein, refers to the number of times the compounds described herein are administered to a subject. Exemplary dosing frequencies include administering the effective amount at discrete times during a day such as, for example, once a day (QD), twice a day (BID), three times a day (TID), four times a day (QID), and others identifiable to a skilled person. Other exemplary dosing frequencies include continuous dosing, for example by intravenous infusion, use of a drug pump, use of a transdermal patch, or other methods of continuous dosing identifiable to a skilled person.

The therapeutically effective amount can be administered at a desired dosing frequency for a time period identifiable to a skilled person. For example, a therapeutically effective can be administered once or twice a day (or at another dosing frequency identifiable to a skilled person) for a set period of time (e.g. seven to fourteen days, two to four weeks, one to six months, or for another time period identifiable to a skilled person). As another example, a therapeutically effective amount can be administered once or twice a day (or at another dosing frequency identifiable to a skilled person) for a non-predetermined period of time. A skilled person can determine at various points during the period of time if the administration of the effective amount is to be continued.

Pharmaceutical compositions of hyaluronidase, collagenase, and/or 4-MU, with or without one or more additional APIs, can be packaged as articles of manufacture containing packaging material, a pharmaceutical composition which is effective for treating puffiness, and a label that indicates that the composition is to be used for treating puffiness. Exemplary of articles of manufacture are containers including single chamber and dual chamber containers. The containers include, but are not limited to, tubes, bottles and syringes. The containers can further include a needle for subcutaneous administration.

The articles of manufacture provided herein contain packaging materials. Packaging materials for use in packaging pharmaceutical products are well known to those of skill in the art. See, for example, U.S. Pat. Nos. 5,323,907, 5,033,252 and 5,052,558, each of which is incorporated herein in its entirety. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment.

A hyaluronidase, collagenase, and/or 4-MU, with or without one or more additional APIs, composition may optionally comprise an anesthetic agent. An anesthetic agent may be a local anesthetic agent, including an anesthetic agent that causes a reversible local anesthesia or a loss of nociception, such as, e.g., aminoamide local anesthetics and aminoester local anesthetics. Non-limiting examples of anesthetic agents may include lidocaine, ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cyclomethycaine, dibucaine, dimethisoquin, dimethocaine, diperodon, dicyclomine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, combinations thereof, and salts thereof. Non-limiting examples of aminoester local anesthetics include procaine, chloroprocaine, cocaine, cyclomethycaine, dimethocaine (larocaine), propoxycaine, procaine (novocaine), proparacaine, tetracaine (amethocaine). Non-limiting examples of aminoamide local anesthetics include articaine, bupivacaine, cinchocaine (dibucaine), etidocaine, levobupivacaine, lidocaine (lignocaine), mepivacaine, piperocaine, prilocaine, ropivacaine, trimecaine, or a combination thereof.

The amount of an anesthetic agent included may be an amount effective to reduce pain experienced by an individual upon administration of the composition, such as about 0.1%, about 0.2%, about 0.3%, about 0.4%, about 0.5%, about 0.6%, about 0.7%, about 0.8% about 0.9%, about 1.0%, about 2.0%, about 3.0%, about 4.0%, about 5.0%, about 6.0%, about 7.0%, about 8.0%, about 9.0%, about 10%, at least about 0.1%, at least about 0.2%, at least about 0.3%, at least about 0.4%, at least about 0.5%, at least about 0.6%, at least about 0.7%, at least about 0.8% at least about 0.9%, at least about 1.0%, at least about 2.0%, at least about 3.0%, at least about 4.0%, at least about 5.0%, at least about 6.0%, at least about 7.0%, at least about 8.0%, at least about 9.0%, at least about 10%, at most about 0.1%, at most about 0.2%, at most about 0.3%, at most about 0.4%, at most about 0.5%, at most about 0.6%, at most about 0.7%, at most about 0.8% at most about 0.9%, at most about 1.0%, at most about 2.0%, at most about 3.0%, at most about 4.0%, at most about 5.0%, at most about 6.0%, at most about 7.0%, at most about 8.0%, at most about 9.0%, at most about 10%, about 0.1% to about 0.5%, about 0.1% to about 1.0%, about 0.1% to about 2.0%, about 0.1% to about 3.0%, about 0.1% to about 4.0%, about 0.1% to about 5.0%, about 0.2% to about 0.9%, about 0.2% to about 1.0%, about 0.2% to about 2.0%, about 0.5% to about 1.0%, or about 0.5% to about 2.0%.

Some hyaluronidase, collagenase, and/or 4-MU, with or without one or more additional APIs, compositions may comprise lidocaine, in free base or salt form (e.g. lidocaine HCl) in an amount of about 0.05% w/w to about 1% w/w; about 0.1% w/w to about 0.5% w/w, or about 0.3% w/w.

Additionally, compositions of hyaluronidase, collagenase, and/or 4-MU, with or without one or more additional APIs, may have a physiologically-acceptable osmolarity, e.g., about 100 mOsm/L, about 150 mOsm/L, about 200 mOsm/L, about 250 mOsm/L, about 300 mOsm/L, about 350 mOsm/L, about 400 mOsm/L, about 450 mOsm/L, about 500 mOsm/L, at least about 100 mOsm/L, at least about 150 mOsm/L, at least about 200 mOsm/L, at least about 250 mOsm/L, at most about 300 mOsm/L, at most about 350 mOsm/L, at most about 400 mOsm/L, at most about 450 mOsm/L, at most about 500 mOsm/L, about 100 mOsm/L to about 500 mOsm/L, about 200 mOsm/L to about 500 mOsm/L, about 200 mOsm/L to about 400 mOsm/L, about 300 mOsm/L to about 400 mOsm/L, about 270 mOsm/L to about 390 mOsm/L, about 225 mOsm/L to about 350 mOsm/L, about 250 mOsm/L to about 325 mOsm/L, about 275 mOsm/L to about 300 mOsm/L, or about 285 mOsm/L to about 290 mOsm/L. Osmolality agents may be used to adjust osmolality. Examples include, but are not limited to, salts such as, e.g., sodium chloride and potassium chloride; and glycerin.

In some embodiments, a composition comprising hyaluronidase, collagenase, and/or 4-MU, with or without one or more additional APIs, is injectable through a needle of, e.g., about 27 gauge; about 30 gauge; about 32 gauge; about 22 gauge or smaller; about 27 gauge or smaller; about 30 gauge or smaller; about 32 gauge or smaller; about 22 gauge to about 35 gauge; about 22 gauge to about 34 gauge; about 22 gauge to about 33 gauge; about 22 gauge to about 32 gauge; about 22 gauge to about 27 gauge; or about 27 gauge to about 32 gauge.

A hyaluronidase, collagenase, and/or 4-MU, with or without one or more additional APIs, composition may be substantially stable at room temperature, e.g., for about 3 months, about 6 months, about 9 months, about 12 months, about 15 months, about 18 months, about 21 months, about 24 months, about 27 months, about 30 months, about 33 months, about 36 months, at least about 3 months, at least about 6 months, at least about 9 months, at least about 12 months, at least about 15 months, at least about 18 months, at least about 21 months, at least about 24 months, at least about 27 months, at least about 30 months, at least about 33 months, at least about 36 months, about 3 months to about 12 months, about 3 months to about 18 months, about 3 months to about 24 months, about 3 months to about 30 months, about 3 months to about 36 months, about 6 months to about 12 months, about 6 months to about 18 months, about 6 months to about 24 months, about 6 months to about 30 months, about 6 months to about 36 months, about 9 months to about 12 months, about 9 months to about 18 months, about 9 months to about 24 months, about 9 months to about 30 months, about 9 months to about 36 months, about 12 months to about 18 months, about 12 months to about 24 months, about 12 months to about 30 months, about 12 months to about 36 months, about 18 months to about 24 months, about 18 months to about 30 months, or about 18 months to about 36 months.

Duration of treatment may be determined based on the cosmetic and/or clinical effect desired by the individual and/or physician and the body part or region being treated. For some treatments, administration of a composition comprising hyaluronidase, collagenase, and/or 4-MU, with or without one or more additional APIs, can effectively treat a soft tissue condition for, e.g., about 1 month, 2 months, about 3 months, about 4 months, about 5 months, about 6 months, about 7 months, about 8 months, about 9 months, about 10 months, about 11 months, about 12 months, about 13 months, about 14 months, about 15 months, about 18 months, or about 24 months, at least about 6 months, at least about 7 months, at least about 8 months, at least about 9 months, at least about 10 months, at least about 11 months, at least about 12 months, at least about 13 months, at least about 14 months, at least about 15 months, at least about 18 months, or at least about 24 months, about 6 months to about 12 months, about 6 months to about 15 months, about 6 months to about 18 months, about 6 months to about 21 months, about 6 months to about 24 months, about 9 months to about 12 months, about 9 months to about 15 months, about 9 months to about 18 months, about 9 months to about 21 months, about 6 months to about 24 months, about 12 months to about 15 months, about 12 months to about 18 months, about 12 months to about 21 months, about 12 months to about 24 months, about 15 months to about 18 months, about 15 months to about 21 months, about 15 months to about 24 months, about 18 months to about 21 months, about 18 months to about 24 months, or about 21 months to about 24 months.

A hyaluronidase, collagenase, and/or 4-MU, with or without one or more additional APIs, may be injected at between about 2 and about 5 sites. In an embodiment, the hyaluronidase, collagenase, and/or 4-MU, with or without one or more additional APIs, is injected at between about 5 and about 10 sites. In an embodiment, the hyaluronidase, collagenase, and/or 4-MU, with or without one or more additional APIs, is injected at between about 10 to about 30 sites. In an embodiment, the hyaluronidase, collagenase, and/or 4-MU, with or without one or more additional APIs, is injected at between about 10 to about 50 sites. At least two of the sites can be separated by a distance of approximately 100 microns to about 5,000 microns. In an embodiment, the distance between injection sites is about 400 to about 600 microns. In an embodiment, the distance between injections sites is about 100 to about 200 microns, about 200 to about 300 microns, about 300 to about 400 microns, about 400 to about 500 microns, about 500 to about 600 microns, about 600 to about 700 microns, about 700 to about 800 microns, about 800 to about 900 microns, or about 900 to about 1,000 microns. In an embodiment, the distance between injection sites is about 1,000 to about 2,000 microns, about 2,000 to about 3,000 microns, about 3,000 to about 4,000 microns, or about 4,000 to about 5,000 microns.

In some embodiments of any of the aforementioned methods, the hyaluronidase, collagenase, and/or 4-MU, with or without one or more additional APIs, is administered once. In some embodiments of any of the aforementioned methods, administration of an initial dose the hyaluronidase, collagenase, and/or 4-MU, with or without one or more additional APIs, is followed by the administration of one or more subsequent doses of the hyaluronidase, collagenase, and/or 4-MU, with or without one or more additional APIs. Examples of dosing regimens (e.g., an interval between the first dose and one or more subsequent doses) that can be used in the methods of the disclosure include an interval of about once every week to about once every 12 months, an interval of about once every two weeks to about once every 6 months, an interval of about once every month to about once every 6 months, an interval of about once every month to about once every 3 months, or an interval of about once every 3 months to about once every 6 months. In some embodiments, administration is monthly, every two months, every three months, every four months, every five months, every six months, or upon disease recurrence.

The formulations disclosed herein comprise a protein having hyaluronidase, collagenase, and/or 4-MU activity, with or without one or more additional APIs, and optionally a buffering agent (e.g., about 1 to 100 mM of a buffering agent providing a pH of 5.5±2.0), a stabilizer (e.g., about 1 to 500 mM of a stabilizer), and/or a nonionic surfactant (e.g., about 0.01 to 0.1% of a surfactant). In a further embodiment, the formulation is free of or substantially free of albumin. In other embodiments, the formulation may optionally comprise a vasoconstrictor, such as epinephrine. In yet other embodiments, the formulation may optionally comprise an anesthetic agent or a salt thereof.

In some embodiments, the formulations of hyaluronidase, collagenase, and/or 4-MU, with or without one or more additional APIs, are preferably free of or substantially free of albumin. The inventors have discovered that allergies may lead to peri-orbital puffiness and that formulations that comprise albumin may lead to an increased allergic response. Hence, by reducing or eliminating albumin from a formulation as disclosed herein the puffiness is not aggravated and may decrease the potential for an allergic reaction near the eye. In some embodiments, the reduction in albumin results in greater safety of treatment.

In embodiments, the formulation further comprises one or more of an anesthetic agent, a prostaglandin, a prostaglandin analog, and a vasoconstrictor. In some embodiments, the formulation comprises an anesthetic agent. In some embodiments, the formulation comprises a prostaglandin. In some embodiments, the formulation comprises a prostaglandin analog. In some embodiments, the formulation comprises a vasoconstrictor. In some embodiments, the formulation comprises an anesthetic agent and a prostaglandin. In some embodiments, the formulation comprises an anesthetic agent and a prostaglandin analog. In some embodiments, the formulation comprises an anesthetic agent and a vasoconstrictor. In some embodiments, the formulation comprises a prostaglandin and a vasoconstrictor. In some embodiments, the formulation comprises a prostaglandin analog and a vasoconstrictor. In some embodiments, the formulation comprises an anesthetic agent and a vasoconstrictor. In some embodiments, the formulation comprises an anesthetic agent, a prostaglandin, and a vasoconstrictor. In some embodiments, the formulation comprises an anesthetic agent, a prostaglandin analog, and a vasoconstrictor.

In some embodiments, the formulation comprises an anesthetic agent, a prostaglandin analog, and a vasoconstrictor. In some embodiments, the anesthetic agent is lidocaine, the prostaglandin analog is bimatoprost, and the vasoconstrictor is epinephrine.

In some embodiments, the formulation comprises a vasoconstrictor. In some embodiments, the vasoconstrictor is epinephrine. In some embodiments, the epinephrine is administered topically to a subject using a topical regimen. In some embodiments, the epinephrine is administered by injection to a subject using an injection regimen. In some embodiments, the topical regimen of epinephrine is between about 1:500 to about 1:1,000, between about 1:1,000 to about 1:1,500, between about 1:1,500 to about 1:2,000, between about 1:2,000 to about 1:2,500, between about 1:2,500 to about 1:3,000, between about 1:3,000 to about 1:3,500, between about 1:3,500 to about 1:4,000, between about 1:4,000 to about 1:4,500, or between about 1:4,500 to about 1:5,000. In some embodiments, the injection regimen of epinephrine is between about 1:50,000 to about 1:70,000, between about 1:70,000 to about 1:90,000, between about 1:90,000 to about 1:110,000, between about 1:110,000 to about 1:130,000, between about 1:130,000 to about 1:150,000, or between about 1:150,000 to about 1:200,000.

In some embodiments, the addition of a vasoconstrictor, such as epinephrine to the formulation, enables the hyaluronidase, collagenase, and/or 4-MU, with or without one or more additional APIs, to remain more confined to the tissue into which it is injected, thereby protecting the eye. In some embodiments, the use of epinephrine allows the hyaluronidase, collagenase, and/or 4-MU, with or without one or more additional APIs, to have a more concentrated effect and potentially greater impact, which may result in greater improvement of the aesthetic outcome. In some embodiments, the addition of a vasoconstrictor, such as epinephrine to the formulation, may result in greater safety of treatment.

In some embodiments, the formulations comprising a protein having hyaluronidase, collagenase, and/or 4-MU activity, with or without one or more additional APIs, comprise an anesthetic agent that is preferably a local anesthetic agent, i.e., an anesthetic agent that causes a reversible local anesthesia and a loss of nociception, such as, e.g., aminoamide local anesthetics and aminoester local anesthetics. The amount of an anesthetic agent included in a formulation disclosed herein is an amount effective to mitigate pain experienced by an individual upon administration of the composition. As such, the amount of an anesthetic agent included in a composition disclosed in the present specification is between about 0.1% to about 5% by weight of the total composition.

The present disclosure also provides a formulation that comprises a prostaglandin analog including, for example, bimatoprost.

In some embodiments, the addition of an anesthetic, such as lidocaine, to the formulation decreases patient discomfort. In some embodiments, the decrease in patient discomfort allows for a more comfortable treatment, which may prevent inadvertent patient movement during the injection of the formulation.

The formulation may comprise an anesthetic agent in an amount of, e.g., about 0.1%, about 0.2%, about 0.3%, about 0.4%, about 0.5%, about 0.6%, about 0.7%, about 0.8% about 0.9%, about 1.0%, about 2.0%, about 3.0%, about 4.0%, about 5.0%, about 6.0%, about 7.0%, about 8.0%, about 9.0%, or about 10% by weight of the total formulation. In yet other aspects, a formulation disclosed herein comprises an anesthetic agent in an amount of, e.g., at least 0.1%, at least 0.2%, at least 0.3%, at least 0.4%, at least 0.5%, at least 0.6%, at least 0.7%, at least 0.8% at least 0.9%, at least 1.0%, at least 2.0%, at least 3.0%, at least 4.0%, at least 5.0%, at least 6.0%, at least 7.0%, at least 8.0%, at least 9.0%, or at least 10% by weight of the total formulation. In still other aspects, a formulation disclosed herein comprises an anesthetic agent in an amount of, e.g., at most 0.1%, at most 0.2%, at most 0.3%, at most 0.4%, at most 0.5%, at most 0.6%, at most 0.7%, at most 0.8% at most 0.9%, at most 1.0%, at most 2.0%, at most 3.0%, at most 4.0%, at most 5.0%, at most 6.0%, at most 7.0%, at most 8.0%, at most 9.0%, or at most 10% by weight of the total formulation. In further aspects, a formulation disclosed herein comprises an anesthetic agent in an amount of, e.g., about 0.1% to about 0.5%, about 0.1% to about 1.0%, about 0.1% to about 2.0%, about 0.1% to about 3.0%, about 0.1% to about 4.0%, about 0.1% to about 5.0%, about 0.2% to about 0.9%, about 0.2% to about 1.0%, about 0.2% to about 2.0%, about 0.5% to about 1.0%, or about 0.5% to about 2.0% by weight of the total formulation.

Non-limiting examples of anesthetic agents include lidocaine, ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dicyclomine, ecgonidine, ecgonine, ethyl chloride, etidocaine, beta-eucaine, euprocin, fenalcomine, fomocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, combinations thereof, and salts thereof. Non-limiting examples of am inoester local anesthetics include procaine, chloroprocaine, cocaine, cyclomethycaine, dimethocaine (larocaine), propoxycaine, procaine (novocaine), proparacaine, tetracaine (amethocaine). Non-limiting examples of aminoamide local anesthetics include articaine, bupivacaine, cinchocaine (dibucaine), etidocaine, levobupivacaine, lidocaine (lignocaine), mepivacaine, piperocaine, prilocaine, ropivacaine, and trimecaine.

Some hyaluronidase, collagenase, and/or 4-MU, with or without one or more additional APIs, compositions may comprise lidocaine, in free base or salt form (e.g. lidocaine HCl) in an amount of about 0.05% w/w to about 1% w/w; about 0.1% w/w to about 0.5% w/w, or about 0.3% w/w.

In some embodiments, the formulation comprises a prostaglandin or a prostaglandin analog. Such an analog may be used to reduce and/or eliminate vitreous humor that is present in the peri-orbital region. In some embodiments, the formulation comprises a prostaglandin. In some embodiments, the prostaglandin is selected from prostaglandin 12, prostaglandin D2, prostaglandin E2, and prostaglandin F20. In some embodiments, the formulation comprises a prostaglandin analog. In some embodiments, the prostaglandin analog is selected from travaprost, bimatoprost, and latanoprost. In some embodiments, the prostaglandin analog is bimatoprost. In some embodiments, the prostaglandin or prostaglandin analog comprises between about 0.001% and about 0.1% weight per volume of the formulation. In some embodiments, the prostaglandin or prostaglandin analog comprises between about 0.001% and about 0.005% weight per volume of the formulation. In some embodiments, the prostaglandin or prostaglandin analog comprises between about 0.005% and about 0.01% weight per volume of the formulation. In some embodiments, the prostaglandin or prostaglandin analog comprises between about 0.01% and about 0.02% weight per volume of the formulation. In some embodiments, the prostaglandin or prostaglandin analog comprises between about 0.02% and about 0.04% weight per volume of the formulation. In some embodiments, the prostaglandin or prostaglandin analog comprises between about 0.04% and about 0.06% weight per volume of the formulation. In some embodiments, the prostaglandin or prostaglandin analog comprises between about 0.06% and about 0.08% weight per volume of the formulation. In some embodiments, the prostaglandin or prostaglandin analog comprises between about 0.08% and about 0.1% weight per volume of the formulation.

In some embodiments, the formulation comprising hyaluronidase, collagenase, and/or 4-MU, with or without one or more additional APIs, comprises a buffering agent. The preferred concentration of the buffering agent is about 1 to about 50 mM, more preferably about 10 to about 30 mM; the most preferred concentration is about 20 mM. Various buffering agents are known to the person skilled in the art as outlined above. The preferred buffering agent is selected from the group consisting of a histidine buffer, acetic acid buffer, and citric acid buffer, most preferably a L-histidine/HCl buffer. The histidine-buffer according to the invention is used in an amount of about 1 mM to about 50 mM, preferably of about 10 mM to about 30 mM and still more preferably of about 20 mM. The acetic acid buffer according to the invention is preferably of about 10 mM to about 30 mM and most preferably of about 20 mM. The citric acid buffer according to the invention is preferably of about 10 mM to about 30 mM and most preferably of about 20 mM.

Independently from the buffer used, the pH is adjusted to a value comprising about 4.5 to about 7.0 and preferably about 5.5 to about 6.5, also preferably selected from the group consisting of 5.5, 6.0, 6.1 and 6.5. This pH can be obtained by adjustment with an acid or base known in the art or by using adequate mixtures of buffer components or both.

In some embodiments, the formulation comprising hyaluronidase, collagenase, and/or 4-MU, with or without one or more additional APIs, comprises a stabilizer. The stabilizer(s) (used synonymously with the term “stabilizing agent” in the present patent description) is/are preferably selected from the group consisting of a salt, a carbohydrate, saccharide and amino acid(s), more preferably a carbohydrate or saccharide, more preferably a sugar admitted by the authorities as a suitable additive or excipient in pharmaceutical formulations, most preferably selected from the group consisting of α,α-trehalose dihydrate, NaCl and methionine. The preferred concentration of the stabilizer is 15 to 250 mM, or more preferably 150 to 250 mM. Most preferred is a concentration of about 210 mM. The formulation may contain a secondary stabilizer, whereby this secondary stabilizer is preferably methionine, preferably in a concentration of 5 to 25 mM, more preferably in a concentration of 5 to 15 mM. The most preferred methionine concentration is about 10 mM.

In some embodiments, the formulation comprising hyaluronidase, collagenase, and/or 4-MU, with or without one or more additional APIs, comprises a nonionic surfactant. The nonionic surfactant is preferably a polysorbate, more preferably is selected from the group of polysorbate 20, polysorbate 80 and polyethylene-polypropylene copolymer. The concentration of the nonionic surfactant is 0.01 to 0.1% (w/v), or 0.02 to 0.08% (w/v) and preferably 0.02 to 0.06% (w/v), most preferably about 0.06% (w/v).

In certain aspects, the formulations and methods disclosed herein relate to a pharmaceutical composition. In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as Pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, Remington's Pharmaceutical Sciences, 18th Edition, (A. R. Gennaro, ed.), 1990, Mack Publishing Company.

In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions can influence the physical state, stability, rate of in vivo release and rate of in vivo clearance of the antigen binding proteins disclosed. In certain embodiments, the primary vehicle or carrier in a pharmaceutical composition can be either aqueous or non-aqueous in nature. For example, a suitable vehicle or carrier can be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and can further include sorbitol or a suitable substitute.

Additional pharmaceutical compositions will be evident to those skilled in the art, including formulations involving sustained- or controlled-delivery. Techniques for formulating a variety of other sustained- or controlled-delivery means, such as liposome carriers, bio-erodible microparticles or porous beads and depot injections, are also known to those skilled in the art. See, for example, International Patent Application No. PCT/US93/00829, which is incorporated herein by reference and describes controlled release of porous polymeric microparticles for delivery of pharmaceutical compositions. Sustained-release preparations can include semipermeable polymer matrices in the form of shaped articles, e.g., films, or microcapsules. Sustained release matrices can include polyesters, hydrogels, polylactides (as disclosed in U.S. Pat. No. 3,773,919 and European Patent Application Publication No. EP 058481, each of which is incorporated herein by reference), copolymers of L-glutamic acid and gamma ethyl-L-glutamate (Sidman et al., 1983, Biopolymers 2:547-556), poly (2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., 1981, supra) or poly-D(−)-3-hydroxybutyric acid (European Patent Application Publication No. EP 133,988). Sustained release compositions can also include liposomes that can be prepared by any of several methods known in the art. See, e.g., Eppstein et al., 1985, Proc. Natl. Acad. Sci. U.S.A. 82:3688-3692; European Patent Application Publication Nos. EP 036,676; EP 088,046 and EP 143,949, incorporated herein by reference.

Pharmaceutical compositions used for in vivo administration are typically provided as sterile preparations. Sterilization can be accomplished by filtration through sterile filtration membranes. When the composition is lyophilized, sterilization using this method can be conducted either prior to or following lyophilization and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

Once the pharmaceutical composition has been formulated, it can be stored in sterile vials as a solution, suspension, gel, emulsion, solid, crystal, or as a dehydrated or lyophilized powder. Such formulations can be stored either in a ready-to-use form or in a form (e.g., lyophilized) that is reconstituted prior to administration. Kits for producing a single-dose administration unit are also provided. Certain kits contain a first container having a dried protein and a second container having an aqueous formulation. In certain embodiments, kits containing single and multi-chambered pre-filled syringes (e.g., liquid syringes and lyosyringes) are provided. The therapeutically effective amount of an antigen binding protein-containing pharmaceutical composition to be employed will depend, for example, upon the therapeutic context and objectives. One skilled in the art will appreciate that the appropriate dosage levels for treatment will vary depending, in part, upon the molecule delivered, the indication for which the antigen binding protein is being used, the route of administration, and the size (body weight, body surface or organ size) and/or condition (the age and general health) of the patient. In certain embodiments, the clinician can titer the dosage and modify the route of administration to obtain the optimal therapeutic effect.

The pharmaceutical compositions can be formulated into any suitable pharmaceutical preparations for subcutaneous administration such as solutions, suspensions, powders, or sustained release formulations. Compositions provided herein typically are formulated for administration by subcutaneous route, although other routes of administration are contemplated, such as any route known to those of skill in the art. Formulations suited for such routes are known to one of skill in the art. Administration can be local, topical or systemic depending upon the locus of treatment. Local administration to an area in need of treatment can be achieved by, for example, but not limited to, local infusion during surgery, topical application, transdermal patch, or by injection. Compositions also can be administered with other biologically active agents, either sequentially, intermittently or in the same composition. Thus, in one example, local administration can be achieved by injection, such as from a syringe or other article of manufacture containing an injection device such as a needle or an injection device containing multiple needles. In another example, local administration can be achieved by infusion, which can be facilitated by the use of a pump or other similar device, or by a transdermal patch. Pharmaceutical compositions can be formulated in dosage forms appropriate for each route of administration.

Subcutaneous administration, generally characterized by injection or infusion, is contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. The pharmaceutical compositions may contain other minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. The percentage of active compound contained in such compositions is highly dependent on the specific nature thereof, as well as the activity of the compound and the needs of the subject.

Injectables are designed for local and systemic administration. For purposes herein, local administration is desired for direct administration to the affected area. The solutions may be either aqueous or nonaqueous.

In one example, a pharmaceutical preparation can be in liquid form, for example, solutions, syrups or suspensions. If provided in liquid form, the pharmaceutical preparations can be provided as a concentrated preparation to be diluted to a therapeutically effective concentration before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). In another example, pharmaceutical preparations can be presented in lyophilized form for reconstitution with water or other suitable vehicle before use.

In some embodiments, the formulation of the pharmaceutically active compound, namely a protein having hyaluronidase, collagenase, and/or 4-MU activity, with or without one or more additional APIs, is administered to a subject in the form of an injection. The concentration of the pharmaceutically active compound is adjusted so that an injection provides an effective amount to produce the desired pharmacological effect. The exact dose depends on the age, weight and condition of the patient or animal as is known in the art. The unit-dose parenteral preparations are packaged in an ampoule, a vial or a syringe with a needle. The volume of liquid solution or reconstituted powder preparation, containing the pharmaceutically active compound, is a function of the disease to be treated and the particular article of manufacture chosen for package.

A hyaluronidase, collagenase, and/or 4-MU, with or without one or more additional APIs, may be injected at between about 2 and about 5 sites. In an embodiment, the hyaluronidase, collagenase, and/or 4-MU, with or without one or more additional APIs, is injected at between about 5 and about 10 sites. In an embodiment, the hyaluronidase, collagenase, and/or 4-MU, with or without one or more additional APIs, is injected at between about 10 to about 30 sites. In an embodiment, the hyaluronidase, collagenase, and/or 4-MU, with or without one or more additional APIs, is injected at between about 10 to about 50 sites. At least two of the sites can be separated by a distance of approximately 100 microns to about 5,000 microns. In an embodiment, the distance between injection sites is about 400 to about 600 microns. In an embodiment, the distance between injections sites is about 100 to about 200 microns, about 200 to about 300 microns, about 300 to about 400 microns, about 400 to about 500 microns, about 500 to about 600 microns, about 600 to about 700 microns, about 700 to about 800 microns, about 800 to about 900 microns, or about 900 to about 1,000 microns. In an embodiment, the distance between injection sites is about 1,000 to about 2,000 microns, about 2,000 to about 3,000 microns, about 3,000 to about 4,000 microns, or about 4,000 to about 5,000 microns.

In an embodiment, the protein having hyaluronidase, collagenase, and/or 4-MU activity, with or without one or more additional APIs, is injected into a lateral eyelid fat pad, a medial eyelid fat pad, and/or a central eyelid fat pad. In a further embodiment, 1 to 15 injections, preferably 1 to 3 injections, are made into each of the lateral eyelid fat pad, medial eyelid fat pad, and/or central eyelid fat pad. Preferably, the injections into the medial eyelid fat pad are made at a depth of between about 3 to about 10 mm, more preferably about 5 to about 8 mm. Additionally, the injections into the central eyelid fat pad are preferably made at a depth of between about 3 to about 12 mm, more preferably about 5 to about 10 mm. Furthermore, the injections into the lateral eyelid fats pad are preferably made at a depth of between about 3 to about 11 mm, more preferably about 5 to about 8 mm.

In an embodiment, the protein having hyaluronidase, collagenase, and/or 4-MU activity, with or without one or more additional APIs, is injected into a malar region. In a further embodiment, 1 to 15 injections, preferably 3 to 7 injections, are made into the malar region. Preferably, the injections into the malar region are made at a depth of between about 2 to about 13 mm, more preferably about 5 to about 10 mm.

In a preferred embodiment, administration of an initial dose the hyaluronidase, collagenase, and/or 4-MU, with or without one or more additional APIs, is followed by the administration of one or more subsequent doses of the hyaluronidase, collagenase, and/or 4-MU, with or without one or more additional APIs, every one or two weeks for a total of four treatment sessions followed by a treatment once every three months.

The present disclosure is further illustrated by the following examples, which should not be construed as limiting in any way. The materials and methods as used in the following experimental examples are described below

### EXAMPLES

### Example 1: Assessment of a Patient's Peri-Orbital Puffiness Due to Peri-Orbital Edema Using Peri-Orbital and Eyelid Fullness Assessment Scale (PEFAS or Sweis-Scale)

A patient presented with pseudoherniation of her medial upper eyelid fat pads (bilaterally), edema of her right lower eyelid region, and edema with pseudoherniation of the three left lower eyelid fat pads. Using the scorecard below the patient is given an PEFAS score of R: 1/0 and L: 1/3E. L represents the left eyelid with the “/” number on the left of the “/” indicating the score assigned to the upper left eyelid and the number to the right of the “/” indicating the score assigned to the lower left eyelid. Likewise, R represents the right eyelid with the “/” number on the left of the “/” indicating the score assigned to the upper right eyelid and the number to the right of the “/” indicating the score assigned to the lower right eyelid.

TABLE 4
Sample Peri-orbital and Eyelid Fullness Assessment Scale

| | | Left | Left | Right | Right |
|---|---|---|---|---|---|
| Peri-orbital and Eyelid Fullness | | Eye | Eye | Eye | Eye |
| Assessment Scale (PEFAS) | | (upper) | (lower) | (upper) | (lower) |
| Grade | No pseudoherniation of the | | | | |
| 0 | upper or lower eyelid fat pads | | | | |
| | Presence of edema of the fat | | | | X |
| | pads only | | | | |
| | Presence of edema of | | | | |
| | peri-orbital tissue only | | | | |
| | Presences of edema of the fat | | | | |
| | pads and peri-orbital tissue | | | | |
| | [symbol] festoons due to edema | | | | |
| | Presence of localized malar | | | | |
| | puffiness due to edema | | | | |
| Grade | Pseudoherniation of one fat | X | | X | |
| 1 | pad | | | | |
| 1E | Pseudoherniation of one fat | | | | |
| | pad with | | | | |
| | edema of the fat pad(s) | | | | |
| | edema of the peri-orbital | | | | |
| | tissue | | | | |
| | edema of the pat pads and of | | | | |
| | the peri-orbital tissue | | | | |
| Grade | Pseudoherniation of two fat | | | | |
| 2 | pads | | | | |
| 2E | Pseudoherniation of two fat | | | | |
| | pads wiith | | | | |
| | edema of the fat pads | | | | |
| | edema of the peri-orbital tissue | | | | |
| | edema of the fat pads and of the | | | | |
| | peri-orbital tissue | | | | |
| Grade | Pseudoherniation of the three | | | | |
| 3 | lower eyelid fat pads (medial, | | | | |
| | central and lateral fat pads) | | | | |
| 3E | Pseudoherniation of the three | | X | | |
| | lower eyelid fat pads with | | | | |
| | edema of the fat pads | | | | |
| | edema of the peri-orbital tissue | | | | |
| | edema of the fat pads and of the | | | | |
| | peri-orbital tissue | | | | |

[symbol]

indicates data missing or illegible when filed

### Example 2: Treatment of Peri-Orbital Puffiness Due to Peri-Orbital Edema Using a Hyaluronidase

A patient with peri-orbital puffiness due to edema (edema not due to a hyaluronic acid filler) of the peri-orbital soft tissues or edema of the eyelid fat pads was treated with a composition comprising a hyaluronidase (e.g., Hylenex). Briefly, four injections were administered in the peri-orbital soft tissues and edematous orbital fat pads using a dosage of 10 units injected in each fat pad (in the case of edema of the fat pads). The injections were performed using a 0.5 ml syringe having a 32-gauge needle inserted parallel to the globe, angling away from the globe, to avoid injury to the globe.

An improvement in the extent of peri-orbital puffiness due to peri-orbital edema was observed within the first 15-60 minutes after the injections. The patient was followed for 6 months and exhibited an overall reduction in peri-orbital puffiness due to peri-orbital edema (as compared to the peri-orbital puffiness due to peri-orbital edema measured pre-treatment) up to 3 months without any adverse effects. Although there was recurrence of some of the edema, there was an improvement up to 3 months.

### Example 3: Treatment of Edema Using a Formulation Comprising a Hyaluronidase

Patients with edema (Patient A, B, and C) on their feet were treated with Hylenex and the change in severity of their edema was measured after treatment. Briefly, the severity of edema on a target area of each patient's feet was first graded on the following scale: 4+(severe) to 3+, 2+, 1+ or 0 (described above). In particular, the depth of the pitting, the general appearance of the extremities (e.g., the feet, ankles, hands, etc.), the amount of time required for the pit to disappear, and/or the amount of force required to form the pit were measured. Patient A scored a 4+ prior to treatment, Patient B scored a 4+ prior to treatment, and Patient C scored a 3+ prior to treatment.

Next, Hylenex was injected at five different points into an area on each patient's feet. The injections were performed using a 0.5 ml syringe having a 32-gauge needle. All patients were followed post injection, at one day, one week, one, three, and six months and graded using the PCSS. Patient A scored a 2+ prior to treatment, Patient B scored a 3+ prior to treatment, and Patient C scored a 1+ one week after treatment.

### Example 4: Treatment of Cancer Using a Combination of Hyaluronidase and Rituximab

ATCC-CCL-113 cells (derived from a 29 year old Caucasian having non-Hodgkin's lymphoma) are grown to confluence, passaged, and then plated onto three separate plates at a density of 3×10⁶ cells/mL. Plate 1 is treated with Hylenex, Plate B is treated with rituximab, and Plate C is treated with a combination of Hylenex and rituximab. 48 hours after treatment, cell vilabitiy is accessed by trypan blue exclusion. Notably, a significantly increased percentage of the cells are killed after treatment with rituximab and hylenex suggesting that the two APIs act synergistically to kill tumorigenic cells.

### Example 5: Treatment of Peri-Orbital Puffiness Due to Peri-Orbital Edema Using 4-methylumbelliferone (4-MU)

A patient with peri-orbital puffiness due to edema (edema not due to a hyaluronic acid filler) of the peri-orbital soft tissues or edema of the eyelid fat pads was treated with a composition comprising 25 mg/mL 4-methylumbelliferone (4-MU). Briefly, four injections were administered in the peri-orbital soft tissues and edematous orbital fat pads. The injections were performed using a 0.5 ml syringe having a 32-gauge needle inserted parallel to the globe, angling away from the globe, to avoid injury to the globe.

An improvement in the extent of peri-orbital puffiness due to peri-orbital edema was observed within the first 15-60 minutes after the injections. The patient was followed for 6 months and exhibited an overall reduction in peri-orbital puffiness due to peri-orbital edema (as compared to the peri-orbital puffiness due to peri-orbital edema measured pre-treatment) up to 3 months without any adverse effects. Although there was recurrence of some of the edema, there was an improvement up to 3 months.

### Example 6: Treatment of Inflammation Using a Formulation Comprising a Hyaluronidase

Patients with inflammation due to stasis dermatitis (Patient A, B, and C) on their lower legs were treated with Hylenex and the change in severity of their inflammation was measured after treatment.

Briefly, the severity of inflammation on a target area of each patient's ankle was first graded on the following scale: 4 (severe), 3 (moderate), 2 (mild), 1 (quiescent), or 0 (normal). Patient A scored a 4 prior to treatment, Patient B scored a 3 prior to treatment, and Patient C scored a 3 prior to treatment.

Next, 500 units of hylenex was injected at five different points into an area on each patient's ankle. The injections were performed using a 0.5 ml syringe having a 32-gauge needle. All patients were followed post injection, at one day and one week. Patient A's inflammation was classified as a 2 one week after treatment, Patient B's inflammation was classified as a 2 one week after treatment, and Patient C's inflammation was classified as a 2 one week after treatment.

### Example 7: Treatment of Lower Eyelid Puffiness Using a Formulation Comprising a Hyaluronidase and Collagenase

Patient A (a 46 year old female), Patent B (a 51 year old female), and Patent C (a 48 year old female) presented with lower eyelid puffiness.

Briefly, Patient A was administered a composition comprising Hylenex by injections of 100 U to each of five sites in the lower eyelid fat pads, Patient B was administered a composition comprising a collagenase by injections of 100 U to each of five sites in the lower eyelid fat pads, and Patient C was administered a composition comprising Hylenex and a collagenase by injections of 100 U to each of five sites in the lower eyelid fat pads. The injections were performed using a 0.5 ml syringe having a 32-gauge needle.

Each patient's lower eyelid puffiness was visually assessed 2 weeks, 1 month, 2 months, 4 months, 6 months, and 1 year after administration of the compositions. Patient A exhibited a reduction in lower eyelid puffiness, Patient B did not exhibit any reduction in lower eyelid puffiness, and Patient C exhibited an elimination of lower eyelid puffiness. Surprisingly, Patent C's lower eyelid puffiness was eliminated while Patent A and B still exhibited some puffiness suggesting that Hylenex and collagenase function synergistically to eliminate the appearance of eyelid puffiness.

### Example 8: Treatment of Fibrosis Using a Formulation Comprising a Hyaluronidase

Five patients with scleroderma on their arms were treated with Hylenex and the change in severity of their fibrosis was measured after treatment. Briefly, the severity of scleroderma on a target area of on each patient was first graded using as either severe, moderate, mild, or none. Overall scores for patients' arms prior to treatment averaged as moderate.

Next, Hylenex was injected at five different points into a 10×10 cm area of scleroderma on the patient's arm. The injections were performed using a 0.5 ml syringe having a 32-gauge needle. All patients were followed post injection, at one day, one week, one, three, and six months. Overall scores post-treatment averaged as mild indicating that the severity of the scleroderma decreased after treatment with Hylenex.

### Example 9: Treatment of Demyelinated Neurons Using a Formulation Comprising a Hyaluronidase

Demyelinated neurons were grown to confluence in culture and then transferred to a 24-well cell culture plate (Sigma) in a volume of 2 mL. 10 U/mL of Hylenex was added to each well. After 14 days the neurons were evaluated by microscopy and found to have remyelinated neurons.

### Example 10: Treatment of Jowls Using a Formulation Comprising a Hyaluronidase

Patients with jowls (Patient A, B, and C) were treated with Hylenex and the change in severity of their jowls was measured after treatment. Briefly, the severity of jowls in Patients A-C was assessed on a scale of severe, moderate or mild. Patient A and B presented with severe jowls while Patient C presented with mild jowls.

Next, Hylenex was injected at seven different points into an area of skin on each patients with jowls. The injections were performed using a 0.5 ml syringe having a 32-gauge needle. All patients were followed post injection, at one day, one week, one, three, and six months. Patients A, B, and C were all scored as having mild jowls one day after treatment with effecting lasting more than six months.

### Example 11: Assessment of a Patient's Peri-Orbital Puffiness Due to Peri-Orbital Edema Using Eyelid Fullness Assessment Scale (PEFAS or Sweis-Scale)

A 46 year old male's right and left eyes were examined—i.e., the upper and lower eyelid fat pads for both the right and left eye. The eyelid fat pads were examined for pseudoherniation. Additionally, the patient was examined for one or more of the following conditions: edema of the eyelid fat pads only, edema along the soft tissue of the upper and/or lower eyelids, malar mounds due to edema, malar edema, and mild festoons aggravated by edema.

Upon examination, the patients left eye (upper) did not exhibit any pseudoherniation of the upper eyelid fat pads. However, this eye did present with malar edema. Additionally, the patient's left eye (lower) exhibited pseudoherniation of only two of the eyelid fat pads and the presence of malar mounds due to edema. Furthermore, the patients right eye (upper) did not exhibit any pseudoherniation of the upper eyelid fat pads and the presence of mild festoons aggravated by edema. Last, the patients right eye (lower) showed pseudoherniation of the three lower eyelid fat pads and the presence of malar mounds due to edema.

Using the scorecard below the patient is given a PEFAS score of L: 0/2E and R: 0/3E. L represents the left eyelid with the “/” number on the left of the “/” indicating the score assigned to the upper left eyelid and the number to the right of the “/” indicating the score assigned to the lower left eyelid. Likewise, R represents the right eyelid with the “/” number on the left of the “/” indicating the score assigned to the upper right eyelid and the number to the right of the “/” indicating the score assigned to the lower right eyelid.

| | | Left | Left | Right | Right |
|---|---|---|---|---|---|
| Peri-orbital and Eyelid Fullness | | Eye | Eye | Eye | Eye |
| Assessment Scale (PEFAS) | | (upper) | (lower) | (upper) | (lower) |
| Grade 0 | No pseudoherniation of the | X | | X | |
| | upper and/or lower eyelid | | | | |
| | fat pads and no associated | | | | |
| | peri-orbital, malar or eyelid | | | | |
| | edema | | | | |
| Grade 0E | No pseudoherniation of the | | | | |
| | eyelid fat pads, | | | | |
| | and one or more of the | | | | |
| | following: | | | | |
| | i. presence of edema of the | | | | |
| | eyelid fat pads; | | | | |
| | ii. presence of edema along | | | | |
| | the soft tissue of the upper | | | | |
| | or lower eyelids; | | | | |
| | iii. presence of malar | | | | |
| | mounds due to edema; | | | | |
| | iv. presence of malar | | | | |
| | edema; | | | | |
| | v. presence of festoons | | | | |
| | aggravated by edema | | | | |
| Grade 1 | Pseudoherniation of only | | | | |
| | one of the eyelid fat pads | | | | |
| | without any associated | | | | |
| | peri-orbital, malar and/or | | | | |
| | eyelid edema | | | | |
| Grade 1E | Pseudoherniation of only | | | | |
| | one of the eyelid fat pads, | | | | |
| | and one or more of the | | | | |
| | following: | | | | |
| | i. presence of edema of the | | | | |
| | fat pad; | | | | |
| | ii. presence of edema along | | | | |
| | the soft tissue of the upper | | | | |
| | and/or lower eyelids; | | | | |
| | iii. presence of malar | | | | |
| | mounds due to edema; | | | | |
| | iv. presence of malar | | | | |
| | edema; | | | | |
| | v. presence of mild festoons | | | | |
| | aggravated by edema | | | | |
| Grade 2 | Pseudoherniation of only | | | | |
| | two of the eyelid fat pads | | | | |
| | without any associated | | | | |
| | peri-orbital, malar and/or | | | | |
| | eyelid edema | | | | |
| Grade 2E | Pseudoherniation of only | | X | | |
| | two of the eyelid fat pads, | | | | |
| | and one or more of the | | | | |
| | following: | | | | |
| | i. presence of edema of one | | | | |
| | or more of the fat pads; | | | | |
| | ii. presence of edema along | | | | |
| | the soft tissue of the upper | | | | |
| | and/or lower eyelids; | | | | |
| | iii. presence of malar | | | | |
| | mounds due to edema; | | | | |
| | iv. presence of malar | | | | |
| | edema; | | | | |
| | v. presence of mild festoons | | | | |
| | aggravated by edema | | | | |
| Grade 3 | Pseudoherniation of the | | | | |
| | three lower eyelid fat pads | | | | |
| | without any associated | | | | |
| | peri-orbital, malar and/or | | | | |
| | eyelid edema | | | | |
| Grade 3E | Pseudoherniation of the | | | | X |
| | three lower eyelid fat pads, | | | | |
| | and one or more of the | | | | |
| | following: | | | | |
| | i. presence of edema of one | | | | |
| | or more of the fat pads; | | | | |
| | ii. presence of edema along | | | | |
| | the soft tissue of the upper | | | | |
| | and/or lower eyelids; | | | | |
| | iii. presence of malar | | | | |
| | mounds due to edema; | | | | |
| | iv. presence of malar | | | | |
| | edema; | | | | |
| | v. presence of mild festoons | | | | |
| | aggravated by edema | | | | |
| Grade 3+ | Pseudoherniation of the | | | | |
| | three lower eyelid fat pads | | | | |
| | with moderate to severe | | | | |
| | festoons and/or malar | | | | |
| | mounds secondary to fat | | | | |
| | deposition or | | | | |
| | descent/hypertrophy of the | | | | |
| | orbicularis oculi muscle, | | | | |
| | and without any associated | | | | |
| | peri-orbital, malar or eyelid | | | | |
| | edema | | | | |
| Grade 3 + E | Pseudoherniation of the | | | | |
| | three lower eyelid fat pads | | | | |
| | with moderate to severe | | | | |
| | festoons and/or malar | | | | |
| | mounds secondary to fat | | | | |
| | deposition or | | | | |
| | descent/hypertrophy of the | | | | |
| | orbicularis oculi muscle with | | | | |
| | one or more of the following: | | | | |
| | i. presence of edema of one | | | | |
| | or more of the fat pads; | | | | |
| | ii. presence of edema along | | | | |
| | the soft tissue of the upper | | | | |
| | and/or lower eyelids; | | | | |
| | iii. presence of malar | | | | |
| | mounds aggravated by | | | | |
| | edema; | | | | |
| | iv. presence of localized | | | | |
| | malar edema; | | | | |
| | v. presence of festoons | | | | |
| | aggravated by edema | | | | |

### Example 12: Treatment of Pen-Orbital Puffiness Due to Pen-Orbital Edema Using HYLENEX

Four patients (Patients A-D) with peri-orbital puffiness due to edema (as identified in Example 1) of the eyelid fat pads were scored as a Grade 1E, 2E, 3E, or 3+E on the Peri-orbital Fullness Assessment Scale. A score was given to the upper and lower eyelids in the patient's left and right eye. Patient A's eyes were scored on the PEFAS scale as a L 0/3E (left eye; upper eyelid over lower eyelid) and a R 0/3E (right eye; upper eyelid over lower eyelid). Patient B was scored as a L 2E/2E and a R 2E/2E. Patient C was scored as a L 1/3E and a R 0/3E and Patient D was scored as a L 2E/3+E and a R 2E/3+E.

The patients were subsequently treated with HYLENEX. Briefly, one injection of 30U HYLENEX was made per peri-orbital fat pad exhibiting edema and optionally four injections were administered in the peri-orbital soft tissues using the same dosage. The injections were performed using a 0.5 ml syringe having a 31-gauge needle inserted parallel to the globe, angling away from the globe, to avoid injury to the globe. Specifically, three injections were made into the medial eyelid fat pad, two into the central eyelid fat pad, and three into the lateral eyelid fat pad at a depth of about 6 mm, 8 mm, and 6 mm, respectively. Additionally Patients B and C received 3 to 5 injections in the peri-orbital soft tissues (e.g., the malar region) at a depth of between about 5 to about 10 mm.

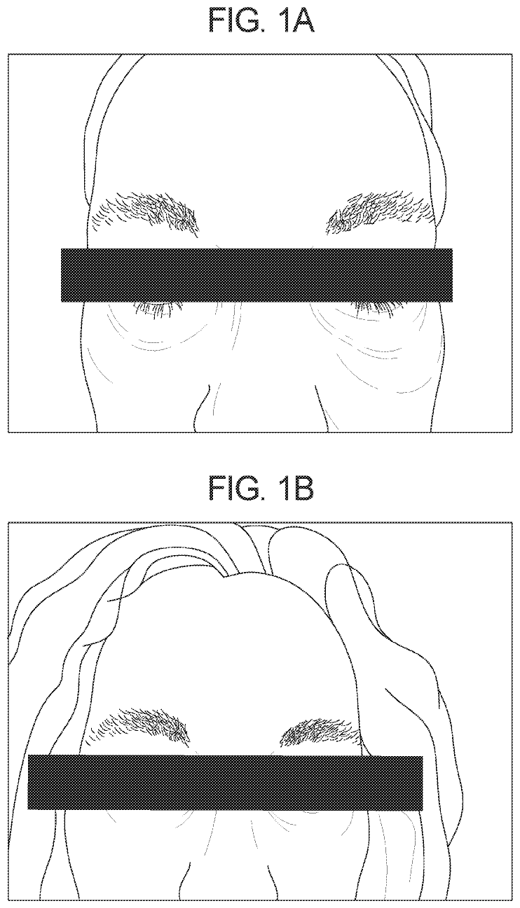

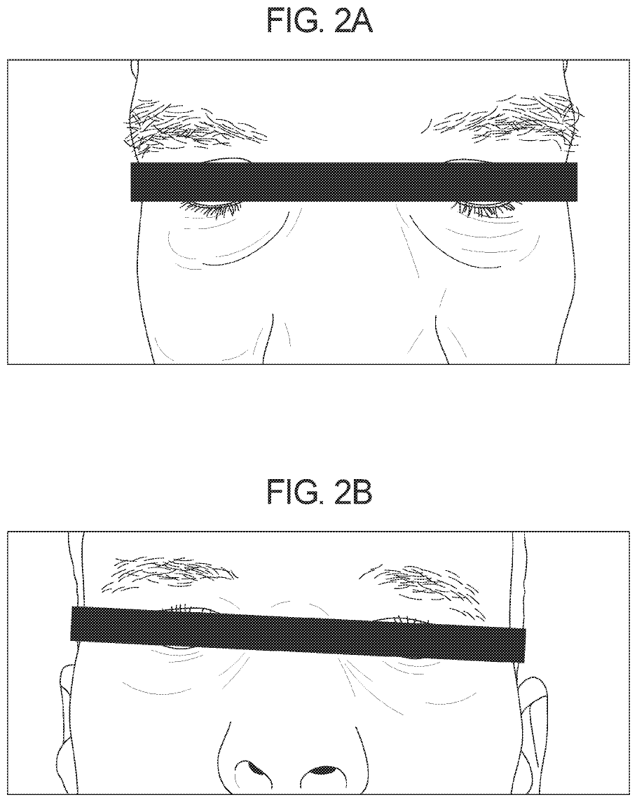

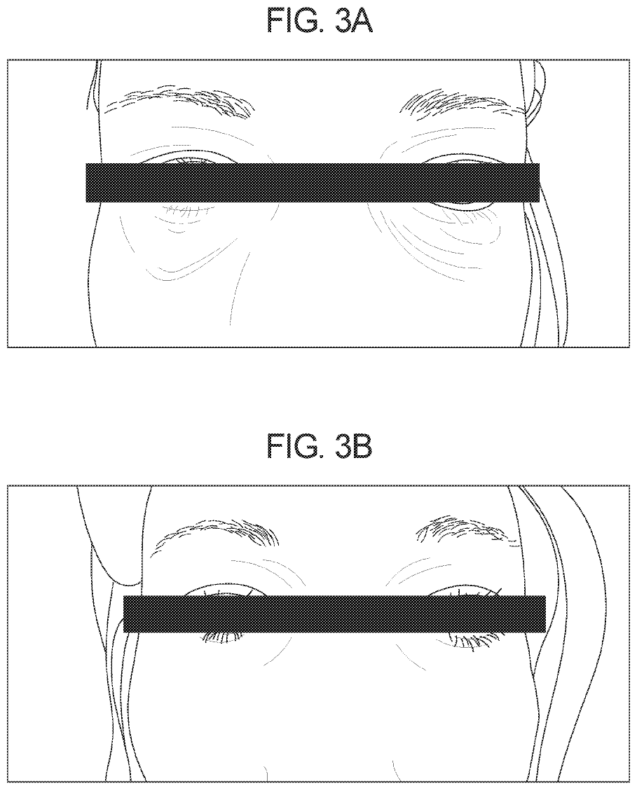

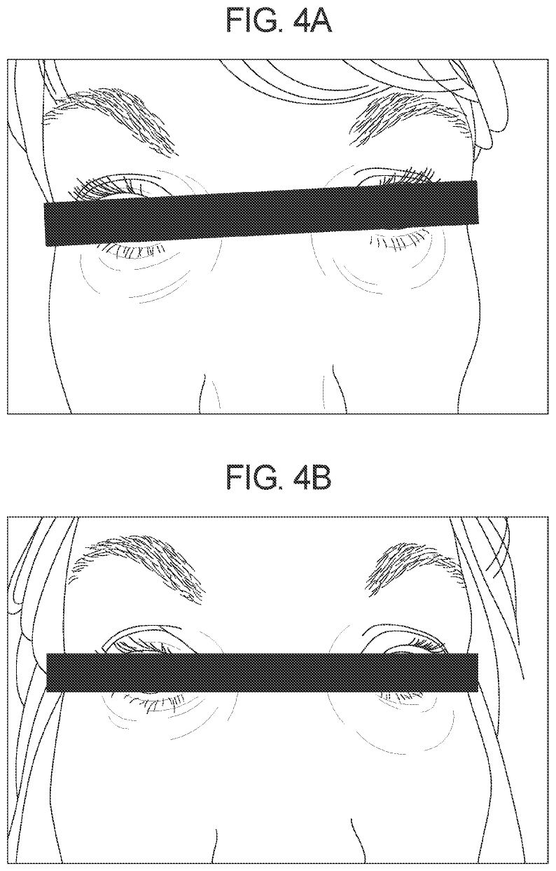

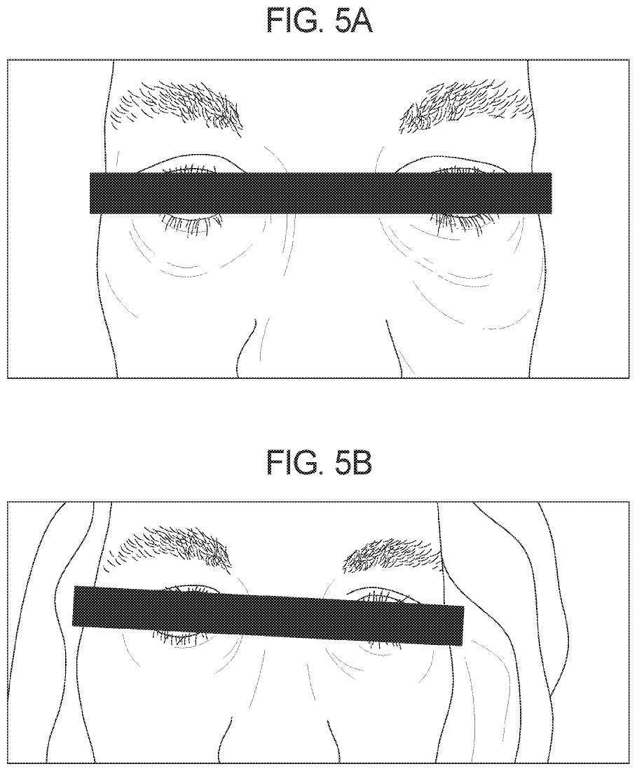

Each patient exhibited an improvement in the extent of peri-orbital puffiness within the first 15-60 minutes after the injections. After treatment, Patient C was followed for 1 month, Patient D was followed for 2 months, and Patients A and B were followed for 3 months. Each patient exhibited a marked reduction in per-orbital (eye) puffiness as compared to the peri-orbital puffiness that exhibited pre-treatment (see, FIGS. 1-5). The patients were followed for up to 6 months and still exhibit a reduction in peri-orbital puffiness measured pre-treatment without any adverse effects.

After treatment, the patient's eyes were scored on the PEFAS scale. Patient A's eyes were scored on the PEFAS scale as a L 0/0 (left eye; upper eyelid over lower eyelid) and a R 0/0E (right eye; upper eyelid over lower eyelid). Patient B was scored as a L 2E/1E and a R 2E/1. Patient C was scored as a L 1E/1E and a R 0/1 and Patient D was scored as a L 2E/0E and a R 2E/0. Thus, each patient demonstrated an improvement in peri-orbital edema by a decrease in the grade assigned to the peri-orbital puffiness assigned to both their left and right eyes.

Moreover, an additional 12 patients that exhibit peri-orbital puffiness were treated according to the regimen described above. Each patient was given an initial Subjective Patient Score of 10. The Subjective Patient Score is based on a scale of 1 to 10 with 10 being the subject's observed starting amount of peri-orbital puffiness and 0 being no puffiness. Each of the patients scored their puffiness between as a 3 or 4 at 2 weeks, 3 months, and 6 months after their initial treatment.

### EMBODIMENTS

Embodiment 1. A method of treating or preventing a condition such as a cosmetic condition in a subject in need thereof by administering a composition that comprises a protein having hyaluronidase activity (e.g., a hyaluronidase such as Hylenex, Amphadase, or Vitrase) to the peri-orbital region and/or mid-face of the subject, wherein the cosmetic condition may be due to edema (e.g., peri-orbital puffiness due to peri-orbital edema).

Embodiment 2. A method of treating peri-orbital puffiness due to peri-orbital edema in a subject in need thereof by administering a composition that comprises a protein having hyaluronidase activity to the peri-orbital region of the subject.

Embodiment 3. The method of any of the above or below embodiments, wherein the step of administering is performed by one or more injections.

Embodiment 4. The method of any of the above or below embodiments, wherein the step of administering is performed by one or more injections to the peri-orbital region.

Embodiment 5. The method of any of the above or below embodiments, wherein the step of administering is performed by one or more injections into a peri-orbital soft tissue and/or one or more injections into one or more peri-orbital fat pads.

Embodiment 6. The method of any of the above or below embodiments, wherein the method comprises administering a composition that comprises a protein having hyaluronidase activity to a region of skin having cellulite on the subject.

Embodiment 7. The present disclosure provides a method of reducing the appearance of cellulite in a subject in need thereof, the method comprising administering a composition that comprises a protein having hyaluronidase activity to a region of skin having cellulite on the subject, wherein the administration results in a reduction in a grade of severity of the cellulite.

Embodiment 8. The method of any of the above or below embodiments, wherein the region of skin is located on the back of a leg, a buttock, an arm, a thigh, and/or an abdomen.

Embodiment 9. The method of any of the above or below embodiments, wherein the region of skin is located on the back of a leg or a buttock.

Embodiment 10. The method of any of the above or below embodiments, wherein the method further comprises tightening the region of skin having cellulite after administration of the composition.

Embodiment 11. The method of any of the above or below embodiments, wherein the skin is tightened by treatment with a radiofrequency.

Embodiment 12. The method of any of the above or below embodiments, wherein the skin is tightened by treatment with ultrasound.

Embodiment 13. The method of any of the above or below embodiments, wherein the skin is tightened by high intensity focused electromagnetic technology.

Embodiment 14. The method of any of the above or below embodiments, wherein the step of administering the composition is performed by one or more injections to the region of skin having cellulite.

Embodiment 15. The method of any of the above or below embodiments, wherein the step of administering the composition comprises applying a patch or a cream to the region of skin having cellulite.

Embodiment 16. The method of any of the above or below embodiments, wherein the method further comprises administering retinol cream to the region of skin having cellulite.

Embodiment 17. The method of any of the above or below embodiments, wherein the method further comprises treating the region of skin having cellulite with acoustic wave therapy, laser treatment, ultrasonic liposculpting, laser-assisted liposuction, and/or radiotherapy.

Embodiment 18. The method of any of the above or below embodiments, wherein the administration of the composition reduces the appearance of the cellulite.

Embodiment 19. The method of any of the above or below embodiments, wherein the administration reduces a number of depressions, depth of depressions, clinical appearance of evident raised lesions, presence of flaccidity, and/or grade of the cellulite.

Embodiment 20. The method of any of the above or below embodiments, wherein the cellulite prior to treatment with the composition is classified as mild, moderate, or severe cellulite.

Embodiment 21. The method of any of the above or below embodiments, wherein the cellulite after treatment with the composition is classified as mild or moderate cellulite.

Embodiment 22. The method of any of the above or below embodiments, wherein the severity grade is based on the Cellulite Severity Scale (CSS).

Embodiment 23. The method of any of the above or below embodiments, wherein the method further comprises administering a retinol cream.

Embodiment 24. The method of any of the above or below embodiments, wherein treating the area of skin having cellulite with an acoustic wave therapy, laser treatment, ultrasonic liposculpting, laser-assisted liposuction, and/or radiotherapy.

Embodiment 25. The method of any of the above or below embodiments, wherein the region of skin in the subject in need thereof is located on the back of a leg, a buttock, an arm, a thigh, and/or an abdomen.

Embodiment 26. A method of assessing peri-orbital fullness in a subject, the method comprising: a. determining if the subject exhibits pseudoherniation of one or more upper eyelid fat pads and/or one or more lower eyelid fat pads; b. determining if the subject exhibits edema in one or more of the upper eyelid fat pads and/or the one or more of the lower eyelid fat pads; c. determining if the subject exhibits edema in the peri-orbital tissue; and d. scoring the peri-orbital fullness from the determinations of steps a. to c.

Embodiment 27. The method of any of the above or below embodiments, wherein the peri-orbital fullness is due to peri-orbital edema.

Embodiment 28. The method of any of the above or below embodiments, wherein the peri-orbital fullness is unrelated to the use of a hyaluronic acid filler in the peri-orbital region.

Embodiment 29. The method of any of the above or below embodiments, wherein the lower eyelid fat pad is a lower lateral fat pad, a lower middle fat pad, or a lower medial fat pad.

Embodiment 30. The method of any of the above or below embodiments, wherein the lower eyelid fat pad is selected from one or more of a lower lateral fat pad, a lower middle fat pad, or a lower medial fat pad.

Embodiment 31. The method of any of the above or below embodiments, wherein the upper eyelid fat pad is an upper middle fat pad or an upper medial fat pad.

Embodiment 32. The method of any of the above or below embodiments, wherein the subject is scored as Grade 0 where the subject does not exhibit pseudoherniation of any of the upper or lower eyelid fat pads.

Embodiment 33. The method of any of the above or below embodiments, wherein the subject exhibits edema of the one or more upper and/or lower eyelid fat pads, edema along the soft tissue of the upper and/or lower eyelids, presence of malar mounds due to edema, presence of malar edema, presence of mild festoons, or any combination thereof.

Embodiment 34. The method of any of the above or below embodiments, wherein the subject is scored as Grade 1 where the subject exhibits pseudoherniation of one or more of the upper and/or lower eyelid fat pads without any peri-orbital malar or eyelid edema.

Embodiment 35. The method of any of the above or below embodiments, wherein the subject is scored as Grade 1 E where the subject exhibits pseudoherniation of one or more of the upper and/or lower eyelid fat pads, and the subject exhibits edema of the one or more upper and/or lower fat pads, presence of edema along the soft tissue of the upper or lower eyelids, presence of malar mounds due to edema, presence of malar edema, presence of mild festoons, or any combination thereof.

Embodiment 36. The method of any of the above or below embodiments, wherein the subject is scored as Grade 2 where the subject exhibits pseudoherniation of two of the upper and/or lower eyelid fat pads without peri-orbital, malar or eyelid edema.

Embodiment 37. The method of any of the above or below embodiments, wherein the subject is scored as Grade 2E where the subject exhibits pseudoherniation of two of the upper and/or lower eyelid fat pads, and the subject exhibits edema of the one or more upper and/or lower fat pads, presence of edema along the soft tissue of the upper or lower eyelids, presence of malar mounds due to edema, presence of malar edema, presence of mild festoons, or any combination thereof.

Embodiment 38. The method of any of the above or below embodiments, wherein the subject is scored as Grade 3 where the subject exhibits pseudoherniation of three lower eyelid fat pads without any associated peri-orbital, malar or eyelid edema.

Embodiment 39. The method of any of the above or below embodiments, wherein the subject is scored as Grade 3E where the subject exhibits pseudoherniation of three lower eyelid fat pads, and the subject exhibits edema of the fat pads, presence of edema along the soft tissue of the upper and/or lower eyelids, presence of malar mounds due to edema, presence of malar edema, presence of mild festoons, or any combination thereof.

Embodiment 40. The method of any of the above or below embodiments, wherein the subject is scored as Grade 3+ where the subject exhibits pseudoherniation of the three lower eyelid fat pads with moderate to severe festoons and/or malar mounds secondary to fat deposition or hypertrophy of the orbicularis oculi muscle, without any peri-orbital, malar or eyelid edema.

Embodiment 41. The method of any of the above or below embodiments, wherein the subject is scored as 3E+ where the subject exhibits pseudoherniation of the three lower eyelid fat pads with moderate to severe festoons and/or malar mounds secondary to fat deposition or descent/hypertrophy of the orbicularis oculi muscle, and the subject exhibits edema of the fat pad, presence of edema along the soft tissue of the upper or lower eyelids, presence of malar mounds due to edema, presence of malar edema, presence of mild festoons, or any combination thereof.

Embodiment 42. The method of any of the above or below embodiments, wherein the subject scored as Grade 0 is administered a composition that comprises a protein having hyaluronidase activity to the peri-orbital region of the subject.

Embodiment 43. The method of any of the above or below embodiments, wherein the subject scored as Grade 1, Grade 2 or Grade 3 is treated by surgical removal of a portion of one or more of the upper and/or lower fat pads.

Embodiment 44. The method of any of the above or below embodiments, wherein the subject scored as Grade 1E, 2E or 3E is administered a composition that comprises a protein having hyaluronidase activity to the peri-orbital region of the subject, and optionally treated by surgical removal of a portion of one or more of the upper and/or lower fat pads.

Embodiment 45. A method of determining if peri-orbital fullness is due to edema or a structural change in a subject, the method comprising: a. administering a composition that comprises a protein having hyaluronidase activity to the peri-orbital region of the subject; b. assessing the peri-orbital region at a predetermined amount of time after the administration of the composition; and c. determining whether there is an improvement in the peri-orbital fullness.

Embodiment 46. The method of any of the above or below embodiments, wherein the structural change is a herniation.

Embodiment 47. The method of any of the above or below embodiments, wherein the predetermined time is 5 minutes, 15 minutes, 30 minutes, 1 hour, 24 hours, or 1 week after administration of the composition.

Embodiment 48. The method of any of the above or below embodiments, wherein no improvement in peri-orbital puffiness indicates that the peri-orbital puffiness is due to a structural change.

Embodiment 49. The method of any of the above or below embodiments, wherein a partial improvement in peri-orbital puffiness indicates that the peri-orbital puffiness is secondary to both edema and a structural change.

Embodiment 50. The method of any of the above or below embodiments, wherein improvement in peri-orbital puffiness indicates that the peri-orbital puffiness is secondary to edema.

Embodiment 51. The method of any of the above or below embodiments, wherein an improvement includes a reduction in the peri-orbital puffiness.

Embodiment 52. A method for treating a subject with peri-orbital fullness, the method comprising: a. determining if peri-orbital fullness is due to edema or a structural change in a subject by: i. administering a composition that comprises a protein having hyaluronidase activity to the peri-orbital region of the subject; ii. assessing the peri-orbital region at a predetermined amount of time after the administration of the composition; and iii. determining whether there is an improvement in the peri-orbital fullness; and b. surgically resecting a portion of one or more of an upper and/or lower eyelid fat pads where there is no improvement in peri-orbital fullness after administration of the composition.

Embodiment 53. A method for minimizing peri-orbital hollowness from surgical resection of one or more peri-orbital fat pads in a subject, the method comprising: a. administering a composition that comprises a protein having hyaluronidase activity to the peri-orbital region of the subject; b. assessing the peri-orbital region at a predetermined amount of time after administration of the composition; and c. determining an amount of fat to surgically resect from the one or more eyelid fat pads.

Embodiment 54. The method of any of the above or below embodiments, wherein the method further comprises surgically resecting an amount of the one or more peri-orbital fat fads.

Embodiment 55. The method of any of the above or below embodiments, wherein the one or more peri-orbital fat pads include the upper eyelid fat pads and the lower eyelid fat pads.

Embodiment 56. The method of any of the above or below embodiments, wherein the amount of fat to surgically resect from the one or more fat pads is determined by a visual examination of the one or more fat pads after administration of the composition.

Embodiment 57. The method of any of the above or below embodiments, wherein the step of administering the composition is performed by one or more injections to the region of the subject having edema.

Embodiment 58. A method for determining an amount of fat to be resected surgically from one or more per-orbital fat pads, the method comprising: administering a composition that comprises a protein having hyaluronidase activity to the peri-orbital region of the subject having peri-orbital puffiness; and determining an amount of fat to surgically resect from the one or more fat pads at a predetermined amount of time after administration of the composition.

Embodiment 59. The method of any of the above or below embodiments, wherein the method comprising: a. administering a composition that comprises a protein having hyaluronidase activity to the peri-orbital region of the subject; b. assessing the peri-orbital region at a predetermined amount of time after administration of the composition; c. determining an amount of fat to surgically resect from the one or more eyelid fat pads; and d. resecting a portion of the one or more eyelid fat pads.

Embodiment 60. The method of any of the above or below embodiments, wherein the one or more eyelid fat pads include the upper eyelid fat pads and the lower eyelid fat pads.

Embodiment 61. The method of any of the above or below embodiments, wherein the lower eyelid fat pad is selected from one or more of a lower lateral fat pad, a lower middle fat pad, a lower medial fat pad.

Embodiment 62. A method for determining if a subject with peri-orbital fullness is a candidate for treatment with a glycosaminoglycan based dermal filler along a depressed tear trough, the method comprising: a. administering a composition that comprises a protein having hyaluronidase activity to the peri-orbital region of the subject; b. assessing the peri-orbital region of the subject at a predetermined period of time after administration of the composition; and c. determining if there is an improvement in the peri-orbital puffiness, wherein the subject is a candidate for treatment with the glycosaminoglycan based dermal filler along the depressed tear trough where the subject does not exhibit an improvement in peri-orbital fullness after administration of the composition.

Embodiment 63. The method of any of the above or below embodiments, wherein the peri-orbital region includes the eyelid fat pads.

Embodiment 64. The method of any of the above or below embodiments, wherein the one or more eyelid fat pads include the upper eyelid fat pads and/or the lower eyelid fat pads.

Embodiment 65. The method of any of the above or below embodiments, wherein the step of administering the composition is performed by one or more injections to the peri-orbital region of the subject.

Embodiment 66. A method for treating a subject having peri-orbital puffiness and a depressed tear trough, the method comprising: a. administering a composition that comprises a protein having hyaluronidase activity to the peri-orbital region of the subject; b. assessing the peri-orbital region of the subject at a predetermined period of time after administration of the composition; c. determining if there is an improvement in the peri-orbital puffiness; and d. injecting a glycosaminoglycan based filler to and area of skin having the depressed tear trough if the subject does not exhibit an improvement in peri-orbital puffiness after administration of the composition.

Embodiment 67. A method for diagnosing an etiology of upper and/or lower eyelid puffiness, the method comprising: a. examining a subject with squinted eyes; and b. determining if upper and/or lower eyelid puffiness does not improve, improves, partially improves, or worsens, wherein the etiology of the upper and/or lower eyelid puffiness is diagnosed to be anterior to the orbicularis oculi muscle if the puffiness does not improve, wherein the etiology of the upper and/or lower eyelid puffiness is diagnosed to be posterior to the orbicularis oculi muscle if the puffiness improves, wherein the etiology of the upper and/or lower eyelid puffiness is diagnosed to be anterior and posterior to the orbicularis oculi muscle if the puffiness partially improves, or wherein the puffiness is diagnosed to be secondary to hypertrophy of the orbicularis muscle or if the puffiness worsens.

Embodiment 68. The method of any of the above or below embodiments, wherein the subject is in an upright position with head in a Frankfort horizontal plane.

Embodiment 69. The method of any of the above or below embodiments, wherein the method further comprises the step of instructing the subject to squint or tighten the orbicularis oculi muscle.

Embodiment 70. The method of any of the above or below embodiments, wherein the etiology of the upper and/or lower eyelid puffiness is determined to be anterior to the orbicularis oculi muscle, and wherein the method further comprises administering a protein having hyaluronidase activity into the soft tissue anterior to the orbicularis oculi muscle.

Embodiment 71. The method of any of the above or below embodiments, wherein the etiology of the upper and/or lower eyelid puffiness is determined to be posterior to the orbicularis oculi muscle, and wherein the method further comprises the step of determining if the upper and/or lower eyelid puffiness is secondary to pseudoherniation of upper and/or lower eyelid fat pads, edema of upper and/or lower eyelid fat pads, or upper and/or lower eyelid fat pad pseudoherniation and edema.

Embodiment 72. The method of any of the above or below embodiments, wherein the puffiness of the lower eyelid fat pads are assessed by asking the subject to look straight up, look up and to the right, and look up and to the left.

Embodiment 73. The method of any of the above or below embodiments, wherein the puffiness of the lower eyelid fat pads is due to pseudoherniation of the lower eyelid fat pads and surgery is indicated if the lower eyelid fat pads protrude and are individually isolated.

Embodiment 74. The method of any of the above or below embodiments, wherein the puffiness is due to pseudoherniation and edema of the lower eyelid fat pads if the lower eyelid fat pads protrude and are not individually isolated.

Embodiment 75. The method of any of the above or below embodiments, wherein a protein having hyaluronidase activity is injected into the lower eyelid fat pads to determine the extent of edema of the lower eyelid fat pads.

Embodiment 76. The method of any of the above or below embodiments, wherein the method further comprises resecting a portion of the lower eyelid fat pads.

Embodiment 77. The method of any of the above or below embodiments, wherein the puffiness of the upper eyelid fat pads are assessed by asking the subject to look straight down, look down and to the right, and look down and to the left.

Embodiment 78. The method of any of the above or below embodiments, wherein the puffiness of the upper eyelid fat pads is due to pseudoherniation of upper eyelid fat pads and surgery is indicated if the upper eyelid fat pads protrude and are individually isolated.

Embodiment 79. The method of any of the above or below embodiments, wherein the puffiness is due to pseudoherniation and edema of upper eyelid fat pads if the upper eyelid fat pads protrude and are not individually isolated.

Embodiment 80. The method of any of the above or below embodiments, wherein a protein having hyaluronidase activity is injected into the upper eyelid fat pads to determine the extent of edema of the eyelid fat pads.

Embodiment 81. The method of any of the above or below embodiments, wherein the method further comprises resecting a portion of the upper eyelid fat pads.

Embodiment 82. The method of any of the above or below embodiments, wherein the etiology of the upper and/or lower eyelid puffiness is determined to be anterior and posterior to the orbicularis oculi muscle, and wherein the method further comprises injecting a protein having hyaluronidase activity into the upper and/or lower eyelid fat pads.

Embodiment 83. The method of any of the above or below embodiments, wherein the etiology of the upper and/or lower eyelid puffiness is determined to be anterior and posterior to the orbicularis oculi muscle, and wherein the method further comprises assessing whether the puffiness is partially due to pseudoherniation of eyelid fat pads or edema of the fat pads, or eyelid fat pad pseudoherniation and edema.

Embodiment 84. The method of any of the above or below embodiments, wherein the puffiness is due to pseudoherniation of the lower eyelid fat pads and surgery is indicated if the lower eyelid fat pads protrude and are individually isolated.

Embodiment 85. The method of any of the above or below embodiments, wherein a protein having hyaluronidase activity is injected into the lower eyelid fat pads to determine the extent of edema of the eyelid fat pads.

Embodiment 86. The method of any of the above or below embodiments, wherein the puffiness of the upper eyelid fat pads is due to pseudoherniation of the upper eyelid fat pads and surgery is indicated if the upper eyelid fat pads protrude and are individually isolated.

Embodiment 87. The method of any of the above or below embodiments, wherein the puffiness of the upper eyelid fat pads is due to pseudoherniation and edema of the upper eyelid fat pads if the upper eyelid fat pads protrude and are not individually isolated.

Embodiment 88. The method of any of the above or below embodiments, wherein a protein having hyaluronidase activity is injected into the upper eyelid fat pads to determine the extent of edema of the upper eyelid fat pads.

Embodiment 89. The method of any of the above or below embodiments, wherein a neuromodulator is indicated if the puffiness is determined to be secondary to hypertrophy of the orbicularis muscle or if the puffiness worsens.

Embodiment 90. A method for determining an etiology of peri-orbital puffiness, the method comprising: performing an eyelid squint test; and observing an impact of a movement of an orbicularis oculi muscle on protrusion of eyelid fat pads, wherein the etiology of the upper and/or lower eyelid puffiness is diagnosed to be anterior to the orbicularis oculi muscle if the puffiness does not improve, wherein the etiology of the upper and/or lower eyelid puffiness is diagnosed to be posterior to the orbicularis oculi muscle if the puffiness improves, wherein the etiology of the upper and/or lower eyelid puffiness is diagnosed to be anterior and posterior to the orbicularis oculi muscle if the puffiness partially improves, or wherein the puffiness is diagnosed to be secondary to hypertrophy of the orbicularis muscle or if the puffiness worsens.

Embodiment 91. A method of treating lower extremity edema in a subject in need thereof, the method comprising administering a composition that comprises a protein having hyaluronidase activity to a region of skin having the lower extremity edema on the subject, wherein the lower extremity edema is from venous stasis disease.

Embodiment 92. The method of any of the above or below embodiments, wherein the edema is pitting edema.

Embodiment 93. The method of any of the above or below embodiments, wherein the edema is non-pitting edema.

Embodiment 94. A method of reducing lower extremity edema in a subject in need thereof, the method comprising administering a composition that comprises a protein having hyaluronidase activity to the region of the subject having edema, wherein the administration of the composition reduces severity of the lower extremity edema, wherein the lower extremity edema is from venous stasis disease.

Embodiment 95. A method of treating edema in a subject in need thereof, the method comprising administering a composition that comprises a protein having hyaluronidase activity to a region of the subject having edema.

Embodiment 96. The method of any of the above or below embodiments, wherein the peri-orbital puffiness is unrelated to the use of a hyaluronic acid filler in the peri-orbital region.

Embodiment 97. The method of any of the above or below embodiments, wherein the peri-orbital puffiness is unrelated to the prior use of a hyaluronic acid filler in the peri-orbital region.

Embodiment 98. The method of any of the above or below embodiments, wherein the protein having hyaluronidase activity is hyaluronidase.

Embodiment 99. The method of any of the above or below embodiments, wherein the protein having hyaluronidase activity is Hylenex, Amphadase, or Vitrase.

Embodiment 100. The method of any of the above or below embodiments, wherein the protein having hyaluronidase activity is Hylenex.

Embodiment 101. The method of any of the above or below embodiments, wherein the protein having hyaluronidase activity is Amphadase.

Embodiment 102. The method of any of the above or below embodiments, wherein the protein having hyaluronidase activity is Vitrase.

Embodiment 103. The method of any of the above or below embodiments, wherein the hyaluronidase is a recombinant hyaluronidase.

Embodiment 104. The method of any of the above or below embodiments, wherein the hyaluronidase is a bovine or a human hyaluronidase.

Embodiment 105. The method of any of the above or below embodiments, wherein the hyaluronidase is a human hyaluronidase.

Embodiment 106. The method of any of the above or below embodiments, wherein the hyaluronidase is a bovine hyaluronidase.

Embodiment 107. The method of any of the above or below embodiments, wherein the one or more peri-orbital fat pads include at least one lower peri-orbital fat pad.

Embodiment 108. The method of any of the above or below embodiments, wherein the lower peri-orbital fat pad is a lower lateral fat pad, a lower middle fat pad, or a lower medial fat pad.

Embodiment 109. The method of any of the above or below embodiments, wherein the lower peri-orbital fat pad is a lower lateral fat pad, a lower middle fat pad, and a lower medial fat pad.

Embodiment 110. The method of any of the above or below embodiments, wherein the one or more peri-orbital fat pads include at least one upper peri-orbital fat pad.

Embodiment 111. The method of any of the above or below embodiments, wherein the upper peri-orbital fat pad is an upper middle fat pad or an upper medial fat pad.

Embodiment 112. The method of any of the above or below embodiments, wherein the upper peri-orbital fat pad is an upper middle fat pad and an upper medial fat pad.

Embodiment 113. The method of any of the above or below embodiments, wherein the step of administering comprises applying a patch, an ointment or a cream to a surface of the skin in the peri-orbital region.

Embodiment 114. The method of any of the above or below embodiments, wherein the subject has not previously been treated with a dermal filler in the orbital region.

Embodiment 115. The method of any of the above or below embodiments, wherein the dermal filler is a hyaluronic acid filler.

Embodiment 116. The method of any of the above or below embodiments, wherein the subject has not been treated with a dermal filler 1 month, 2 months, 3 months, 4 months, 5 months, 6 months, 7 months, 8 months, 9 months, 10 months, 11 months, 12 months, 2 years, 3 years, 4 years, or 5 years before the administration of the composition that comprises the protein having hyaluronidase activity.

Embodiment 117. The method of any of the above or below embodiments, wherein the protein having hyaluronidase activity is administered in a therapeutically effective amount.

Embodiment 118. The method of any of the above or below embodiments, wherein the composition is administered in an amount effective to treat the peri-orbital puffiness due to peri-orbital edema.

Embodiment 119. The method of any of the above or below embodiments, wherein the treatment reduces the peri-orbital puffiness due to peri-orbital edema.

Embodiment 120. The method of any of the above or below embodiments, wherein each injection into the peri-orbital soft tissue and/or the one or more peri-orbital fat pads includes about 1 to about 50 Units of the protein having hyaluronidase activity.

Embodiment 121. The method of any of the above or below embodiments, wherein each injection into the peri-orbital soft tissue and/or the one or more peri-orbital fat pads includes about 5 to about 15 Units of the protein having hyaluronidase activity.

Embodiment 122. The method of any of the above or below embodiments, wherein each injection is performed using a 0.5 mL syringe.

Embodiment 123. The method of any of the above or below embodiments, wherein the 0.5 mL syringe comprises a 32-gauge needle.

Embodiment 124. The method of any of the above or below embodiments, wherein the treatment lasts from about 4 to about 12 weeks.

Embodiment 125. A method of assessing peri-orbital puffiness due to peri-orbital edema in a subject by determining if the subject exhibits pseudoherniation of one or more upper peri-orbital fat pads and one or more lower peri-orbital fat pads; determining if the subject exhibits edema in one or more of the upper peri-orbital fat pads and the one or more of the lower peri-orbital fat pads; determining if the subject exhibits edema in the peri-orbital tissue; and scoring the peri-orbital puffiness due to peri-orbital edema from each of the prior three determinations.

Embodiment 126. The method of any of the above or below embodiments, wherein the lower peri-orbital fat pad is selected from one or more of a lower lateral fat pad, a lower middle fat pad, a lower medial fat pad.

Embodiment 127. The method of any of the above or below embodiments, wherein the subject is scored as Grade 0 where the subject does not exhibit pseudoherniation of any of the upper or lower peri-orbital fat pads.

Embodiment 128. The method of any of the above or below embodiments, wherein the subject is scored as Grade 0 where the subject exhibits edema of one or more of the upper or lower peri-orbital fat pads.

Embodiment 129. The method of any of the above or below embodiments, wherein the subject is scored as Grade 0 where the subject exhibits edema of the peri-orbital tissue.

Embodiment 130. The method of any of the above or below embodiments, wherein the subject is scored as Grade 0 where the subject exhibits festoons due to edema.

Embodiment 131. The method of any of the above or below embodiments, wherein the subject is scored as Grade 0 where the subject exhibits localized malar puffiness due to edema.

Embodiment 132. The method of any of the above or below embodiments, wherein the subject is scored as Grade 1 where the subject exhibits pseudoherniation of the upper medial fat pad or the lower medial fat pad.

Embodiment 133. The method of any of the above or below embodiments, wherein the subject is scored as Grade 1 E where the subject exhibits pseudoherniation of the upper medial fat pad or the lower medial fat pad, and exhibits edema of any of the per-orbital fat pads and/or peri-orbital tissue.

Embodiment 134. The method of any of the above or below embodiments, wherein the subject is scored as Grade 2 where the subject exhibits pseudoherniation of the upper eyelid and/or lower eyelid medial and central fat pads.

Embodiment 135. The method of any of the above or below embodiments, wherein the subject is scored as Grade 2E where the subject exhibits pseudoherniation of the upper eyelid and/or lower eyelid medial and central fat pads, and exhibits edema of the fat pads and/or peri-orbital tissue.

Embodiment 136. The method of any of the above or below embodiments, wherein the subject is scored as Grade 3 where the subject exhibits pseudoherniation of the medial, central and lateral lower eyelid fat pads.

Embodiment 137. The method of any of the above or below embodiments, wherein the subject is scored as Grade 3E where the subject exhibits pseudoherniation of the medial, central and lateral lower eyelid fat pads, and exhibits edema of the fat pads and/or peri-orbital tissue.

Embodiment 138. The method of any of the above or below embodiments, wherein the subject scored as Grade 1, Grade 2 or Grade 3 is treated by surgical removal of a portion of one or more of the fat pads.

Embodiment 139. The method of any of the above or below embodiments, wherein the subject scored as Grade 1E, 2E or 3E is administered a composition that comprises a protein having hyaluronidase activity to the peri-orbital region of the subject, and optionally treated by surgical removal of a portion of one or more of the fat pads to increase the degree of improvement in the peri-orbital puffiness.

Embodiment 140. A method to assess whether surgical intervention is needed to treat peri-orbital puffiness not due to peri-orbital edema in a subject in need thereof, the method comprising administering (e.g., injecting) a composition comprising a hyaluronidase to the peri-orbital region of the subject and determining that surgical intervention is required to treat the peri-orbital puffiness not due to peri-orbital edema where the administration of the composition comprising hyaluronidase does not treat (e.g., reduce) the peri-orbital puffiness not due to peri-orbital edema. Such methods may additionally comprise the step of surgically removing a portion of one or more fat pads in the upper and/or lower eyelids of the subject.

Embodiment 141. A method of treating a cosmetic condition in a subject in need thereof due to (e.g., caused by) edema by administering a composition that comprises a protein having hyaluronidase activity to a region on the subject's face having the condition (e.g., a peri-orbital region and/or a mid-face region of the subject).

Embodiment 142. The method of any of the above or below embodiments, wherein the cosmetic condition is a festoon, malar puffiness, or peri-orbital puffiness.

Embodiment 143. The method of any of the above or below embodiments, wherein the subject has not previously been treated with a dermal filler in the peri-orbital region and/or the mid-face.

Embodiment 144. The method of any of the above or below embodiments, wherein after the administration of the composition the edema presents as a 2 mm depression with an immediate rebound time.

Embodiment 145. The method of any of the above or below embodiments, wherein after the administration of the composition the edema presents as a 3 to 4 mm depression with a rebound time of 15 seconds or less.

Embodiment 146. The method of any of the above or below embodiments, wherein after administration of the composition the edema presents as a 5 to 6 mm depression with a rebound time of 10 to 30 seconds.

Embodiment 147. The method of any of the above or below embodiments, wherein before administration of the composition the edema presents as a 8 mm depression with a rebound time of more than 20 seconds.

Embodiment 148. The method of any of the above or below embodiments, wherein the one or more peri-orbital fat pads include the upper eyelid fat pads and the lower eyelid fat pads.

Embodiment 149. The method of any of the above or below embodiments, wherein the edema is peripheral edema.

Embodiment 150. The method of any of the above or below embodiments, wherein the peripheral edema is present in a leg, foot, ankle, and/or arm.

Embodiment 151. The method of any of the above or below embodiments, wherein the peripheral edema is present in a foot.

Embodiment 152. The method of any of the above or below embodiments, wherein the edema is pedal edema.

Embodiment 153. The method of any of the above or below embodiments, wherein the edema is present is a lower leg and/or a foot.

Embodiment 154. The method of any of the above or below embodiments, wherein the protein having hyaluronidase activity is a hyaluronidase.

Embodiment 155. The method of any of the above or below embodiments, wherein the step of administering is performed by one or more injections to the region of the subject having edema.

Embodiment 156. The method of any of the above or below embodiments, wherein the step of administering comprises applying a patch or a cream to a region of the subject having edema.

Embodiment 157. The method of any of the above or below embodiments, wherein each injection includes about 1 to about 1,000 Units of the protein having hyaluronidase activity.

Embodiment 158. The method of any of the above or below embodiments, wherein each injection includes about 1 to about 50 Units of the protein having hyaluronidase activity.

Embodiment 159. The method of any of the above or below embodiments, wherein each injection includes about 5 to about 15 Units of the protein having hyaluronidase activity.

Embodiment 160. A method of reducing edema in a subject in need thereof, the method comprising administering a composition that comprises a protein having hyaluronidase activity to the region of the subject having edema, wherein the administration of the composition reduces severity of the edema.

Embodiment 161. The method of any of the above or below embodiments, wherein the severity of the edema is reduced by at least one grade.

Embodiment 162. The method of any of the above or below embodiments, wherein the severity of the edema is reduced by at least two grades.

Embodiment 163. The method of any of the above or below embodiments, wherein after the administration of the composition the edema is scored as grade 3.

Embodiment 164. The method of any of the above or below embodiments, wherein after the administration of the composition the edema is scored as grade 2.

Embodiment 165. The method of any of the above or below embodiments, wherein after the administration of the composition the edema is scored as grade 1.

Embodiment 166. The method of any of the above or below embodiments, wherein after the administration of the composition the edema is characterized as having a 2 mm depression with an immediate rebound time.

Embodiment 167. The method of any of the above or below embodiments, wherein after the administration of the composition the edema is characterized as having a 3 to 4 mm depression with a rebound time of 15 seconds or less.

Embodiment 168. The method of any of the above or below embodiments, wherein after administration of the composition the edema is characterized as having a 5 to 6 mm depression with a rebound time of 10 to 30 seconds.

Embodiment 169. The method of any of the above or below embodiments, wherein before administration of the composition the edema is characterized as having a 8 mm depression with a rebound time of more than 20 seconds.

Embodiment 170. The method of any of the above or below embodiments, wherein the method further comprise administering a diuretic to the subject.

Embodiment 171. A composition that comprise a protein having hyaluronidase activity and an additional active pharmaceutical ingredient (API) and methods of use thereof including, for example, to treat and/or prevent a disease or disorder.

Embodiment 172. The composition of any of the above or below embodiments, wherein the active pharmaceutical ingredient is a small molecule or a biologic.

Embodiment 173. The composition of any of the above or below embodiments, wherein the additional active pharmaceutical ingredient is used to treat acne, ADHD, AIDS/HIV, allergies, Alzheimer's, angina, anxiety, arthritis, asthma, bipolar disorder, bronchitis, cancer, cholesterol, colds & flu, constipation, COPD, depression, diabetes (Type 1), diabetes (Type 2), diarrhea, eczema, erectile dysfunction, fibromyalgia, gastrointestinal, GERE (Heartburn), gout, hair loss, hayfever, heart disease, hepatitis A, hepatitis B, herpes, hypertension, hypothyroidism, incontinence, IBD (Bowel), insomnia, menopause, mental health, migraine, osteoarthritis, osteoporosis, pain, psoriasis, rheumatoid arthritis, schizophrenia, seizures, sexual health, stroke, swine flu, UTI, or weight loss.

Embodiment 174. The composition of any of the above or below embodiments, wherein the additional active pharmaceutical ingredient (API) is selected from the group consisting of: 0.5-alpha-reductase inhibitors, 5-am inosalicylates, 5HT3 receptor antagonists, ACE inhibitors with calcium channel blocking agents, ACE inhibitors with thiazides, adamantane antivirals, adrenal cortical steroids, adrenal corticosteroid inhibitors, adrenergic bronchodilators, agents for hypertensive emergencies, agents for pulmonary hypertension, aldosterone receptor antagonists, alkylating agents, allergenics, alpha-glucosidase inhibitors, alternative medicines, amebicides, aminoglycosides, aminopenicillins, am inosalicylates, AMPA receptor antagonists, amylin analogs, analgesic combinations, analgesics, androgens and anabolic steroids, Angiotensin Converting Enzyme Inhibitors, angiotensin II inhibitors with calcium channel blockers, angiotensin II inhibitors with thiazides, angiotensin receptor blockers, angiotensin receptor blockers and neprilysin inhibitors, anorectal preparations, anorexiants, antacids, anthelmintics, anti-angiogenic ophthalmic agents, anti-CTLA-4 monoclonal antibodies, anti-infectives, anti-PD-1 monoclonal antibodies, antiadrenergic agents (central) with thiazides, antiadrenergic agents (peripheral) with thiazides, antiadrenergic agents, centrally acting, antiadrenergic agents, peripherally acting, antiandrogens, antianginal agents, antiarrhythmic agents, antiasthmatic combinations, antibiotics/antineoplastics, anticholinergic antiemetics, anticholinergic antiparkinson agents, anticholinergic bronchodilators, anticholinergic chronotropic agents, anticholinergics/antispasmodics, anticoagulant reversal agents, anticoagulants, anticonvulsants, antidepressants, antidiabetic agents, antidiabetic combinations, antidiarrheals, antidiuretic hormones, antidotes, antiemetic/antivertigo agents, antifungals, antigonadotropic agents, antigout agents, antihistamines, antihyperlipidemic agents, antihyperlipidemic combinations, antihypertensive combinations, antihyperuricemic agents, antimalarial agents, antimalarial combinations, antimalarial quinolones, antimanic agents, antimetabolites, antimigraine agents, antineoplastic combinations, antineoplastic detoxifying agents, antineoplastic interferons, antineoplastics, antiparkinson agents, antiplatelet agents, antipseudomonal penicillins, antipsoriatics, antipsychotics, antirheumatics, antiseptic and germicides, antithyroid agents, antitoxins and antivenins, antituberculosis agents, antituberculosis combinations, antitussives, antiviral agents, antiviral boosters, antiviral combinations, antiviral interferons, anxiolytics, sedatives, and hypnotics, aromatase inhibitors, atypical antipsychotics, azole antifungals, Abilify, Ablysinol, Absorica, Acanya, Accolate, Accrufer, AccuNeb, Accupril, Accuretic, Acetadote, Achromycin V, Aciphex, Aciphex Sprinkle, Acthrel, Acticlate, Actigall, Actiq, Activella, Actonel, ActoPlus Met, Actos, Acular, Acular LS, Acuvail, Aczone, Adalat CC, Adasuve, Adcirca, Adderall, Adderall XR, Addyi, Adempas, Adhansia XR, Adlyxin, Admelog, Adrenaclick, AdreView, Advair Diskus, Advair HFA, Adzenys ER, Adzenys XR-ODT, Aemcolo, Afinitor, Afinitor Disperz, Aggrastat, Aggrenox, Agrylin, AirDuo Digihaler, AirDuo Respiclick, AK-Fluor, Aklief, Akovaz, Akten, Akynzeo, Albenza, Aldactazide, Aldactone, Aldara, Alecensa, Alfenta, Alimta, Alinia, Aliqopa, Alkeran, Alocril, Alomide, Aloprim, Alora, Aloxi, Alphagan, Alrex, Altabax, Altace, Altafluor Benox, Altoprev, Altreno, Alunbrig, Alvesco, Amaryl, Ambien, Ambien CR, Ameluz, Amerge, Amicar, Amidate, Amitiz, Ammonul, Amphadase, Ampyra, Amrix, Amyvid, Amzeeq, Anadrol-50, Anafranil, Anaprox-DS, Ancobon, Androderm, AndroGel, Anectine, Angeliq, bacterial vaccines, barbiturate anticonvulsants, barbiturates, BCR-ABL tyrosine kinase inhibitors, benzodiazepine anticonvulsants, benzodiazepines, beta blockers with calcium channel blockers, beta blockers with thiazides, beta-adrenergic blocking agents, beta-lactamase inhibitors, bile acid sequestrants, biologicals, bisphosphonates, bone morphogenetic proteins, bone resorption inhibitors, bronchodilator combinations, bronchodilators, Bactrim, Bactrim DS, Balcoltra, Balversa, Banzel, Baqsimi, Baraclude, Basaglar, Baxdela, Belbuca, Beleodaq, Belrapzo, Belsomra, Belviq, Belviq XR, Bendeka, Benicar, Benicar HCT, Bentyl, Benzaclin, Benzamycin, Bepreve, Besivance, Beta-Val, Betagan, Betapace, Betapace AF, Bethkis, Betimol, Betoptic, Betoptic S, Bevespi Aerosphere, Bevyxxa, Beyaz, Bicillin L-A, BiCNU, BiDil, Bijuva, Biktarvy, Biltricide, Binosto, Biorphen, Blephamide, Bloxiverz, Boniva, Bonjesta, Bonsity, Bosulif, Braftovi, Breo Ellipta, Brethine, Brevibloc, Brevital Sodium, Bridion, Brilinta, Brisdelle, Briviact, BromSite, Brovana, Bryhali, Bumex, Bunavail, Buphenyl, Buprenex, Busulfex, Butisol Sodium, Butrans, Bydureon, Bydureon BCise, Byetta, Bystolic, Calcimimetics, calcineurin inhibitors, calcitonin, calcium channel blocking agents, carbamate anticonvulsants, carbapenems, carbapenems/beta-lactamase inhibitors, carbonic anhydrase inhibitor anticonvulsants, carbonic anhydrase inhibitors, cardiac stressing agents, cardioselective beta blockers, cardiovascular agents, catecholamines, cation exchange resins, CD20 monoclonal antibodies, CD30 monoclonal antibodies, CD33 monoclonal antibodies, CD38 monoclonal antibodies, CD52 monoclonal antibodies, CDK 4/6 inhibitors, central nervous system agents, cephalosporins, cephalosporins/beta-lactamase inhibitors, cerumenolytics, CFTR combinations, CFTR potentiators, CGRP inhibitors, chelating agents, chemokine receptor antagonist, chloride channel activators, cholesterol absorption inhibitors, cholinergic agonists, cholinergic muscle stimulants, cholinesterase inhibitors, CNS stimulants, coagulation modifiers, colony stimulating factors, contraceptives, corticotropin, coumarins and indandiones, cox-2 inhibitors, Cabometyx, Caduet, Cafcit, Calan, Calan SR, Calcium Disodium Versenate, Caldolor, Calquence, Cambia, Campral, Camptosar, Canasa, Cancidas, Capastat Sulfate, Capex, Caprelsa, Carac, Carafate, Carbaglu, Carbatrol, Carbocaine, Cardiogen-82, Cardizem, Cardizem CD, Cardizem LA, Cardura, Cardura XL, Carnitor, Carnitor SF, CaroSpir, Casodex, Casporyn HC, Catapres, Catapres-TTS, Caverject, Caverject Impulse, Cayston, Cefotan, Ceftin, Celebrex, Celestone Soluspan, Celexa, CellCept, Celontin, Centany, Cequa, Cerdelga, Cerebyx, Cerezyme, Cesamet, Cetraxal, Cetrotide, Chantix, Chemet, ChiRhoStim, Cholbam, Cialis, Ciloxan, Cimduo, Cinvanti, Cipro, Cipro HC, Ciprodex, Clarinex, Clarinex-D 12 Hour, Clenpiq, Cleocin Phosphate, Cleocin T, Cleocin Vaginal, Cleviprex, Climara, Climara Pro, lindagel, Clindesse, Clobex, Cloderm, Clolar, Clom id, Clorotekal, Clozaril, Coartem, Cogentin, Colazal, Colcrys, Colestid, Coly Mycin M, Coly-Mycin S, Combigan, CombiPatch, Combivent Respimat, Combivir, Cometriq, Complera, Comtan, Concerta, Condylox, Conray, Consensi, Contrave, ConZipD, Copaxone, Copegus, Copiktra, Cordarone, Cordran, Cordran SP, Coreg, Coreg CR, Corgard, Corlanor, Corlopam, Corphedra, Cortef, Cortenema, Cortifoam, Cortisporin Cream, Cortisporin Ointment, Cortrosyn, Corvert, Corzide, Cosmegen, Cosopt, Cosopt PF, Cotellic, Cotempla XR-ODT, Coumadin, Cozaar, Cresemba, Crestor, Crixivan, Cubicin, Cubicin RF, Cuprimine, Curosurf, Cutivate, Cuvposa, Cyanokit, Cyclessa, Cycloset, Cyklokapron, Cymbalta, Cystadane, Cystagon, Cystaran, Cysto-Conray II, Cystografin, Cytomel, Cytotec, Cytovene, Decongestants, dermatological agents, diagnostic radiopharmaceuticals, diarylquinolines, dibenzazepine anticonvulsants, digestive enzymes, dipeptidyl peptidase 4 inhibitors, diuretics, dopaminergic, antiparkinsonism agents, drugs used in alcohol dependence, D.H.E. 45, Dacogen, Daliresp, Dalvance, Dantrium, Daraprim, DaTscan, Daurismo, Daypro, Daytrana, DDAVP, Definity, Defitelio, Delestrogen, Delstrigo, Delzicol, Demadex, Demerol, Demser, Denavir, Depacon, Depakote, Depakote ER, Depen, Depo-Medrol, Depo-Provera, Depo-subQ provera 104, Derma-Smoothe/FS, Dermatop, DermOtic Oil, Descovy, Desferal, Desonate, DesOwen, Desoxyn, Detrol, Detrol LA, Dexedrine, Dexilant, Dextenza, DiaBeta, Diabinese, Diacomit, Diastat, Diastat AcuDial, Dibenzyline, Diclegis, Differin, Dificid, Diflucan, Dilantin, Dilatrate-SR, Dilaudid, Diovan, Diovan HCT, Dipentum, Diprivan, Diprolene, Diprolene AF, Ditropan XL, Diuril, Divigel, Docefrez, Dolophine, Dopram, Doptelet, Doral, Doryx, Doryx MPC, Dotarem, Dovato, Dovonex, Drisdol, Drizalma Sprinkle, Droxia, Dsuvia, Duac, Duaklir Pressair, Duavee, Duetact, Duexis, Dulera, Duobrii, DuoDote, Duopa, Duraclon, Duragesic, Duramorph PF, Durezol, Durlaza, Dutoprol, Dyanavel XR, Dyazide, Dyrenium, Echinocandins, EGFR inhibitors, estrogen receptor antagonists, estrogens, expectorants, E-Z-HD, E-Z-Paque, EC-Naprosyn, Ecoza, Edarbi, Edarbyclor, Edecrin, Edex, Edluar, Edurant, Effexor, Effexor XR, Effient, Efudex, Egaten, Egrifta, Elcys, Elelyso, Elepsia XR, Elestat, Elestrin, Elidel, Eligard, Elimite, Eliquis, Ella, Ellence, Elmiron, Elocon, Eloxatin, Embeda, Emcyt, Emend, Emflaza, Emla, Emsam, Emtriva, Enablex, Endari, Endometrin, Enstilar, Entereg, Entocort EC, Entresto, Envarsus XR, Eovist, Epaned, Epclusa, Epidiolex, Epiduo, Epiduo Forte, EpiPen, EpiPen Jr, Epivir, Epivir-HBV, Epzicom, Equetro, Eraxis, Erivedge, Erleada, Ertaczo, Eryc, Erygel, EryPed, Erythrocin, Esbriet, Estrogel, Estrostep Fe, Ethamolin, Ethyol, Eucrisa, Eurax, Euthyrox, Evamist, Evekeo ODT, Evista, Evoclin, Evomela, Evotaz, Evoxac, Evzio, Exelderm, Exelon, Exforge, Exforge HCT, Exjade, Exondys 51, Extina, Ezallor, factor Xa inhibitors, fatty acid derivative anticonvulsants, fibric acid derivatives, first generation cephalosporins, fourth generation cephalosporins, functional bowel disorder agents, Fabior, Factive, Famvir, Fanapt, Fareston, Farxiga, Farydak, Faslodex, Felbatol, Feldene, Femara, Femhrt, Femring, Fenoglide, Fentora, Feraheme, Ferriprox, Ferrlecit, Fetzima, Fiasp, Fibricor, Finacea, Fiorinal, Firazyr, Firdapse, Firmagon, Flagyl, Flarex, Flector Patch, Flolan, FloLipid, Flomax, Flovent HFA, Flumadine, Fluorescite, Fluoroplex, FML, FML Forte Liquifilm, Focalin, Focalin XR, Follistim AQ, Folotyn, Forane, Forfivo XL, Fortamet, Fortaz, Forteo, Fortesta, Fosamax, Fosamax Plus D, Foscavir, Fosrenol, Fragmin, Frova, Furadantin, Fusilev, Fuzeon, Fycompa, gallstone solubilizing agents, gamma-aminobutyric acid analogs, gamma-aminobutyric acid reuptake inhibitors, gastrointestinal agents, general anesthetics, genitourinary tract agents, GI stimulants, glucocorticoids, glucose elevating agents, glycopeptide antibiotics, glycoprotein platelet inhibitors, glycylcyclines, gonadotropin releasing hormones, gonadotropin-releasing hormone antagonists, gonadotropins, group I antiarrhythmics, group II antiarrhythmics, group III antiarrhythmics, group IV antiarrhythmics, group V antiarrhythmics, growth hormone receptor blockers, growth hormones, guanylate cyclase-C agonists, Gabitril, Gablofen, Gadavist, Galafold, Galzin, Gastrocrom, Gastrografin, Gelnique, Gemzar, Genotropin, Genvoya, Geodon, Giapreza, Gilenya, Gilotrif, Gleevec, Gleolan, Gleostine, Gliadel, Gloperba, GlucaGen, Glucophage, Glucophage XR, Glucotrol, Glucotrol XL, Glumetza, Glynase, Glyrx-PF, Glyset, Glyxambi, Gocovri, Gonal-f, Gonal-f RFF, Gonal-f RFF Redi-ject, GoNitro, Gralise, Gris-PEG, Gvoke, H. pylori eradication agents, H2 antagonists, hedgehog pathway inhibitors, hematopoietic stem cell mobilizer, heparin antagonists, heparins, HER2 inhibitors, herbal products, histone deacetylase inhibitors, hormones, hormones/antineoplastics, hydantoin anticonvulsants, hydrazide derivatives, H.P. Acthar Gel, Halaven, Halcion, Haldol, Halog, Harvoni, Hectorol, Hemabate, Hemady, Hemangeol, Hepsera, Hetlioz, Hicon, Hiprex, Horizant, Humalog, Humalog KwikPen, Humalog Mix 50/50, Humalog Mix 50/50 KwikPen, Humalog Mix 75/25, Humalog Mix 75/25 KwikPen, Humatrope, Hycamtin, Hycodan, Hydrea, Hysingla ER, Hyzaar, illicit (street) drugs, immune globulins, immunologic agents, immunostimulants, immunosuppressive agents, impotence agents, in vivo diagnostic biologicals, incretin mimetics, inhaled anti-infectives, inhaled corticosteroids, inotropic agents, insulin, insulin-like growth factors, integrase strand transfer inhibitor, interferons, interleukin inhibitors, interleukins, intravenous nutritional products, iodinated contrast media, ionic iodinated contrast media, iron products, Ibrance, Ibsrela, IC-Green, Iclusig, Idamycin PFS, Idhifa, Ifex, Ilevro, Iluvien, Imbruvica, Imdur, Imitrex, Imitrex Statdose, Impavido, Impoyz, Imuran, Inapsine, Inbrija, Increlex, Incruse Ellipta, Inderal LA, Indocin, Infed, Infugem, Infumorph, Ingrezza, Injectafer, Inlyta, InnoPran XL, INOmax, Inrebic, Inspra, Integrilin, Intelence, Intuniv, Invanz Invega, Invega Sustenna, Invega Trinza, Inveltys, Invirase, Invokamet, Invokamet XR, Invokana, Iopidine, Iressa, Isentress, Isentress HD, Isopto Atropine, Isopto Carpine, Isordil, Isovue-200, Isovue-250, Isovue-300, Isovue-370, Istalol, Istodax, Isuprel, Jadenu, Jadenu Sprinkle, Jakafi, Jalyn, Janumet, Janumet XR, Januvia, Jardiance, Jatenzo, Jentadueto, Jentadueto XR, Jornay PM, Jublia, Juluca, Juxtapid, Jynarque, Ketolides, K-Tab, Kadian, Kaletra, Kalydeco, Kapspargo Sprinkle, Kapvay, Karbinal ER, Katerzia, Kazano, Keflex, Kenalog, Kenalog-10, Kenalog-40, Kengreal, Keppra, Keppra XR, Kerydin, Ketalar, Keveyis, Khapzory, Kinevac, Kisqali, Kitabis Pak, Klaron, Klonopin, Klor-Con, Kombiglyze XR, Korlym, Krintafel, Kuvan, Kybella, Kyleena, Kyprolis, Laxatives, leprostatics, leukotriene modifiers, lincomycin derivatives, local injectable anesthetics, local injectable anesthetics with corticosteroids, loop diuretics, lung surfactants, lymphatic staining agents, lysosomal enzymes, Lacrisert, Lamictal, Lamictal CD, Lamictal ODT, Lamictal XR, Lamisil, Lanoxin, Lantus, Lantus SoloStar, Lasix, Lastacaft, Latisse, Latuda, Lazanda, Lenvima, Lescol XL, Letairis, Leukeran, Levemir, Levitra, Levlite, Levophed, Levulan Kerastick, Lexapro, Lexette, Lexiscan, Lexiva, Lialda, Librax, Lidex, Lidex-E, Lidoderm, Liletta, Lincocin, Linzess, Lioresal, Lipiodol, Lipitor, Lipofen, Lithobid, Lithostat Livalo, Lo Loestrin Fe, Locoid, Locoid Lipocream, Lodosyn, Loestrin 211.5/30, Loestrin 211/20, Loestrin 24 Fe, Loestrin Fe 1.5/30, Loestrin Fe 1/20, Lokelma, Lomotil, Lonsurf, Lopid, Lopressor, Lopressor HCT, Loprox, Lorbrena, LoSeasonique, Lotemax, Lotemax SM, Lotensin, Lotensin HCT, Lotrel, Lotrisone, Lotronex, Lovaza, Lovenox, Lucemyra, Lumason, Lumigan, Lunesta, Lupaneta Pack, Lupron Depot, Lupron Depot-PED, Lutathera, Luvox, Luxiq, Luzu, Lybrel, Lynparza, Lyrica, Lyrica CR, Lysodren, Lysteda, macrolide derivatives, macrolides, magnetic resonance imaging contrast media, malignancy photosensitizers, mast cell stabilizers, medical gas, meglitinides, metabolic agents, methylxanthines, mineralocorticoids, minerals and electrolytes, miscellaneous agents, miscellaneous analgesics, miscellaneous antibiotics, miscellaneous anticonvulsants, miscellaneous antidepressants, miscellaneous antidiabetic agents, miscellaneous antiemetics, miscellaneous antifungals, miscellaneous, ntihyperlipidemic agents, miscellaneous antihypertensive combinations, miscellaneous antimalarials, miscellaneous antineoplastics, miscellaneous antiparkinson agents, miscellaneous antipsychotic agents, miscellaneous antituberculosis agents, miscellaneous antivirals, miscellaneous anxiolytics, sedatives and hypnotics, miscellaneous bone resorption inhibitors, miscellaneous cardiovascular agents, miscellaneous central nervous system agents, miscellaneous coagulation modifiers, miscellaneous diagnostic dyes, miscellaneous diuretics, miscellaneous genitourinary tract agents, miscellaneous GI agents, miscellaneous hormones, miscellaneous metabolic agents, miscellaneous ophthalmic agents, miscellaneous otic agents, miscellaneous respiratory agents, miscellaneous sex hormones, miscellaneous topical agents, miscellaneous uncategorized agents, miscellaneous vaginal agents, mitotic inhibitors, monoamine oxidase inhibitors, mouth and throat products, mTOR inhibitors, mucolytics, multikinase inhibitors, muscle relaxants, mydriatics, Macrilen, Macrobid, Macrodantin, Makena, Malarone, Malarone Pediatric, Marcaine, Marinol, Marplan, Matulane, Mavenclad, Mavyret, Maxalt, Maxalt-MLT, Maxidex, Maxipime, Maxitrol, Maxzide, Maxzide-25, Mayzent, Medrol, Megace ES, Mekinist, Mektovi, MembraneBlue, Menostar, Mentax, Mephyton, Mepron, Merrem, Mesnex, Mestinon, Metadate CD, Metastron, Methadose, Methergine, Methylin, Metopirone, MetroCream, MetroGel, MetroGel-Vaginal, MetroLotion, Miacalcin, Micardis, Micardis HCT, Micro-K, Micro-K 10, Microzide, Midamor, Mifeprex, Migranal, Minastrin 24 Fe, Minipress, Minirin, Minivelle, Minocin, Minolira, Miochol-E, Miostat, Mirapex, Mirapex ER, Mircette, Mirena, Mirvaso, Mitigare, Mobic, Monistat 3, Monoket, Monurol, MorphaBond ER, Motegrity, Motofen, Movantik, Moxeza, Mozobil, MS Contin, Mulpleta, Multaq, Multihance, Muse, Myambutol, Mycamine, Mycobutin, Mydayis, Myfortic, Myleran, Myrbetriq, Mysoline, Mytesi, narcotic analgesic combinations, narcotic analgesics, nasal anti-infectives, nasal antihistamines and decongestants, nasal lubricants and irrigations, nasal preparations, nasal steroids, natural penicillins, neprilysin inhibitors, neuraminidase inhibitors, neuromuscular blocking agents, neuronal potassium channel openers, next generation cephalosporins, NHE3 inhibitors, nicotinic acid derivatives, NK1 receptor antagonists, NNRTIs, non-cardioselective beta blockers, non-iodinated contrast media, non-ionic iodinated contrast media, non-sulfonylureas, Nonsteroidal anti-inflammatory drugs, NS5A inhibitors, nucleoside reverse transcriptase inhibitors (NRTIs), nutraceutical products, nutritional products, Naftin, Nalfon, Namenda, Namenda XR, Namzaric, Naprelan, Naprosyn, Narcan Nasal Spray, Nardil, Naropin, Nascobal, Natacyn, Natazia, Natesto, Natroba, Nayzilam, Nebupent, NeoProfen, Neoral, Nerlynx, Nesacaine, Nesacaine-MPF, Nesina, Netspot, Neupro, Neuraceq, Neurontin, Nevanac, Nexavar, Nexium, Nexium IV, Nexplanon, Nexterone, Niaspan, Nicotrol Inhaler, Nilandron, Nimbex, Ninlaro, Nipent, Niravam, Nithiodote, Nitro-Dur, Nitrolingual Pumpspray, NitroMist, Nitrostat, Nityr, Nizoral Topical, Nocdurna, Nor-QD, Nordette-28, Norditropin FlexPro, Noritate, Norpace, Norpace CR, Norpramin, Northera, Norvasc, Norvir, Nourianz, Novolog, NovoLog FlexPen, NovoLog Mix 70/30, NovoLog Mix 70/30 FlexPen, NovoLog PenFill, Noxafil, Nubeqa, Nucynta, Nucynta ER, Nuedexta, Nuplazid, Nutropin AQ, NuvaRing, Nuvessa, Nuvigil, Nuzyra, Nymalize, ophthalmic anesthetics, ophthalmic anti-infectives, ophthalmic anti-inflammatory agents, ophthalmic antihistamines and decongestants, ophthalmic diagnostic agents, ophthalmic glaucoma agents, ophthalmic lubricants and irrigations, ophthalmic preparations, ophthalmic steroids, ophthalmic steroids with anti-infectives, ophthalmic surgical agents, oral nutritional supplements, other immunostimulants, other immunosuppressants, otic anesthetics, otic anti-infectives, otic preparations, otic steroids, otic steroids with anti-infectives, oxazolidinedione anticonvulsants, oxazolidinone antibiotics, Ocaliva, Octreoscan, Ocufen, Ocuflox, Odefsey, Odomzo, Ofev, Ofirmev, Olumiant, Olux, Omnicef, Omnipaque 12, Omnipaque 140, Omnipaque 180, Omnipaque 240, Omnipaque 300, Omnipaque 350, Omnipaque 9, Omnipred, Omniscan, Omnitrope, Onexton, Onfi, Onglyza, Onmel, Onpattro, Onzetra Xsail, Opana, Opsumit, Optiray 240, Optiray 300, Optiray 320, Optiray 350, Orabloc, Oracea, Oraltag, Orapred ODT, Oraqix, OraVerse, Oravig, Orbactiv, Orenitram, Orfadin, Orilissa, Orkambi, Ortho Tri-Cyclen, Ortho Tri-Cyclen Lo, Ortho-Novum 7/7/7, Oseni, Osmolex ER, OsmoPrep, Osphena, Otezla, Otiprio, Otovel, Otrexup, Ovidrel, Oxaydo, Oxistat, Oxsoralen-Ultra, Oxtellar XR, OxyContin, Oxytrol, Ozempic, Ozobax, Ozurdex, parathyroid hormone and analogs, PARP inhibitors, PCSK9 inhibitors, penicillinase resistant penicillins, penicillins, peripheral opioid receptor antagonists, peripheral opioid receptor mixed gonists/antagonists, peripheral vasodilators, peripherally acting antiobesity agents, phenothiazine antiemetics, phenothiazine antipsychotics, phenylpiperazine antidepressants, phosphate binders, PI3K inhibitors, plasma expanders, platelet aggregation inhibitors, platelet-stimulating agents, polyenes, potassium sparing diuretics with thiazides, potassium-sparing diuretics, probiotics, progesterone receptor modulators, progestins, prolactin inhibitors, prostaglandin D2 antagonists, protease inhibitors, protease-activated receptor-1 antagonists, proteasome inhibitors, proton pump inhibitors, psoralens, psychotherapeutic agents, psychotherapeutic combinations, purine nucleosides, pyrrolidine anticonvulsants, Pamelor, Pandel, Panretin, Paremyd, Parlodel, Parnate, Parsabiv, Pataday, Patanol, Paxil, Paxil CR, Pazeo, PediaPred, Peganone, Penlac, Pennsaid, Pentam, Pentasa, Pepcid, Percodan, Perforomist, Peridex, PerioChip, Persantine, Pexeva, Phoslyra, Phospholine Iodide, Photofrin, Picato, Pifeltro, Piqray, Pitocin, Plaquenil, Plavix, Pliaglis, Polytrim, Pomalyst, Ponstel, Pradaxa, Pravachol, Pre-Pen, Precedex, Pred Forte, Pred Mild, Pred-G, Pregnyl, Premarin, Premphase 14/14, Prempro, Prepidil, Prestalia, Prevacid, Prevymis, Prezcobix, Prezista, Prialt, Priftin, Prilosec, Primsol, Prinivil, Pristiq, ProAir Digihaler, ProAir HFA, ProAir RespiClick, Probuphine, Procardia, Procardia XL, Procysbi, Proglycem, Prograf, Prohance, Prolensa, Promacta, Prometrium, Propecia, Proscar, Prostin E2, Prostin VR Pediatric, Protonix, Protonix IV, Protopam Chloride, Protopic, Provayblue, Provera, Provigil, Provocholine, Prozac, Prozac Weekly, Pulmicort Flexhaler, Pulmicort Respules, Purinethol, Purixan, Pylerac, Quinolones, Qbrelis, Qbrexza, Qmiiz ODT, QNASL, Qoliana, Qsymia, Qtern, Qternmet XR, Quadramet, Qualaquin, Quartette, Qudexy XR, Quelicin, QuilliChew ER, Quillivant XR, Qutenza, Quzyttir, Qvar RediHaler, radiocontrast agents, radiologic adjuncts, radiologic agents, radiologic conjugating agents, radiopharmaceuticals, recombinant human erythropoietins, renin inhibitors, respiratory agents, respiratory inhalant products, rifamycin derivatives, R-Gene 10, Radicava, Ranexa, Rapaflo, Rapamune, Rapivab, Rasuvo, Ravicti, Rayaldee, Rayos, Razadyne, Razadyne ER, Readi-Cat 2, Rebetol, Recarbrio, Reclast, Rectiv, Reglan, Regonol, Relenza, Relistor, Relpax, Remeron, Remeron SolTab, Remodulin, Renagel, Renova, Renvela, Requip, Requip XL, Rescriptor, Restasis, Restoril, Retin-A, Retisert, Retrovir, Revatio, Revlimid, Rexulti, Reyataz, Rhofade, Rhopressa, Ridaura, Rifadin, Rifater, Rilutek, Rimactane, Rimso-50, Rinvoq, Riomet, Riomet ER, Risperdal, Risperdal Consta, Ritalin, Ritalin LA, Robaxin, Robaxin-750, Rocaltrol, Rocklatan, Rowasa, Roxicodone, Rozerem, Rozlytrek, Rubraca, Ruby-Fill, Ruzurgi, Ryanodex, Rybelsus, Rydapt, Rytary, Rythmol, Rythmol SR, salicylates, sclerosing agents, second generation cephalosporins, selective estrogen receptor modulators, selective immunosuppressants, selective phosphodiesterase-4 inhibitors, selective serotonin reuptake inhibitors, serotonin-norepinephrine reuptake inhibitors, serotoninergic neuroenteric modulators, sex hormone combinations, sex hormones, SGLT-2 inhibitors, skeletal muscle relaxant combinations, skeletal muscle relaxants, smoking cessation agents, somatostatin and somatostatin analogs, spermicides, statins, sterile irrigating solutions, streptogramins, streptomyces derivatives, succinimide anticonvulsants, sulfonamides, sulfonylureas, synthetic ovulation stimulants, Sabril, Safyral, Saizen, Salagen, Samsca, Sancuso, Sandimmune, Sandostatin, Saphris, Sarafem, Savaysa, Savella, Saxenda, Scenesse, Sclerosol, Seasonale, Seasonique, Secuado, Segluromet, Seizalam, Selzentry, Semprex-D, Sensipar, Sensorcaine, Septocaine, Septra, Septra DS, Serevent Diskus, Sernivo, Seroquel, Seroquel XR, Serostim, Seysara, Signifor, Siklos, Silenor, SilQ Vanilla, Silvadene, Simbrinza, Sinemet, Sinemet CR, Singulair, Sirturo, Sitavig, Sivextro, Skelaxin, Sklice, Skyla, Slynd, Smoothie Readi-Cat 2, Solaraze, Soliqua, Solodyn, Solosec, Soltamox, Solu-Cortef, Solu-Medrol, Soma, Somatuline Depot, Somavert, Sonata, Soolantra, Soriatane, Sorilux, Sotylize, Sovaldi, Spectazole, Spinraza, Spiriva, Spiriva Respimat, Sporanox, Spritam, Sprycel, SSD, Stalevo 100, Stalevo 125, Stalevo 150, Stalevo 200, Stalevo 50, Stalevo 75, Staxyn, Steglatro, Steglujan, Stendra, Steritalc, Stiolto Respimat, Stivarga, Strattera, Stribild, Striverdi Respimat, Stromectol, Sublocade, Suboxone, Subsys, Sucraid, Sular, Sulfamylon, Sulfatrim Pediatric, Sunosi, Supprelin LA, Suprane, Suprane, Suprax, Suprep Bowel Prep Kit, Survanta, Sustiva, Sustol, Sutent, Symbicort, Symbyax, Symfi, Symfi Lo, Symjepi, Symlin, Symmetrel, Sympazan, Symproic, Symtuza, Synalar, Synarel, Syndros, Synera, Synercid, Synjardy, Synjardy XR, Synribo, SyprineSuprax, Suprep Bowel Prep Kit, Survanta, Sustiva, Sustol, Sutent, Symbicort, Symbyax, Symfi, Symfi Lo, Symjepi, Symlin, Symmetrel, Sympazan, Symproic, Symtuza, Synalar, Synarel, Syndros, Synera, Synercid, Synjardy, Synjardy XR, Synribo, Syprine, tetracyclic antidepressants, tetracyclines, therapeutic radiopharmaceuticals, therapeutic vaccines, thiazide diuretics, thiazolidinediones, thioxanthenes, third generation cephalosporins, thrombin inhibitors, thrombolytics, thyroid drugs, TNF alfa inhibitors, tocolytic agents, topical acne agents, topical agents, topical allergy diagnostic agents, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical depigmenting agents, topical emollients, topical keratolytics, topical non-steroidal anti-inflammatories, topical photochemotherapeutics, topical rubefacient, topical steroids, topical steroids with anti-infectives, transthyretin stabilizers, triazine anticonvulsants, tricyclic antidepressants, trifunctional monoclonal antibodies, Taclonex, Tafinlar, Tagitol V, Tagrisso, Talzenna, Tambocor, Tamiflu, Tarceva, Targretin, Targretin Gel, Tarka, Tasigna, Tasmar, Tavalisse, Taxotere, Taytulla, Tazorac, Tecfidera, Teflaro, Tegretol, Tegretol XR, Tegsedi, Tekturna, Tekturna HCT, Temixys, Temodar, Tenoretic 100, Tenoretic 50, Tenormin, Tepadina, Tessalon, Testim, Thalomid, Thermazene, Thiola, Thiola EC, Thyrogen, Tiazac, Tibsovo, Tigan, Tikosyn, Timoptic, Timoptic-XE, Tindamax, Tirosint, Tirosint-Sol, Tivicay, Tivorbex, Tobi, TOBI Podhaler, TobraDex, Tobradex ST, Tobrex, Tolak, Tolsura, Topamax, Topicort, Toprol-XL, Torisel, Tosymra, Totect, Toujeo Max SoloStar, Toujeo SoloStar, Toviaz, TPDXX, Tracleer, Tradjenta, Trandate, Transderm-Scop, Tranxene, Travatan Z, Treanda, Trecator, Trelegy Ellipta, Trelstar, Tresiba, Treximet, Tri-Luma, Tribenzor, TriCor, Triferic, Triglide, Trileptal, Trilipix, Trintellix, Triostat, Triphasil-28, Trisenox, Triumeq, Trizivir, Trokendi XR, Trulance, Trusopt, Truvada, Tudorza Pressair, Turalio, Tuxarin ER, Tuzistra XR, Twynsta, Tybost, ultrasound contrast media, upper respiratory combinations, urea anticonvulsants, urea cycle disorder agents, urinary anti-infectives, urinary antispasmodics, urinary pH modifiers, uterotonic agents, Uceris, Uloric, Ultane, Ultiva, Ultracet, Ultram, Ultravate, Ultravist, Unasyn, Uptravi, Urex, Urocit-K, Uroxatral, Urso, Urso Forte, Uvadex, vaccine combinations, vaginal anti-infectives, vaginal preparations, vasodilators, vasopressin antagonists, vasopressors, VEGF/VEGFR inhibitors, viral vaccines, viscosupplementation agents, vitamin and mineral combinations, vitamins, VMAT2 inhibitors, Vabomere, Vagifem, Valchlor, Valcyte, Valium, Valstar, Valtrex, Vandazole, Vaniqa, Vanos, Vantas, Varibar Honey, Varibar Nectar, Varibar Pudding, Varibar Thin Honey, Varubi, Vascepa, Vaseretic, Vasostrict, Vasotec, Vazculep, Vectical, Velcade, Veletri, Velphoro, Veltassa, Veltin, Vemlidy, Venclexta, Venofer, Ventavis, Ventolin HFA, Verdeso, Veregen, Verelan, Verelan PM, Versacloz, Verzenio, VESIcare, Vfend, Viagra, Vibativ, Viberzi, Vibramycin, Victoza, Vidaza, Videx, Videx EC, Vigamox, Viibryd, Vimovo, Vimpat, Viracept, Viramune, Viramune XR, Virazole, Viread, Viroptic, VisionBlue, Vistaril, Vistogard, Visudyne, Vitrakvi, Vitrase, Vivelle-Dot, Vivitrol, Vivlodex, Vizamyl, Vizimpro, Vogelxo, Voltaren Ophthalmic, Vosevi, Vosol, Vosol HC, Votrient, VPRIV, Vraylar, Vumerity, Vyndamax, Vyndaqel, Vytorin, Vyvanse, Vyzulta, Wakix, Welchol, Wellbutrin, Wellbutrin SR, Wellbutrin XL, Xadago, Xalatan, Xalkori, Xanax, Xanax XR, Xarelto, Xatmep, Xeljanz, Xeljanz XR, Xeloda, Xelpros, Xenazine, Xenical, Xenleta, Xepi, Xerava, Xerese, Xermelo, Xifaxan, Xigduo XR, Xiidra, Ximino, Xofigo, Xofluza, Xolegel, Xopenex, Xopenex HFA, Xospata, Xpovio, Xtampza ER, Xtandi, Xultophy, Xuriden, Xyrem, Xyzal, Yasmin, Yaz, Yondelis, Yonsa, Yosprala, Yupelri, Yutiq, Zanaflex, Zanosar, Zantac, Zantac 300, Zarontin, Zaroxolyn, Zavesca, Zegerid, Zejula, Zelapar, Zelboraf, Zelnorm, Zembrace SymTouch, Zemdri, Zemplar, Zepatier, Zerbaxa, Zerit, Zerviate, Zestoretic, Zestril, Zetia, Zetonna, Ziac, Ziagen, Ziana, Zilretta, Zinacef, Zinecard, Zioptan, Zipsor, Zirgan, Zithromax, Zocor Zofran, Zofran ODT, Zohydro ER, Zoladex, Zolinza, Zoloft, Zolpimist, Zomacton, Zometa, Zomig, Zomig-ZMT, Zonalon, Zonegran, Zontivity, Zorbtive, Zortress, Zorvolex, Zosyn, Zovirax, Zovirax Ointment, ZTlido, Zubsolv, Zulresso, Zuplenz, Zyclara, Zydelig, Zyflo, Zyflo CR, Zykadia, Zylet, Zyloprim, Zymar, Zymaxid, Zypitamag, Zyprexa, Zyprexa Relprevv, Zyprexa Zydis, Zytiga, or Zyvox.

Embodiment 175. The composition of any of the above or below embodiments, wherein the active ingredient is prostaglandin or a prostaglandin analog.

Embodiment 176. The composition of any of the above or below embodiments, wherein the prostaglandin analog is selected from the group consisting of: bimatoprost, latanoprost, tafluprost, and travoprost.

Embodiment 177. The composition of any of the above or below embodiments, wherein the protein having hyaluronidase activity is a human recombinant hyaluronidase such as HYLENEX and the active ingredient is a prostaglandin analog selected from the group consisting of: bimatoprost, latanoprost, tafluprost, and travoprost.

Embodiment 178. A composition comprising an amount of a protein having hyaluronidase activity and an amount of an additional active pharmaceutical ingredient (API).

Embodiment 179. The composition of any of the above or below embodiments, wherein the additional API is used to treat acne, ADHD, AIDS/HIV, allergies, Alzheimer's, angina, anxiety, arthritis, asthma, bipolar disorder, bronchitis, cancer, cholesterol, colds & flu, constipation, COPD, depression, diabetes (Type 1), diabetes (Type 2), diarrhea, eczema, erectile dysfunction, fibromyalgia, gastrointestinal, GERE (Heartburn), gout, hair loss, hayfever, heart disease, hepatitis A, hepatitis B, herpes, hypertension, hypothyroidism, incontinence, IBD (Bowel), insomnia, menopause, mental health, migraine, osteoarthritis, osteoporosis, pain, psoriasis, rheumatoid arthritis, schizophrenia, seizures, sexual health, stroke, swine flu, UTI, or weight loss.

Embodiment 180. A method of treating and/or preventing a disease or disorder, the method comprising administering to a subject in need thereof any of the compositions disclosed herein.

Embodiment 181. A method of treating or preventing a condition such as a cosmetic condition in a subject in need thereof by administering a composition that comprises 4-methylumbelliferone (4-MU) to the peri-orbital region and/or mid-face of the subject. The cosmetic condition may be due to edema (e.g., peri-orbital puffiness due to peri-orbital edema).

Embodiment 182. A method of treating peri-orbital puffiness due to peri-orbital edema in a subject in need thereof by administering a composition that comprises 4-methylumbelliferone (4-MU) to the peri-orbital region of the subject.

Embodiment 183. The method of any of the above or below embodiments, wherein embodiments, the subject has not been treated with a dermal filler 1 month, 2 months, 3 months, 4 months, 5 months, 6 months, 7 months, 8 months, 9 months, 10 months, 11 months, 12 months, 2 years, 3 years, 4 years, or 5 years before the administration of the composition that comprises 4-methylumbelliferone (4-MU).

Embodiment 184. The method of any of the above or below embodiments, wherein the 4-methylumbelliferone (4-MU) is administered in a therapeutically effective amount.

Embodiment 185. A method of treating peri-orbital puffiness due to peri-orbital edema in a subject in need thereof by administering a composition that comprises 4-methylumbelliferone (4-MU) to an eye of the subject.

Embodiment 186. The method of any of the above or below embodiments, wherein an eye drop that comprises 4-methylumbelliferone (4-MU) is administered to the eye of the subject, including for example directly to the surface of the eye.

Embodiment 187. The method of any of the above or below embodiments, wherein the subject scored as Grade 0 is administered a composition that comprises 4-methylumbelliferone (4-MU) to the peri-orbital region of the subject.

Embodiment 188. The method of any of the above or below embodiments, wherein the subject scored as Grade 1E, 2E or 3E is administered a composition that comprises 4-methylumbelliferone (4-MU) to the peri-orbital region of the subject, and optionally treated by surgical removal of a portion of one or more of the fat pads to increase the degree of improvement in the peri-orbital puffiness.

Embodiment 189. A method to assess whether surgical intervention is needed to treat peri-orbital puffiness not due to peri-orbital edema in a subject in need thereof, the method comprising administering (e.g., injecting) a composition comprising 4-methylumbelliferone (4-MU) to the peri-orbital region of the subject and determining that surgical intervention is required to treat the peri-orbital puffiness not due to peri-orbital edema where the administration of the composition comprising 4-methylumbelliferone (4-MU) does not treat (e.g., reduce) the peri-orbital puffiness not due to peri-orbital edema. Such methods may additionally comprise the step of surgically removing a portion of one or more fat pads in the upper and/or lower eyelids of the subject.

Embodiment 190. A method of treating a cosmetic condition in a subject in need thereof due to (e.g., caused by) edema by administering a composition that comprises 4-methylumbelliferone (4-MU) to a region on the subject's face having the condition (e.g., a peri-orbital region and/or a mid-face region of the subject).

Embodiment 191. The method of any of the above or below embodiments, wherein the peri-orbital puffiness due to peri-orbital edema is unrelated to the use of a hyaluronic acid filler in the peri-orbital region.

Embodiment 192. The method of any of the above or below embodiments, wherein the one or more peri-orbital fat pads include at least one lower peri-orbital fat pad.

Embodiment 193. The method of any of the above or below embodiments, wherein the step of administering comprises applying a patch or a cream to a surface of the skin in the peri-orbital region.

Embodiment 194. The method of any of the above or below embodiments, wherein the composition is administered in an amount effective to treat the peri-orbital puffiness due to peri-orbital edema.

Embodiment 195. A method of assessing peri-orbital puffiness due to peri-orbital edema in a subject, the method comprising: a. determining if the subject exhibits pseudoherniation of one or more upper eyelid fat pads and one or more lower eyelid fat pads; b. determining if the subject exhibits edema in one or more of the upper eyelid fat pads and the one or more of the lower eyelid fat pads; c. determining if the subject exhibits edema in the peri-orbital tissue; and d. scoring the peri-orbital puffiness due to peri-orbital edema from the determinations made in steps a. to c.

Embodiment 196. The method of any of the above or below embodiments, wherein the peri-orbital puffiness due to peri-orbital edema is unrelated to the use of a hyaluronic acid filler in the peri-orbital region.

Embodiment 197. The method of any of the above or below embodiments, wherein the upper eyelid fat pad is an upper middle fat pad and an upper medial fat pad.

Embodiment 198. The method of any of the above or below embodiments, wherein the subject is scored as Grade 0 where the subject does not exhibit pseudoherniation of any of the upper or lower eyelid fat pads.

Embodiment 199. The method of any of the above or below embodiments, wherein the subject is scored as Grade 0 where the subject exhibits edema of one or more of the upper or lower eyelid fat pads.

Embodiment 200. The method of any of the above or below embodiments, wherein the subject is scored as Grade 1 where the subject exhibits pseudoherniation of one of the upper eyelid fat pads and/or one of the lower eyelid fat pads.

Embodiment 201. The method of any of the above or below embodiments, wherein the subject is scored as Grade 1 E where the subject exhibits pseudoherniation of one of the upper eyelid fat pads and/or one of the lower eyelid fat pads and exhibits edema of the fat pads and/or peri-orbital tissue.

Embodiment 202. The method of any of the above or below embodiments, wherein the subject is scored as Grade 2 where the subject exhibits pseudoherniation of two of the upper eyelid and/or two of the lower eyelid fat pads.

Embodiment 203. The method of any of the above or below embodiments, wherein the subject is scored as Grade 2E where the subject exhibits pseudoherniation of two of the upper eyelid fat pads and/or two of the lower eyelid fat pads and exhibits edema of the fat pads and/or peri-orbital tissue.

Embodiment 204. The method of any of the above or below embodiments, wherein the subject is scored as Grade 3 where the subject exhibits pseudoherniation of three of the lower eyelid fat pads.

Embodiment 205. The method of any of the above or below embodiments, wherein the subject is scored as Grade 3E where the subject exhibits pseudoherniation of the three lower eyelid fat pads and exhibits edema of the fat pads and/or peri-orbital tissue.

Embodiment 206. A method of treating a cosmetic condition due to edema in a subject in need thereof, the method comprising administering a composition that comprises 4-methylumbelliferone (4-MU) to the peri-orbital region and/or mid-face of the subject.

Embodiment 207. The method of any of the above or below embodiments, wherein the subject has not previously been treated with a dermal filler in the peri-orbital region and/or the mid-face.

Embodiment 208. A method of treating peri-orbital puffiness due to peri-orbital edema in a subject in need thereof, the method comprising administering a composition that comprises 4-methylumbelliferone (4-MU) to an eye of the subject.

Embodiment 209. A method of treating and/or preventing inflammation in a subject in need thereof by administering a composition that comprises a protein having hyaluronidase activity (e.g., a hyaluronidase such as Hylenex, Amphadase, or Vitrase) to a region of the subject having inflammation. Such methods advantageously may be used to treat inflammation and/or inflammation associated with stasis dermatitis.

Embodiment 210. The method of any of the above or below embodiments, wherein the inflammation is associated with stasis dermatitis.

Embodiment 211. The method of any of the above or below embodiments, wherein the stasis dermatitis is present in a leg, foot, and/or ankle.

Embodiment 212. The method of any of the above or below embodiments, wherein the step of administering is performed by one or more injections to the region of the subject having inflammation.

Embodiment 213. The method of any of the above or below embodiments, wherein the step of administering comprises applying a patch or a cream to a region of the subject having inflammation.

Embodiment 214. A method of reducing inflammation in a subject in need thereof by administering a composition that comprises a protein having hyaluronidase activity to the region of the subject having inflammation, wherein the administration of the composition reduces severity of the inflammation.

Embodiment 215. The method of any of the above or below embodiments, wherein the severity of the inflammation is reduced by at least one grade.

Embodiment 216. The method of any of the above or below embodiments, wherein the severity of the inflammation is reduced by at least two grades.

Embodiment 217. The method of any of the above or below embodiments, wherein after the administration of the composition the inflammation is scored as moderate.

Embodiment 218. The method of any of the above or below embodiments, wherein after the administration of the composition the inflammation is scored as mild.

Embodiment 219. The method of any of the above or below embodiments, wherein after the administration of the composition the inflammation is scored as quiescent.

Embodiment 220. A method of treating fibrosis in a subject in need thereof, the method comprising administering a composition that comprises a protein having hyaluronidase activity to the subject.

Embodiment 221. A composition comprising a protein having hyaluronidase activity and a collagenase and its use in methods for the treatment of upper and/or lower eyelid puffiness/fullness.

Embodiment 222. The composition of any of the above or below embodiments, wherein the collagenase is a bovine or human recombinant collagenase.

Embodiment 223. The composition of any of the above or below embodiments, wherein the protein having hyaluronidase activity is present in an amount of about 1 to about 1,000 pg/mL.

Embodiment 224. The composition of any of the above or below embodiments, wherein the protein having hyaluronidase activity is present in an amount of about 1 to about 1,000 U/mL.

Embodiment 225. The composition of any of the above or below embodiments, wherein the collagenase is present in an amount of about 1 to about 1,000 pg/mL.

Embodiment 226. The composition of any of the above or below embodiments, wherein the collagenase is present in an amount of about 1 to about 1,000 U/mL.

Embodiment 227. A method of treating upper and/or lower eyelid puffiness/fullness in a subject, the method comprising administering a therapeutically effective amount of a composition having a protein with hyaluronidase activity and a collagenase to one or more of the upper and/or lower eyelid fat pads.

Embodiment 228. The method of any of the above or below embodiments, wherein the method may reduce (or eliminate) the appearance of upper and/or lower eyelid puffiness/fullness.

Embodiment 229. The composition of any of the above or below embodiments, wherein the composition includes about 1 to about 1,000 Units of the protein having hyaluronidase activity.

Embodiment 230. The composition of any of the above or below embodiments, wherein the composition includes about 5 to about 15 Units of the protein having hyaluronidase activity.

Embodiment 231. A method for treating and/or preventing fibrosis (e.g., dermal fibrosis) in a subject in need thereof by administering a composition that comprises a protein having hyaluronidase activity (e.g., a hyaluronidase such as Hylenex, Amphadase, or Vitrase) to the subject.

Embodiment 232. The method of any of the above or below embodiments, wherein the fibrosis is a fibrotic skin disease such as scleroderma, nephrogenic fibrosing dermopathy, mixed connective tissue disease, scleromyxedema, scleredema, or eosinophilic fasciitis.

Embodiment 233. The method of any of the above or below embodiments, wherein the fibrosis is selected from the group consisting of: pulmonary fibrosis, idiopathic pulmonary fibrosis, cirrhosis, endomyocardial fibrosis, mediastinal fibrosis, myelofibrosis, retroperitoneal fibrosis, progressive massive fibrosis, nephrogenic systemic fibrosis, Crohn's Disease intestine, keloid, myocardial infarction, scleroderma/systemic sclerosis, arthrofibrosis, and adhesive capsulitis.

Embodiment 234. The method of any of the above or below embodiments, wherein the protein having hyaluronidase activity region is administered to a region of the subject having the fibrosis.

Embodiment 235. The method of any of the above or below embodiments, wherein the protein having hyaluronidase activity region is injected into the fibrosis.

Embodiment 236. The method of any of the above or below embodiments, wherein the protein having hyaluronidase activity region is applied to the fibrosis.

Embodiment 237. A method of reducing fibrosis in a subject in need thereof by administering a composition that comprises a protein having hyaluronidase activity to a region of fibrosis on the subject, wherein the administration results in a reduction in a grade of severity of the fibrosis.

Embodiment 238. The method of any of the above or below embodiments, wherein the fibrosis prior to treatment with the composition is classified as mild fibrosis, moderate fibrosis, or severe fibrosis.

Embodiment 239. The method of any of the above or below embodiments, wherein the fibrosis after treatment with the composition is classified as no fibrosis, mild fibrosis, or moderate fibrosis.

Embodiment 240. The method and/or composition of any of the above or below embodiments, wherein the method and/or composition is used to cover trap door scars in a subject in need thereof.

Embodiment 241. The method and/or composition of any of the above or below embodiments, wherein trap door scars trap fluid.

Embodiment 242. The method and/or composition of any of the above or below embodiments, wherein the method and/or composition is used at surgical sites in a subject in need thereof.

Embodiment 243. The method and/or composition of any of the above or below embodiments, wherein the method and/or composition is used for treating survical wounds in a subject in need thereof.

Embodiment 244. The method and/or composition of any of the above or below embodiments, wherein the method and/or composition is used to decrease the degree of burn classification in a subject in need thereof.

Embodiment 245. The method and/or composition of any of the above or below embodiments, wherein the decrease in the degree of burn classification is from fourth to third.

Embodiment 246. The method and/or composition of any of the above or below embodiments, wherein the decrease in the degree of burn classification is from third to second.

Embodiment 247. The method and/or composition of any of the above or below embodiments, wherein the decrease in the degree of burn classification is from second to first.

Embodiment 248. In some embodiments, the methods and compositions disclosed herein are used to treat and/or reduce scarring from burns in a subject in need thereof.

Embodiment 249. In some embodiments, the methods and compositions disclosed herein are used during interventional cardiology and radiology in a subject in need thereof.

Embodiment 250. The method and/or composition of any of the above or below embodiments, wherein the method and/or composition is used to dissolve fibrosis by limiting the execution of the procedure to access and remove a medical device in a subject in need thereof.

Embodiment 251. The method and/or composition of any of the above or below embodiments, wherein the method and/or composition is used to permit improved access to the target organ, including removal of a foreign object from the body of the subject in need thereof.

Embodiment 252. The method and/or composition of any of the above or below embodiments, wherein the method and/or composition is used during or instead of a fasciotomy to reduce swelling and/or to save one or more limbs.

Embodiment 253. The method and/or composition of any of the above or below embodiments, wherein the method and/or composition does not comprise hyaluronidase.

Embodiment 254. The method and/or composition of any of the above or below embodiments, wherein the method and/or composition comprises elastinase and do not comprise hyaluronidase.

Embodiment 255. The method and/or composition of any of the above or below embodiments, wherein the method and/or composition is used in a subject who is HIV positive and/or has AIDS.

Embodiment 256. The method and/or composition of any of the above or below embodiments, wherein the method and/or composition comprises protease inhibitors that cause fat atrophy in the body of a subject thereof.

Embodiment 257. The method and/or composition of any of the above or below embodiments, wherein the method and/or composition comprising protease inhibitors that cause fat atrophy in the body of a subject thereof is used to decrease fullness around the eyes.

Embodiment 258. A method of restoring myelination to axons (or neurons) in a subject in need thereof, the method comprising administering a composition that comprises a protein having hyaluronidase activity (e.g., a hyaluronidase such as Hylenex, Amphadase, or Vitrase) to a region of the subject having demyelinated axons (or neurons).

Embodiment 259. The method of any of the above or below embodiments, wherein the protein having hyaluronidase activity region is injected into the region of the subject having demyelinated axons.

Embodiment 260. The method of any of the above or below embodiments, wherein the protein having hyaluronidase activity region is applied to the region of the subject having demyelinated axons.

Embodiment 261. A method of treating a disease or disorder characterized by demyelinated neurons in a subject in need thereof by administering a composition that comprises a protein having hyaluronidase activity to a region of the subject having demyelinated neurons.

Embodiment 262. The method of any of the above or below embodiments, wherein the disease is a neurodegenerative disease.

Embodiment 263. The method of any of the above or below embodiments, wherein the neurodegenerative disease is multiple sclerosis, acute disseminated encephalomyelitis, neuromyelitis optica, transverse myelitis, chronic inflammatory demyelinating polyneuropathy, Guillain-Barré syndrome, or central pontine myelinosis.

Embodiment 264. A method of treating and/or preventing anterior facial fullness, a jowl, and/or a labiomandibular fold in a subject in need thereof by administering a composition that comprises a protein having hyaluronidase activity (e.g., a hyaluronidase such as Hylenex, Amphadase, or Vitrase) to a region of the subject's face having anterior facial fullness, jowl, or labiomandibular fold, respectively. Advantageously, such methods may be used to restore symmetry to a face where one side exhibits more fullness than the other side by administering the protein having hyaluronidase activity to only one side of the face of the subject.

Embodiment 265. The method of any of the above or below embodiments, wherein the protein having hyaluronidase activity is administered to both sides of the face of the subject.

Embodiment 266. The method of any of the above or below embodiments, wherein the anterior facial fullness is severe fullness.

Embodiment 267. The method of any of the above or below embodiments, wherein the anterior facial fullness is moderate fullness.

Embodiment 268. The method of any of the above or below embodiments, wherein the anterior facial fullness is mild fullness.

Embodiment 269. The method of any of the above or below embodiments, wherein the jowl is a severe jowl.

Embodiment 270. The method of any of the above or below embodiments, wherein the jowl is a moderate jowl.

Embodiment 271. The method of any of the above or below embodiments, wherein the jowl is a mild jowl.

Embodiment 272. The method of any of the above or below embodiments, wherein the labiomandibular fold is a severe labiomandibular fold.

Embodiment 273. The method of any of the above or below embodiments, wherein the labiomandibular fold is a moderate labiomandibular fold.

Embodiment 274. The method of any of the above or below embodiments, wherein the labiomandibular fold is a mild labiomandibular fold.

Embodiment 275. The method of any of the above or below embodiments, wherein the step of administering is performed by one or more injections to the area of skin on the subject having the anterior facial fullness, the jowl, and/or the labiomandibular fold.

Embodiment 276. The method of any of the above or below embodiments, wherein the step of administering comprises applying a patch or a cream to the area of skin of on subject having the anterior facial fullness, the jowl, and/or the labiomandibular fold.

Embodiment 277. The method of any of the above or below embodiments, wherein the protein having hyaluronidase activity is administered in a therapeutically effective amount to the area of skin having the anterior facial fullness, the jowl, and/or the labiomandibular fold.

Embodiment 278. A method of reducing or improving the appearance of anterior facial fullness in a subject in need thereof, the method comprising administering a composition that comprises a protein having hyaluronidase activity to an area of skin on the subject, wherein the area of skin is along an anterior cheek bordered medially by a nasolabial fold, laterally by a midface groove, superiorly by a nasojugal groove and inferiorly by a jowl, wherein the administration of the composition reduces severity (e.g., improves the appearance) of the anterior facial fullness.

Embodiment 279. A method of reducing or improving the appearance of a jowl in a subject in need thereof, the method comprising administering a composition that comprises a protein having hyaluronidase activity to an area of skin on the subject having the jowl, wherein the administration of the composition reduces severity (e.g., improves the appearance) of the jowl.

Embodiment 280. A method of reducing or improving the appearance of a labiomandibular fold in a subject in need thereof, the method comprising administering a composition that comprises a protein having hyaluronidase activity to an area of skin on the subject having the labiomandibular fold, wherein the administration of the composition reduces severity (e.g., improves) of the labiomandibular fold.

Embodiment 281. The method of any of the above or below embodiments, wherein the severity of the anterior facial fullness, the jowl, and/or the labiomandibular fold is reduced by at least one grade on a scale that measures the severity of the anterior facial fullness, the jowl, and/or the labiomandibular fold, respectively.

Embodiment 282. The method of any of the above or below embodiments, wherein the severity of the anterior facial fullness, the jowl, and/or the labiomandibular fold is reduced from a severe grade to a moderate grade.

Embodiment 283. The method of any of the above or below embodiments, wherein the severity of the anterior facial fullness, the jowl, and/or the labiomandibular fold is reduced from a severe grade to a mild grade.

Embodiment 284. The method of any of the above or below embodiments, wherein the severity of the anterior facial fullness, the jowl, and/or the labiomandibular fold is reduced from a moderate grade to a mild grade.

Embodiment 285. A method of treating anterior facial fullness in a subject in need thereof, the method comprising administering a composition that comprises a protein having hyaluronidase activity to an area of skin on the subject, wherein the area of skin is along an anterior cheek bordered medially by a nasolabial fold, laterally by a midface groove, superiorly by a nasojugal groove and inferiorly by a jowl.

Embodiment 286. The method of any of the above or below embodiments, wherein the step of administering is performed by one or more injections to the area of skin on the subject.

Embodiment 287. The method of any of the above or below embodiments, wherein the step of administering comprises applying a patch or a cream to the area of skin of on subject.

Embodiment 288. A method of treating a jowl in a subject in need thereof, the method comprising administering a composition that comprises a protein having hyaluronidase activity to an area of skin on the subject having the jowl.

Embodiment 289. The method of any of the above or below embodiments, wherein the step of administering comprises applying a patch or a cream to the area of skin of on subject having the jowl.

Embodiment 290. A method of treating a labiomandibular fold in a subject in need thereof, the method comprising administering a composition that comprises a protein having hyaluronidase activity to an area of skin on the subject having the labiomandibular fold.

Embodiment 291. The method of any of the above or below embodiments, wherein the step of administering is performed by one or more injections to the area of skin on the subject having the labiomandibular fold.

Embodiment 292. The method of any of the above or below embodiments, wherein the severity of the anterior facial fullness is reduced by at least one grade.

Embodiment 293. The method of any of the above or below embodiments, wherein the severity of the anterior facial fullness is reduced from severe to moderate fullness.

Embodiment 294. The method of any of the above or below embodiments, wherein the severity of the anterior facial fullness is reduced from severe to mild fullness.

Embodiment 295. The method of any of the above or below embodiments, wherein the severity of the anterior facial fullness is reduced from moderate to mild fullness.

Embodiment 296. A formulation for cosmetic and/or non-cosmetic applications that comprise a protein having hyaluronidase activity (e.g., a hyaluronidase such as HYLENEX, Amphadase, or Vitrase) and/or a prostaglandin analog and methods of using same to treat and/or prevent a cosmetic conditions such as peri-orbital puffiness (peri-orbital fullness). For example, the cosmetic condition may be due to edema (e.g., peri-orbital puffiness due to peri-orbital edema).

Embodiment 297. A formulation comprising a protein having hyaluronidase activity, wherein the formulation is free of or substantially free of albumin.

Embodiment 298. The formulation of any of the above or below embodiments, wherein the albumin is human albumin.

Embodiment 299. The formulation of any of the above or below embodiments, wherein the proteins having hyaluronidase activity are hyaluronidases.

Embodiment 300. The formulation of any of the above or below embodiments, wherein the hyaluronidases are recombinant hyaluronidases.

Embodiment 301. The formulation of any of the above or below embodiments, wherein the protein have hyaluronidase activity is selected from one or more of Hyaluronidase 1 (Hyal1), Hyaluronidase 2 (Hyal2), Hyaluronidase 3 (Hyal3), Hyaluronidase 4 (Hyal4), Hyaluronidase 5 (Hyal5), and Hyaluronidase 6 (Hyal1).

Embodiment 302. The formulation of any of the above or below embodiments, wherein the protein having hyaluronidase activity is crosslinked (e.g., between about 10% to about 90% of the protein having hyaluronidase activity comprise inter-protein cross-links).

Embodiment 303. The formulation of any of the above or below embodiments, wherein the protein having hyaluronidase activity is crosslinked with 1,4-butanediol diglycidyl ether (BDDE) or lysine.

Embodiment 304. The formulation of any of the above or below embodiments, wherein the inter-protein cross-links comprise disulfide bonds.

Embodiment 305. The formulation of any of the above or below embodiments, wherein the inter-protein cross-links are formed by covalently linking two or more reactive groups on the proteins using one or more crosslinkers.

Embodiment 306. The formulation of any of the above or below embodiments, wherein the two or more reactive groups on the proteins are selected from lysine residues, aspartic acid residues, glutamic acid residues, and cysteine residues.

Embodiment 307. The formulation of any of the above or below embodiments, wherein the one or more crosslinkers are selected from glutaraldehyde, genipin, methylgloxal, proanthrocyanidin, tannic acid, and 1-ethyl-3-(3-dimethylaminopropyl)-carbodiimide.

Embodiment 308. The formulation of any of the above or below embodiments, wherein the formulation further comprises one or more of an anesthetic agent, a prostaglandin, a prostaglandin analog, and a vasoconstrictor.

Embodiment 309. The formulation of any of the above or below embodiments, wherein the anesthetic agent is lidocaine.

Embodiment 310. The formulation of any of the above or below embodiments, wherein the prostaglandin analog is bimatoprost.

Embodiment 311. The formulation of any of the above or below embodiments, wherein the vasoconstrictor is epinephrine.

Embodiment 312. The formulation of any of the above or below embodiments, wherein the formulation further comprises a surfactant.

Embodiment 313. The formulation of any of the above or below embodiments, wherein the surfactant is polysorbate 80.

Embodiment 314. The formulation of any of the above or below embodiments, wherein the formulation further comprises a buffer.

Embodiment 315. The formulation of any of the above or below embodiments, wherein the buffer is a histidine buffer, a citrate buffer, a gluconate buffer, a succinate buffer, or a phosphate buffer.

Embodiment 316. The formulation of any of the above or below embodiments, wherein the formulation further comprises a stabilizer.

Embodiment 317. The formulation of any of the above or below embodiments, wherein the stabilizer is an amino acid or a saccharide.

Embodiment 318. A formulation that comprises a prostaglandin analog such as bimatoprost and the use thereof for the treatment of peri-orbital fullness.

Embodiment 319. A method of treating peri-orbital puffiness in a subject in need thereof, the method comprising: administering a formulation disclosed herein to a peri-orbital region of the subject.

Embodiment 320. The method of any of the above or below embodiments, wherein the peri-orbital puffiness is due to peri-orbital edema.

Embodiment 321. The method of any of the above or below embodiments, wherein the peri-orbital puffiness is unrelated to a use of a hyaluronic acid filler in the peri-orbital region.

Embodiment 322. The method of any of the above or below embodiments, wherein the step of administering is performed by one or more injections into a peri-orbital soft tissue and/or one or more injections into one or more peri-orbital fat pads.

Embodiment 323. The method of any of the above or below embodiments, wherein the one or more peri-orbital fat pads include at least one lower peri-orbital fat pad.

Embodiment 324. The method of any of the above or below embodiments, wherein the lower peri-orbital fat pad is a lower lateral fat pad, a lower middle fat pad, or a lower medial fat pad.

Embodiment 325. The method of any of the above or below embodiments, wherein the one or more peri-orbital fat pads include at least one upper peri-orbital fat pad.

Embodiment 326. The method of any of the above or below embodiments, wherein the upper peri-orbital fat pad is an upper middle fat pad or an upper medial fat pad.

Embodiment 327. The method of any of the above or below embodiments, wherein each injection into the peri-orbital soft tissue and/or the one or more peri-orbital fat pads includes about 1 to about 1,000 Units of the protein having hyaluronidase activity.

Embodiment 328. The method of any of the above or below embodiments, wherein the peri-orbital puffiness is reduced for up to 6 months.

Embodiment 329. The method of any of the above or below embodiments, wherein the hyaluronidase is selected from one or more of Hyaluronidase 1 (Hyal1), Hyaluronidase 2 (Hyal2), Hyaluronidase 3 (Hyal3), Hyaluronidase 4 (Hyal4), Hyaluronidase 5 (Hyal5), and Hyaluronidase 6 (Hyal1).

Embodiment 330. The method of any of the above or below embodiments, wherein about 10% to about 90% of the protein having hyaluronidase activity comprises inter-protein cross-links.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. A method of treating edema in a subject in need thereof, the method comprising administering a composition that comprises a protein having hyaluronidase activity to a region of the subject having edema.

2. The method of claim 1, wherein the edema is peripheral edema.

3. The method of claim 2, wherein the peripheral edema is present in a leg, foot, ankle, and/or arm.

4. The method of claim 3, wherein the peripheral edema is present in a foot.

5. The method of claim 1, wherein the edema is pedal edema.

6. The method of claim 5, wherein the edema is present is a lower leg and/or a foot.

7. The method of claim 1, wherein the protein having hyaluronidase activity is hyaluronidase.

8. The method of claim 1, wherein the hyaluronidase is a recombinant hyaluronidase.

9. The method of claim 1, wherein the step of administering is performed by one or more injections to the region of the subject having edema.

10. The method of claim 1, wherein the step of administering comprises applying a patch or a cream to a region of the subject having edema.

11. The method of claim 1, wherein the protein having hyaluronidase activity is administered in a therapeutically effective amount.

12. The method of claim 9, wherein each injection includes about 1 to about 1,000 Units of the protein having hyaluronidase activity.

13. The method of claim 12, wherein each injection includes about 1 to about 50 Units of the protein having hyaluronidase activity.

14. The method of claim 12, wherein each injection includes about 5 to about 15 Units of the protein having hyaluronidase activity.

15. The method of claim 13, wherein each injection is performed using a 0.5 mL syringe.

16. The method of claim 15, wherein the 0.5 mL syringe comprises a 32-gauge needle.

17. A method of reducing edema in a subject in need thereof, the method comprising administering a composition that comprises a protein having hyaluronidase activity to the region of the subject having edema, wherein the administration of the composition reduces severity of the edema.

18. The method of claim 17, wherein the severity of the edema is reduced by at least one grade.

19. The method of claim 17, wherein the severity of the edema is reduced by at least two grades.

20. The method of claim 17, wherein after the administration of the composition the edema is scored as grade 3.

21. The method of claim 17, wherein after the administration of the composition the edema is scored as grade 2.

22. The method of claim 17, wherein after the administration of the composition the edema is scored as grade 1.

23. The method of claim 17, wherein after the administration of the composition the edema presents as a 2 mm depression with an immediate rebound time.

24. The method of claim 17, wherein after the administration of the composition the edema presents as a 3 to 4 mm depression with a rebound time of 15 seconds or less.

25. The method of claim 17, wherein after administration of the composition the edema presents as a 5 to 6 mm depression with a rebound time of 10 to 30 seconds.

26. The method of claim 17, wherein before administration of the composition the edema presents as a 8 mm depression with a rebound time of more than 20 seconds.

27. The method of claim 17, wherein the protein having hyaluronidase activity is hyaluronidase.

28. The method of claim 17 further comprising administering a diuretic to the subject.
